(19) United States
(12) Patent Application Publication
(10) Pub. No.: US 2022/0117265 A1
(43) Pub. Date: Apr. 21, 2022

(54) METHODS AND COMPOSITIONS FOR TREATING INTESTINAL DYSBIOSIS

(71) Applicant: MARS, INCORPORATED, McLean, VA (US)

(72) Inventors: Zoe V. MARSHALL-JONES, Melton Mowbray, Leicestershire (GB); Ruth STAUNTON, Fordingbridge, Hampshire (GB); Phillip WATSON, Melton Mowbray, Leicestershire (GB); David ALEXANDER, Wodonga (AU); Alice DEAL, Verden (DE)

(73) Assignee: MARS, INCORPORATED, McLean, VA (US)

(21) Appl. No.: 17/423,774

(22) PCT Filed: Jan. 17, 2020
(86) PCT No.: PCT/US2020/014201

§ 371 Date: Jul. 16, 2021

Related U.S. Application Data
(60) Provisional application No. 62/794,542, filed on Jan. 18, 2019.

(51) Int. Cl. A23K 10/18; A23K 20/163; A23K 50/40; A23K 10/33

(52) U.S. Cl. CPC A23K 10/18; A23K 10/33; A23K 50/40; A23K 20/163

(57) ABSTRACT

Methods for assessing the health of the microbiome and compositions and food products for improving intestinal health, treating intestinal dysbiosis, and/or treating an intestinal disorder in a companion animal, e.g., a dog are provided.

### CROSS-REFERENCE TO RELATED APPLICATION

This application claims priority to U.S. Provisional Application No. 62/794,542, filed on Jan. 18, 2019, the contents of which are incorporated by reference in its entirety.

### FIELD

The presently disclosed subject matter relates to methods, compositions and food products for assessing intestinal health, improving intestinal health, treating intestinal dysbiosis and/or treating an intestinal disorder in a companion animal, e.g., a dog.

### BACKGROUND

Faeces consistency and particularly the extremes of loose or dry faeces are key indicators to owners of pet health. As such, the impact of diet and ingredients on faeces quality in dogs is of interest for the optimisation of pet health and nutrition. Dietary intake, including both dry matter volume and nutrient content can impact faeces consistency. Altered faeces quality and the composition of microbial populations linked to gut health, as well as of bacterial fermentation products such as butyrate, have been associated with fibre intake (Wakshlag et al., 2011). Extensive meta-analyses of human research uncovered links between high fibre intake (particularly whole grain and cereal derived fibre) and reduced incidence of colorectal cancer suggesting potential links between the gut microbiome and long term clinical health in human subjects. However, research on the gut microbiome and links with animal health is limited. Thus, there is a need for understanding the relation between the gut microbiome and animal health. Furthermore, there is a need for novel methods and compositions for treating intestinal disorders that are associated with the microbiome.

### SUMMARY OF THE INVENTION

The presently disclosed subject matter provides a pet food product comprising sugar beet pulp at a concentration between about 0.5% w/w and about 6% w/w or a daily dose between 0.5 g and 90 g depending on the size and food consumption of a companion animal, e.g. a dog. In certain embodiments, the sugar beet pulp is at a concentration between about 0.5% w/w and about 1% w/w. In certain embodiments, the sugar beet pulp is at a concentration of about 0.8% w/w. The pet food product can be a topper, and in some embodiments, the topper is fed to the companion animal such that the companion animal receives a dosage of between about 0.5 g/day to about 90 g/day of the sugar beet pulp.

In certain embodiments, the pet food product further comprises an additional prebiotic. In certain embodiments, the pet food product further comprises an additional fiber. In certain embodiments, the pet food product further comprises probiotic.

The presently disclosed subject matter provides a pet food product comprising a bacterium selected from the group consisting of Lachnospiraceae sp., Faecalibacterium prausnitzii, Bacteroides plebeius, Holdemania [Eubacterium]biforme, Dorea sp., Ruminococcaceae sp., Bacteroides sp., Blautia sp., Erysipelotrichaceae sp., Lachnospiraceae sp. and any combination thereof in an amount effective to improve intestinal health and/or fecal quality in an companion animal. In certain embodiments, the bacterium is selected from the group consisting of Faecalibacterium prausnitzii, Bacteroides plebeius, Holdemania [Eubacterium] biforme and any combination thereof. In certain embodiments, the bacterium is selected from the group consisting of any bacteria comprising a 16S rRNA comprising a nucleotide sequence having at least about 95% sequence identity or identical to any of SEQ ID NOs: 1-14, and any combination thereof. In certain embodiments, the bacterium is selected from the group consisting of denovo1184, denovo1244, denovo1696, denovo2407, denovo2451, denovo283, denovo3487, denovo4154, denovo4328, denovo4681, denovo498, denovo5338, denovo6995, denovo943, and any combination thereof. In certain embodiments, the amount of the bacterium is between about 1×10⁴ CFU and about 1×10¹⁴ CFU.

In certain embodiments, the pet food product further comprises sugar beet pulp. The pet food product can include sugar beet pulp in an amount effective to improve intestinal health and/or fecal quality in the companion animal. In certain embodiments, the sugar beet pulp is present in the pet food product at a concentration between about 0.5% w/w and about 6% w/w or between a dose of about 0.5 g and 90 g per day. In certain embodiments, the sugar beet pulp is present in the pet food product at a concentration about 0.5% w/w and about 1% w/w. In certain embodiments, the sugar beet pulp is present in the pet food product at a concentration of about 0.8% w/w.

In certain embodiments, the pet food product further comprises a probiotic in addition to the bacterium and/or a prebiotic. In certain embodiments, the pet food product improves intestinal health in a companion animal within about 14 days after administering the pet food product to the companion animal.

In certain embodiments, the pet food product is a dietary supplement. In certain embodiments, the dietary supplement is added to the top of the pet food as a topper. In certain embodiments, the dietary supplement is subsequently mixed throughout the product. In certain embodiments, the pet food product is a dog food product. In some embodiments of any of the preceding methods, the companion animal is a dog.

Furthermore, the presently disclosed subject matter also provides a method of treating an intestinal dysbiosis and/or improving intestinal health in a companion animal in need thereof. In certain embodiments, the method comprises administering to the companion animal an effective amount of any pet food product disclosed herein to treat the intestinal dysbiosis and/or improve the intestinal health in the companion animal.

The presently disclosed subject matter provides a method for determining an intestinal health status in a companion animal. In certain embodiments, the presently disclosed subject matter provides a method for determining an intestinal health status in a companion animal in need thereof such as an animal with poor faeces quality or with diarrhea or with intestinal dysbiosis. In certain embodiments, the method comprises: measuring a first amount of the intestinal microorganism in the companion animal and detecting the bacteria denovo1184, denovo1244, denovo1696, denovo2407, denovo2451, denovo283, denovo3487, denovo4154, denovo4328, denovo4681, denovo498, denovo5338, denovo6995, denovo943; and further the bacteria denovo1214, denovo1400, denovo1762, denovo2014, denovo2197, denovo2368, denovo3663, denovo4206, denovo4485, denovo6368, denovo7117, denovo4881, and using the relative amount of these bacteria to determine the health of the companion animal.

In certain embodiments, the method comprises:
a) measuring a first amount of a first intestinal microorganism and/or a second amount of a second intestinal microorganism in the companion animal;
b) comparing the first amount of the intestinal microorganism with a first reference amount of the first intestinal microorganism, and/or comparing the second amount of the intestinal microorganism with a second reference amount of the second intestinal microorganism, wherein the reference amounts of the intestinal microorganisms are determined based on the amounts of the intestinal microorganisms in a plurality of healthy companion animals; and
c) determining the intestinal health status in the companion animal when the first amount of the first intestinal microorganism is higher than the first reference amount of the first intestinal microorganism, and/or when the second amount of the second intestinal microorganism is lower than the second reference amount of the second intestinal microorganism.

In some embodiments, the method for determining an intestinal health status in a companion animal comprises: a) measuring a first amount of a first intestinal microorganism from a first sample collected from the companion animal; and b) comparing the first amount of the first intestinal microorganism with a first reference amount of the first intestinal microorganism; and/or c) measuring a second amount of a second intestinal microorganism from a second sample collected from the companion animal; and d) comparing the second amount of the second intestinal microorganism with a second reference amount of the second intestinal microorganism; wherein the intestinal health status is determined to be healthy in the companion animal when the first amount of the intestinal microorganism is higher than the first reference amount of the first intestinal microorganism and/or when the second amount of the second intestinal microorganism is lower than the second reference amount of the second intestinal microorganism; and wherein the first reference amount of the first intestinal microorganisms and the second reference amount of the second intestinal microorganisms are determined based on the amounts of the intestinal microorganisms in a plurality of healthy companion animals.

In certain embodiments, the first intestinal microorganism is one or more bacterium comprising a 16S rRNA comprising a nucleotide sequence having at least about 95% sequence identity or identical to any of SEQ ID NOs: 1-14.

In certain embodiments, the first intestinal microorganism is selected from the group consisting of denovo1184, denovo1244, denovo1696, denovo2407, denovo2451, denovo283, denovo3487, denovo4154, denovo4328, denovo4681, denovo498, denovo5338, denovo6995, denovo943 and any combination thereof. In certain embodiments, the first intestinal microorganism is selected from the group consisting of Faecalibacterium prausnitzii, Bacteroides plebeius, Holdemania [Eubacterium] biforme and any combination thereof.

In certain embodiments, the second intestinal microorganism is one or more bacterium comprising a 16S rRNA comprising a nucleotide sequence having at least about 95% sequence identity or identical to any of SEQ ID NOs: 15-26.

In certain embodiments, the second intestinal microorganism is selected from the group consisting of denovo1214, denovo1400, denovo1762, denovo2014, denovo2197, denovo2368, denovo3663, denovo4206, denovo4485, denovo6368, denovo7117, denovo4881 and any combination thereof.

In certain embodiments, the method further comprises providing a customized recommendation of a treatment regimen, and/or further monitoring the intestinal microorganism, when the first amount of the first intestinal microorganism is lower than the first reference amount of the first intestinal microorganism, and/or when the second amount of the second intestinal microorganism is higher than the second reference amount of the second intestinal microorganism. In some embodiments, the treatment regimen is a pet food product as disclosed herein.

In certain embodiments, the amount of the first and/or second intestinal bacterium is measured from a fecal sample of the subject.

In certain embodiments, the method measures the amounts of at least 3 microorganisms. In certain embodiments, the method measures the amounts of at least 10 microorganisms. In certain embodiments, the method measures the amounts of between about 5-26 microorganisms. In certain embodiments, the method measures the amounts of between about 10-26 microorganisms.

The presently disclosed subject matter provides a method for treating an intestinal dysbiosis and/or improving intestinal health in a companion animal in need thereof. In certain embodiments, the method comprises:

a) measuring a first amount of one or more intestinal or fecal microorganisms in the companion animal;

b) administering a treatment regimen to the companion animal for treating the intestinal disorder and/or improving intestinal health;

c) measuring a second amount of the intestinal microorganism in the subject after step b); and

d) determining an intestinal health status of the animal or responsiveness to the treatment regimen.

In certain embodiments, the method further comprises continuing administering the treatment regimen, when the second amount of the intestinal microorganism is changed compared to the first amount of the intestinal microorganism.

In another aspect, the present disclosure features a method for treating an intestinal dysbiosis and/or improving intestinal health in a companion animal in need thereof, the method comprising:

a) measuring a first amount of an intestinal microorganism from a sample collected from the companion animal; and

b) administering a treatment regimen to the companion animal to treat the intestinal dysbiosis and/or improve intestinal health;

wherein the effectiveness of the treatment regimen is determined by measuring a second amount of the intestinal microorganism in the subject after performing step b) and determining an intestinal health status of the animal or responsiveness to the treatment regimen based on the difference between the first amount of the intestinal microorganism and the second amount of the intestinal microorganism.

In certain embodiments, the intestinal microorganism is one or more bacterium comprising a 16S rRNA comprising a nucleotide sequence having at least about 95% sequence identity or identical to any of SEQ ID NOs: 1-14. In certain embodiments, the intestinal microorganism is selected from the group consisting of denovo1184, denovo1244, denovo1696, denovo2407, denovo2451, denovo283, denovo3487, denovo4154, denovo4328, denovo4681, denovo498, denovo5338, denovo6995, denovo943 and any combination thereof. In some embodiments, the method further comprises: c) continuing to administer the treatment regimen after step b) if the second amount of the intestinal microorganism is increased compared to the first amount of the intestinal microorganism. In certain embodiments, the intestinal microorganism is selected from the group consisting of Faecalibacterium prausnitzii, Bacteroides plebeius, Holdemania [Eubacterium] biforme and any combination thereof. In certain embodiments, the second amount of the intestinal bacterium is measured between about 3 days or about 7 days and about 14 days after step b).

In other embodiments, the intestinal microorganism is one or more bacterium comprising a 16S rRNA comprising a nucleotide sequence having at least about 95% sequence identity or identical to any of SEQ ID NOs: 15-26. In certain embodiments, the intestinal microorganism is selected from the group consisting of denovo1214, denovo1400, denovo1762, denovo2014, denovo2197, denovo2368, denovo3663, denovo4206, denovo4485, denovo6368, denovo7117, denovo4881 and any combination thereof. In some embodiments, the method further comprises: c) continuing to administer the treatment regimen after step b) if the second amount of the intestinal microorganism is decreased compared to the first amount of the intestinal microorganism.

In certain embodiments, the intestinal microorganism is one or more bacterium comprising a 16S rRNA comprising a nucleotide sequence that is at least about 95% homologous or identical to any of SEQ ID NOs: 1-26, and wherein the method further comprises continuing administering the treatment regimen, when the second amount of the intestinal microorganism is increased for a bacterium comprising a 16S rRNA comprising a nucleotide sequence that is at least about 95% homologous or identical to any of SEQ ID NOs: 1-14, and/or decreased for a bacterium comprising a 16S rRNA comprising a nucleotide sequence that is at least about 95% homologous or identical to any of SEQ ID NOs: 15-26 compared to the first amount of the intestinal microorganism.

In certain embodiments, the second amount of the intestinal bacterium is measured between about 3 days or about 7 days and about 14 days after step b). In certain embodiments, the treatment regimen comprises a dietary regimen. In certain embodiments, the dietary regimen comprises administering an effective amount of any pet food product disclosed herein. In particular embodiments, the companion animal is a dog.

In some embodiments of any of the preceding methods, the sample is a fecal sample collected from the companion animal.

In certain embodiments, the amount of the intestinal microorganism is determined using a microarray.

In certain embodiments, the method measures the amounts of at least 3 microorganisms. In certain embodiments, the method measures the amounts of at least 10 microorganisms. In certain embodiments, the method measures the amounts of between about 5-26 microorganisms. In certain embodiments, the method measures the amounts of between about 10-26 microorganisms.

In another aspect, the disclosed herein is a pet food product comprising sugar beet pulp for use in treating an intestinal dysbiosis in a companion animal, wherein the sugar beet pulp is at a concentration between about 0.1% w/w and about 10% w/w in the pet food product.

In some embodiments, the pet food product is a dietary supplement or a functional food. In some embodiments, the pet food product is a topper. In some embodiments the topper is fed to the companion animal at a dosage of between about 0.5 g/day to about 90 g/day of the sugar beet pulp. In some embodiments, the sugar beet pulp is at a concentration between about 0.5% w/w and about 6% w/w in the pet food product.

In another aspect, the disclosure features any of the pet food products disclosed herein for use in treating an intestinal dysbiosis or improving intestinal health in a companion animal.

In some embodiments of any of the pet food products, wherein the pet food product is a dog food product.

In another aspect, the disclosure features use of sugar beet pulp in a dietary supplement or a pet food product for treating or preventing dysbiosis in a companion animal. In some embodiments, the companion animal is subject to a diet change. In further embodiments, the sugar beet pulp is at a concentration between about 0.5% w/w and about 6.0% w/w in the dietary supplement or the pet food product. In some embodiments, the companion animal is a dog.

In some embodiments, the dietary supplement or the pet food product is fed to the companion animal for at least about 3 days. In some embodiments, the dietary supplement or the pet food product is fed to the companion animal for at least about 7 days.

In another aspect, the disclosure features use of sugar beet pulp in a dietary supplement or a pet food product for treating or preventing dysbiosis in a companion animal, wherein the companion animal is subject to a diet change. In some embodiments, the sugar beet pulp is at a concentration between about 0.1% w/w and about 10% w/w in the dietary supplement or the pet food product.

In yet another aspect, the disclosure features use of any one of the pet foods disclosed herein for treating or prevent an intestinal dysbiosis in a companion animal, or for improving intestinal health in the companion animal.

In some embodiments of any of the disclosed uses, pet food product is a dog food product. In some embodiments, the companion animal is a dog.

In a further aspect, the disclosure features a health assessment tool for monitoring intestinal health status or dysbiosis in a companion animal, comprising one or more probe for detecting the amount of one or more microorganisms comprising a 16S rRNA comprising a nucleotide sequence having at least about 95% sequence identity or identical to the nucleotide sequence of any of SEQ ID NOs: 1-26.

In some embodiments, the health assessment tool comprises a microarray of the one or more probe. In some embodiments, the probe detects a 16S rRNA sequence of the one or more microorganism. In further embodiments, the health assessment tool comprises probes for detecting at least about 3 of the one or more microorganisms. In some embodiments, the health assessment tool comprises probes for detecting between about 5 to about 26 of the one or more microorganisms.

In further embodiments of the health assessment tool, the one or more microorganisms is selected from the group consisting of denovo1184, denovo1244, denovo1696, denovo2407, denovo2451, denovo283, denovo3487, denovo4154, denovo4328, denovo4681, denovo498, denovo5338, denovo6995, denovo943, denovo1214, denovo1400, denovo1762, denovo2014, denovo2197, denovo2368, denovo3663, denovo4206, denovo4485, denovo6368, denovo7117 denovo4881 and any combination thereof. In some embodiments, the amount of the microorganism is measured from a fecal sample of the companion animal. In some embodiments, the health assessment tool monitoring intestinal health status or dysbiosis by comparing the amount of the one or more microorganism with a reference amount of the one or more microorganism. In further embodiments, the companion animal is a dog.

### BRIEF DESCRIPTION OF THE DRAWINGS

FIG. 1 depicts study design for comparison of the effects of three digestive heath ingredients on faeces quality and the fecal microbiome.

FIGS. 2A and 2B depict the faeces scoring system used in Example 1.

FIGS. 3A and 3B depict mean faeces quality score from pens overnight across FIG. 3A all 21 days of the feeding phase per diet and FIG. 3B within the last 7 days of the 21-day feeding phase (***Possible adjustment to SBP in the last 7 days).

FIGS. 4A and 4B depict percentage of total faeces with unacceptable faeces scores from pens overnight (all +3.75) across FIG. 4A all 21 days of the feeding phase per diet and FIG. 4B within the last 7 days of the 21-day feeding phase.

FIG. 5 depicts PLS correlation plot of abundance data for the 26 OTUs with variable importance in projection (VIP) scores >1. Sample and OTU descriptors have been removed for ease of visualisation and replaced with a colour guide for orientation purposes (samples represented on the y axis by diet A, reference diet; B, sugar beet pulp; C, cellulose and D, pea protein. Faeces samples are represented in individual horizontal rows and are clustered according to likeness while bacterial OTUs are represented by individual columns within the heat plot. Colour coding within the plot is indicative of the degree and direction of correlation.

### DETAILED DESCRIPTION OF THE INVENTION

To date, there remains a need for novel methods and compositions for treating intestinal dysbiosis and other intestinal disorders that target gut microbiome. The present application relates to methods, compositions and food products for improving intestinal health, treating intestinal dysbiosis and/or treating an intestinal disorder in a companion animal, which is based, at least in part, on the discovery that animal food products comprising sugar beet pulp can promote intestinal health, and that changes of intestinal microorganisms are associated to intestinal health status.

For clarity and not by way of limitation, the detailed description of the presently disclosed subject matter is divided into the following subsections:

1. Definitions;

2. Intestinal bacteria and health assessment tools relating to the same;

3. Food products; and

4. Health assessment and treatment methods.

### 1. Definitions

The terms used in this specification generally have their ordinary meanings in the art, within the context of this invention and in the specific context where each term is used. Certain terms are discussed below, or elsewhere in the specification, to provide additional guidance to the practitioner in describing the methods and compositions of the invention and how to make and use them.

As used herein, the use of the word “a” or “an” when used in conjunction with the term “comprising” in the claims and/or the specification may mean “one,” but it is also consistent with the meaning of “one or more,” “at least one,” and “one or more than one.” Still further, the terms “having,” “including,” “containing” and “comprising” are interchangeable and one of skill in the art is cognizant that these terms are open ended terms.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

The term “effective treatment” or “effective amount” of a substance means the treatment or the amount of a substance that is sufficient to effect beneficial or desired results, including clinical results, and, as such, an “effective treatment” or an “effective amount” depends upon the context in which it is being applied. In the context of administering a composition, e.g., a pet food product, to improve immunity, digestive function and/or decreasing inflammation, an effective amount of a composition described herein is an amount sufficient to improving faeces quality, digestive health, immunity, digestive function and/or decreasing inflammation, as well as decrease the symptoms and/or reduce the likelihood of a digestive disorder and/or inflammation. An effective treatment described herein is a treatment sufficient to improving the microbiome, faeces quality, digestive health, immunity, digestive function and/or decreasing inflammation, as well as decrease the symptoms and/or reduce the likelihood of a digestive disorder and/or inflammation. The decrease can be an about 0.01%, about 0.1%, about 1%, about 5%, about 10%, about 20%, about 30%, about 40%, about 50%, about 60%, about 70%, about 80%, about 90%, about 95%, about 98% or about 99% decrease in severity of symptoms of a digestive disorder or inflammation, or the likelihood of a digestive disorder or inflammation. An effective amount can be administered in one or more administrations. A likelihood of an effective treatment described herein is a probability of a treatment being effective, i.e., sufficient to alter the microbiome, or treat or ameliorate a digestive disorder and/or inflammation, as well as decrease the symptoms.

As used herein, and as well-understood in the art, “treatment” is an approach for obtaining beneficial or desired results, including clinical results. For purposes of this subject matter, beneficial or desired clinical results include, but are not limited to, alleviation or amelioration of one or more symptoms, diminishment of extent of a disorder, stabilized (i.e., not worsening) state of a disorder, prevention of a disorder, delay or slowing of the progression of a disorder, and/or amelioration or palliation of a state of a disorder. The decrease can be an about 0.01%, about 0.1%, about 1%, about 5%, about 10%, about 20%, about 30%, about 40%, about 50%, about 60%, about 70%, about 80%, about 90%, about 95%, about 98% or about 99% decrease in severity of complications or symptoms. “Treatment” can also mean prolonging survival as compared to expected survival if not receiving treatment.

As used herein, and as well-understood in the art, a “probiotic” is a preparation or composition comprising microorganisms that can provide health benefits when consumed. The microorganisms include, but are not limited to bacteria, fungi, yeasts and archaea. In certain embodiments, the probiotic can modify the microbiome in the GI system to enhance the balance of the microbiome in GI system, e.g., by acting as an inoculum for an increased population of beneficial microbes, and/or by antagonizing growth of deleterious microbes. In certain embodiments, the probiotic is an animal probiotic, e.g., a feline probiotic or a canine probiotic.

As used herein, and as well-understood in the art, a “prebiotic” is a substance or a composition that can induce the growth or activity of one or more beneficial microorganism (e.g., one or more probiotics, e.g., bacteria, fungi, yeasts and archaea). In certain embodiments, the prebiotic can modify the microbiome in the GI system to enhance the balance of the microbiome in GI system. In certain embodiments, the prebiotic is indigestible to an animal. In certain embodiments, the prebiotic can induce the growth or activity of one or more animal probiotics, e.g., a feline probiotic or a canine probiotic.

The term “pet food” or “pet food composition” or “pet food product” or “final pet food product” means a product or composition that is intended for consumption by a companion animal, such as a cat, a dog, a guinea pig, a rabbit, a bird or a horse. For example, but not by way of limitation, the companion animal can be a “domestic” dog, e.g., Canis lupus familiaris. In certain embodiments, the companion animal can be a “domestic” cat such as Felis domesticus. A “pet food” or “pet food composition” or “pet food product” or “final pet food product” includes any food, feed, snack, food supplement, liquid, beverage, treat, toy (chewable and/or consumable toys), meal substitute or meal replacement.

An “individual” or “subject” herein is a vertebrate, such as a human or non-human animal, for example, a mammal. Mammals include, but are not limited to, humans, non-human primates, farm animals, sport animals, rodents and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys.

### 2. Intestinal Microorganisms and Health Assessment Tools Relating to the Same

The presently disclosed subject matter provides intestinal microorganisms and combinations thereof, which is based, at least in part, on the discovery that changes of intestinal microorganism populations within the microbiome are associated to intestinal health status and to feces quality in a subject. The subject can be, for example, a companion animal, such as a dog.

In certain embodiments, the intestinal microorganism can be used to indicate intestinal health in a subject. In certain embodiments, the intestinal microorganism is associated to a healthy status or an intestinal dysbiosis in a subject. For example, the subject can be a companion animal, e.g., a dog.

In certain embodiments, the intestinal microorganism indicates a healthy intestine status in a subject. In some embodiments, the subject is a companion animal, e.g., a dog. For example, the healthiness of the companion animal can be determined based on a reference value of a plurality of healthy companion animals of the type same species. In certain embodiments, the intestinal microorganism is a bacterium comprising a 16S ribosomal RNA (rRNA) comprising a nucleotide sequence having at least about 95% sequence identity to the nucleotide sequence of any of SEQ ID NOs: 1-26, and any combination thereof.

In certain embodiments, the intestinal microorganism a bacterium selected from the group consisting of Lachnospiraceae sp., Faecalibacterium prausnitzii, Bacteroides plebeius, Holdemania [Eubacterium] biforme, Dorea sp., Ruminococcaceae sp., Bacteroides sp., Blautia sp., Erysipelotrichaceae sp., Lachnospiraceae sp. and any combination thereof. In certain embodiments, the bacterium is selected from the group consisting of Faecalibacterium prausnitzii, Bacteroides plebeius, Holdemania [Eubacterium] biforme, and any combination thereof. In further embodiment, the intestinal microorganism is an organism comprising a nucleotide sequence having at least about 95%, about 96%, about 97%, about 98%, about 99%, or about 100% sequence identity to the 16S rRNA sequence identity to the 16S rRNA sequence of any of the bacteria disclosed herein, or any of the nucleotide sequences of SEQ ID NOs: 1-26.

In certain embodiments, the intestinal microorganism is a bacterium is selected from the group consisting of denovo1184, denovo1244, denovo1696, denovo2407, denovo2451, denovo283, denovo3487, denovo4154, denovo4328, denovo4681, denovo498, denovo5338, denovo6995, denovo943 and any combination thereof.

In certain embodiments, each of denovo1184, denovo1244, denovo1696, denovo2407, denovo2451, denovo283, denovo3487, denovo4154, denovo4328, denovo4681, denovo498, denovo5338, denovo6995 and denovo943 comprises a 16S rRNA comprising a nucleotide sequence having at least about 80% (e.g., at least about 85%, at least about 90%, or at least about 95%, at least about 96%, at least about 97%, at least about 98%, at least about 99%, at least about 99.5%, or at least about 99.9%) sequence identity to the nucleotide sequence of SEQ ID NOs: 1-14, respectively.

In certain embodiments, each of denovo1184, denovo1244, denovo1696, denovo2407, denovo2451, denovo283, denovo3487, denovo4154, denovo4328, denovo4681, denovo498, denovo5338, denovo6995 and denovo943 comprises a 16S rRNA comprising the nucleotide sequence of SEQ ID NOs: 1-14, respectively.

The intestinal microorganism can indicate the health of the microbiome or an intestinal dysbiotic status in the microbiome of a companion animal. In certain embodiments, the intestinal microorganism comprises a bacterium selected from the group consisting of Clostridiales sp., Clostridia sp., Mogibacteriaceae sp., Lachnospiraceae sp., Clostridiaceae sp., Peptostreptococcaceae sp., and any combination thereof.

In certain embodiments, the bacterium is selected from the group consisting of denovo1214, denovo1400, denovo1762, denovo2014, denovo2197, denovo2368, denovo3663, denovo4206, denovo4485, denovo6368, denovo7117, denovo4881 and any combination thereof.

In certain embodiments, each of denovo1214, denovo1400, denovo1762, denovo2014, denovo2197, denovo2368, denovo3663, denovo4206, denovo4485, denovo6368, denovo7117 and denovo4881 comprises a 16S rRNA comprising a nucleotide sequence having at least about 80% (e.g., at least about 85%, at least about 90%, or at least about 95%, at least about 96%, at least about 97%, at least about 98%, at least about 99%, at least about 99.5%, or at least about 99.9%) sequence identity to the nucleotide sequence of SEQ ID NOs: 15-26, respectively.

In certain embodiments, each of denovo1214, denovo1400, denovo1762, denovo2014, denovo2197, denovo2368, denovo3663, denovo4206, denovo4485, denovo6368, denovo7117 and denovo4881 comprises a 16S rRNA comprising the nucleotide sequence set forth in SEQ ID NOs: 15-26, respectively.

denovo1184 [SEQ ID NO: 1]:
CCTGTTTGCTCCCCACGCTTTCGAGCCTCAACGTCAGTCTCTGTCCAGTAAGCCGCCTTCGCCACTGGTGTTCCTCC
TAATATCTACGCATTTCACCGCTACACTAGGAATTCCACTTACCTCTCCAGTACTCTAGCTCAACAGTTTCCAAAGC
AGTCCCGTGGTTAAGCCTCGGGCTTTCACTTCAGACTTGCCGAGCCGTCTACGCTCCCTTTACACCCAGTAAATCCG
GATAACGCTCGCCCCCTACGTATTACCGCGGCTGCTGGCACGTAGTTAGCCGGGGCTTCTTAGTCAGGTACCGTCAT
TCTCTTCCCTGCTGATAGAGCTTTACGTACCGAAGTACTTCTTCACTCACGCGGCGTCGCTGCATCAGGGTTTCCCC
CATTGTGCAATATCCCCCA
denovo1244 [SEQ ID NO: 2]:
CCTGTTTGCTACCCACACTTTCGAGCCTCAGCGTCAGTTGGTGCCCAGTAGGCCGCCTTCGCCACTGGTGTTCCTCC
CGATATCTACGCATTCCACCGCTACACCGGGAATTCCGCCTACCTCTGCACTACTCAAGAAAAACAGTTTTGAAAGC
AGTTTATGGGTTGAGCCCATAGATTTCACTTCCAACTTGTCTTCCCGCCTGCGCTCCCTTTACACCCAGTAATTCCG
GACAACGCTTGTGACCTACGTTTTACCGCGGCTGCTGGCACGTAGTTAGCCGTCACTTCCTTGTTGGGTACCGTCAT
TATCTTCCCCAACAACAGGAGTTTACAATCCGAAGACCTTCTTCCTCCACGCGGCGTCGCTGCATCAGGGTTTCCCC
CATTGTGCAATATTCCCCA
denovo1696 [SEQ ID NO: 3]:
CCTGTTTGATACCCACACTTTCGAGCATGAACGTCAGTTACGGCTTAGTGTGCTGCCTTCGCAATCGGAGTTCTTCG
TGATATCTAAGCATTTCACCGCTACACCACGAATTCCGCACACCTCAACCGCACTCAAGGACGCCAGTATCAACTGC
AATTTTAAGGTTGAGCCCCAAACTTTCACAGCTGACTTAACGACCCGTCTGCGCTCCCTTTAAACCCAATAAATCCG
GATAACGCTCGCATCCTCCGTATTACCGCGGCTGCTGGCACGGAGTTAGCCGATGCTTATTCATAAGGTACATACAA
GCTCCCACACGTGGGAGGTTTTATTCCCTTATAAAAGAAGTTTACAATCCGTAGGACCTTCATCCTTCACGCTACTT
GGCTGGTTCAGACTCTCGTCCATTGACCAATATTCCTCA
denovo2407 [SEQ ID NO: 4]:
CCTATTTGCTCCCCACGCTTTCGTGCTTCAGTGTCAGAATCCAGACCAGACGGCCGCCTTCGCCACCGGTGTTCTTC
CATATATCTACGCATTTTACCGCTACACATGGAGTTCCGCCGTCCTCTTCTGTTCTCTAGCTGATCAGTTTCCAGAG
CAAGTACGGGTTGAGCCCATACCTTTTACTCCAGACTTGATCTGCCACCTACGCACCCTTTACGCCCAATCATTCCG
GATAACGCTCGCCACCTACGTATTACCGCGGCTGCTGGCACGTAGTTAGCCGTGACTTTCTGGTAAGATACCATCAC
TCACTCATCATTCCCTATGAGTGCCGTTTTTCTCTTACAACAGAGCTTTACGATCCGAAGACCTTCCTCACTCACGC
GGCATTGCTCGTTCAGGGTTCCCCCCATTGACGAAAATTCCCTA
denovo2451 [SEQ ID NO: 5]:
CCTGTTTGCTCCCCACGCTTTCGAGCCTCAACGTCAGTTATCGTCCAGTAAGCCGCCTTCGCCACTGATGTTCCTCC
TAATATCTACGCATTTCACCGCTACACTAGGAATTCCGCTTACCTCTCCGACACTCTAGAAGCACAGTTTCCAAAGC
AGTCACGGGGTTGAGCCCCGGGCTTTCACTTCAGACTTGCACTTCCGTCTACGCTCCCTTTACACCCAGTAAATCCG
GATAACGCTTGCCCCCTACGTATTACCGCGGCTGCTGGCACGTAGTTAGCCGGGGCTTCTTAGTCAGGTACCGTCAT
TTTCTTCCCTGCTGATAGAAGTTTACATACCGAAATACTTCTTCCTTCACGCGGCGTCGCTGCATCAGGGTTTCCCC
CATTGTGCAATATTCCCCA
denovo283 [SEQ ID NO: 6]:
CCTGTTTGCTACCCATGCTTTCGAGCCTCAGCGTCAGTTAGTGCCCAGCAGGCCGCCTTCGCCACTGGTGTTCCTCC
CGATATCTACGCATTCCACCGCTACACCGGGAATTCCGCCTGCCTCTGCACCACTCAAGATTTGCAGTTTTGAATGC
GAGAAGGGGTTGAGCCCCTCCATTAAACATCCAACTTGCAAACCCGCCTGCGCTCCCTTTACACCCAGTAATTCCGG
ACAACGCTTGCCACCTACGTATTACCGCGGCTGCTGGCACGTAGTTAGCCGTGGCTTCCTCCTCGGGTACCGTCATA
CTTCGTCCCCGAAGACAGAGGTTTACAATCCGAAAATCTTCTTCCCTCACGCGGCGTCGCTGCATCAGAGTTTCCTC
CATTGTGCAATATTCCCCA
denovo3487 [SEQ ID NO: 7]:
CCTGTTTGATACCCGCACTTTCGAGCATCAGCGTCAGTTACGGTCCAGTAAGCTGCCTTCGCAATCGGAGTTCTTCG
TGATATCTAAGCATTTCACCGCTACACCACGAATTCCGCCTACCTATACCGCACTCAAGAAATCCAGTATCAACTGC
AATTTTACGGTTGAGCCGCAAACTTTCACAACTGACTTAAACTTCCGCCTACGCTCCCTTTAAACCCAATAAATCCG
GATAACGCTCGGATCCTCCGTATTACCGCGGCTGCTGGCACGGAGTTAGCCGATCCTTATTCATACGGTACATACAA
AAAAGCACACGTGCTTCACTTTATTCCCGTATAAAAGAAGTTTACAACCCATAGGGCAGTCATCCTTCACGCTACTT
GGCTGGTTCAGACTCTCGTCCATTGACCAATATTCCTCA
denovo4154 [SEQ ID NO: 8]:
CCTGTTTGCTCCCCACGCTTTCGAGCCTCAACGTCAGTTACCGTCCAGTAAGCCGCCTTCGCCACTGGTGTTCCTCC
TAATATCTACGCATTTCACCGCTACACTAGGAATTCCGCTTACCCCTCCGGTACTCAAGATCAACAGTTTCCAATGC
AGTCCAGGGGTTGAGCCCCTGCCTTTCACATCAGACTTGCTGCTCCGTCTACGCTCCCTTTACACCCAGTAAATCCG
GATAACGCTTGCCCCCTACGTATTACCGCGGCTGCTGGCACGTAGTTAGCCGGGGCTTCTTAGTCAGGTACCGTCAT
TATCTTCCCTGCTGATAGAAGTTTACATACCGAGATACTTCTTCCTTCACGCGGCGTCGCTGCATCAGGGTTTCCCC
CATTGTGCAATATTCCCCA
denovo4328 [SEQ ID NO: 9]:
CCTGTTTGATACCCACACTTTCGAGCATGAACGTCAGTTACAGTTTAGCAAGCTGCCTTCGCAATCGGGGTTCTTCG
TGATATCTAAGCATTTCACCGCTACACCACGAATTCCGCCTGCCTCAACTGCACTCAAGGAAACCAGTATCAACTGC
AATTTTACGGTTGAGCCGCAAACTTTCACAACTGACTTAATCTCCCGTCTGCGCTCCCTTTAAACCCAATAAATCCG
GATAACGCTCGCATCCTCCGTATTACCGCGGCTGCTGGCACGGAGTTAGCCGATGCTTATTCATACGGTACATACAA
AATCCTACACGTAGGAAACTTTATTCCCGTATAAAAGAAGTTTACAATCCGTAGGACCTTCATCCTTCACGCTACTT
GGCTGGTTCAGGCTCTCGCCCATTGACCAATATTCCTCA
denovo4681 [SEQ ID NO: 10]:
CCTATTTGCTCCCCACGCTTTCGTGCCTGAGCGTCAGTTACAGACTAGCAAGCCGCCTTCGCCACCGGTGTTCCTCC
ATATATCTATGCATTTTACCGCTACACATGGAATTCCACTTGCCCCTTCTGCACTCTAGTTCACCAGTTTCTAAGCC
TGGATGGGGTTGAGCCCCACAATTTAAGCTTAAACTTAATAAACCGCCTGCGCACCCTTTACGCCCAATAATTCCGG
ATAACGCTCGTCACCTTCGTATTACCGCGGCTGCTGGCACGAAGTTAGCCGTGACTTTCTGGTAAAGTACCGTCACT
TACGTAGTTGATACGTAACATTCTTCCTTTACAACAGAGCTTTACAAGCCGAAGACCTTCATCACTCACGCGGCATT
GCTCGGTCAGGCTTGCGCCCATTGCCGAAAATTCCCTA
denovo498 [SEQ ID NO: 11]:
CCTGTTTGATACCCACACTTTCGAGCCTCAATGTCAGTTGCAGCTTAGCAGGCTGCCTTCGCAATCGGAGTTCTTCG
TGATATCTAAGCATTTCACCGCTACACCACGAATTCCGCCTGCCTCAACTGCACTCAAGATATCCAGTATCAACTGC
AATTTTACGGTTGAGCCGCAAACTTTCACAACTGACTTAAACATCCATCTACGCTCCCTTTAAACCCAATAAATCCG
GATAACGCTCGGATCCTCCGTATTACCGCGGCTGCTGGCACGGAGTTAGCCGATCCTTATTCATAAAGTACATGCAA
ACGGGTATGCATACCCGACTTTATTCCTTTATAAAAGAAGTTTACAACCCATAGGGCAGTCATCCTTCACGCTACTT
GGCTGGTTCAGGCTCTCGCCCATTGACCAATATTCCTCA
denovo5338 [SEQ ID NO: 12]:
CCTGTTTGCTCCCCACGCTTTCGAGCCTCAACGTCAGTTACTGTCCAGTAAGCCGCCTTCGCCACTGGTGTTCCTCC
TAATATCTACGCATTTCACCGCTACACTAGGAATTCCACTTACCTCTCCAGCACTCTAGCAGAACAGTTTCCAAAGC
AGTCCCGGGGTTGAGCCCCGGGCTTTCACTTCAGACTTGCTCCGCCGTCTACGCTCCCTTTACACCCAGTAAATCCG
GATAACGCTTGCCCCCTACGTATTACCGCGGCTGCTGGCACGTAGTTAGCCGGGGCTTCTTAGTCAGGTACCGTCAT
TTTCTTCCCTGCTGATAGAGCTTTACATACCGAGATACTTCTTCACTCACGCGGCGTCGCTGCATCAGGGTTTCCCC
CATTGTGCAATATTCCCCA
denovo6995 [SEQ ID NO: 13]:
CCTGTTCGCTCCCCACGCTTTCGAGTCTCAGCGTCAGTTATAGTCCAGCAAGCCGCCTTCGCCACCGGTGTTCTTCC
TGATATCTACGCATTTCACCGCTACACCAGGAATTCCGCTTGCCCTTCCTACACTCTAGCTGTACAGTTTCAAAAGC
AGTCTTGGGGTTGAGCCCCAAGTTTTCACTCCTGACTTGTACTGCCGCCTACACTCCCTTTACACCCAGTAAATCCG
GATAACGCTTGCCCCATACGTATTACCGCGGCTGCTGGCACGTATTTAGCCGGGGCTTCTTCTGTAGGTACCGTCAT
TATCTTCCCTACTGATAGAGCTTTACATACCGAAATACTTCTTCACTCACGCGGCGTCGCTGCATCAGAGTTGCCTC
CATTGTGCAATATTCCCCA
denovo943 [SEQ ID NO: 14]:
CCTGTTTGATACCCACACTTTCGAGCCTCAATGTCAGTTGCAGCTTGGCGGACTGCTTTCGCAATCGGGGTTCTTCG
TGATATCTAAGCATTTCACCGCTACACCACGAATTCCATCCGCCTCAAATGCACTCAAGAAAACCAGTATCAACTGC
AATTTTACGGTTGAGCCGCAAACTTTCACAGCTGACTTAATCTCCCATCTACGCTCCCTTTAAACCCAATAAATCCG
GATAACGCTCGCATCCTCCGTATTACCGCGGCTGCTGGCACGGAGTTAGCCGATGCTTATTCATAAGGTACATACAA
ACAGGTACACGTACCTGACTTTATTCCCTTATAAAAGAAGTTTACAACCCATAGGGCAGTCTTCCTTCACGCTACTT
GGCTGGTTCAGACTCTCGTCCATTGACCAATATTCCTCA
denovo1214 [SEQ ID NO: 15]:
CCTGTTTGCTCCCCACGCTTTCGTACCTCAGTGTCAGTTACAGTCCAGAAAGCCGCCTTCGCCACTGGTGTTCCTCC
TAATATCTACGCATTTCACCGCTACACTAGGAATTCCGCTTTCCTCTCCTGCACTCAAGTTTGCCAGTTCGCAGGGC
GAACAATGGTTGAGCCATTGCCTTAAACCCTGCGCTTGGTAAACCACCTACGTACCCTTTACGCCCAATAATTCCGG
ATAACGCTTGCCCCCTCCGTATTACCGCGGCTGCTGGCACGGAGTTAGCCGGGGCTTCCTCCAAGGGTACCGTCATT
TGTTTCTTCCCCAAGGACAGAGCTTTACGACCCAAAGGCCTTCATCGCTCACGCGGCGTTGCTGCATCAGGCTTGCG
CCCATTGTGCAATATTCCCCA
denovo1400 [SEQ ID NO: 16]:
CCTGTTCGCTCCCCACGCTTTCGCACCTCAGTGTCAGTATAAGCCTGGCAGACCGCCTTCGCCTCCGGTATTCCTCC
TGATCTCTGCGCATTTCACCGCTACACCAGGAATTCTGCCTGCCTCGACAATACTCCAGCTGCCCGGTTTGCGGTGA
CCTCCCAGGGTTGAGCCCTGGACTTTTACACCGCACCTAAACAACCACCTGCGTGCCCTTTACGCCCAATAATTCCG
GATAACGCTCGCCCCCTACGTATTACCGCAGCTGCTGGCACGTAGTTGGCTGGGGCTTGCTTACCGGGTACCGTCAT
CGTCTTCCCCGGTAACAGAGCTTTACAGAACGAATCCCTTCTTCACTCACGCGGCATCGCTGCGTCAGAGTTGCCTC
CATTGCGCAATATTCCCCA
denovo1762 [SEQ ID NO: 17]:
CCTGTTTGCTCCCCACGCTTTCGAGCCTCAACGTCAGTTACCGTCCAGTAAGCCGCCTTCGCCACTGGTGTTCCTCC
TAATATCTACGCATTTCACCGCTACACTAGGAATTCCGCTTACCTCTCCGGCACTCAAGTCCCACAGTTTCCAATGC
AATCCAGAAGTTGAGCCTCTGCCTTTCACATCAGACTTGCAGAACCGTCTACGCTCCCTTTACACCCAGTAAATCCG
GATAACGCTTGCCCCCTACGTATTACCGCGGCTGCTGGCACGTAGTTAGCCGGGGCTTCTTAGTCAGGTACCGTCAT
TTTCTTCCCTGCTGATAGAGCTTTACATCACGAATGACTTCTTCACTCACGCGGCGTCGCTGCATCAGGGTTTCCCC
CATTGTGCAATATTCCCCA
denovo2014 [SEQ ID NO: 18]:
CCTGTTCGCTCCCCGCGCTCTCGCGCCTCAGCGTCAGTGTCCGTCCGGCGGGCCGCCTTCGCCTCCGGTGTTCCTCC
TGGCCTCTGCGCATTTCACCGCTACGCCAGGAGTTCCGCCCGCCCCTCCGGCACTCCAGCCGCCCGGTCCGGGGCGC
TTGCCCGGGGTTGGGCCCCGGCTTTTCACACCCCGCCTAAGCGGCCGCCTGCGCGCGCTTTACGCCCAGTGATTCCG
GACAACGCTCGCCCCCCACGTATTGCCGCGGCTGCTGGCACGTGGTTGGCCGGGGCTTTCGCGCCGGGTTCAGTCAT
TTCTTCCTCCCCGGCTACGGGGCTTTACGGGCCGAGGCCCTTCGTCGCCCACGCGGCGTCGCTGCGTCAGAGTTCCC
TCCATTGCGCAATATTCCCCA
denovo2197 [SEQ ID NO: 19]:
CCTGTTTGCTCCCCACGCTTTCGAGCCTCAGCGTCAGTTACAGTCCAGAGAATCGCCTTCGCCACTGGTGTTCTTCC
TAATCTCTACGCATTTCACCGCTACACTAGGAATTCCATTCTCCTCTCCTGCACTCTAGACTTCCAGTTTGAAATGC
AGCACCCAAGTTGAGCCCGGGTATTTCACATCTCACTTAAAAGTCCGCCTACGCTCCCTTTACGCCCAGTAAATCCG
GACAACGCTCGCCACCTACGTATTACCGCGGCTGCTGGCACGTAGTTAGCCGTGGCTTCCTCCTCAGGTACCGTCAT
TATCGTCCCTGAAGACAGAGCTTTACAACCCGAAGGCCGTCATCACTCACGCGGCGTTGCTGCATCAGGGTTTCCCC
CATTGTGCAATATTCCCCA
denovo2368 [SEQ ID NO: 20]:
CCTGTTCGCTCCCCGCGCTCTCGCGCCTCAGCGTCAGTGTCCGTCCGGCGGGCCGCCTTCGCCTCCGGTGTTCCTCC
CGGCCTCTGCGCATTTCACCGCTACGCCGGGAGTTCCGCCCGCCCCTCCGGCGCTCCAGCCGCCCGGTCCGGGGCGC
TTGCCCGGGGTTGGGCCCCGGCTTTTCACACCCCGCCTAAGCGGCCGCCTGCGCGCGCTTTACGCCCAGTGATTCCG
GACAACGCTCGCCCCCCACGTATTGCCGCGGCTGCTGGCACGTGGTTGGCCGGGGCTTTCGTGCCGGGTTCAGTCAT
TTCCTTCCTCCCCGGCGGCGGGGCTTTACGGGCCTGGGCCCTTCATCGCCCACGCGGCGTCGCTGCGTCAGAGTTGC
CTCCATTGCGCAATATTCCCCA
denovo3663 [SEQ ID NO: 21]:
CCTGTTCGCTCCCCGCGCTCTCGCGCCTTAGCGTCAGTGTGCGTCCGGCGGGCCGCCTTCGCCTCCGGTGTTCCCCC
TGGCCTCTGCGCATTTCACCGCTACGCCAGGGGTTCCGCCCGCCTCTCCGCCACTCCAGCCGCCCGGTCCGGGGCGC
TTGCCCGGGGTTGGGCCCCGGCTTTTAACGCCCCGCCTAAGCGGCCGCCTGCGCGCGCTTTACGCCCAGTGATTCCG
GACAACGCTCGCCCCCCACGTATTGCCGCGGCTGCTGGCACGTGGTTGGCCGGGGCTTTCTTGCCGGGTTGCGTCAT
TTTTTTCCTCCCCGGCGGCGGGGCTTTACGGGCCTGAGCCCTTCATCGCCCACGCGGCATCGCTGCGTCAGAGTTCC
CTCCATTGCGCAATATTCCCCA
denovo4206 [SEQ ID NO: 22]:
CCTGTTTGCTCCCCACGCTTTCGTGCCTCAGTGTCAGTTACAGTCCAGAAAGCCGCCTTCGCTACTGGTGTTCCTCC
TAATATCTACGCATTTCACCGCTACACTAGGAATTCCACTTTCCTCTCCTGCACTCAAGTTTCCCAGTTTCAAGAGC
TTACTACGGTTAAGCCGTAGCCTTTCACTCCTGACTTAAGAAACCACCTACGCACCCTTTACGCCCAGTAAATCCGG
ATAACGCTAGCCCCCTACGTATTACCGCGGCTGCTGGCACGTAGTTAGCCGGGGCTTCCTCCTCAAGTACCGTCATT
ATCTTCCTTGAGGACAGAGTTTTACGACCCGAAGGCCTTCATCACTCACGCGGCGTTGCTGCATCAGGCTTTCGCCC
ATTGTGCAATATTCCCCA
denovo4485 [SEQ ID NO: 23]:
CCTGTTTGCTCCCCACGCTCTCGCGCCTTAGCGTCAGTCTCTTCCCGGCAGGCCGCCTTCGCCTCCGGTATTCCTCC
TGATATCTGCGCATTTCACCGCTACACCAGGAATTCTGCCTGCCTCTAAAGGACTCCAGCCTCTCGGTTCCTGTCGC
CTCCCCGGGTTGGGCCCGGGACTTTTACAACAGGCCTTCTAGGCCGCCTACGCGCGCTTTACGCCCAGTAATTCCGG
ACAACGCTCGCCCCCTACGTATTACCGCGGCTGCTGGCACGTAGTTAGCCGGGGCTTTAAAGACGGGTGACATCTCT
CTTCTCCCCGTCGACTGAGCTCTGCGGGACGAATCCCTTCCTCACTCACGCGGCATCGCTGCGTCAGAGTTTCCTCC
ATTGCGCAATATTCCCCA
denovo6368 [SEQ ID NO: 24]:
CCTGTTTGCTCCCCACGCTCTCGCGCCTGAGCGTCAGTCTCTCTCCAGCAGGCCGCCTTCGCCTCCGGTATTCCTCC
TGATCTCTGCGTATTTCACCACTACACCAGGAATTCTGCCTGCCTCTAGAGCACTCTAGTCAGCTGGTTCTTCCTGC
TTACCCGGGGTGGGCCCGGGCCTTTTACAAAAAGCCTCTCTGACCGCCTGCGCGCGCTTTACGCCCAATGATTCCGG
ACAACGCTCGCCCCCTACGTATTGCCGCGGCTGCTGGCACGTAGTTAGCCGGGGCTTAAAGTCAGGTAGTCTCTCTC
TTGCCTGACGACTGAGCTCTGCGGGACGAATCCCTTCCTCACTCACGCGGCATCGCTGCGTCAGAGTTGCCTCCATT
GCGCAATATTCCCCA
denovo7117 [SEQ ID NO: 25]:
CCTGTTTGCTCCCCACGCTTTCGTGCCTCAGTGTCAGTTACAGTCCAGAGAGCCGCCTTCGCAACTGGTATTCCTCC
TAATATCTACGCATTTCACCGCTACACTAGGAATTCTACTCTCCTCTCCTGCACTCAAGTTTCTCAGTTTCAAAGGC
TTACTACGGTTGAGCCGTAGCCTTTCACCTCTGACTTAAGAAACCACCTACGCACCCTTTACGCCCAGTAATTCCGG
ATAACGCTAGCCCCCTACGTATTACCGCGGCTGCTGGCACGTAGTTAGCCGGGGCTTCCTCCTCAAGTACCGTCATT
ATCTTCCTTGAGGACAGAGCTTTACGACCCGAAGGCCTTCATCGCTCACGCGGCGTTGCTGCATCAGGCTTTCGCCC
ATTGTGCAATATTCCCCA
denovo4881 [SEQ ID NO: 26]:
CCTGTTTGCTCCCCACGCTCTCGCGCCTGAGCGTCAGTGTCTGTCCGGCAGGCCGCCTTCGCCTCTGGTATTCCTCC
TGATCTCTGCGCATTTCACCGCTACACCAGGAATTCTGCCTGCCTCTCCAGCACTCGAGCCGCGCGGTTCGGGATGC
CCGAACAGGGTTGGGCCCTGATCTTTCACATCCCGCCTTCGCGGCCGCCTGCGCGCCCTTTACGCCCAGTCATTCCG
GACAACGCTCGCCCCCTACGTATTACCGCGGCTGCTGGCACGTAGTTAGCCGGGGCTTGCTTGTCAGGTACCGTCTC
TCTCTTCCCTGACAACAGAGCTTTACGGGACGAATCCCTTCTTCGCTCACGCGGCATTGCTGCGTCAGAGTTGCCTC
CATTGCGCAATATTCCCCA

By “percentage of identity” or “sequence identity” between two sequences, e.g., nucleic acid or amino acid sequences, in the sense of the present invention, it is intended to indicate a percentage of nucleotides or of identical amino acid residues between the two sequences to be compared, obtained after the best alignment (optimum alignment), this percentage being purely statistical and the differences between the two sequences being distributed randomly and over their entire length. The comparisons of sequences between two nucleic acid or amino acid sequences are traditionally carried out by comparing these sequences after having aligned them in an optimum manner, said comparison being able to be carried out by segment or by “comparison window”. The optimum alignment of the sequences for the comparison can be carried out, in addition to manually, by means of the local homology algorithm of Smith and Waterman (1981) [Ad. App. Math. 2:482], by means of the local homology algorithm of Neddleman and Wunsch (1970) [J. Mol. Biol. 48: 443], by means of the similarity search method of Pearson and Lipman (1988) [Proc. Natl. Acad. Sci. USA 85:2444), by means of computer software using these algorithms (GAP, BESTFIT, FASTA and TFASTA in the Wisconsin Genetics Software Package, Genetics Computer Group, 575 Science Dr., Madison, Wis., or else by BLAST N or BLAST P comparison software).

The percentage of identity between two nucleic acid or amino acid sequences is determined by comparing these two sequences aligned in an optimum manner and in which the nucleic acid or amino acid sequence to be compared can comprise additions or deletions with respect to the reference sequence for an optimum alignment between these two sequences. The percentage of identity is calculated by determining the number of identical positions for which the nucleotide or the amino acid residue is identical between the two sequences, by dividing this number of identical positions by the total number of positions in the comparison window and by multiplying the result obtained by 100 in order to obtain the percentage of identity between these two sequences.

For example, it is possible to use the BLAST program, “BLAST 2 sequences” (Tatusova et al., “Blast 2 sequences—a new tool for comparing protein and nucleotide sequences”, FEMS Microbiol Lett. 174:247-250) available on the site www.ncbi.nlm.nih.gov, the parameters used being those given by default (in particular for the parameters “open gap penalty”: 5, and “extension gap penalty”: 2; the matrix chosen being, for example, the matrix “BLOSUM 62” proposed by the program), the percentage of identity between the two sequences to be compared being calculated directly by the program. It is also possible to use other programs such as “ALIGN” or “Megalign” (DNASTAR) software.

By amino acid sequence having at least about 80%, preferably at least about 85%, at least about 90%, at least about 95%, and at least about 98% identity with a reference amino acid sequence, those having, with respect to the reference sequence, certain modifications, in particular a deletion, addition or substitution of at least one amino acid, a truncation or an elongation are preferred. In the case of a substitution of one or more consecutive or nonconsecutive amino acid(s), the substitutions are preferred in which the substituted amino acids are replaced by “equivalent” amino acids. The expression “equivalent amino acids” is aimed here at indicating any amino acid capable of being substituted with one of the amino acids of the base structure without, however, essentially modifying the biological activities of the corresponding antibodies and such as will be defined later, especially in the examples. These equivalent amino acids can be determined either by relying on their structural homology with the amino acids which they replace, or on results of comparative trials of biological activity between the different antibodies capable of being carried out.

By way of non-limiting example, Table 1 represents the possibilities of substitution capable of being carried out without resulting in a profound modification of the biological activity of a corresponding modified amino acid sequence, the reverse substitutions being naturally envisageable under the same conditions.

TABLE 1

| Original residue | Substitution(s) |
|---|---|
| Ala (A) | Val, Gly, Pro |
| Arg (R) | Lys, His |
| Asn (N) | Gln |
| Asp (D) | Glu |
| Cys (C) | Ser |
| Gln (Q) | Asn |
| Glu (G) | Asp |
| Gly (G) | Ala |
| His (H) | Arg |
| Ile (I) | Leu |
| Leu (L) | Ile, Val, Met |
| Lys (K) | Arg |
| Met (M) | Leu |
| Phe (F) | Tyr |
| Pro (P) | Ala |
| Ser (S) | Thr, Cys |
| Thr (T) | Ser |
| Trp (W) | Tyr |
| Tyr (Y) | Phe, Trp |
| Val (V) | Leu, Ala |

The presently disclosed subject matter provides a health assessment tool relating to the microorganisms disclosed herein. In certain embodiments, the health assessment tool is for monitoring intestinal health status or an intestinal dysbiosis of a subject. The subject can be, in certain non-limiting embodiments, a companion animal (e.g., a dog). In certain embodiments, the health assessment tool comprises one or more probe for detecting an amount of one or more microorganisms disclosed herein. In certain embodiments, the health assessment tool comprises a microarray of one or more probe for detecting an amount of one or more microorganism disclosed herein. In certain embodiments, the probe comprises a nucleic acid probe for detecting a signature gene of a microorganism disclosed herein. In certain embodiments, the probe detects a 16S rRNA sequence of a microorganism disclosed herein, e.g., a 16S rRNA sequence having at least about 80% (e.g., at least about 85%, at least about 90%, or at least about 95%, at least about 96%, at least about 97%, at least about 98%, at least about 99%, at least about 99.5%, or at least about 99.9%) or 100% sequence identity to the nucleotide sequence of any one of SEQ ID NO: 1-26. In certain embodiments, the probe comprises an antibody, e.g., a monoclonal antibody. The antibody can bind to a surface protein/antigen of a microorganism disclosed herein, and can be, for instance, a naturally-occurring or synthetic antibody.

In certain embodiments, the amount of the microorganism is measured from a fecal sample of the subject (e.g., a companion animal, such as a dog). In certain embodiments, the health assessment tool monitoring intestinal health status or dysbiosis by comparing the amount of the one or more microorganism with a reference amount of the one or more microorganism.

In certain embodiments, the health assessment tool comprises probes for detecting at least about 1, at least about 2, at least about 3, at least about 4, at least about 5, at least about 6, at least about 7, at least about 8, at least about 9, at least about 10, at least about 12, at least about 14, at least about 26 or more microorganisms disclosed herein. In certain embodiments, the health assessment tool comprises probes for detecting about 1, about 2, about 3, about 4, about 5, about 6, about 7, about 8, about 9, about 10, about 12, about 14, or about 26 microorganisms disclosed herein. In certain embodiments, the health assessment tool comprises probes for detecting between about 1 to about 500, between about 1 to about 100, between about 1 to about 26, between about 5 to about 100, between about 5 to about 26, between about 10 to about 26, between about 15 to about 50, or between about 50 to about 100 microorganisms disclosed herein.

In certain embodiments, the one or more microorganism comprises a bacterium comprising a 16S rRNA comprising a nucleotide sequence that is at least about 80% (e.g., at least about 85%, at least about 90%, or at least about 95%, at least about 96%, at least about 97%, at least about 98%, at least about 99%, at least about 99.5%, or at least about 99.9%) homologous or identical to any sequence in Table 5 (e.g., any one of SEQ ID NO: 27-293).

In certain embodiments, the one or more microorganism comprises a bacterium comprising a 16S rRNA comprising a nucleotide sequence having at least about 80% (e.g., at least about 85%, at least about 90%, or at least about 95%, at least about 96%, at least about 97%, at least about 98%, at least about 99%, at least about 99.5%, or at least about 99.9%) or 100% sequence identity to the nucleotide sequence of any of SEQ ID NOs: 1-26.

In particular embodiments, the one or more microorganism comprises a bacterium comprising a 16S rRNA comprising a nucleotide sequence having at least about 80% (e.g., at least about 85%, at least about 90%, or at least about 95%, at least about 96%, at least about 97%, at least about 98%, at least about 99%, at least about 99.5%, or at least about 99.9%) or 100% sequence identity to the nucleotide sequence of any of SEQ ID NOs: 1-14.

In other embodiments, the one or more microorganism comprises a bacterium comprising a 16S rRNA comprising a nucleotide sequence having at least about 80% (e.g., at least about 85%, at least about 90%, or at least about 95%, at least about 96%, at least about 97%, at least about 98%, at least about 99%, at least about 99.5%, or at least about 99.9%) or 100% sequence identity to the nucleotide sequence of any of SEQ ID NOs: 15-26.

In certain embodiments, the bacterium is selected from the group consisting of denovo1184, denovo1244, denovo1696, denovo2407, denovo2451, denovo283, denovo3487, denovo4154, denovo4328, denovo4681, denovo498, denovo5338, denovo6995, denovo943, denovo1214, denovo1400, denovo1762, denovo2014, denovo2197, denovo2368, denovo3663, denovo4206, denovo4485, denovo6368, denovo7117 denovo4881 and any combination thereof.

### 3. Pet Food Products

The presently disclosed subject matter provides a pet food product for improving intestinal health in a subject. The subject can be a companion animal, e.g., a dog or a cat.

In certain embodiments, the pet food product comprises an effective amount of sugar beet pulp. In certain embodiments, the sugar beet pulp is raw sugar beet pulp. In certain embodiments, the sugar beet pulp is cooked or sterilized or included with an extruded or a processed product.

In certain embodiments, the sugar beet pulp is in the pet food product at a concentration between about 0.01% w/w and about 10% w/w, between about 0.1% w/w and about 5% w/w, between about 0.5% w/w and about 4% w/w, between about 0.5% w/w and about 3% w/w, between about 0.5% w/w and about 2% w/w, between about 0.5% w/w and about 1.5% w/w, between about 0.5% w/w and about 1.2% w/w, between about 0.5% w/w and about 1% w/w, between about 0.5% w/w and about 0.9% w/w, or between about 0.5% w/w and about 0.8% w/w. In certain embodiments, the sugar beet pulp is in the pet food product at a concentration between about 0.1% w/w and about 10% w/w, between about 0.1% w/w and about 5% w/w, between about 0.5% w/w and about 4% w/w, between about 0.8% w/w and about 3% w/w, between about 0.8% w/w and about 2% w/w, between about 0.8% w/w and about 1.5% w/w, between about 0.8% w/w and about 1% w/w, between about 1% w/w and about 10% w/w, between about 1% w/w and about 5% w/w, between about 2% w/w and about 5% w/w, or between about 1% w/w and about 2% w/w. In certain embodiments, the sugar beet pulp is in the pet food product at a concentration of about 0.8% w/w.

The pet food product can be fed to a companion animal, such as, but not limited to, a dog or a cat. In certain embodiments, the intake of sugar beet pulp of the companion animal is between about 0.5 g/day and about 90 g/day.

Additionally disclosed herein is a pet food product including an effective amount of an intestinal microorganism, e.g., any bacterium disclosed herein, that is associated to healthy intestine status in the companion animal. As used herein, the term “healthy” refers to a companion animal that has not been diagnosed with a disease that is known to affect the microbiome. Examples of such diseases include, but are not limited to, irritable bowel syndrome, ulcerative colitis, Crohn's and inflammatory bowel disease. Preferably, the healthy companion animal does not suffer from dysbiosis. Dysbiosis refers to a microbiome imbalance inside the body, resulting from an insufficient level of keystone bacteria (e.g., bifidobacteria, such as B. longum subsp. infantis) or an overabundance of harmful bacteria in the gut. Methods for detecting dysbiosis are well known in the art.

The effective amount of the intestinal microorganism disclosed herein in the pet food product refers to the amount necessary to improve immunity, digestive function, and/or decrease inflammation; to improve faeces quality, digestive health, immunity, digestive function, and/or decrease inflammation; to decrease the symptoms and/or reduce the likelihood of a digestive disorder and/or inflammation; to improve the microbiome, faeces quality, digestive health, immunity, digestive function, and/or decreasing inflammation; and/or to decrease the symptoms and/or reduce the likelihood of a digestive disorder and/or inflammation in the companion animal when ingested or consumed by the companion animal (e.g., dog). In certain embodiments, the intestinal microorganism is selected from the group consisting of any bacterium comprising a 16S rRNA comprising a nucleotide sequence having at least about 95% sequence identity to the nucleotide sequence of any of SEQ ID NOs: 1-14, and any combination thereof. In certain embodiments, the bacterium is selected from the group consisting of Lachnospiraceae sp., Faecalibacterium prausnitzii, Bacteroides plebeius, Holdemania [Eubacterium] biforme, Dorea sp., Ruminococcaceae sp., Bacteroides sp., Blautia sp., Erysipelotrichaceae sp., Lachnospiraceae sp., and any combination thereof. In certain embodiments, the bacterium is selected from the group consisting of Faecalibacterium prausnitzii, Bacteroides plebeius, Holdemania [Eubacterium] biforme and any combination thereof. In certain embodiments, the bacterium is selected from the group consisting of denovo1184, denovo1244, denovo1696, denovo2407, denovo2451, denovo283, denovo3487, denovo4154, denovo4328, denovo4681, denovo498, denovo5338, denovo6995, denovo943 and any combination thereof.

In certain embodiments, the bacterium comprised in the pet food product is in an amount between about 1 thousand CFU and about 100 trillion CFU. In certain embodiments, the bacterium is between about 1 thousand CFU and about 10 trillion CFU, between about 1 million CFU and about 1 trillion CFU, between about 100 million CFU and about 100 billion CFU, between about 1 billion CFU and about 1 trillion CFU, between about 1 billion CFU and about 100 billion CFU, between about 100 million CFU and about 100 billion CFU, between about 1 billion CFU and about 50 billion CFU, between about 100 million CFU and about 50 billion CFU, or between about 1 billion CFU and about 10 billion CFU. In certain embodiments, the bacterium comprised in the pet food product is at least about 1 thousand CFU, at least about 1 million CFU, at least about 10 million CFU, at least about 100 million CFU, at least about 1 billion CFU, at least about 10 billion CFU, at least about 100 billion CFU or more.

In certain embodiments, the pet food product further comprises an effective amount of sugar beet pulp. The effective amount of the sugar beet pulp refers to the amount necessary to improve immunity, digestive function, and/or decrease inflammation; to improve faeces quality, digestive health, immunity, digestive function, and/or decrease inflammation; to decrease the symptoms and/or reduce the likelihood of a digestive disorder and/or inflammation; to improve the microbiome, faeces quality, digestive health, immunity, digestive function, and/or decreasing inflammation; and/or to decrease the symptoms and/or reduce the likelihood of a digestive disorder and/or inflammation in the companion animal when ingested or consumed by the subject (e.g., the companion animal, such as a dog).

In certain embodiments, the pet food product is a dietary supplement, for example, applied on top of the pet food product, as a pet food topper or subsequently mixed throughout the product. In certain embodiments, the pet food product is a treat product or a chew or a kibble based treat or complementary product. In certain embodiments, the pet food product is a cat food product or a dog food product. In certain embodiments, the food product is a dog food product. In certain embodiments, the pet food product is a dry pet food product. In certain embodiments, the pet food product is a wet pet food product.

In certain embodiments, the sugar beet pulp in the wet pet food product is at a concentration between about 0.01% w/w and about 10% w/w, between about 0.1% w/w and about 5% w/w, between about 0.5% w/w and about 4% w/w, between about 0.5% w/w and about 3% w/w, between about 0.5% w/w and about 2% w/w, between about 0.5% w/w and about 1.5% w/w, between about 0.5% w/w and about 1.2% w/w, between about 0.5% w/w and about 1% w/w, between about 0.5% w/w and about 0.9% w/w, or between about 0.5% w/w and about 0.8% w/w. In certain embodiments, the sugar beet pulp is at a concentration between about 0.1% w/w and about 10% w/w, between about 0.1% w/w and about 5% w/w, between about 0.5% w/w and about 4% w/w, between about 0.8% w/w and about 3% w/w, between about 0.8% w/w and about 2% w/w, between about 0.8% w/w and about 1.5% w/w, between about 0.8% w/w and about 1% w/w, between about 1% w/w and about 10% w/w, between about 1% w/w and about 5% w/w, between about 2% w/w and about 5% w/w, or between about 1% w/w and about 2% w/w. In certain embodiments, the sugar beet pulp is at a concentration of about 0.8% w/w.

In certain embodiments, any of the pet food products disclosed herein can further comprise an additional active agent. Non-limiting examples of additional active agents that can be present within a formulation of the presently disclosed subject matter include a nutritional agent (e.g., amino acids, peptides, proteins, fatty acids, carbohydrates, sugars, nucleic acids, nucleotides, vitamins, minerals, etc.), a prebiotic, a probiotic, an antioxidant, and/or an agent that enhances the microbiome, improves gastrointestinal health and improves animal health.

In certain embodiments, the pet food product comprises one or more probiotic. In certain embodiments, the probiotic is an animal probiotic. In certain embodiments, the animal probiotic is a feline probiotic. In other embodiments, the animal probiotic is a canine probiotic. In certain embodiments, the probiotic is Bifidobacterium, Lactobacillus, lactic acid bacterium and/or Enterococcus. In certain embodiments, the probiotic is selected from the group consisting of any organism from lactic acid bacteria and more specifically from the following bacterial genera; Lactococcus spp., Pediococcus spp., Bifidobacterium spp. (e.g., B. longum B. bifidum, B. pseudolongum, B. animalis, B infantis), Lactobacillus spp. (e.g. L. bulgaricus, L. acidophilus, L. brevis, L casei, L. rhamnosus, L. plantarum, L. reuteri, L. fermentum, Enterococcus spp. (e.g. E. faecium), Prevotella spp., Fusobacterium spp, Alloprevotella spp, and any combination thereof. In certain embodiments, the probiotic is comprised in the pet food product that is administered to a companion animal, wherein the amount of probiotic administered to the companion animal is from about 1 colony forming unit (CFU) to about 100 billion CFUs per day for the maintenance of the GI microflora or the microbiome or gastrointestinal health. In certain embodiments, the probiotic is administered to a companion animal in an amount of from about 1 colony forming unit (CFU) to about 20 billion CFUs per day for the maintenance the GI microflora or the microbiome or gastrointestinal health. In certain embodiments, the probiotic is administered to a companion animal in an amount of from about 1 billion CFUs to about 20 billion CFUs per day for the maintenance of GI microflora. In certain embodiments, the probiotic is administered to a companion animal in amounts of from about 0.01 billion to about 100 billion live bacteria per day. In certain embodiments, the probiotic is administered to a companion animal in amounts of from about 0.1 billion to about 10 billion live bacteria per day. In certain embodiments, the probiotic is administered to a companion animal in amounts of from about 1×10⁴ CFU to 1×10¹⁴ CFU per day.

In further embodiments, an additional prebiotic can be included, such as fructooligosaccharides (FOS), xylooligosaccharides (XOS), galactooligosaccharides (GOS), glucans, galactans, arabinogalactan, inulin and/or mannooligosaccharides. The additional prebiotic can be administered via the pet food product in amounts sufficient to positively stimulate the microbiome or the GI microflora and/or cause one or more probiotic to proliferate in the companion animal.

In certain embodiments, the pet food product can further contain additional additives that are present in the pet food product in an amount that does not impair the purpose and effect provided by the presently disclosed subject matter. Examples of contemplated additives include, but are not limited to, substances that are functionally beneficial to improving health, substances with a stabilizing effect, organoleptic substances, processing aids, substances that enhance palatability, coloring substances, and substances that provide nutritional benefits. In certain embodiments, the stabilizing substances include, but are not limited to, substances that can increase the shelf life of the product. Such substances include, but are not limited to, preservatives, synergists and sequestrants, packaging gases, stabilizers, emulsifiers, thickeners, gelling agents, and humectants. Examples of emulsifiers and/or thickening agents include, but are not limited to, gelatin, cellulose ethers, starch, starch esters, starch ethers, and modified starches.

Other exemplary additives include additives for coloring, palatability, and nutritional purposes, such as colorants; iron oxide, sodium chloride, potassium citrate, potassium chloride, and other edible salts; vitamins; minerals; and flavoring. The amount of such additives in a product typically is up to about 5% (dry basis of the product).

The pet food products disclosed herein can be formulated as a dietary supplement for a companion animal. A dietary supplement can be, for example, a feed used with another feed to improve the nutritive balance or performance of the total. The dietary supplement can also be a composition that is fed undiluted as a supplement to other feeds, offered free choice with other parts of an animal's ration that are separately available, or diluted and mixed with an animal's regular feed to produce a complete feed. The AAFCO, for example, provides a discussion relating to supplements in the American Feed Control Officials, Incorp. Official Publication, p. 220 (2003). Supplements can be in various forms including, for example, powders, liquids, syrups, pills, tablets, or encapsulated compositions. Other forms of supplements are known to those of ordinary skill in the art.

The pet food products of the present disclosure can also be formulated as a treat. In certain embodiments, treats include, for example, compositions that are given to an animal to entice the animal to eat during a non-meal time. In certain embodiments, the pet food product is a treat for canines, such as a dog bone. Treats can be nutritional, wherein the product comprises one or more nutrients, and can, for example, have a composition as described above for food. Non-nutritional treats encompass any other treats that are non-toxic.

In certain embodiments, the intestinal microorganism and/or sugar beet pulp of the presently disclosed subject matter can be incorporated into the composition during the processing of the formulation, such as during and/or after mixing of other components of the product. Distribution of these components into the product can be accomplished by conventional means, which are known to those of ordinary skill in the art.

Furthermore, in some embodiments, the pet food products of the presently disclosed subject matter can be prepared in a canned or wet form using conventional companion animal food processes. In such embodiments, ground animal (e.g., mammal, poultry, and/or fish) proteinaceous tissues can be mixed with the other ingredients, such as milk fish oils, cereal grains, other nutritionally balancing ingredients, special purpose additives (e.g., vitamin and mineral mixtures, inorganic salts, cellulose and beet pulp, bulking agents, and the like); and water that sufficient for processing is also added. These ingredients are mixed in a vessel suitable for heating while blending the components. Heating of the mixture can be effected using any suitable manner, such as, for example, by direct steam injection or by using a vessel fitted with a heat exchanger. Following the addition of the last ingredient, the mixture is heated to a temperature range of from about 50° F. to about 212° F. Temperatures outside this range are acceptable but can be commercially impractical without use of other processing aids. When heated to the appropriate temperature, the material will typically be in the form of a thick liquid. The thick liquid is filled into cans. A lid is applied, and the container is hermetically sealed. The sealed can is then placed into conventional equipment designed to sterilize the contents. This is usually accomplished by heating to temperatures of greater than about 230° F. for an appropriate time, which is dependent on, for example, the temperature used and the composition.

In certain embodiments, pet food products of the presently disclosed subject matter can be prepared in a dry form using conventional processes. Dry ingredients used in dry pet food products include, for example, animal protein sources, plant protein sources, grains, etc., which are ground and mixed together. Moist or liquid ingredients, including fats, oils, animal protein sources, water, etc., can then be added to and mixed with the dry mix. The mixture can then processed into kibbles or similar dry pieces. In some embodiments, the pet food product is kibble. Kibble can be formed by using an extrusion process in which the mixture of dry and wet ingredients is subjected to mechanical work at a high pressure and temperature and forced through small openings and cut off into kibble by a rotating knife. The wet kibble can then be dried and optionally coated with one or more topical coatings, such as flavors, fats, oils, powders, and the like. In certain embodiments, kibble can also be made from the dough using a baking process, rather than extrusion, wherein the dough is placed into a mold before dry-heat processing.

In certain embodiments, treats of the presently disclosed subject matter can be prepared by, for example, an extrusion or baking process similar to those described above for dry food.

### 4. Treatment Methods and Health Assessment

The disclosure also provides for methods for enhancing or improving the microbiome, for improving intestinal health and/or treating an intestinal dysbiosis of a subject in need thereof. In certain embodiments, the subject is a companion animal, e.g., a dog or a cat. In certain embodiments, the method can improve immunity, digestive function and/or reduce dysbiosis of a companion animal.

Such methods include administering to the subject an effective amount of any pet food product disclosed herein. The method may also further include monitoring an intestinal microorganism in the companion animal, e.g., any one or more of the intestinal microorganisms disclosed herein. In certain embodiments, the intestinal microorganism is measured in a fecal sample of the subject. In certain other embodiments, the intestinal microorganism is measured in a sample from the intestines of the subject. In some embodiments, the subject is a companion animal, e.g., a dog.

In certain embodiments, the pet food product can be administered to a subject from about 20 times per day to once per day, from about 10 times per day to once per day, or from about 5 times per day to once per day. In certain embodiments, the pet food product can be administered to a subject once per day, twice per day, thrice per day, 4 times per day, 5 times per day, 6 times per day, 7 times per day, 8 times per day, 9 times per day, 10 or more times per day. In certain embodiments, the pet food product can be administered to a subject once per every two days, once per every three days, once per every four days, once per every five days, once per every six days, once a week, once per every two weeks, once per every three weeks, or once a month. In certain embodiments, the pet food product can be administered to an animal in a constant manner, e.g., where the animal grazes on a constantly available supply of the subject food product.

In certain embodiments, the dosage of the pet food product is between about 1 mg/kg body weight per day and about 5000 mg/kg body weight per day. In certain embodiments, the dosage of the pet food product is between about 5 mg/kg body weight per day and about 1000 mg/kg body weight per day, between about 10 mg/kg body weight per day and about 500 mg/kg body weight per day, between about 10 mg/kg body weight per day and about 250 mg/kg body weight per day, between about 10 mg/kg body weight per day and about 200 mg/kg body weight per day, between about 20 mg/kg body weight per day and about 100 mg/kg body weight per day, between about 20 mg/kg body weight per day and about 50 mg/kg body weight per day or any intermediate range thereof. In certain embodiments, the dosage of the pet food product is at least about 1 mg/kg body weight per day, at least about 5 mg/kg body weight per day, at least about 10 mg/kg body weight per day, at least about 20 mg/kg body weight per day, at least about 50 mg/kg body weight per day, at least about 100 mg/kg body weight per day, at least about 200 mg/kg body weight per day or more. In certain embodiments, the dosage of the pet food product is no more than about 5 mg/kg body weight per day, no more than about 10 mg/kg body weight per day, no more than about 20 mg/kg body weight per day, no more than about 50 mg/kg body weight per day, no more than about 100 mg/kg body weight per day, no more than about 200 mg/kg body weight per day, no more than about 500 mg/kg body weight per day or more.

In certain embodiments, the amount of the pet food product decreases over the course of feeding a companion animal. In certain embodiments, the concentration of the pet food product increases over the course of feeding a companion animal. In certain embodiments, the concentration of the pet food product is modified based on the age of the companion animal.

In certain non-limiting embodiments, the presently disclosed subject matter provides for a method for determining an intestinal health status in a companion animal in need thereof. In certain embodiments, the method comprises:

a) measuring a first amount of a first intestinal microorganism and/or a second amount of a second intestinal microorganism in the companion animal;

b) comparing the first amount of the first intestinal microorganism with a first reference amount of the first intestinal microorganism, and/or comparing the second amount of the second intestinal microorganism with a second reference amount of the second intestinal microorganism, wherein the reference amounts of the intestinal microorganisms are determined based on the amounts of the intestinal microorganisms in a plurality of healthy companion animals; and

c) determining the intestinal health status in the companion animal when the first amount of the intestinal microorganism is higher than the first reference amount of the first intestinal microorganism, and/or when the second amount of the second intestinal microorganism is lower than the second reference amount of the second intestinal microorganism.

In certain embodiments, the first intestinal microorganism is one or more bacterium comprising a 16S rRNA comprising a nucleotide sequence having at least about 95% sequence identity to the nucleotide sequence of any of SEQ ID NOs: 1-14.

In certain embodiments, the first intestinal microorganism is selected from the group consisting of denovo1184, denovo1244, denovo1696, denovo2407, denovo2451, denovo283, denovo3487, denovo4154, denovo4328, denovo4681, denovo498, denovo5338, denovo6995, denovo943 and any combination thereof. In certain embodiments, the first intestinal microorganism is selected from the group consisting of Faecalibacterium prausnitzii, Bacteroides plebeius, Holdemania [Eubacterium] biforme and any combination thereof.

In certain embodiments, the second intestinal microorganism is one or more bacterium comprising a 16S rRNA comprising a nucleotide sequence having at least about 95% sequence identity to the nucleotide sequence any of SEQ ID NOs: 15-26.

In certain embodiments, the second intestinal microorganism is selected from the group consisting of denovo1214, denovo1400, denovo1762, denovo2014, denovo2197, denovo2368, denovo3663, denovo4206, denovo4485, denovo6368, denovo7117, denovo4881, and any combination thereof.

In certain non-limiting embodiments, the presently disclosed subject matter provides for a method for treating an intestinal dysbiosis and/or improving intestinal health in a companion animal in need thereof. In certain embodiments, the method comprises:

a) measuring a first amount of one or more intestinal microorganism in the companion animal;

b) administering a treatment regimen to the companion animal for treating the intestinal disorder and/or improving intestinal health;

c) measuring a second amount of the intestinal microorganism in the subject after step b); and

d) determining an intestinal health status of the animal or responsiveness to the treatment regimen.

In certain embodiments, the method further comprises continuing administering the treatment regimen, when the second amount of the intestinal microorganism is changed compared to the first amount of the intestinal microorganism.

In certain embodiments, the first intestinal microorganism is one or more bacterium comprising a 16S rRNA comprising a nucleotide sequence having at least about 95% sequence identity to the nucleotide sequence of any of SEQ ID NOs: 1-14, and wherein step d) comprises continuing administering the treatment regimen, when the second amount of the intestinal microorganism is increased compared to the first amount of the intestinal microorganism.

In certain embodiments, the first intestinal microorganism is selected from the group consisting of denovo1184, denovo1244, denovo1696, denovo2407, denovo2451, denovo283, denovo3487, denovo4154, denovo4328, denovo4681, denovo498, denovo5338, denovo6995, denovo943 and any combination thereof. In certain embodiments, the first intestinal microorganism is selected from the group consisting of Faecalibacterium prausnitzii, Bacteroides plebeius, Holdemania [Eubacterium] biforme and any combination thereof.

In certain embodiments, the second amount of the intestinal microorganism is measured between about 7 days and about 14 days after step b). In certain embodiments, an amount of the intestinal microorganism is increased within about 21 days, within about 14 days, within about 12 days, within about 10 days, within about 7 days, within about 6 days, within about 5 days, within about 4 days, within about 3 days, within about 2 days, or within about 1 day after step b). In certain embodiments, an amount of the intestinal bacterium is increased within about 1 day to about 21 days, within about 1 days to about 14 days, within about 3 days to about 14 days, within about 5 days to about 14 days, within about 7 days to about 14 days, within about 10 days to about 14 days, or within about 7 days to about 21 days after step b).

In certain embodiments, the second intestinal microorganism is one or more bacterium comprising a 16S rRNA comprising a nucleotide sequence that is at least about 95% homologous or identical to any of SEQ ID NOs: 15-26, wherein step d) comprises continuing administering the treatment regimen, when the second amount of the intestinal microorganism is decreased compared to the first amount of the intestinal microorganism.

In certain embodiments, the second intestinal microorganism is selected from the group consisting of denovo1214, denovo1400, denovo1762, denovo2014, denovo2197, denovo2368, denovo3663, denovo4206, denovo4485, denovo6368, denovo7117, denovo4881 and any combination thereof.

In certain embodiments, the second amount of the intestinal microorganism is measured between about 7 days and about 14 days after step b). In certain embodiments, an amount of the intestinal microorganism is decreased within about 21 days, within about 14 days, within about 12 days, within about 10 days, within about 7 days, within about 6 days, within about 5 days, within about 4 days, within about 3 days, within about 2 days, or within about 1 day after step b). In certain embodiments, an amount of the intestinal bacterium is decreased within about 1 day to about 21 days, within about 1 days to about 14 days, within about 3 days to about 14 days, within about 5 days to about 14 days, within about 7 days to about 14 days, within about 10 days to about 14 days, or within about 7 days to about 21 days after step b).

In certain embodiments, the reference amount of an intestinal microorganism is a mean amount of the intestinal microorganism in a plurality of healthy companion animals. In certain embodiments, the reference amount of an intestinal microorganism is within about three standard deviations of a mean amount of the intestinal microorganism in a plurality of healthy companion animals. In certain embodiments, the reference amount of an intestinal microorganism is within about two standard deviations of a mean amount of the intestinal microorganism in a plurality of healthy companion animals. In certain embodiments, the reference amount of an intestinal microorganism is within about one standard deviation of a mean amount of the intestinal microorganism in a plurality of healthy companion animals.

In certain embodiments, the amount of an intestinal microorganism can be determined by any method known in the art. In certain embodiments, the method includes, but is not limited to, antibody-based detection methods detecting a protein/antigen associated with the microorganism, e.g., an enzyme-linked immunosorbent assay (ELISA), flow cytometry, western blot; and methods for detecting a 16s rRNA associated with the microorganism, e.g., real-time polymerase chain reaction (RT-PCR), quantitative polymerase chain reaction (qPCR), DNA sequencing and microarray analyses. In certain embodiments, the microarray comprises probes for detecting any of the intestinal microorganism disclosed herein.

In certain embodiments, the treatment regimen can be any treatment regimen of dysbiosis known in the art. In certain embodiments, the treatment regimen comprises a treatment method disclosed herein.

In certain embodiments, the amount of the intestinal bacterium is measured from a fecal sample of the subject.

### EXAMPLES

The presently disclosed subject matter will be better understood by reference to the following Example, which is provided as exemplary of the invention, and not by way of limitation.

### Example 1

### Introduction

Uncovering whether associations exist between the microbiome, faeces quality and dietary factors in dogs and if they do, uncovering the nature of such associations may be possible with the development of deep sequencing approaches. This knowledge is of interest to support the understanding of health and characteristics of a healthy microbiome as well as dietary manipulation of the microbiome towards a healthy gastrointestinal tract.

Three ingredients were each applied to a wet pet food chunk in gravy product previously identified for faeces quality upgrades. The digestive health ingredients included sugar beet pulp (0.8% w/w), cellulose (0.5% w/w) and pea protein (0.25% w/w). The three diets were compared to a commercial control diet containing 0.5% sugar beet pulp, in a feeding study in 24 dogs. Primary measures for the study included faeces quality and assessment of the fecal microbiota. Studies on faeces quality require the accurate determination of fecal form and therefore scales of faeces consistency are used for the assessment of faeces quality. One such scale is the WALTHAM faeces scoring system, which categorises fecal form according to a 17-point scale from 1, represented by hard and dry faeces to 5, watery diarrhoea (Moxham, 2001).

### Methods

### Study Design

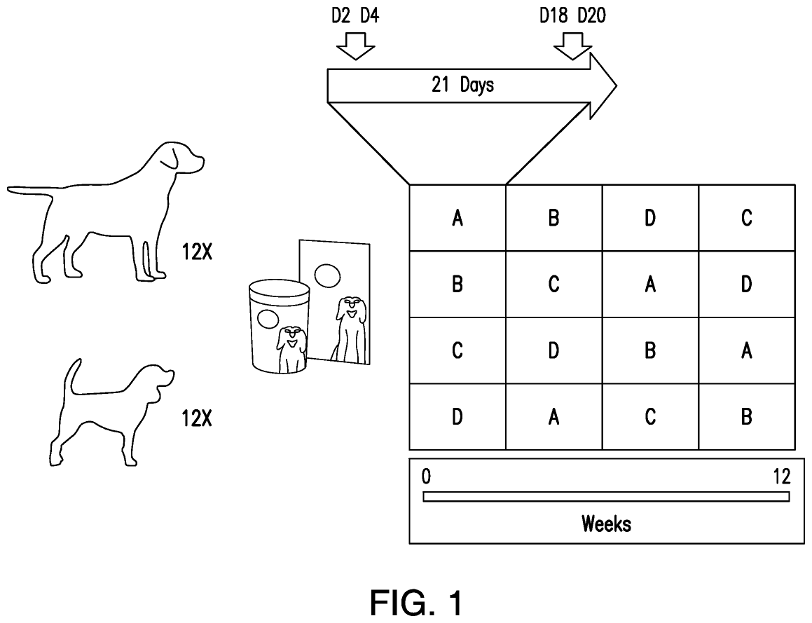

A cohort of 24 pair housed dogs were recruited. A wet pet food chunk in gravy pouch diet based on a commercial recipe and three diets comprising the same base recipe with increased levels of sugar beet pulp, cellulose or pea protein levels were produced for the study. Each diet was received by the dogs for a period of 21 days to allow for sufficient faeces quality data and for stabilisation of the gastrointestinal microbiota following the dietary change. Dogs were fed the commercial recipe diet and each of 3 test diets within a balanced Latin square study design to allow for sequence effects whereby each dog received each diet (FIG. 1).

Faeces quality scores, the number of defecations and fecal wet weight from defecations produced overnight were recorded daily and fresh fecal samples were collected in duplicate from each individual dog at the start of phase (days 2, 4[+1]) and the end of phase (days 18, 20 [+1]).

### Animals

The cohort comprised 12 Beagle dogs and 12 Labrador Retrievers. Animals were aged between 2.0 and 6.8 years (mean 4.16 years) at the start of the study. All dogs, with the exception of one female dog, were neutered. The study cohort had a sex split of 17 females and 7 males.

### Diet

Animals received their daily feed in 3 equal meals at standard times. All four diets were based on the same wet pet food chunk in gravy pouch product and included a reference and three test diets with altered sugar beet pulp, cellulose or pea protein levels. Each pouch contained approximately 100 g product.

Dogs were weaned from their dry format diets onto a standard commercial pouch format chunk in gravy Chicken & Veg diet (100 g pouch). Weaning was conducted over a 14 day period. Test diets were fed according a diet rotation to generate the balanced Latin square study design.

Dogs were allocated to one of four feeding groups 1, 2, 3 or 4 in triplicate pen pairs to form diet groups of six dogs (Table 2). Dogs within these groups received the diets in the same rotation order and hence also formed exclusive socialisation and exercise groups throughout the study period.

TABLE 2
Feeding groups and diet rotation across the study cohort

| Diet Rotation | Diet Phase1 | Diet Phase 2 | Diet Phase 3 | Diet Phase 4 | |
|---|---|---|---|---|---|
| Group | 17 Dec-6 Jan | 7 Jan-27 Jan | 28 Jan-17 Feb | 18 Feb-10 Mar | Dog Name |
| 1 | A | B | D | C | Oasis |
| 1 | A | B | D | C | Phoebe |
| 1 | A | B | D | C | Whitney |
| 1 | A | B | D | C | Hiccup |
| 2 | B | C | A | D | Bubbles |
| 2 | B | C | A | D | Wurzle |
| 2 | B | C | A | D | Krista |
| 2 | B | C | A | D | Bronte |
| 3 | C | D | B | A | Keisha |
| 3 | C | D | B | A | Merlin |
| 4 | D | A | C | B | Indigo |
| 4 | D | A | C | B | Bellatrix |
| 1 | A | B | D | C | Tortuga |
| 1 | A | B | D | C | Rosie |
| 2 | B | C | A | D | Dixie |
| 2 | B | C | A | D | Trinity |
| 3 | C | D | B | A | Daisy |
| 3 | C | D | B | A | Dorothy |
| 3 | C | D | B | A | Darcy |
| 3 | C | D | B | A | Dillon |
| 4 | D | A | C | B | Dash |
| 4 | D | A | C | B | Dora |
| 4 | D | A | C | B | Doodles |
| 4 | D | A | C | B | Deacon |

### Data Collection

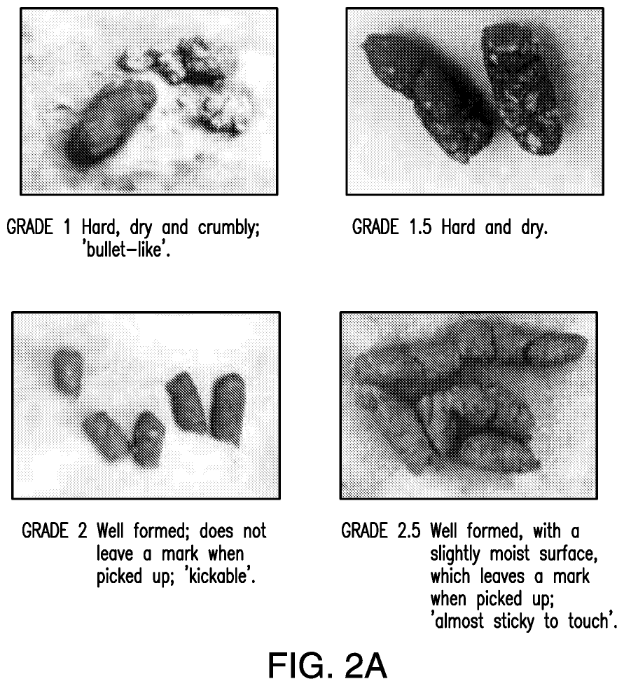

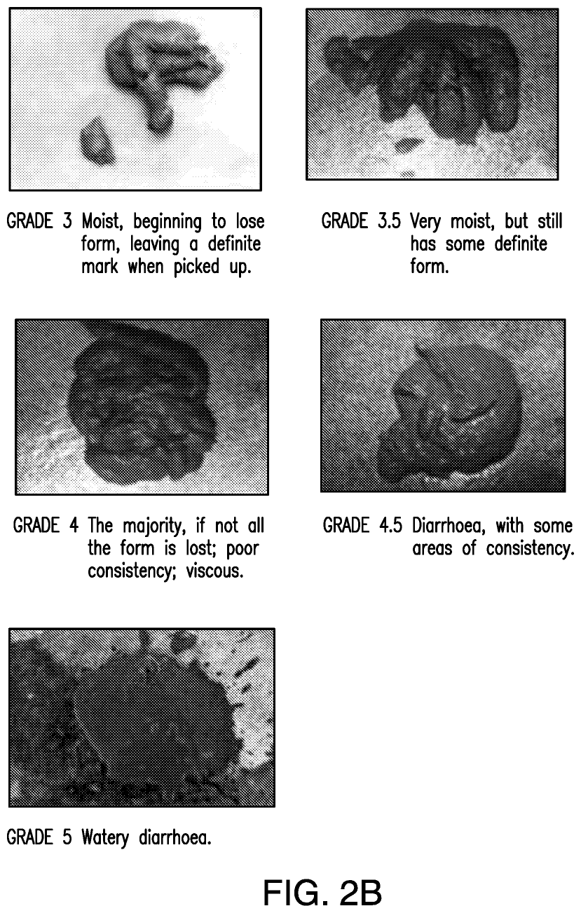

Throughout the study all overnight defecations were scored for faeces quality daily using a 17-point faeces quality scale and incidences of poor faeces (outside of the acceptable range 1.5-3.75) were recorded (FIGS. 2A and 2B). Faeces samples for assessment of the microbiome were collected on days 2, 4, 18 and 20 (or +1 if samples were not produced on the target days).

During the study, data on the following co-variates were collected for inclusion in analyses to establish whether differences in the microbiome were associated with adult, senior and geriatric life stages.
Daily and overnight faeces scores per pair*
Daily food intake
Bodyweight and body condition score

All faeces collected were scored using the 17-point faeces quality scale and incidences of poor faeces (outside of the acceptable range 1.5-3.75) were recorded.

### Faeces Sample Collection and Processing

Fresh fecal samples were collected with the samples collected most frequently representing the first defecation of the day to ensure the sample was secured. The majority of samples were freshly produced samples in grass paddocks or on walks. Samples were collected immediately, but no more than 15 minutes after defecation. Following collection, faeces were portioned into 3 aliquots of 100 mg and 2 aliquots of 400 mg faeces in sterile 2 ml Lo-Bind Eppendorf tubes. Samples were stored at −80 degrees centigrade

Faeces processing: a 100 mg portion of the faeces was processed to extract DNA from the faeces using the QIAamp Power Fecal DNA kit (Qiagen), which reflects manufacturers instructions. After DNA extraction, DNA concentrations achieved per sample were determined by standard nanodrop DNA quantification methods. Fecal DNA was then diluted 1:10 prior to preparation of Illumina high throughput DNA sequencing libraries by PCR amplification of the 16SrDNA locus (V4-6 region; Fadrosh et al., 2014). DNA sequencing was carried out using a Miseq Illumina system (chemistry v.3; 2×300 bp paired end sequencing) at a depth of 160 samples/run.

Quality thresholds of a minimum of 1,000 sequence reads per sample were defined and where sequence data did not reach this level it was removed from the analysis. Sequence data was de-noised to remove chimeras and was clustered into putative taxa based on 98% sequence identity. The resulting operational taxonomic unit (OTU) data was reduced to the non-rare portion through the removal of taxa representing <0.01% of the sequences in <2 animals from any one group. Following reduction to the non-rare portion of the population, the identification of OTUs based on a single taxon reference sequence selected as the most representative sequence of the cluster was analysed again. The sequences were used to interrogate the curated Greengenes (McDonald et al., 2012) and Silva (release 132; Yilmaz et al., 2014) databases to identify sequences in these databases with similarity criteria within 98% identity compared to the non-rare taxon reference sequences. Taxonomic assignments were then made based on sequence identity to the top database hit having first assessed the top hit against the top 10 hits resulting from database searches for each reference sequence. In the case of discrepancies between searches the Greengenes assignments were used.

Identification of group contrasts was based on the relative abundance of individual taxa detected compared to the total sequences within the sample.

### Statistical Methods

Prior to analysis rare OTUs in the data were grouped into a single pseudo-OTU. Non-rares (abundant sequences) were classified as OTUs with a proportion greater than 0.01% of the total sequences in at least 2 samples from any diet. The multivariate methods of multi-group principal components analysis (mgPCA), multiple factor analysis (MFA) and partial least squares discriminant analysis (PLSDA) were applied to the OTU data after converting to proportions, using +2 and +4 to the numerator and denominator, and then log 10 transforming.

mgPCA with no variance scaling was applied using animal ID as the grouping variable in order to stop particularly variable animal(s) from dominating the results.

MFA, from which the spider plot was created, was applied to reformatted data where the rows corresponded to diet and time and the columns were the OTU proportions in blocks corresponding to each animal with each block mean centred but not variance scaled.

PLSDA was applied to the OTU data with the response a combined diet and time variable and multilevel correction for each animal. The number of components was tuned using 3-fold cross validation and the influential OTUs selected by identifying those with a variable importance in projection (VIP) score greater than 1. The results of the PLSDA were visualised using a clustered image map.

Univariate analysis was performed using generalised linear mixed effects models with binomial error distributions and logit link functions applied to individual OTUs. The response of each model was OTU count+2 and total sample count−OTU count+4 (i.e. successes and failures). Fixed effects included were diet, time and their interaction and a random effect was animal to account for repeated measures. An observation level random effect was also included to account for overdispersion.

Univariate generalised linear mixed models with the same fixed and random structure described above were also fit to each Phylum, Family and Genus level, replacing the OTU count with the sum of the OTU counts within that level.

Shannon diversity and sample total reads were modelled using linear mixed effects models with diversity/total reads as the response, fixed effects of diet, time and their interaction and a random effect of animal to account for repeated measures.

From all models means were estimated, with 95% confidence intervals, for all diet and time combinations. The following contrasts were also performed,
Between diets at each time point
Between time points for each diet
Between diets between time points, i.e. the diet slopes

For the univariate analyses multiple comparisons correction was performed using the Benjamini-Hochberg procedure to maintain a false discovery rate of 5%.

All analyses were performed using R version 3.5.1 with the lme4, multcomp, optimx, FactoMineR (MFA), and mixOmics (mgPCA and PLSDA) libraries.

### Results

Analyses of Fecal Consistency with Diet

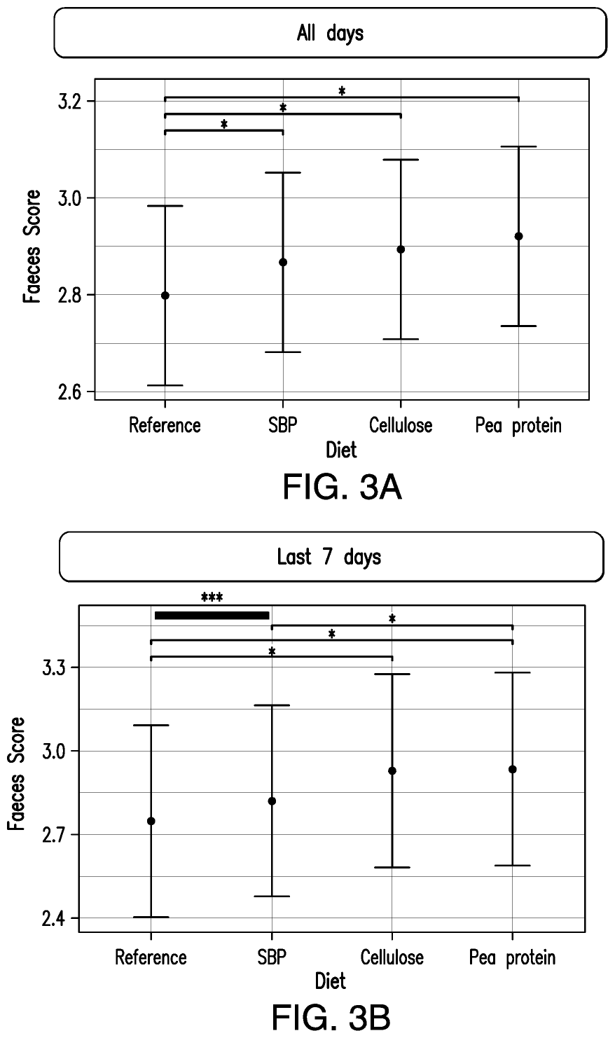

Analysis of faeces quality included assessment of faeces consistency (quality) score; the proportion of unacceptable faeces; number of defecations and overnight fecal wet weight. Analyses were conducted on the faeces scores produced by pen pairs overnight over the entire 21 day feeding phase to achieve a faeces scores throughout the phase. Additionally, the data was assessed for contrasts between groups within the last 7 days of the phase. For all of the diets containing the added digestive health ingredients (0.8% w/w sugar beet pulp, 0.5% w/w cellulose and 0.25% w/w pea protein), the group mean faeces scores were significantly increased from the optimal score of 2.5 compared to the commercial diet containing 0.5% sugar beet pulp (FIG. 3A). After a period of adjustment, when considering data from the last 7 days of the feeding phase, mean faeces quality scores for the reference control diet and SBP were numerically lower, and no significant difference was detected when dogs received the diet containing sugar beet pulp compared to the reference diet. Group mean faeces scores remained significantly higher having shifted away from the optimal faeces score for the cohort when they received the diets containing cellulose and pea protein even in the last 7 days (FIG. 3B).

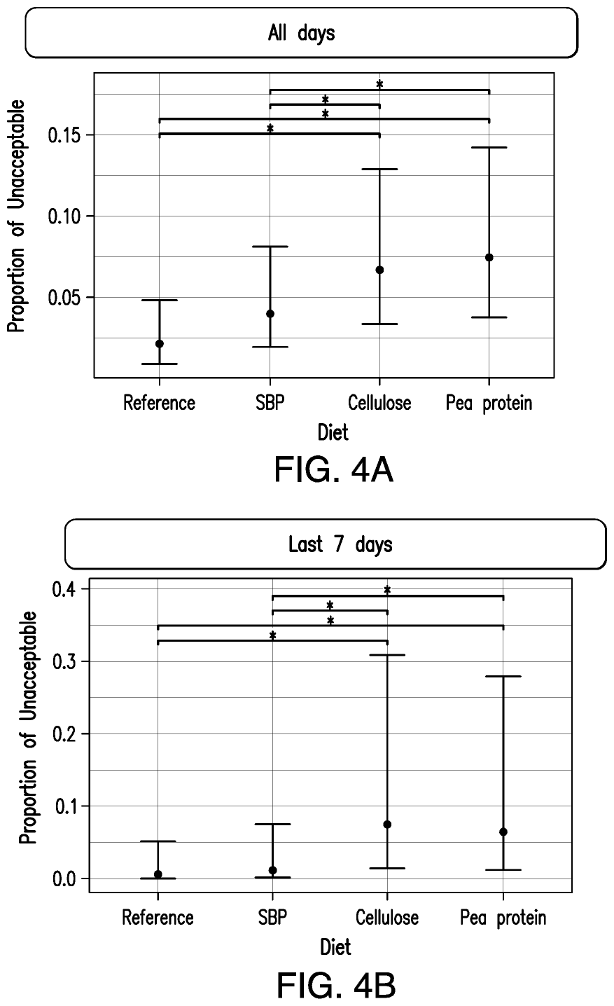

Throughout the study all unacceptable faeces were over the consistency score of 3.75 (i.e., diarrhoea), and no faeces with consistency scores of 1.5 (i.e., dry faeces) or below (unacceptable dry) were produced by the cohort. In line with the data describing mean faeces score, the dogs produced significantly lower numbers of defecations of 3.75 and above when receiving the reference diet compared to the diet containing cellulose and that containing pea protein. No significant difference in the proportion of unacceptable faeces was detected when the animals received the diet containing sugar beet pulp compared to the reference diet (FIGS. 4A and 4B). However, the proportion of unacceptable faeces over a consistency score of 3.75, consistent with diarrhea, was significantly increased compared to standard diet alone when supplemented with cellulose or with pea protein, but not with sugar beet pulp. When animals received the sugar beet pulp, the levels of diarrheal faeces were significantly reduced compared to the cellulose and pea protein. These same patterns were detected in the full 21 days as well as the last 7 days of feeding with very similar levels of unacceptable faeces for the animals, when they received the reference diet compared to the diet containing Sugar Beet Pulp.

Analyses of Microbial Correlations with Diet and in Dietary Change

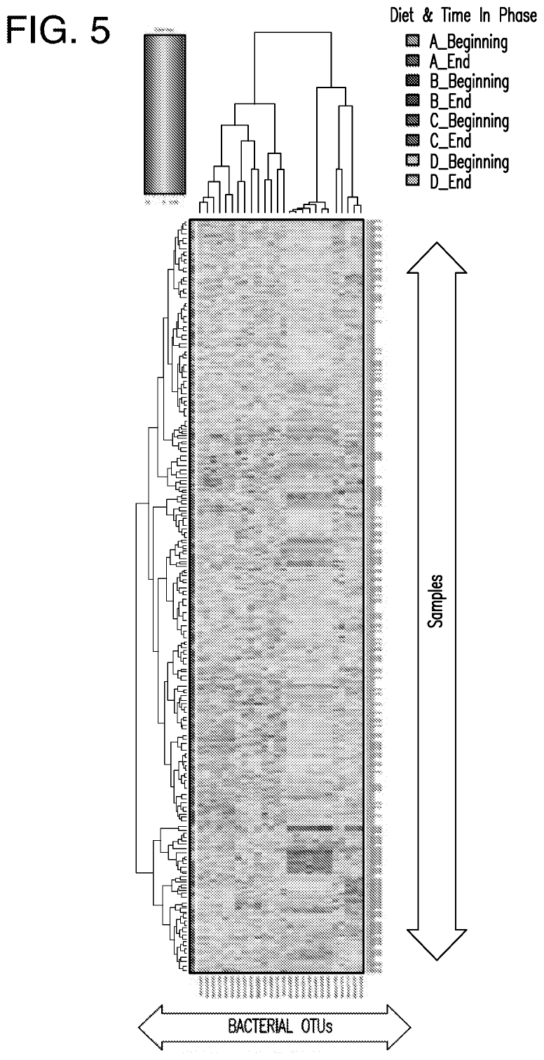

A total of 340 species level bacterial operational taxonomic units (OTUs) were identified. Abundance data for the 340 individual OTUs as well as the ‘rare’ group were analysed by partial least squares discriminate analysis (PLSDA) producing a correlation plot. When the bacterial taxa least influential in driving clustering of the samples were removed (variable importance in projection; VIP score <1), and the PLS-DA analysis was repeated, a subset of 26 taxa remained (FIG. 5). These correlation plots were suggestive that the composition of the microbiota in faeces were more similar when animals were fed the diets containing sugar beet pulp (0.5 and 0.8% w/w), while other clusters comprised samples produced by animals when they were fed the cellulose and pea protein diet. The subset of 26 bacterial taxa identified in the correlation plot contributed most strongly to the compositional differences when the animals received diets containing cellulose and pea protein compared to the diets containing sugar beet pulp. Of these 26 organisms more influential on the clustering of samples in the correlation plot, a subset of 14 OTUs appeared to produce a stronger signal (were represented in higher abundance and hence highlighted as a red signal on the correlation plot) while animals were fed the reference or the sugar beet pulp diet compared to the cellulose and the pea protein diets, yet were lower in abundance (blue signal) in a cluster enriched for beginning of phase samples across all diets (p=<0.01; lower cluster FIG. 3). These included species identified as Lachnospiraceae sp.; Faecalibacterium prausnitzii; Bacteroides plebeius; Holdemania [Eubacterium] biforme; Dorea sp.; Ruminococcaceae sp.; Bacteroides sp.; Blautia sp.; Bacteroides sp. 2; Erysipelotrichaceae sp.; Bacteroides sp. 3; Lachnospiraceae sp.; Bacteroides sp. 4 and Bacteroides sp. 5. Links are reported in the literature for several of these species with health in humans and cats and with the production of short chain fatty acids including butyrates. Health associations in humans, cats and other mammals are described for species from the families Lachnospiraceae, Erysipelotrichaceae and Ruminococcaceae as well as from the genera Dorea and Blautia, and for Faecalibacterium prausnitzii, Bacteroides plebeius and Holdemania [Eubacterium] biforme.

A second set of 12 organisms appeared to be higher in abundance in this cluster enriched for beginning of phase and were also lower in abundance while animals received the diets containing sugar beet pulp (Table 2). Most of these species appeared to be novel organisms from the order Clostridiales, and several were so novel as to not be identifiable beyond the Clostridales order through sequence similarity searches of public databases containing previously detected organisms. All 12 of these organisms were unidentified beyond the family level by comparison to known bacterial species and hence appear to be novel species from the canine gut.

### DISCUSSION

At the dose used, when added to a wet pet food chunk in gravy diet prior to processing, all of the ingredients previously reported to enhance faeces quality towards an optimal score actually resulted in a significant shift away from the optimal consistency compared to when the animals received the commercial diet with 0.5% sugar beet pulp. Of the ingredients used sugar beet pulp appeared to impact faeces scores to a lesser extent than the cellulose and pea protein at the inclusion levels used in the wet pet food chunk in gravy pet food background and after a period of adjustment (FIG. 4B) mean faeces score was not significantly different when the animals received the diets containing sugar beet pulp. No significant difference in the proportion of loose stools classified as diarrheal (>3.75) was observed between the diets containing sugar beet pulp at different levels, however significantly higher levels of diarrheic faeces were observed when the animals received the diets without the sugar beet pulp which contained the levels of cellulose and pea protein fed in these diets

Analysis of the fecal microbiota showed a composition more similar when the animals were fed the diets containing differing levels of sugar beet pulp than that observed when the animals received the diet containing cellulose or pea protein. A subset of 26 microbes were identified that were altered in relative abundance in the faeces when the animals received diets containing cellulose and pea protein compared to the diets containing sugar beet pulp. Several of the 26 bacterial species have previously been identified as having links with health in humans, cats and other mammals. These 26 organisms were also subject to greater changes in their abundance in the first 2-4 days after dietary change suggesting a potential microbial disbalance (dysbiosis) associated with transition between diets even with these relatively minor changes in nutritional intake. The signs of microbial dysbiosis observed mirrored the effects of the diets on the consistency of faeces output (faeces consistency) and the rate of unacceptable faeces with diets containing 0.5% sugar beet pulp supporting more solid faeces compared to the looser stools produced while animals received diets containing pea protein and cellulose. The dogs did not produce significantly higher mean faeces scores (looser stools) after a period of adjustment to the diet containing the higher level of sugar beet pulp (0.8%).

Since dietary change is frequently associated with transient loose stools, poor faeces quality or even diarrhoea, there is an interest in enabling the management of dietary transition. The findings suggest that diet transition can result in shifts in the bacterial composition in the gut, which can be linked to the observed poor faeces quality.

Furthermore, the data suggest that bacterial species detailed in Table 2 can be associated with more solid faeces and fewer incidences of diarrheal faeces in dogs while, those in Table 2 (novel species currently represented by DNA sequences) can represent species associated with poorer faeces quality and diarrhoea episodes in otherwise healthy dogs. Management of these and related bacterial species in the gut microbiome can be used as biological levers to control or reduce diarrheal episodes associated with dietary change or with diet associated to poor faeces quality/diarrhoea.

The bacterial species also represent organisms associated with gut health or healthy stool quality in dogs (Table 2) and reduced faeces quality or diarrheal faeces in otherwise healthy dogs (Table 3). As such the bacterial species represent putative markers of gut health (Table 2) or of poor faeces quality (Table 3) which can be used to assess gut health in healthy dogs.

TABLE 2
Bacterial taxa (OTUs) represented in PLSDA clusters with higher abundance on the diets
containing sugar beet pulp compared to cellulose and pea protein and over represented
at low abundances at the start of phase after diet change. (i.e. in dietary dysbiosis)

| OTU | Phylum | Class | Order | Family | Genus | Species |
|---|---|---|---|---|---|---|
| denovo1184 | Firmicutes | Clostridia | Clostridiales | Lachnospiraceae | Not identified | Not identified |
| denovo1244 | Firmicutes | Clostridia | Clostridiales | Ruminococcaceae | Faecalibacterium | prausnitzii |
| denovo1696 | Bacteroidetes | Bacteroidia | Bacteroidales | Bacteroidaceae | Bacteroides | plebeius |
| denovo2407 | Firmicutes | Erysipelotrichi | Erysipelotrichales | Erysipelotrichaceae | [Eubacterium] | biforme |
| denovo2451 | Firmicutes | Clostridia | Clostridiales | Lachnospiraceae | Dorea | Not identified |
| denovo283 | Firmicutes | Clostridia | Clostridiales | Ruminococcaceae | Not identified | |
| denovo3487 | Bacteroidetes | Bacteroidia | Bacteroidales | Bacteroidaceae | Bacteroides | |
| denovo4154 | Firmicutes | Clostridia | Clostridiales | Lachnospiraceae | Blautia | |
| denovo4328 | Bacteroidetes | Bacteroidia | Bacteroidales | Bacteroidaceae | Bacteroides | |
| denovo4681 | Firmicutes | Erysipelotrichi | Erysipelotrichales | Erysipelotrichaceae | Not identified | |
| denovo498 | Bacteroidetes | Bacteroidia | Bacteroidales | Bacteroidaceae | Bacteroides | |
| denovo5338 | Firmicutes | Clostridia | Clostridiales | Lachnospiraceae | Not identified | |
| denovo6995 | Firmicutes | Clostridia | Clostridiales | Not identified | Not identified | |
| denovo943 | Bacteroidetes | Bacteroidia | Bacteroidales | Bacteroidaceae | Bacteroides | |

TABLE 3
Bacterial taxa (OTUs) with represented in PLSDA clusters with lower abundance on
the diets containing sugar beet pulp compared to cellulose and pea protein and
over represented at higher abundances at the start of phase after diet change.

| OTU | Phylum | Class | Order | Family | Genus | Species |
|---|---|---|---|---|---|---|
| denovo1214 | Firmicutes | Clostridia | Clostridiales | [Mogibacteriaceae] | Not identified | |
| denovo1400 | Firmicutes | Clostridia | Clostridiales | Not identified | | |
| denovo1762 | Firmicutes | Clostridia | Clostridiales | Lachnospiraceae | | |
| denovo2014 | Firmicutes | Clostridia | Clostridiales | Not identified | | |
| denovo2197 | Firmicutes | Clostridia | Clostridiales | Clostridiaceae | | |
| denovo2368 | Firmicutes | Clostridia | Clostridiales | Not identified | | |
| denovo3663 | Firmicutes | Clostridia | Clostridiales | Not identified | | |
| denovo4206 | Firmicutes | Clostridia | Clostridiales | Peptostreptococcaceae | | |
| denovo4485 | Firmicutes | Clostridia | Clostridiales | Not identified | | |
| denovo6368 | Firmicutes | Clostridia | Clostridiales | Not identified | | |
| denovo7117 | Firmicutes | Clostridia | Clostridiales | Peptostreptococcaceae | | |
| denovo4881 | Firmicutes | Clostridia | Clostridia | Not identified | | |

TABLE 4

| Means of | | Taxonomy | | |
|---|---|---|---|---|
| signal ID | OTU | Phylum | Taxonomy Class | Taxonomy Order |
| Univariate | denovo1184 | p_Firmicutes | c_Clostridia | o_Clostridiales |
| significance | | | | |
| Univariate | denovo1214 | p_Firmicutes | c_Clostridia | o_Clostridiales |
| significance | | | | |
| Univariate | denovo1244 | p_Firmicutes | c_Clostridia | o_Clostridiales |
| significance | | | | |
| Univariate | denovo1393 | p_Bacteroidetes | c_Bacteroidia | o_Bacteroidales |
| significance | | | | |
| Univariate | denovo1577 | p_Firmicutes | c_Clostridia | o_Clostridiales |
| significance | | | | |
| Univariate | denovo1696 | p_Bacteroidetes | c_Bacteroidia | o_Bacteroidales |
| significance | | | | |
| Univariate | denovo1762 | p_Firmicutes | c_Clostridia | o_Clostridiales |
| significance | | | | |
| Univariate | denovo2049 | p_Fusobacteria | c_Fusobacteriia | o_Fusobacteriales |
| significance | | | | |
| Univariate | denovo2427 | p_Firmicutes | c_Clostridia | o_Clostridiales |
| significance | | | | |
| Univariate | denovo2451 | p_Firmicutes | c_Clostridia | o_Clostridiales |
| significance | | | | |
| Univariate | denovo2694 | p_Firmicutes | c_Clostridia | o_Clostridiales |
| significance | | | | |
| Univariate | denovo283 | p_Firmicutes | c_Clostridia | o_Clostridiales |
| significance | | | | |
| Univariate | denovo3487 | p_Bacteroidetes | c_Bacteroidia | o_Bacteroidales |
| significance | | | | |
| Univariate | denovo3832 | p_Firmicutes | c_Clostridia | o_Clostridiales |
| significance | | | | |
| Univariate | denovo4154 | p_Firmicutes | c_Clostridia | o_Clostridiales |
| significance | | | | |
| Univariate | denovo4328 | p_Bacteroidetes | c_Bacteroidia | o_Bacteroidales |
| significance | | | | |
| Univariate | denovo4477 | p_Bacteroidetes | c_Bacteroidia | o_Bacteroidales |
| significance | | | | |
| Univariate | denovo498 | p_Bacteroidetes | c_Bacteroidia | o_Bacteroidales |
| significance | | | | |
| Univariate | denovo5529 | p_Firmicutes | c_Clostridia | o_Clostridiales |
| significance | | | | |
| Univariate | denovo6038 | p_Bacteroidetes | c_Bacteroidia | o_Bacteroidales |
| significance | | | | |
| Univariate | denovo641 | p_Firmicutes | c_Clostridia | o_Clostridiales |
| significance | | | | |
| Univariate | denovo6639 | p_Proteobacteria | c_Epsilonproteobacteria | o_Campylobacterales |
| significance | | | | |
| Univariate | denovo6995 | p_Firmicutes | c_Clostridia | o_Clostridiales |
| significance | | | | |
| Univariate | denovo7324 | p_Bacteroidetes | c_Bacteroidia | o_Bacteroidales |
| significance | | | | |
| Univariate | denovo943 | p_Bacteroidetes | c_Bacteroidia | o_Bacteroidales |
| significance | | | | |
| Partial least | denovo1400 | p_Firmicutes | c_Clostridia | o_Clostridiales |
| squares only | | | | |
| Partial least | denovo2014 | p_Firmicutes | c_Clostridia | o_Clostridiales |
| squares only | | | | |
| Partial least | denovo2368 | p_Firmicutes | c_Clostridia | o_Clostridiales |
| squares only | | | | |
| Partial least | denovo2407 | p_Firmicutes | c_Erysipelotrichi | o_Erysipelotrichales |
| squares only | | | | |
| Partial least | denovo3663 | p_Firmicutes | c_Clostridia | o_Clostridiales |
| squares only | | | | |
| Partial least | denovo4206 | p_Firmicutes | c_Clostridia | o_Clostridiales |
| squares only | | | | |
| Partial least | denovo4485 | p_Firmicutes | c_Clostridia | o_Clostridiales |
| squares only | | | | |
| Partial least | denovo4681 | p_Firmicutes | c_Erysipelotrichi | o_Erysipelotrichales |
| squares only | | | | |
| Partial least | denovo4881 | p_Firmicutes | c_Clostridia | o_Clostridiales |
| squares only | | | | |
| Partial least | denovo6368 | p_Firmicutes | c_Clostridia | o_Clostridiales |
| squares only | | | | |
| Partial least | denovo7117 | p_Firmicutes | c_Clostridia | o_Clostridiales |
| squares only | | | | |

| Means of | | Taxonomy | Taxonomy |
|---|---|---|---|
| signal ID | Taxonomy Family | Genus | Species |
| Univariate | f_Lachnospiraceae | g_{—} | s_{—} |
| significance | | | |
| Univariate | f_[Mogibacteriaceae] | g_{—} | s_{—} |
| significance | | | |
| Univariate | f_Ruminococcaceae | g_Faecalibacterium | s_prausnitzii |
| significance | | | |
| Univariate | f_S24-7 | g_{—} | s_{—} |
| significance | | | |
| Univariate | f_Lachnospiraceae | g_{—} | s_{—} |
| significance | | | |
| Univariate | f_Bacteroidaceae | g_Bacteroides | s_plebeius |
| significance | | | |
| Univariate | f_Lachnospiraceae | g_{—} | s_{—} |
| significance | | | |
| Univariate | f_Fusobacteriaceae | g_Cetobacterium | s_somerae |
| significance | | | |
| Univariate | f_Ruminococcaceae | g_{—} | s_{—} |
| significance | | | |
| Univariate | f_Lachnospiraceae | g_Dorea | s_{—} |
| significance | | | |
| Univariate | f_{—} | g_{—} | s_{—} |
| significance | | | |
| Univariate | f_Ruminococcaceae | g_{—} | s_{—} |
| significance | | | |
| Univariate | f_Bacteroidaceae | g_Bacteroides | s_{—} |
| significance | | | |
| Univariate | f_Lachnospiraceae | g_{—} | s_{—} |
| significance | | | |
| Univariate | f_Lachnospiraceae | g_Blautia | s_{—} |
| significance | | | |
| Univariate | f_Bacteroidaceae | g_Bacteroides | s_{—} |
| significance | | | |
| Univariate | f_S24-7 | g_{—} | s_{—} |
| significance | | | |
| Univariate | f_Bacteroidaceae | g_Bacteroides | s_{—} |
| significance | | | |
| Univariate | f_Veillonellaceae | g_Megamonas | s_{—} |
| significance | | | |
| Univariate | f_S24-7 | g_{—} | s_{—} |
| significance | | | |
| Univariate | f_Lachnospiraceae | g_{—} | s_{—} |
| significance | | | |
| Univariate | f_Helicobacteraceae | g_Helicobacter | s_{—} |
| significance | | | |
| Univariate | f_{—} | g_{—} | s_{—} |
| significance | | | |
| Univariate | f_S24-7 | g_{—} | s_{—} |
| significance | | | |
| Univariate | f_Bacteroidaceae | g_Bacteroides | s_{—} |
| significance | | | |
| Partial least | f_{—} | g_{—} | s_{—} |
| squares only | | | |
| Partial least | f_{—} | g_{—} | s_{—} |
| squares only | | | |
| Partial least | f_{—} | g_{—} | s_{—} |
| squares only | | | |
| Partial least | f_Erysipelotrichaceae | g_[Eubacterium] | s_biformc |
| squares only | | | |
| Partial least | f_{—} | g_{—} | s_{—} |
| squares only | | | |
| Partial least | f_Peptostreptococcaceae | g_{—} | s_{—} |
| squares only | | | |
| Partial least | f_{—} | g_{—} | s_{—} |
| squares only | | | |
| Partial least | f_Erysipelotrichaceae | g_{—} | s_{—} |
| squares only | | | |
| Partial least | f_{—} | g_{—} | s_{—} |
| squares only | | | |
| Partial least | f_{—} | g_{—} | s_{—} |
| squares only | | | |
| Partial least | f_Peptostreptococcaceae | g_{—} | s_{—} |
| squares only | | | |

TABLE 5
16S rRNA sequences of intestinal bacteria

| | |
|---|---|
| OTU | >denovo1017 |
| Ref | |
| SEQ ID | CCTGTTCGCTCCCCCAGCTTTCGCGCCTCCGCGTCAGTGGCGGCCCAGAAGGCTGCCTTCGCCGTCGG |
| NO: 27 | TGTTCTTCCCGATATCTGCGCATTTCACCGCTACACCGGGAATTCCGCCTTCCCCTACCGCACTCGAT |
| | CCCCCCAGTTCGGCCCGCTTCCGGGGGTTGAGCCCCCGGCTTCGACGGTGCCGCTTGAAGGGACGCC |
| | TACGCGCGCTTTACGCCCAATGAATCCGGATAACGCTCGCCCCCTACGTATTACCGCGGCTGCTGGC |
| | ACGTAGTTAGCCGGGGCTTCTTCTGCAGGTACCGTCAACTTCTTCCCTGCTGAAAGCGGTTTACGACC |
| | CGAAGGCCTTCGTCCCGCACGCGGCGTTGCTGCGTCAGGGTTGCCCCCATTGCGCAAAATTCCCCA |
| OTU | >denovo1044 |
| Ref | |
| SEQ ID | CCTGTTTGCTCCCCACGCTTTCGTGCCTCAGCGTCAGTTATGGTCCAGAAAGCCGCCTTCGCTACTGG |
| NO: 28 | TGTTCCTTTGAATCTCTACGCATTTCACCGCTACACTCAAAGTTCCACTTTCCTCTCCCACACTCTAG |
| | CTCGGCAGTTTCCGAAGCAGCTCCGGGGTTGAGCCCCGAAATTTCACTTCAGACTTACCAAGCCGCCT |
| | ACGCACCCTTTACACCCAGTAAATCCGGATAACGCTTGCTCCCTACGTATTACCGCGGCTGCTGGCA |
| | CGTAGTTAGCCGGAGCTTCTTAGTTAGGTACCGTCATTTTTTCTTCCCTACTGATAGAAGTTTACAAT |
| | CCGAAGACCTTCTTCCTTCACGCGGCGTTGCTGCATCAGGCTTGCGCCCATTGTGCAATATTCCCCA |
| OTU | >denovo1049 |
| Ref | |
| SEQ ID | CCTGTTCGATCCCCACGCTTTCGTGCCTCAGCGTCAGTCAGGCGCCAGTACGCTGCCTTCGCAATCGG |
| NO: 29 | AGTTCTGCGCAATATCTAAGCATTTCACCGCTACACTGCGCATTCCGCGTACCTCTCGCCTTCTCTAG |
| | CACTGCAGTTTCAACGGCTCGCCGGGGTTGAGCCCCGGGCTTTTACCGCTGACTTGCATCGCCGCCTA |
| | CGCACCCTTTAAACCCAATAAATCCGGATAACGCTCGCATCCTCCGTATTACCGCGGCTGCTGGCAC |
| | GGAGTTAGCCGATGCTTTTTCTTCAGGTACACTCAGACTAGTATACATACTCGCTTTTGTTCCCTGAC |
| | AAAAGAGGTTTACGACCCATAGGGCCTTCCTCCCTCACGCGGCTTGGCTGGTTCAGGCTCCCGCCCA |
| | TTGACCAATATTCCTCA |
| OTU | >denovo1065 |
| Ref | |
| SEQ ID | CCTGTTTGCTACCCACGCTTTCGCGCTTTAGCGTCAGTATCTGTCCAGTAGGCTGGCTTCCCCATCGG |
| NO: 30 | CATTCCTACAAATATCTACGAATTTCACCTCTACACTTGTAGTTCCGCCTACCTCTCCAGTACTCTAGT |
| | TTGGCAGTTTCCAACGCAATACGGAGTTGAGCCCCGCATTTTCACATCAGACTTACCAAACCGCCTA |
| | GACGCGCTTTACGCCCAATAAATCCGGATAACGCTTGCGACATACGTATTACCGCGGCTGCTGGCAC |
| | GTATTTAGCCGTCGCTTCTTCTGTTGGTACCGTCACTTTCTTCTTCCCAACTGAAAGCACTTTACATTC |
| | CGAAAAACTTCATCGTGCACACAGAATTGCTGGATCAGACTTTTGGTCCATTGTCCAATATTCCCCA |
| OTU | >denovo108 |
| Ref | |
| SEQ ID | CCTGTTCGCTCCCCGCGCTCTCGCGCCTCAGCGTCAGTGTCCGTCCGGCGGGCCGCCTTCGCCTCCGG |
| NO: 31 | TGTTCCTCCTGGCCTCTGCGCATTTCACCGCTACGCCAGGAGTTCCGCCCGCCCCTCCGGCACTCCAG |
| | CCGCCCGGTCCGGGGCGCTTGCCCGGGGTTGGGCCCCGGCTTTTCACACCCCGCCCAGGCCGCCGCC |
| | TGCGCGCGCTTTACGCCCAGTGATTCCGGACAACGCTCGCCCCCCACGTGTTGCCGCGGCTGCTGGC |
| | ACGTGGTTGGCCGGGGCTTTCGTGCCGGGTTCAGTCATACCTTCCTCCCCGGCGGCGGGGCTTTACG |
| | GGCCTGGGCCCTTCATCGCCCACGCGGCATCGCTGCGTCAGAGTTGCCTCCATTGCGCAATATTCCCC |
| | A |
| OTU | >denovo1095 |
| Ref | |
| SEQ ID | CCTGTTTGCTCCCCACGCTTTCGAGCCTCAGTGTCAGTTACAGTCCAGTAAGCCGCCTTCGCCACTGG |
| NO: 32 | TGTTCCTCCTAATATCTACGCATTTCACCGCTACACTAGGAATTCCACTTACCTCTCCTGCACTCTAGT |
| | CTGACAGTTTCAAAGGCAGTCCCGGGGTTGAGCCCCGGGCTTTCACCTCTGACTTGTCAGACCACCT |
| | ACACTCCCTTTACACCCAGTAAATCCGGATAACGCTTGCACCATACGTATTACCGCGGCTGCTGGCA |
| | CGTATTTAGCCGGTGCTTCTTAGTCAGGTACCGTCACTATCTTCCCTGCTGATAGAGCTTTACATACC |
| | GAAATACTTCTTCACTCACGCGGCGTCGCTGCATCAGAGTTTCCTCCATTGTGCAATATTCCCCA |
| OTU | >denovo1102 |
| Ref | |
| SEQ ID | CCTGTTCGATACCCGCACTTTCGAGCTTCCGCGTCCGTTGCGCTCCAGTGAGCTGCCTTCGCAATCGG |
| NO: 33 | AGTTCTTCGTGATATCTAAGCATTTCACCGCTACACCACGAATTCCGCCCACTTTGTGCGTACTCAAG |
| | GAAACCAGTTCGCGCTGCAGTGCAGACGTTGAGCGTCTACATTTCACAACACGCTTAATCTCCGGCC |
| | TACGCTCCCTTTAAACCCAATAAATCCGGATAACGCCCGGACCTTCCGTATTACCGCGGCTGCTGGC |
| | ACGGAATTAGCCGGTCCTTATTCATAAGGTACATGCAAAAAGTCTCACGAGACTAACTTTATTCCCTT |
| | ATAAAAGCAGTTTACAACCCATAGGGCCGTCATCCTGCACGCTACTTGGCTGGTTCAGGCTCTCGCC |
| | CATTGACCAATATTCCTCA |
| OTU | >denovo1103 |
| Ref | |
| SEQ ID | CCTGTTTGATCCCCACGCTTTCGTGCTTCAGTGTCAGTTATGGTTTAGTAAGCTGCCTTCGCAATCGG |
| NO: 34 | AGTTCTGCGTGATATCTATGCATTTCACCGCTACACCACGCATTCCGCCTACCTCAAACACACTCAAG |
| | TAACCCAGTTTCAACGGCAATTTTATGGTTGAGCCACAAACTTTCACCGCTGACTTAAATCACCACCT |
| | ACGCACCCTTTAAACCCAATAAATCCGGATAACGCTCGCATCCTCCGTATTACCGCGGCTGCTGGCA |
| | CGGAGTTAGCCGATGCTTATTCATAGGGTACATACAAAAAAGGACACGTCCTCCACTTTATTCCCCT |
| | ATAAAAGAAGTTTACAAACCATAGATCCTTCTTCCTTCACGCGACTTGGCTGGTTCAGCCTCTCGGCC |
| | ATTGACCAATATTCCTCA |
| OTU | >denovo1150 |
| Ref | |
| SEQ ID | CCTGTTTGATCCCCGCACTTTCGAGCCTCAGCGTCAGTAGCACCCCGGATACCTGCCTTCGCGATCGG |
| NO: 35 | AGTTCTTCATGATATCTGAGCATTTCACCGCTACACCATGAATTCCAGTATCCCTGTGTGCACTCAAG |
| | ACTTCCAGTATCAACTGCAGTCCGACGGTTGAGCCGCCGCATTTCACAACTGACTTAAAAATCCGCC |
| | TGCGCTCCCTTTAAACCCAATAAATCCGGATAACGCCCGGACCTTCCGTATTACCGCGGCTGCTGGC |
| | ACGGAATTAGCCGGTCCTTTTTCTGATGGTACATACAAAACAACTCACGAGCTGCACTTTATTCCCAA |
| | CTAAAAGCAGTTTACAACCCGGAGGGCCGTCATCCTGCACGCTACTTGGCTGGTTCAGACTTGCGTC |
| | CATTGACCAATATTCCTCA |
| OTU | >denovo1184 |
| Ref | |
| SEQ ID | CCTGTTTGCTCCCCACGCTTTCGAGCCTCAACGTCAGTCTCTGTCCAGTAAGCCGCCTTCGCCACTGG |
| NO: 36 | TGTTCCTCCTAATATCTACGCATTTCACCGCTACACTAGGAATTCCACTTACCTCTCCAGTACTCTAG |
| | CTCAACAGTTTCCAAAGCAGTCCCGTGGTTAAGCCTCGGGCTTTCACTTCAGACTTGCCGAGCCGTCT |
| | ACGCTCCCTTTACACCCAGTAAATCCGGATAACGCTCGCCCCCTACGTATTACCGCGGCTGCTGGCA |
| | CGTAGTTAGCCGGGGCTTCTTAGTCAGGTACCGTCATTCTCTTCCCTGCTGATAGAGCTTTACGTACC |
| | GAAGTACTTCTTCACTCACGCGGCGTCGCTGCATCAGGGTTTCCCCCATTGTGCAATATCCCCCA |
| OTU | >denovo1208 |
| Ref | |
| SEQ ID | CCTGTTTGCTCCCCACGCTTTCGCGCCTGAGCGTCAGTGTCTGTCCGGCAGGCCGCCTTCGCCTCCGG |
| NO: 37 | TATTCCTCCTGATATCTGCGCATTTCACCGCTACACCAGGAATTCTGCCTGCCTCTCCAGCACTCGAG |
| | CCCTCCGGTTTGAGATGCTCTCCCGGGGTTGGGCCCCGGTCTTTAACACCTCACCTAAAAAGCCGCCT |
| | GCGCGCCCTTTACGCCCAGTAATTCCGGACAACGCTCGCCCCTCACGTCTTACCGCGGCTGCTGGCA |
| | CGTGATTGGCCGGGGCTTTCTTGCCGGGTACTGTCTTCTCTCTTCCCCGGCGGCAGAGCTTTACGGGA |
| | CGAATCCCTTCCTCACTCACGCGGCATCGCTGCGTCAGAGTTGCCTCCATTGCGCAATATTCCCCA |
| OTU | >denovo1210 |
| Ref | |
| SEQ ID | CCTGTTTGCTCCCCACGCTTTCGCACCTGAGCGTCAGTGTCTGTCCGGCAGGCCGCCTTCGCCTCCGG |
| NO: 38 | TATTCCTCCTGATATCTGCGCATTTCACCGCTACACCAGGAATTCTGCCTGCCTCTCCAGCACTCTAG |
| | CTGTGCGGTTCGGGGTGTCCTCTAAGGGTTGGGCCCTCATCTTTTACATCCCCGCCTGCTACAGCCGC |
| | CTGCGTGCCCTTTACGCCCAGTGATTCCGGACAACGCTCGCCCCCTACGTGTTACCGCGGCTGCTGGC |
| | ACGTAGTTAGCCGGGGCTTTCTCGCCAGGTACTGTCTTACTCATCCCTGGCAACAGAGCTTTACGGG |
| | ACGAATCCCTTCTTCACTCACGCGGCATCGCTGCGTCAGAGTTCCCTCCATTGCGCAATATTCCCCA |
| OTU | >denovo1214 |
| Ref | |
| SEQ ID | CCTGTTTGCTCCCCACGCTTTCGTACCTCAGTGTCAGTTACAGTCCAGAAAGCCGCCTTCGCCACTGG |
| NO: 39 | TGTTCCTCCTAATATCTACGCATTTCACCGCTACACTAGGAATTCCGCTTTCCTCTCCTGCACTCAAGT |
| | TTGCCAGTTCGCAGGGCGAACAATGGTTGAGCCATTGCCTTAAACCCTGCGCTTGGTAAACCACCTA |
| | CGTACCCTTTACGCCCAATAATTCCGGATAACGCTTGCCCCCTCCGTATTACCGCGGCTGCTGGCACG |
| | GAGTTAGCCGGGGCTTCCTCCAAGGGTACCGTCATTTGTTTCTTCCCCAAGGACAGAGCTTTACGACC |
| | CAAAGGCCTTCATCGCTCACGCGGCGTTGCTGCATCAGGCTTGCGCCCATTGTGCAATATTCCCCA |
| OTU | >denovo1228 |
| Ref | |
| SEQ ID | CCTATTTGCTCCCCACGCTTTCGTGCCTGAGCGTCAGCAACAGTCCAGGCGGCCGCCTTCGCCACTGG |
| NO: 40 | TGTTCTTCCATATATCTACGCATTTTACCGCTACACATGGAGTTCCACCGCCCTCTCCTGTCCTCGAGC |
| | GCGCCAGTTTCCAAAGCCTGTACAGGTTGAGCCTGTACCTTTCACTTCAGACTTGACGCGCCGCCTGC |
| | GCACCCTTTACGCCCAATCATTCCGGATAACGCTTGCCACCTACGTATTACCGCGGCTGCTGGCACGT |
| | AGTTAGCCGTGACTTTCTGGCGGGGTACCATCAAGAAGAAATCATTTCCTCTTCCTTCCCTTTTTCCC |
| | CCGCAACAGAGCTTTACGATCCGAAGACCTTCCTCACTCACGCGGCATTGCTCGTTCAGGCTTGCGC |
| | CCATTGACGAAAATTCCCTA |
| OTU | >denovo1237 |
| Ref | |
| SEQ ID | CCTGTTTGATACCCACACTTTCGAGCATGAACGTCAGTTACGGCTTAGTGTGCTGCCTTCGCAATCGG |
| NO: 41 | GGTTCTTCGTGATATCTAAGCATTTCACCGCTACACCACGAATTCCGCACACCTCAACCGCACTCAAG |
| | GACGCCAGTATCAACTGCAATTTTACGGTTGAGCCGCAAACTTTCACAGCTGACTTAACATCCCGTCT |
| | GCGCTCCCTTTAAACCCAATAAATCCGGATAACGCTCGCATCCTCCGTATTACCGCGGCTGCTGGCA |
| | CGGAGTTAGCCGATGCTTATTCATACGGTACATACAAGCTCCCACACGTGGGAGGTTTTATTCCCGT |
| | ATAAAAGAAGTTTACAATCCATAGAACCTTCATCCTTCACGCTACTTGGCTGGTTCAGGCTCTCGCCC |
| | ATTGACCAATATTCCTCA |
| OTU | >denovo1244 |
| Ref | |
| SEQ ID | CCTGTTTGCTACCCACACTTTCGAGCCTCAGCGTCAGTTGGTGCCCAGTAGGCCGCCTTCGCCACTGG |
| NO: 42 | TGTTCCTCCCGATATCTACGCATTCCACCGCTACACCGGGAATTCCGCCTACCTCTGCACTACTCAAG |
| | AAAAACAGTTTTGAAAGCAGTTTATGGGTTGAGCCCATAGATTTCACTTCCAACTTGTCTTCCCGCCT |
| | GCGCTCCCTTTACACCCAGTAATTCCGGACAACGCTTGTGACCTACGTTTTACCGCGGCTGCTGGCAC |
| | GTAGTTAGCCGTCACTTCCTTGTTGGGTACCGTCATTATCTTCCCCAACAACAGGAGTTTACAATCCG |
| | AAGACCTTCTTCCTCCACGCGGCGTCGCTGCATCAGGGTTTCCCCCATTGTGCAATATTCCCCA |
| OTU | >denovo1291 |
| Ref | |
| SEQ ID | CCTGTTCGCTCCCCCAGCTTTCGCGCCTCAGCGTCGGTCTCGGCCCAGAGGGCCGCCTTCGCCACCGG |
| NO: 43 | TGTTCCTCCCGATATCTGCGCATTCCACCGCTACACCGGGAATTCCACCCTCCCCTACCGGACCCGAG |
| | CCGCGGGGTTCGGGGGGCGGACCGGGGTTGAGCCCCGGGATTTGACCCCCCGCCTACGCGGCCGCCT |
| | ACGCGCGCTTTACGCCCAATGAATCCGGATAACGCTCGCCCCCTACGTATTACCGCGGCTGCTGGCA |
| | CGTAGTTAGCCGGGGCTTCTTCTGCAGGTACCGTCTTGACTCGTCCCTGCTGAAAGCGGTTTACGACC |
| | CGAAGGCCTTCGTCCCGCACGCGGCGTCGCTGCGTCAGGGTTGCCCCCATTGCGCAAGATTCCCCA |
| OTU | >denovo1310 |
| Ref | |
| SEQ ID | CCTGTTTGATACCCACACTTTCGAGCATCAGTGTCAGTTACAGTCCAGTGAGCTGCCTTCGCAATCGG |
| NO: 44 | AGTTCTTCGTGATATCTAAGCATTTCACCGCTACACCACGAATTCCGCCCACCTCTACTGCACTCAAG |
| | AAAACCAGTATCAACTGCAATTTTACGGTTGAGCCGCAAACTTTCACAGGCTGACTTAATCTTCCAC |
| | CTACGCTCCCTTTAAACCCAATAAATCCGGATAACGCTCGGATCCTCCGTATTACCGCGGCTGCTGGC |
| | ACGGAGTTAGCCGATCCTTATTCATACGGTACATACAAAATTCCACACGTGGAACACTTTATTCCCGT |
| | ATAAAAGAAGTTTACAACCCATAGGGCAGTCATCCTTCACGCTACTTGGCTGGTTCAGACTCTCGTC |
| | CATTGACCAATATTCCTCA |
| OTU | >denovo1338 |
| Ref | |
| SEQ ID | CCTGTTTGCTCCCCACGCTTTCGAGCCTCAGCGTCAGTTACAGACCAGAGAGCCGCTTTCGCCACCGG |
| NO: 45 | TGTTCCTCCATATATCTACGCATTTCACCGCTACACATGGAATTCCACTCTCCCCTTCTGCACTCAAGT |
| | TTAACAGTTTCCAAAGCGAACAATGGTTAAGCCACTGCCTTTAACTTCAGACTTATTAAACCGCCTGC |
| | GCTCGCTTTACGCCCAATAAATCCGGACAACGCTCGGGACCTACGTATTACCGCGGCTGCTGGCACG |
| | TAGTTAGCCGTCCCTTTCTGGTAAGTTACCGTCACTGTGTGAACTTTCCACTCTCACACACATTCTTCT |
| | CTTACAACAGAGCTTTACGATCCGAAAACCTTCTTCACTCACGCGGCGTTGCTCGGTCAGGGTTGCCC |
| | CCATTGCCGAAGATTCCCTA |
| OTU | >denovo1347 |
| Ref | |
| SEQ ID | CCTGTTCGCTCCCCACGCTTTCGCACCTCAGCGTCAGTATTTGTCCAGCAAGCCGCCTTCGCCACTGG |
| NO: 46 | TGTTCCTCCTAATATCTACGCATTTCACCGCTACACTAGGAATTCCACTTGCCTCTCCAATACTCAAG |
| | TCTACCAGTTTCCAATGCACTTCACCGGTTGAGCCGGTACCTTTCACATCAGACTTAATAAACCGCCT |
| | ACGCGCCCTTTACGCCCAGTCATTCCGGACAACGCTCGTCCCCTACGTATTACCGCGGCTGCTGGCAC |
| | GTAGTTAGCCGGGACTTCCTCATTAGGTACCGTCATTATTCTTCCCTAATAACAGAGCTTTACGATCC |
| | GAAAACCTTCTTCACTCACGCGGTATTGCTGCGTCAGGGTTTCCCCCATTGCGCAATATTCCCCA |
| OTU | >denovo1361 |
| Ref | |
| SEQ ID | CCTGTTTGCTCCCCACGCTTTCGAGCCTCAACGTCAGTTACCGTCCAGTAAGCCGCCTTCGCCACTGG |
| NO: 47 | TGTTCCTCCTAATATCTACGCATTTCACCGCTACACTAGGAATTCCGCTTACCTCTCCGGCACTCTAG |
| | ACTGACAGTTTCCAATGCAGTCCAGGGGTTAAGCCCCTGCCTTTCACATCAGACTTGCCATTCCGTCT |
| | ACGCTCCCTTTACACCCAGTAAATCCGGATAACGCTTGCCCCTTACGTATTACCGCGGCTGCTGGCAC |
| | GTAATTAGCCGGGGCTTCTTAGTCAGGTACCGTCATTATCTTCCCTGCTGATAGAAGTTTACATACCG |
| | AGATACTTCTTCCTTCACGCGGCGTCGCTGCATCAGGGTTTCCCCCATTGTGCAATATTCCCCA |
| OTU | >denovo1367 |
| Ref | |
| SEQ ID | CCTGTTTGATACCCACACTTTCGTGCCTCAGCGTCAGTCGCAGTCTGGCAAGCTGCCTACGCAATCGG |
| NO: 48 | AGTTCTTCGTGATATCTAAGCATTTCACCGCTACACCACGAATTCCGCCTGCCTCGTCTGAACTCAAG |
| | GACTCCAGTATCAACTGCAATTTTACGGTTGAGCCGCAAACTTTCACAACTGACTTAAAATCCCGCCT |
| | ACGCACCCTTTAAACCCAATAAATCCGGATAACGCTCGGATCCTCCGTATTACCGCGGCTGCTGGCA |
| | CGGAGTTAGCCGATCCTTATTCATACGGTACATACAGGCACCCACACGTGGATGATGTTATTCCCGT |
| | ATAAAAGAAGTTTACAACCCATAGGGCAGTCATCCTTCACGCTACTTGGCTGGTTCAGGCTCGCGCC |
| | CATTGACCAATATTCCTCA |
| OTU | >denovo1393 |
| Ref | |
| SEQ ID | CCTGTTCGATCCCCGCACTTTCGTGCCTCAGCGTCAGTTGAGCGCCGGCAGGCTGCCTTCGCAATCGG |
| NO: 49 | GGTTCTGCGTGATATCTATGCATTTCACCGCTACACCACGCATTCCGCCTGCTTCTCGCCCACTCAAG |
| | GCTGCCAGTTTCAACGGCCGGACGGGGTTGAGCCCCGAATTTTTACCGCTGACTTAACAGTCCGCCT |
| | ACGCACCCTTTAAACCCAATAAATCCGGATAACGCTCGCATCCTCCGTATTACCGCGGCTGCTGGCA |
| | CGGAGTTAGCCGATGCTTTTTCTCCGGGTACTCTCCTAATCAGGACACGTCCCGATTCTTGCTCCCCG |
| | GCAAAAGAGGTTTACGACCCGTAGGGCCTTATTCCCTCACGCGACTTGGCTGGTTCAGGCTTCCGCC |
| | CATTGACCAATATTCCTCA |
| OTU | >denovo1400 |
| Ref | |
| SEQ ID | CCTGTTCGCTCCCCACGCTTTCGCACCTCAGTGTCAGTATAAGCCTGGCAGACCGCCTTCGCCTCCGG |
| NO: 50 | TATTCCTCCTGATCTCTGCGCATTTCACCGCTACACCAGGAATTCTGCCTGCCTCGACAATACTCCAG |
| | CTGCCCGGTTTGCGGTGACCTCCCAGGGTTGAGCCCTGGACTTTTACACCGCACCTAAACAACCACC |
| | TGCGTGCCCTTTACGCCCAATAATTCCGGATAACGCTCGCCCCCTACGTATTACCGCAGCTGCTGGCA |
| | CGTAGTTGGCTGGGGCTTGCTTACCGGGTACCGTCATCGTCTTCCCCGGTAACAGAGCTTTACAGAA |
| | CGAATCCCTTCTTCACTCACGCGGCATCGCTGCGTCAGAGTTGCCTCCATTGCGCAATATTCCCCA |
| OTU | >denovo1461 |
| Ref | |
| SEQ ID | CCTGTTTGCTACCCATGCTTTCGAGCCTCAGCGTCAGTTAGTGCCCAGTAGGCCGCCTTCGCCACTGG |
| NO: 51 | TGTTCCTCCCGATATCTACGCATTCCACCGCTACACCGGGAATTCCGCCTACCTCTACACCACTCAAG |
| | ATCTGCAGTTTTGAAAGCAGTCCATAGGTTGAGCCCATGGTTTTCACTTCCAACTTGCAAACCCGCCT |
| | GCGCTCCCTTTACACCCAGTAATTCCGGACAACGCTTGCCACCTACGTTTTACCGCGGCTGCTGGCAC |
| | GTAGTTAGCCGTGGCTTTCTTGTAAGGTACCGTCATTATCGTCCCTTACGACAGAAGTTTACAACCCG |
| | AAGGCCTTCTTCCTTCACGCGGCGTCGCTGCATCAGGGTTTCCCCCATTGTGCAATATTCCCCA |
| OTU | >denovo1532 |
| Ref | |
| SEQ ID | CCCGTTCGCTCCCCTGGCTTTCGAGCCTCAGTGTCAGTTACAGTCCAGAAAGCCGCCTTCGCCACTGG |
| NO: 52 | TGTTCCTCCTAATATCTACGCATTTCACCGCTACACTAGGAATTCCGCTTTCCTCTCCTGCACTCAAG |
| | AAAAGTAGTTTCAATCCCCTCACGGGGTTGAGCCCCGCACTTTTAAGACTGACTTACTTTCCCACCTG |
| | CGCTCCCTTTACGCCCAATAATTCCGGACAACGCTCGCCACCTACGTATTACCGCGGCTGCTGGCAC |
| | GTAGTTAGCCGTGGCTTCCTCGAAAGGTACCGTCCTACAAAGCTCATTTCCTAGCCTTGCCTCTCGTC |
| | CCTCTCAACAGAGCTTTACGATCCGAAAACCTTCTTCACTCACGCGGCGTTGCTCCGTCAGGCTTGCG |
| | CCCATTGCGGAAGATTCCCCA |
| OTU | >denovo1539 |
| Ref | |
| SEQ ID | GCCTGTTCGATCCCCGCACTTTCGTGCCTCAGCGTCAGTTGAGCGCCGGTAAGCTGCCTTCGCAATCG |
| NO: 53 | GAGTTCTGCGTGATATCTATGCATTTCACCGCTACACCACGCATTCCGCCTACTTCTCGCTCACTCAA |
| | GGCTCCCAGTTTCAACGGCTCTACGGCGTTGAGCACCGCAATTTTACCGCTGACTTAAGAGCCCGCC |
| | TACGCACCCTTTAAACCCAATAAATCCGGATAACGCTCGCATCCTCCGTATTACCGCGGCTGCTGGC |
| | ACGGAGTTAGCCGATGCTTTTTCTCCGGGTACTCTCCAATCTGGGACACGTCCCAGATCTTGCTCCCC |
| | GGCAAAAGAGGTTTACGACCCATAGGGCCTTATTCCCTCACGCGACTTGGCTGGTTCAGGCTTTCGC |
| | CCATTGACCAATATTCCTCA |
| OTU | >denovo1568 |
| Ref | |
| SEQ ID | CCTGTTCGCTACCCACGCTCTCGTGCTTCAGCGTCAGTAACAGTCTGGTAAGCTGCCTTCGCTATCGG |
| NO: 54 | AGTTCTAAGTTATATCTAAGCATTTCACCGCTACTTAACTTATTCCGCCTACCTCGTCTGTACTCAAG |
| | CTGACCAGTATTAATGGCTGGCCAAGGTTTAGCCATGACATTTTACCACTAACTTAATCAACCGCCTA |
| | CGCACCCTTTAAACCCAATAAATCCGGATAACGCTCGCATCCTCCGTATTACCGCGGCTGCTGGCAC |
| | GGAGTTAGCCGATGCTTATTCATTCGGTACAGGCATCGTTCCACGCGTGAAACTTATTCTTCCCGAAT |
| | AAAAGAGGTTTACAACCCATAGGGCAGTCTTCCCTCACGCGACTTGGCTGGTTCAGCCTTCCGGCCA |
| | TTGACCAATATTCCTCA |
| OTU | >denovo1574 |
| Ref | |
| SEQ ID | CCTGTTCGCTCCCCCAGCTTTCGCGCCTCAGCGTCAGTGGTGGCCCAGAAGGCCGCCTTCGCCACCG |
| NO: 55 | GTGTTCTTCCCGATATCTGCGCATTCCACCGCTACACCGGGAATTCCGCCTTCCCCTACCACACTCGA |
| | GCCCGCCGGTATCGGAGGCTCGCCGGGGTTGAGCCCCGGGCTTTTACCTCCGACCTAACGGGCCGCC |
| | TACGCGCGCTTTACGCCCAATGAATCCGGATAACGCTCGCCCCATACGTATTACCGCGGCTGCTGGC |
| | ACGTATTTAGCCGGGGCTTCTTCTGCAGGTACCGTCACCGTAGCGGCCTCGTCCCTGCTGAAAGCGG |
| | TTTACGACCCGAAGGCCTTCGTCCCGCACGCGGCGTCGCTGCGTCAGGGTTGCCCCCATTGCGCAAG |
| | ATTCCCCA |
| OTU | >denovo1577 |
| Ref | |
| SEQ ID | CCTGTTTGCTCCCCACGCTTTCGAGCCTCAACGTCAGTTACTGTCCAGCAAGCCGCCTTCGCCACTGG |
| NO: 56 | TGTTCCTCCTAATATCTACGCATTTCACCGCTACACTAGGAATTCCGCTTGCCTCTCCAGCACTCTAG |
| | CCCAACAGTTTCCAAAGCAGTTCCCGGGTTGAGCCCGGGGATTTCACTTCAGACTTGCTGTGCCGTCT |
| | ACGCTCCCTTTACACCCAGTAAATCCGGATAACGCTTGCCCCCTACGTATTACCGCGGCTGCTGGCAC |
| | GTAGTTAGCCGGGGCTTCTTAGTCAGGTACCGTCACTTTCTTCCCTGCTGATAGAGCTTTACATACCG |
| | AAATACTTCTTCACTCACGCGGCGTTGCTGCATCAGGGTTTCCCCCATTGTGCAATATTCCCCA |
| OTU | >denovo1603 |
| Ref | |
| SEQ ID | CCTGTTTGATACCCACACTTTCGAGCATCAACGTCAGTTACGCTCCAGCAAGCTGCCTTCGCAATCGG |
| NO: 57 | AGTTCTTCGTGATATCTAAGCATTTCACCGCTACACCACGAATTCCGCCTGCCTCTGCCGCACTCAAG |
| | AATGCCAGTTTCAACTGCAATTTTAAGGTTGAGCCCCAAACTTTCACAGCTGACTTAACATCCCGTCT |
| | ACGCTCCCTTTAAACCCAATAAATCCGGATAACGCTCGCATCCTCCGTATTACCGCGGCTGCTGGCA |
| | CGGAGTTAGCCGATGCTTATTCATAAGGTACATGCAAAACACTACACGTAATGTCTGTTATTCCCTTA |
| | TAAAAGAAGTTTACAATCCATAGAACCTTCATCCTTCACGCTACTTGGCTGGTTCAGGCTCTCGCCCA |
| | TTGACCAATATTCCTCA |
| OTU | >denovo161 |
| Ref | |
| SEQ ID | CCTGTTTGCTCCCCACGCTTTCGTGCCTCAGTGTCAGTTTCAGTCCAGAAAGCCGCCTTCGCCACTGG |
| NO: 58 | TGTTCCTCCTAATATCTACGCATTTCACCGCTACACTAGGAATTCCGCTTTCCTCTCCTGCACTCAAGT |
| | AGCCCAGTTCGCAGGGCATACAATGGTTGGGCCATTGCCTTAAACCCTGCGCTTAGGTTACCACCTA |
| | CGCACTCTTTACGCCCAATAATTCCGGATAACGCTTGCCCCCTACGTATTACCGCGGCTGCTGGCACG |
| | TAGTTAGCCGGGGCTTCCTCCTTAGGTACCGTCAGTTCTTCTTCCCTAAGGACAGAAGTTTACGACCC |
| | GAAGGCCTTCTTCCTTCACGCGGCGTTGCTGCATCAGGCTTTCGCCCATTGTGCAATATTCCCCA |
| OTU | >denovo164 |
| Ref | |
| SEQ ID | CCTGTTCGCTCCCCACGCTCTCGCGCCTCAGCGTCAGTGTCCGTCCGGCGGGCCGCCTTCGCCTCTGG |
| NO: 59 | TGTTCCTCCCGGCCTCTGCGCATTTCACCGCTACGCCGGGAGTTCCGCCCGCCCCTCCGGCACTCCAG |
| | CCGCTCGGTTCGGGGCGCTTTCCCGGGGTTGGGCCCCGGATTTTCACACCCCGCCCTAGCGGCCGCCT |
| | GCGCGCGCTTTACGCCCAGTGATTCCGGACAACGCTCGCCCCCCACGTATTGCCGCGGCTGCTGGCA |
| | CGTGGTTGGCCGGGGCTTTCGCGCCAGGTACCGTCTCTGCTCTTCCCTGGCTACGGGGCTTTACGGGC |
| | CGAGGCCCTTCCTCGCCCACGCGGCATCGCTGCGTCAGAGTTTCCTCCATTGCGCAATATTCCCCA |
| OTU | >denovo1663 |
| Ref | |
| SEQ ID | CCTGTTTGCTCCCCACGCTTTCGCGCCTCAGTGTCAGTTACAGACCAGGAAGCCGCCTTCGCCACTGG |
| NO: 60 | TGTTCCTCCATATCTCTACGCATTTCACCGCTACACATGGAATTCCACTTCCCTCTTCTGCACTCAAGT |
| | CGACCAGTTTCCAATGACCCTCCACGGTTAAGCCGTGGGCTTTCACATCAGACTTAATCAACCACCT |
| | GCGCGCTCTTTACGCCCAATAATTCCGGATAACGCTCGCCACCTACGTATTACCGCGGCTGCTGGCA |
| | CGTAGTTAGCCGTGGCTTTCTCATAAGGTACCGTCACACTCTAGCCATTTCCTACTAAAGTCGTTCTT |
| | CCCTTATAACAGAATTTTACAACCCGAAGGCCTTCATCATTCACGCGGCGTTGCTCGGTCAGGCTTTC |
| | GCCCATTGCCGAAGATTCCCTA |
| OTU | >denovo1696 |
| Ref | |
| SEQ ID | CCTGTTTGATACCCACACTTTCGAGCATGAACGTCAGTTACGGCTTAGTGTGCTGCCTTCGCAATCGG |
| NO: 61 | AGTTCTTCGTGATATCTAAGCATTTCACCGCTACACCACGAATTCCGCACACCTCAACCGCACTCAAG |
| | GACGCCAGTATCAACTGCAATTTTAAGGTTGAGCCCCAAACTTTCACAGCTGACTTAACGACCCGTC |
| | TGCGCTCCCTTTAAACCCAATAAATCCGGATAACGCTCGCATCCTCCGTATTACCGCGGCTGCTGGCA |
| | CGGAGTTAGCCGATGCTTATTCATAAGGTACATACAAGCTCCCACACGTGGGAGGTTTTATTCCCTTA |
| | TAAAAGAAGTTTACAATCCGTAGGACCTTCATCCTTCACGCTACTTGGCTGGTTCAGACTCTCGTCCA |
| | TTGACCAATATTCCTCA |
| OTU | >denovo176 |
| Ref | |
| SEQ ID | CCTGTTTGCTACCCACACTTTCGAGCCTCAGCGTCAGTTAGTGCCCAGTAGGCCGCCTTCGCCACTGG |
| NO: 62 | TGTTCCTCCCGATATCTACGCATTCCACCGCTACACCGGGAATTCCGCCTACCTCTGCACCACTCAAG |
| | AAAAGCAGTTTTGAAAGCAATTTACGGGTTGAGCCCGTAGATTTCACTTCCAACTTACCTTCCCGCCT |
| | GCGCTCCCTTTACACCCAGTAATTCCGGACAACGCTTGTGACCTACGTTTTACCGCGGCTGCTGGCAC |
| | GTAGTTAGCCGTCACTTTCTTGTAAGGTACCGTCATTATCGTCCCTTACAACAGGAGTTTACAATCCG |
| | AAGACCTTCTTCCTCCACGCGGCGTCGCTGCATCAGGGTTTCCCCCATTGTGCAATATTCCCCA |
| OTU | >denovo1762 |
| Ref | |
| SEQ ID | CCTGTTTGCTCCCCACGCTTTCGAGCCTCAACGTCAGTTACCGTCCAGTAAGCCGCCTTCGCCACTGG |
| NO: 63 | TGTTCCTCCTAATATCTACGCATTTCACCGCTACACTAGGAATTCCGCTTACCTCTCCGGCACTCAAG |
| | TCCCACAGTTTCCAATGCAATCCAGAAGTTGAGCCTCTGCCTTTCACATCAGACTTGCAGAACCGTCT |
| | ACGCTCCCTTTACACCCAGTAAATCCGGATAACGCTTGCCCCCTACGTATTACCGCGGCTGCTGGCAC |
| | GTAGTTAGCCGGGGCTTCTTAGTCAGGTACCGTCATTTTCTTCCCTGCTGATAGAGCTTTACATCACG |
| | AATGACTTCTTCACTCACGCGGCGTCGCTGCATCAGGGTTTCCCCCATTGTGCAATATTCCCCA |
| OTU | >denovo1781 |
| Ref | |
| SEQ ID | CCTGTTTGCTCCCCACGCTTTCGAGCCTCAACGTCAGTCATCGTCCAGTAAGCCGCCTTCGCCACTGG |
| NO: 64 | TGTTCCTCCTAATATCTACGCATTTCACCGCTACACTAGGAATTCCACTTACCTCTCCGACACTCTAG |
| | AAAAACAGTTTCCAATGCAGTCCCGGGGTTGAGCCCCGGGTTTTCACATCAGACTTGCCTCTCCGTCT |
| | ACGCTCCCTTTACACCCAGTAAATCCGGATAACGCTTGCACCATACGTATTACCGCGGCTGCTGGCA |
| | CGTATTTAGCCGGTGCTTCTTAGTCAGGTACCGTCATTTTCTTCCCTGCTGATAGAGCTTTACATACC |
| | GAAATACTTCATCGCTCACGCGGCGTCGCTGCATCAGGGTTTCCCCCATTGTGCAATATTCCCCA |
| OTU | >denovo1854 |
| Ref | |
| SEQ ID | CCTGTTTGCTCCCCTAGCTTTCGCGCCTGAGCGTCAGTCATGGCCCAGAAGGCCGCCTTCGCCACCGG |
| NO: 65 | TGTTCCTCCCGATATCTGCGCATTCCACCGCTACACCGGGAGTTCCGCCTTCCCCTACCAGACTCAAG |
| | CCTGCCGGTATCGAGAGCGGCCCGGGGTTGAGCCCCGGGATTAGACCCTCGACCTAACAGGCCGCCT |
| | ACGCGCGCTTTACGCCCAATGAATCCGGATAACGCTCGCCCCCTACGTGTTACCGCGGCTGCTGGCA |
| | CGTAGTTAGCCGGAGCTTCTTCTGCAGGTACCGTCAGCTTTCGCCCTCGTCCCTGCTGAAAGCGGTTT |
| | ACGATCCGAAGACCTTCATCCCGCACGCGGCGTCGCTGCGTCAGGGTTGCCCCCATTGCGCAAGATT |
| | CCCCA |
| OTU | >denovo1908 |
| Ref | |
| SEQ ID | CCTATTTGCTCCCCACGCTTTCGTGCCTCAGCGTCAGCTGCAGGCCAGACGGCCGCCTTCGCCACTGG |
| NO: 66 | TGTTCCTCCATATATCTACGCATTTTACCGCTACACATGGAATTCCGCCGTCCTCTCCTGTGCTCCAGC |
| | TCCCCAGTTTCCAGGGCTCGCCATGGTTGAGCCATGGGCTTTTACCCCGGACTTAGGGAGCCGCCTG |
| | CGCACCCTTTACGCCCAATGATTCCGGATAACGCTCGCCACCTACGTATTACCGCGGCTGCTGGCAC |
| | GTAGTTAGCCGTGGCTTTCTGGCGGGCTACCGTCCTGCTCCGGGCATTTCCTCCCGCAGCCTCTCTTC |
| | GCCCGCAACAGAGCTTTACAGCCCGAAGGCCGTCTTCGCTCACGCGGCATTGCTCGGTCAGGGTTTC |
| | CCCCATTGCCGAAAATTCCCTA |
| OTU | >denovo191 |
| Ref | |
| SEQ ID | CCTGTTTGCTCCCCACGCTTTCGAGCCTCAGCGTCAGTTTCGGTCCAGCAAGCCGCCTTCGCCACTGG |
| NO: 67 | TGTTCCTCCTAATATCTATGCATTTCACCGCTACACTAGGAATTCCGCTTGCCTCTCCCGTACTCAAG |
| | CTATGCAGTTTTCCATGCAACCCCCAGGTTAAGCCCGGGTATTTCACATGAAACTTGCATTGCCGCCT |
| | ACGCTCTCTTTACGCCCAGTAATTCCGGACAACGCTCGCCCCCTACGTATTACCGCGGCTGCTGGCAC |
| | GTAGTTAGCCGGGGCTTTCTTCTATGGTACCGTCACTTCCTTCTTCCCATAGAACAGAATTTTACAAT |
| | CCTAAGACCTTCTTCATTCACGCGGCATTGCTGGGTCAGGCTTTCGCCCATTGCCCAATATTCCCCA |
| OTU | >denovo1910 |
| Ref | |
| SEQ ID | CCTGTTCGATACCCACGCTTTCGTGCCTCAGCGTCAGTAAGAGGTTGGTACGCTGCCTTCGCAATCGG |
| NO: 68 | AGTTCTGCGTGATATCTATGCATTTCACCGCTACACCACGCATTCCGCATACCTCACTTCCACTCTAG |
| | CCCGACAGTTTCAACGGCAGGCCGGGGTTGAGCCCCGACATTTGACCGCTGACTTGCCAGACAGCCT |
| | ACGCACCCTTTAAACCCAATAAATCCGGATAACGCTCGCATCCTCCGTATTACCGCGGCTGCTGGCA |
| | CGGAGTTAGCCGATGCTTATTCTTGCGGTACGCTCAACTGGCCACACGTGGCCGATTTGTTCCCGCAC |
| | TAAAGAAGTTTACAATCCATAGAACCTTCTTCCTTCACGCGACTTGGCTGGTTCAGGCTCGCGCCCAT |
| | TGACCAATATTCCTCA |
| OTU | >denovo1929 |
| Ref | |
| SEQ ID | CCTGTTTGCTCCCCACGCTTTCGTGCCTCAGCGTCAGTTATGGTCCAGAAAGCCGCCTTCGCTACTGG |
| NO: 69 | TGTTCCTTTGAATCTCTACGCATTTCACCGCTACACTCAAAGTTCCACTTTCCTCTCCCACACTCTAGC |
| | CTCTCAGTTTCGGTAGCAGCTCCGGGGTTGAGCCCCGAAATTTCACTTCCGACTTAAAAGGCCGCCT |
| | ACGCACCCTTTACACCCAGTAAATCCGGATAACGCTTGCTCCCTACGTATTACCGCGGCTGCTGGCA |
| | CGTAGTTAGCCGGAGCTTCTTAGTCAGGTACCGTCATTTCTTCTTCCCTGCTGATAGAAGTTTACAAT |
| | CCGAAGACCTTCTTCCTTCACGCGGCGTTGCTGCATCAGGCTTTCGCCCATTGTGCAATATTCCCCA |
| OTU | >denovo1951 |
| Ref | |
| SEQ ID | CCTGTTTGCTCCCCACGCTTTCGCACCTCAGCGTCAGTATTTGCCCAGGAGACCGCCTTCGCCTCTGG |
| NO: 70 | TGTTCTTTCAGATATCTACGCATTCCACCGCTACACCTGAAATTCCATCTCCCTCTGCAATACTCTAG |
| | AAAGGCAGTTTAGAATGACCAACCCAGGTTGAGCCCGGGTATTTCACATCCTACTTACCTTTCCGCCT |
| | ACATGCCCTTTACGCCCAGTTATTCCGATTAACGCTTGCACCCTCCGTATTACCGCGGCTGCTGGCAC |
| | GGAGTTAGCCGGTGCTTCTTCTGTGGGTAACGTCAGTGTTTCCTCCCCACTGAAAGTGCTTTACAACC |
| | CGAAGGCCTTCTTCACACACGCGGCATGGCTGCATCAGGGTTTCCCCCATTGTGCAATATTCCCTA |
| OTU | >denovo1994 |
| Ref | |
| SEQ ID | CCTGTTTGCTCCCCACGCTTTCGAGCCTCAGTGTCAGTTACAGTCCAGTGAGCCGCCTTCGCCTCCGG |
| NO: 71 | TGTTCCTCCTAATATCTACGCATTTCACCGCTACACTAGGAATTCCGCTCACCCCTCCTGCACTCTAG |
| | CATGACAGTTCCAAAAGCAATCCCGCGGTTGAGCCCCGGGTTTTCACTTCTGGCTTGCCTTGCCACCT |
| | ACGCTCCCTTTACACCCAGTAAATCCGGATAACGCTTGCACCATACGTATTACCGCGGCTGCTGGCA |
| | CGTATTTAGCCGGTGCTTCTTAGTCAGGTACCGTCATTTTCTTCCCTGCTGATAGAGCTTTACATACC |
| | GAAATACTTCTTCACTCACGCGGCGTCGCTGCATCAGAGTTTCCTCCATTGTGCAATATTCCCCA |
| OTU | >denovo2 |
| Ref | |
| SEQ ID | CCTGTTTGCTACCCACGCTTTCGAGCCTCAGCGTCAGTTACAGCCCAGTAGGCCGCCTTCGCCACTGG |
| NO: 72 | TGTTCCTCCTAATATCTACGCATTTCACCGCTACACTAGGAATTCCGCCTACCTCTACTGCACTCAAG |
| | AGTGATAGTTTTGAACGCGACTATCAGTTAAGCCGATAGTTTAAACATTCAACTTGTCTCCCCGCCTA |
| | CGCTCCCTTTACACCCAGTAATTCCGGACAACGCTTGCCACCTACGTATTACCGCGGCTGCTGGCACG |
| | TAGTTAGCCGTGGCTTCCTCCTTGGGTACCGTCATTTTTTCGTCCCCAAAGACAGAGGTTTACAATCC |
| | GAAAACCGTCTTCCCTCACGCGGCGTCGCTGCATCAGGGTTTCCCCCATTGTGCAATATCCCCCA |
| OTU | >denovo2014 |
| Ref | |
| SEQ ID | CCTGTTCGCTCCCCGCGCTCTCGCGCCTCAGCGTCAGTGTCCGTCCGGCGGGCCGCCTTCGCCTCCGG |
| NO: 73 | TGTTCCTCCTGGCCTCTGCGCATTTCACCGCTACGCCAGGAGTTCCGCCCGCCCCTCCGGCACTCCAG |
| | CCGCCCGGTCCGGGGCGCTTGCCCGGGGTTGGGCCCCGGCTTTTCACACCCCGCCTAAGCGGCCGCC |
| | TGCGCGCGCTTTACGCCCAGTGATTCCGGACAACGCTCGCCCCCCACGTATTGCCGCGGCTGCTGGC |
| | ACGTGGTTGGCCGGGGCTTTCGCGCCGGGTTCAGTCATTTCTTCCTCCCCGGCTACGGGGCTTTACGG |
| | GCCGAGGCCCTTCGTCGCCCACGCGGCGTCGCTGCGTCAGAGTTCCCTCCATTGCGCAATATTCCCCA |
| OTU | >denovo2020 |
| Ref | |
| SEQ ID | CCTATTTGCTCCCCACGCTTTCGTGCCTCAGCGTCAGATGCAGGCCAGACGGCCGCCTTCGCCACCGG |
| NO: 74 | TGTTCTTCCATATATCTACGCATTTTACCGCTACACATGGAGTTCCACCGTCCTCTCCTGCTCTCCAGC |
| | CGGACAGTTTCCGCAGCCCCTGAAGGTTGAGCCTCCAGTTTTTACTGCGGACTTGCCCGGCCGCCTGC |
| | GCACCCTTTACGCCCAATCATTCCGGATAACGCTCGCCACCTACGTATTACCGCGGCTGCTGGCACGT |
| | AGTTAGCCGTGACTTCCTCGCCGGGTACCATCACTCAGAAAGCTTTCCACTCTTTCTGCCTTTTGTCC |
| | CCGGCAACAGAGCTTTACGATCCGAAGACCTTCCTCGCTCACGCGGCATTGCTCGTTCAGGGTTCCC |
| | CCCATTGACGAAAATTCCCTA |
| OTU | >denovo2049 |
| Ref | |
| SEQ ID | CCTGTTTGCTCCCCACGCTTTCGCGCTTCAGCGTCAGTATCTGTCCAGTGAGCTGACTTCTCCATCGG |
| NO: 75 | CATTCCTACAAATATCTACGAATTTCACCTCTACACTTGTAGTTCCGCCCACCTCTCCAGTACTCTAG |
| | AAAGACAGTTTCCAACGCAATACGGAGTTGAGCCCCGCATTTTCACATCAGACTTATCTTTCCGCCTA |
| | GACGCGCTTTACGCCCAATAAATCCGGATAACGCTTGCGACATACGTATTACCGCGGCTGCTGGCAC |
| | GTATTTAGCCGTCGCTTCTTCTGTTGGTACCGTCACTTTCTTCTTCCCAACTGAAAGCACTTTACAATC |
| | CGAAGACCTTCATCGTGCACACAGAATTGCTGGATCAGGCTTGTGGCCCATTGTCCAATATTCCCCA |
| OTU | >denovo2061 |
| Ref | |
| SEQ ID | CCTATTTGCTCCCCACGCTTTCGTGCCTGAGCGTCAGTAACAGTCCAGGCGGCCGCCTTCGCCACTGG |
| NO: 76 | TGTTCTTCCATATATCTACGCATTTTACCGCTACACATGGAGTTCCACCGCCCTCTCCTGTCCTCGAGC |
| | GTGACAGTTTCCAAAGCCTGTACAGGTTGAGCCCGTACCTTTCACTTCAGACTTGCCACGCCGCCTGC |
| | GCACCCTTTACGCCCAATCATTCCGGATAACGCTTGCCACCTACGTATTACCGCGGCTGCTGGCACGT |
| | AGTTAGCCGTGACTTTCTGGCAGGTTACTATCAAAAGAAAAGCATTTCCTCTTCCCTTCTTTTCTGAC |
| | CTGCAACAGAGCTTTACGATCCGAAGACCTTCCTCACTCACGCGGCATTGCTCGTTCAGGCTTGCGCC |
| | CATTGACGAAAATTCCCTA |
| OTU | >denovo2075 |
| Ref | |
| SEQ ID | CCTGTTCGATACCCGCACTTTCGAGCTTCAGCGTCAGTTGCGCTCCAGTGAGCTGCCTTCGCAATCGG |
| NO: 77 | AGTTCTTCGTGATATCTAAGCATTTCACCGCTACACCACGAATTCCGCCCACTTTGTGCGTACTCAAG |
| | GAAACCAGTTCGCGCTGCAGTTCAGATGTTGAGCATCTACATTTCACAACACGCTTAATCTCCGGCCT |
| | ACGCTCCCTTTAAACCCAATAAATCCGGATAACGCCCGGACCTTCCGTATTACCGCGGCTGCTGGCA |
| | CGGAATTAGCCGGTCCTTATTCATAAGGTACATGCAAAAAGTCTCACGAGACTCACTTTATTCCCTTA |
| | TAAAAGCAGTTTACAACCCATAGGGCCGTCTTCCTGCACGCTACTTGGCTGGTTCAGACTCTCGTCCA |
| | TTGACCAATATTCCTCA |
| OTU | >denovo2085 |
| Ref | |
| SEQ ID | CCTGTTTGCTACCCATGCTTTCGAGCCTCAGCGTCAGTTAGTGCCCAGCAGGCCGCCTTCGCCACTGG |
| NO: 78 | TGTTCCTCCCGATATCTACGCATTCCACCGCTACACCGGGAATTCCGCCTGCCTCTGCACCACTCAAG |
| | ATCTGCAGTTTTGAATGCACCTATGGGTTGAGCCCATAGTTTAAACATCCAACTTGCAAACCCGCCTG |
| | CGCTCCCTTTACACCCAGTAATTCCGGACAACGCTTGCCACCTACGTATTACCGCGGCTGCTGGCACG |
| | TAGTTAGCCGTGGCTTCCTCCTCGGGTACCGTCATACTTCGTCCCCGAAGACAGAGGTTTACAATCCG |
| | AAAATCTTCTTCCCTCACGCGGCGTCGCTGCATCAGAGTTTCCTCCATTGTGCAATATTCCCCA |
| OTU | >denovo2114 |
| Ref | |
| SEQ ID | CCTGTTCGCTACCCATGCTTTCGAGCCTCAGCGTCAGTTGCAGACCAGAGAGCCGCCTTCGCCACTG |
| NO: 79 | GTGTTCTTCCATATATCTACGCATTCCACCGCTACACATGGAGTTCCACTCTCCTCTTCTGCACTCAA |
| | GTTCAACAGTTTCTGATGCAATTCTCCGGTTGAGCCGAAGGCTTTCACATCAGACTTATTGAACCGCC |
| | TGCACTCGCTTTACGCCCAATAAATCCGGACAACGCTTGCCACCTACGTATTACCGCGGCTGCTGGC |
| | ACGTAGTTAGCCGTGACTTTCTAAGTAATTACCGTCAAATAAAGGCCAGTTACTACCTCTATCTTTCT |
| | TCACTACCAACAGAGCTTTACGAGCCGAAACCCTTCTTCACTCACGCGGCGTTGCTCCATCAGACTTT |
| | CGTCCATTGTGGAAGATTCCCTA |
| OTU | >denovo219 |
| Ref | |
| SEQ ID | CCTGTTTGCTCCCCACGCTTTCGCACCTCAGCGTCAGTATCTTTCCAGCAGGCCGCCTTCGCCTCTGG |
| NO: 80 | TATTCCTCCTGAACTCTACGCATTTCACCGCTACTCCAGGAATTCTGCCTGCCTCTCCAATACTCTAGT |
| | CTACCAGTTCCGGGTGCTCACAGTGGTTGAGCCACTGCCTTTCACATCCAGCTTGATAAACCGCCTAC |
| | GTGCCCTTTACGCCCAGTCATTCCGGACAACGCTTGTCCCCTACGTATTACCGCGGCTGCTGGCACGT |
| | AGTTAGCCGGGACTTCATCTTAGGGTACCGTCACTTGCTTCTTCCCCTATGATAGAGCTTTACAACCC |
| | GAAGGCCTTCTTCACTCACGCGGTATTGCTGCGTCAGAGTTCCCTCCATTGCGCAATATTCCCCA |
| OTU | >denovo2197 |
| Ref | |
| SEQ ID | CCTGTTTGCTCCCCACGCTTTCGAGCCTCAGCGTCAGTTACAGTCCAGAGAATCGCCTTCGCCACTGG |
| NO: 81 | TGTTCTTCCTAATCTCTACGCATTTCACCGCTACACTAGGAATTCCATTCTCCTCTCCTGCACTCTAGA |
| | CTTCCAGTTTGAAATGCAGCACCCAAGTTGAGCCCGGGTATTTCACATCTCACTTAAAAGTCCGCCTA |
| | CGCTCCCTTTACGCCCAGTAAATCCGGACAACGCTCGCCACCTACGTATTACCGCGGCTGCTGGCAC |
| | GTAGTTAGCCGTGGCTTCCTCCTCAGGTACCGTCATTATCGTCCCTGAAGACAGAGCTTTACAACCCG |
| | AAGGCCGTCATCACTCACGCGGCGTTGCTGCATCAGGGTTTCCCCCATTGTGCAATATTCCCCA |
| OTU | >denovo2205 |
| Ref | |
| SEQ ID | CCTGTTTGCTCCCCACGCTTTCGAGCCTCAACGTCAGTTACCGTCCAGTAAGCCGCCTTCGCCACTGG |
| NO: 82 | TGTTCCTCCTAATATCTACGCATTTCACCGCTACACTAGGAATTCCGCTTACCCCTCCGGCACTCAAG |
| | CCTGGCAGTTTCCAATGCAGTCCAGGAGTTGAGCCCCTGCCTTTCACATCAGACTTGCCATGCCGTCT |
| | ACGCTCCCTTTACACCCAGTAAATCCGGATAACGCTTGCCCCCTACGTATTACCGCGGCTGCTGGCAC |
| | GTAGTTAGCCGGGGCTTCTTAGTCAGGTACCGTCATTATCTTCCCTGCTGATAGAAGTTTACATACCG |
| | AGATACTTCTTCCTTCACGCGGCGTCGCTGCATCAGGGTTTCCCCCATTGTGCAATATTCCCCA |
| OTU | >denovo2206 |
| Ref | |
| SEQ ID | CCTGTTTGCTCCCCACGCTTTCGTGCCTCAGCGTCAGTTACAGTCCAGAAAGCCGCCTTCGCTACTGG |
| NO: 83 | TGTTCCTCCCAATATCTACGCATTTCACCGCTACACTGGGAATTCCGCTTTCCTCTCCTGCACTCAAGT |
| | CAGACAGTATCAGGAGCTTACTACGGTTGAGCCGTAGCCTTTAACTCCTGACTTGAAAGACCGCCTA |
| | CGCACCCTTTACGCCCAGTAAATCCGGATAACGCTAGCCCCCTACGTATTACCGCGGCTGCTGGCAC |
| | GTAGTTAGCCGGGGCTTCCTCCTCAAGTACCGTCATTATCTTCCTTGAGGACAGAGCTTTACGACCCG |
| | AAGGCCTTCATCGCTCACGCGGCGTTGCTGCATCAGGCTTGCGCCCATTGTGCAATATTCCCCA |
| OTU | >denovo2247 |
| Ref | |
| SEQ ID | CCTGTTTGCTCCCCACGCTTTCGCGCCTCAGCGTCAGTTAATGTCCAGCAGGCCGCCTTCGCCACTGG |
| NO: 84 | TGTTCCTCCCAATATCTACGCATTTCACCGCTACACTGGGAATTCCGCCTGCCTCTCCATCACTCAAG |
| | ACTCGCAGTTTTGAAAGCAGTTTCGGGGTTAAGCCCCGAGATTTCACTTCCAACTTGCAAGCCCGCCT |
| | ACACGCCCTTTACACCCAGTAAATCCGGATAACGCTTGCTCCCTACGTATTACCGCGGCTGCTGGCA |
| | CGTAGTTAGCCGGAGCTTATTCTTCAGGTACCGTCATTTTCTTCGTCCCTGATTAAAGATTTTTACAAT |
| | CCGAAGACCTTCATCAATCACGCGGCGTTGCTGCGTCAGGGTTGCCCCCATTGCGCAATATTCCCCA |
| OTU | >denovo2275 |
| Ref | |
| SEQ ID | CCTGTTTGCTCCCCACGCTTTCGCTCCTCAGCGTCAGTTAATGTCCAGCAGGCCGCCTTCGCCACTGG |
| NO: 85 | TGTTCCTCCGTATATCTACGCATTTCACCGCTACACACGGAATTCCGCCTGCCTCTCCATCACTCAAG |
| | ACCAGCAGTTTTGAAAGCAGTTTGGGGGTTGAGCCCCCAGATTTCACTTCCAACTTACCAGCCCGCC |
| | TACTCGCCCTTTACACCCAGTAAATCCGGATAACGCTTGCTCCCTACGTATTACCGCGGCTGCTGGCA |
| | CGTAGTTAGCCGGAGCTTATTCTTTAGGTACCGTCAAATTTTCGTCCCTAATTAAAGATCTTTACAAC |
| | CCGAAGGCCTTCTTCAATCACGCGGCGTTGCTGCGTCAGGGTTTCCCCCATTGCGCAATATTCCCCA |
| OTU | >denovo2277 |
| Ref | |
| SEQ ID | CCTGTTTGCTCCCCACGCTTTCGAGCCTCAGCGTCATTGTCAGTCCAGTAAGCCGCCTTCGCCACTGG |
| NO: 86 | TGTTCCTCCTAATATCTACGCATTTCACCGCTACACTAGGAATTCCGCTTACCTCTCCTGAAATCTAG |
| | CCGCACAGTTTCAAAAGCAGTCCCGGAGTTAAGCCCCGGGCTTTCACTTCTGACTTGCGCTGCCGCCT |
| | ACGCTCCCTTTACACCCAGTAAATCCGGATAACGCTTGCCCCCTACGTATTACCGCGGCTGCTGGCAC |
| | GTAGTTAGCCGGGGCTTCTTAGTCAGGTACCGTCGTTATCTTCCCTGCTGATAGAAGTTTACATACCG |
| | AAATACTTCATCCTTCACGCGGCGTTGCTGCATCAGGCTTTCGCCCATTGTGCAATATTCCCCA |
| OTU | >denovo2283 |
| Ref | |
| SEQ ID | CCTGTTTGCTCCCCACGCTTTCGAGCCTCAACGTCAGTTATCGTCCAGTAAGCCGCCTTCGCCACTGG |
| NO: 87 | TGTTCCTCCTAATATCTACGCATTTCACCGCTACACTAGGAATTCCACTTACCCCTCCGACACTCTAG |
| | ACTGACAGTTTCCAATGCAGTCCCGGGGTTGAGCCCCGGGTTTTCACATCAGACTTGCCAGTCCGTCT |
| | ACGCTCCCTTTACACCCAGTAAATCCGGATAACGCTTGCCCCCTACGTATTACCGCGGCTGCTGGCAC |
| | GTAGTTAGCCGGGGCTTCTTAGTCAGGTACCGTCATTTTCTTCCCTGCTGATAGAGCTTTACATACCG |
| | AAATACTTCTTCGCTCACGCGGCGTCGCTGCATCAGGGTTTCCCCCATTGTGCAATATTCCCCA |
| OTU | >denovo2296 |
| Ref | |
| SEQ ID | CCTGTTCGCTACCCATGCTTTCGAGCCTCAGCGTCAGTTGCAGACCAGACAGCCGCCTTCGCCACTGG |
| NO: 88 | TGTTCTTCCATATATCTACGCATTCCACCGCTACACATGGAGTTCCACTGTCCTCTTCTGCACTCAAGT |
| | CGCCCGGTTTCCGATGCACTTCTTCGGTTAAGCCGAAGGCTTTCACATCAGACCTAAGCAACCGCCT |
| | GCGCTCGCTTTACGCCCAATAAATCCGGATAACGCTTGCCACCTACGTATTACCGCGGCTGCTGGCA |
| | CGTAGTTAGCCGTGACTTTCTGGTTGGATACCGTCACTGCGTGAACAGTTACTCTCACGCACGTTCTT |
| | CTCCAACAACAGAGCTTTACGAGCCGAAACCCTTCTTCACTCACGCGGTGTTGCTCCATCAGGCTTGC |
| | GCCCATTGTGGAAGATTCCCTA |
| OTU | >denovo2304 |
| Ref | |
| SEQ ID | CCTGTTTGCTCCCCACGCTTTCGAGCCTCAACGTCAGTCATCGTCCAGTAAGCCGCCTTCGCCACTGG |
| NO: 89 | TGTTCCTCCTAATATCTACGCATTTCACCGCTACACTAGGAATTCCGCTTACCTCTCCGACACTCTAG |
| | CCTGACAGTTCCAAATGCAGTCCCGGGGTTGAGCCCCGGGCTTTCACATCTGGCTTGCCATGCCGTCT |
| | ACGCTCCCTTTACACCCAGTAAATCCGGATAACGCTTGCCCCCTACGTATTACCGCGGCTGCTGGCAC |
| | GTAGTTAGCCGGGGCTTCTTAGTCAGGTACCGTCATTTTCTTCCCTGCTGATAGAGCTTTACATACCG |
| | AAATACTTCATCGCTCACGCGGCGTCGCTGCATCAGGGTTTCCCCCATTGTGCAATATTCCCCA |
| OTU | >denovo2368 |
| Ref | |
| SEQ ID | CCTGTTCGCTCCCCGCGCTCTCGCGCCTCAGCGTCAGTGTCCGTCCGGCGGGCCGCCTTCGCCTCCGG |
| NO: 90 | TGTTCCTCCCGGCCTCTGCGCATTTCACCGCTACGCCGGGAGTTCCGCCCGCCCCTCCGGCGCTCCAG |
| | CCGCCCGGTCCGGGGCGCTTGCCCGGGGTTGGGCCCCGGCTTTTCACACCCCGCCTAAGCGGCCGCC |
| | TGCGCGCGCTTTACGCCCAGTGATTCCGGACAACGCTCGCCCCCCACGTATTGCCGCGGCTGCTGGC |
| | ACGTGGTTGGCCGGGGCTTTCGTGCCGGGTTCAGTCATTTCCTTCCTCCCCGGCGGCGGGGCTTTACG |
| | GGCCTGGGCCCTTCATCGCCCACGCGGCGTCGCTGCGTCAGAGTTGCCTCCATTGCGCAATATTCCCC |
| | A |
| OTU | >denovo2370 |
| Ref | |
| SEQ ID | CCTGTTTGATACCCGCACCTTCGAGCCTCAGCGTCAGTAATGCCCCGGTACAATGCCTGCGCGATCG |
| NO: 91 | GAGTTCCTCGTGATATCTAAGCATTTCACCGCTACACCACGAATTCCATGTACCCTGTGCATACTCAA |
| | GGCTGCCAGTTTCAACGGCGGAACGCGGTTGAGCCGCGCCATTTGACCGCTGACTTGACTGCCCGCC |
| | TACGCTCCCTTTAAACCCAATAAATCCGGATAACGCTCGCATCTTCCGTATTACCGCGGCTGCTGGCA |
| | CGGAATTAGCCGATGCTTATTCATAAGGTACCTGCAAATGGCCATACATGGCCAACTTTATCCCCTTA |
| | TAAAAGGAGTTTACAACCCGTAGGGCCGTCGTCCTCCACGCTACTTGGCTGGTTCAGGCTCGCGCCC |
| | ATTGACCAATATTCCTCA |
| OTU | >denovo2372 |
| Ref | |
| SEQ ID | CCTGTTTGCTCCCCACGCTTTCGCGCTTCAGCGTCAGTATCTGTCCAGTGAGCTGACTTCTCCATCGG |
| NO: 92 | CATTCCTACAAATATCTACGAATTTCACCTCTACACTTGTAGTTCCGCCCACCTCTCCAGTACTCTAG |
| | AAAGACAGTTTCCAACGCAATACGGAGTTGAGCCCCGCATTTTCACATCAGACTTATCAAACCGCCT |
| | AGACGCGCTTTACGCCCAATAAATCCGGATAACGCTTGCGACATACGTATTACCGCGGCTGCTGGCA |
| | CGTATTTAGCCGTCGCTTCTTCTGTTGGTACCGTCACTTTCTTCTTCCCAACTGAAAGCACTTTACATT |
| | CCGAAAAACTTCATCGTGCACACAGAATTGCTGGATCAGACTTTTGGTCCATTGTCCAATATTCCCCA |
| OTU | >denovo2380 |
| Ref | |
| SEQ ID | CCTGTTTGCTCCCCACGCTTTCGTGCATGAGCGTCAGTGTTATCCCAGGGGGCTGCCTTCGCCATCGG |
| NO: 93 | TATTCCTCCACATCTCTACGCATTTCACTGCTACACGTGGAATTCTACCCCCCTCTGACACACTCTAG |
| | CCGTGCAGTCACCAATGCAATTCCCAGGTTAAGCCCGGGGATTTCACATCGGTCTTGCACAACCGCC |
| | TGCGCACGCTTTACGCCCAGTAATTCCGATTAACGCTTGGACCCTACGTATTACCGCGGCTGCTGGCA |
| | CGTAGTTAGCCGGTCCTTATTCTTCCGGTACCGTCATCCACACCTAATATTAGTAAGTGCGATTTCTT |
| | TCCGGACAAAAGTGCTTTACAACCCGAAGGCCTTCTTCACACACGCGGCATTGCTGGATCAGGGTTG |
| | CCCCCATTGTCCAAAATTCCCCA |
| OTU | >denovo2407 |
| Ref | |
| SEQ ID | CCTATTTGCTCCCCACGCTTTCGTGCTTCAGTGTCAGAATCCAGACCAGACGGCCGCCTTCGCCACCG |
| NO: 94 | GTGTTCTTCCATATATCTACGCATTTTACCGCTACACATGGAGTTCCGCCGTCCTCTTCTGTTCTCTAG |
| | CTGATCAGTTTCCAGAGCAAGTACGGGTTGAGCCCATACCTTTTACTCCAGACTTGATCTGCCACCTA |
| | CGCACCCTTTACGCCCAATCATTCCGGATAACGCTCGCCACCTACGTATTACCGCGGCTGCTGGCAC |
| | GTAGTTAGCCGTGACTTTCTGGTAAGATACCATCACTCACTCATCATTCCCTATGAGTGCCGTTTTTC |
| | TCTTACAACAGAGCTTTACGATCCGAAGACCTTCCTCACTCACGCGGCATTGCTCGTTCAGGGTTCCC |
| | CCCATTGACGAAAATTCCCTA |
| OTU | >denovo2427 |
| Ref | |
| SEQ ID | CCTGTTTGCTACCCACGCTTTCGAGCCTCAGCGTCAGTTACAGCCCAGTAGGCCGCCTTCGCCACTGG |
| NO: 95 | TGTTCCTCCTAATATCTACGCATTTCACCGCTACACTAGGAATTCCGCCTACCTCTACTGCACTCAAG |
| | AGTGGCAGTTTTGAACGCGACTATCAGTTGAGCCGATAGTTTAGACATTCAACTTGCCTCCCCGCCTA |
| | CGCTCCCTTTACACCCAGTAATTCCGGACAACGCTTGCCACCTACGTATTACCGCGGCTGCTGGCACG |
| | TAGTTAGCCGTGGCTTCCTCCTCGGGTACCGTCATTTATTCGTCCCCGAAGACAGAGGTTTACAACCC |
| | GAAGGCCGTCTTCCCTCACGCGGCGTCGCTGCATCAGGGTTTCCCCCATTGTGCAATATCCCCCA |
| OTU | >denovo2433 |
| Ref | |
| SEQ ID | CCTGTTCGATACCCACGCTTTCGTGCATGAGCGTCAGTTGAGCGCCGGTATGCTGCCTTCGCAATCGG |
| NO: 96 | AGTTCTGCGTGATATCTATGCATTTCACCGCTACACCACGCATTCCGCATACTTCTCGCTCACTCAAG |
| | AACGCCAGTTTCAACGGCACGACGGGGTTGAGCCCCGAAATTTTACCGCTGACTTGACATTCCGCCT |
| | GCGCACCCTTTAAACCCAATAAATCCGGATAACGCTCGCATCCTCCGTATTACCGCGGCTGCTGGCA |
| | CGGAGTTAGCCGATGCTTTTTCTTCAGGTACTCGCAAAACGCCACACGTGGCGCCCTTTGCTCCCTGA |
| | CAAAAGAGGTTTACAATCCATAGGACCGTCATCCCTCACGCGACTTGGCTGGTTCAGCCCTGCGGCC |
| | ATTGACCAATATTCCTCA |
| OTU | >denovo2451 |
| Ref | |
| SEQ ID | CCTGTTTGCTCCCCACGCTTTCGAGCCTCAACGTCAGTTATCGTCCAGTAAGCCGCCTTCGCCACTGA |
| NO: 97 | TGTTCCTCCTAATATCTACGCATTTCACCGCTACACTAGGAATTCCGCTTACCTCTCCGACACTCTAG |
| | AAGCACAGTTTCCAAAGCAGTCACGGGGTTGAGCCCCGGGCTTTCACTTCAGACTTGCACTTCCGTC |
| | TACGCTCCCTTTACACCCAGTAAATCCGGATAACGCTTGCCCCCTACGTATTACCGCGGCTGCTGGCA |
| | CGTAGTTAGCCGGGGCTTCTTAGTCAGGTACCGTCATTTTCTTCCCTGCTGATAGAAGTTTACATACC |
| | GAAATACTTCTTCCTTCACGCGGCGTCGCTGCATCAGGGTTTCCCCCATTGTGCAATATTCCCCA |
| OTU | >denovo2459 |
| Ref | |
| SEQ ID | CCTATTTGCTCCCCACGCTTTCGGGACTGAGCGTCAGCTGCAGACCAGATCGCCGCCTTCGCCACTGG |
| NO: 98 | TGTTCCTCCATATATCTACGCATTTCACCGCTACACATGGAATTCCGCGATCCTCTTCTGCTCTCTAGC |
| | TATCTGGTTTCCATGGCTTACTGAAGTTGAGCTTCAGTCTTTCACCACAGACCTTTATTGCCGCCTGCT |
| | CCCTCTTTACGCCCAATAATTCCGGATAACGCTTGCCACCTACGTATTACCGCGGCTGCTGGCACGTA |
| | GTTAGCCGTGGCTTTCTCATAAAGTACCGTCACTTATATATCATTCCCTATATATACCGTTCTTCCTTT |
| | ATAACAGAAGTTTACAATCCGAAGACCTTCTTCCTTCACGCGGCGTTGCTCGGTCAGGGTTCCCCCCA |
| | TTGCCGAAAATTCCCTA |
| OTU | >denovo25 |
| Ref | |
| SEQ ID | CCTGTTCGCTCCCCACGCTTTCGTCCTTTAGCGTCCGAGTCCGCCCAGCAGGCCGCCTTCGCCTCTGG |
| NO: 99 | TATTCCTCCTGATCTCTGCGCATTTCACCGCTACACCAGGAATTCTGCCTGCCTCTTCGGCTCTCTAGC |
| | TAGGGGGTCCTGGGCACCGCCCTGATGTTTCACCAGTCTTTTAATGCCCACTCCCCTTAGCCGCCTTC |
| | AGACGCTTTAAGCCCAGTGATTCCGGACAACGCTCGCCCCCTACGTATTACCGCGGCTGCTGGCACG |
| | TAGTTGGCCGGGGCTTGCTTGCCGGGTACCGTCTCTCTCTTCCCCGGCTACAGCGCTTTACGGTACGA |
| | ATACCTTCTTCGCGCACGCGGCATCGCTGCGTCAGAGTTGCCTCCATTGCGCAAGATTCCCCA |
| OTU | >denovo2587 |
| Ref | |
| SEQ ID | CCTGTTTGCTCCCCACGCTTTCGAGCCTCAACGTCAGTTACCGTCCAGTAGGCCGCCTTCGCCACCGG |
| NO: 100 | TGTTCTTCCTGATATCTACGCATTTCACCGCTACACCAGGAATTCCGCCTACCCCTCCGGTACTCAAG |
| | ATCAACAGTTTCCAAAGCAGTTACAGGGTTGAGCCCTATGATTTCACTTCAGACTTGCTGTTCCGTCT |
| | ACGCTCCCTTTACACCCAGTAAATCCGGATAACGCTTGCCCCCTACGTATTACCGCGGCTGCTGGCAC |
| | GTAGTTAGCCGGGGCTTCTTAGTCAGGTACCGTCATTTTCTTCCCTGCTGATAGGGCTTTACATTCCG |
| | AAAAACTTCTTCACCCACGCGGCGTCGCTGCATCAGGCTTTCGCCCATTGTGCAATATTCCCCA |
| OTU | >denovo2622 |
| Ref | |
| SEQ ID | CCTGTTTGCTCCCCACGCTTTCGAGCCTCAACGTCAGTTACAGTCCAGTAAGCCGCCTTCGCCACTGG |
| NO: 101 | TGTTCCTCCTAATATCTACGCATTTCACCGCTACACTAGGAATTCCACTTACCTCTCCTGCACTCTAGT |
| | CCTACAGTTTCCAAAGCAGTCCCGGGGTTAAGCCCCGGGCTTTCACTTCAGACTTGCAAAACCGTCT |
| | ACGCTCCCTTTACACCCAGTAAATCCGGATAACGCTTGCCCCCTACGTATTACCGCGGCTGCTGGCAC |
| | GTAGTTAGCCGGGGCTTCTTAGTCAGGTACCGTCATTTTCTTTCCTGCTGATAGAGCTTTACATACCG |
| | AAATACTTCTTCACTCACGCGGCGTTGCTGCATCAGGGTTTCCCCCATTGTGCAATATTCCCCA |
| OTU | >denovo2694 |
| Ref | |
| SEQ ID | CCTGTTTGCTCCCCACGCTTTCGAGCCTCAGCGTCAGTTTCAGTCCAGAAAGCCGCCTTCGCCACTGG |
| NO: 102 | TGTTCTTCCTAATATCTACGCATTTCACCGCTACACTAGGAATTCCGCTTTCCTCTCCTGTACTCTAGC |
| | TTGATAGTTCCAAATGCAATCCTCGAAGTTAAGCTTCGGGCTTTCACATCTGGCTTACCATGCCGCCT |
| | ACGCTCCCTTTACACCCAGTAATTCCGGATAACGCTTGCCCCCTACGTATTACCGCGGCTGCTGGCAC |
| | GTAGTTAGCCGGGGCTTCTTATTTAGGTACCGTCATTTTTTTCGTCCCTATTGATAGAAGTTTACGATC |
| | CGAAAACCTTCATCCTTCACGCGGCGTTGCTGCATCAGGGTTTCCCCCATTGTGCAATATTCCCCA |
| OTU | >denovo2714 |
| Ref | |
| SEQ ID | CCTGTTTGCTCCCCACGCTTTCGCGCCTCAGCGTCAGTTATCGTCCAGCAAGCCGCCTTCGCCACTGG |
| NO: 103 | TGTTCCTCCTAATATCTACGCATTTCACCGCTACACTAGGAATTCCGCTTGCCTCTCCGACACTCAAG |
| | GGGTACAGTTTTGAAAGCAGTTCCGGGGTTGAGCCCCGGGATTTCACTCCCAACTTGCACCTCCGCC |
| | TACACGCCCTTTACACCCAGTAAATCCGGACAACGCTTGCAACCTACGTATTACCGCGGCTGCTGGC |
| | ACGTAGTTAGCCGTTGCTTTTTCTCCAGGTACCGTCACGTTCTTCGTCCCTGGTAAAAGAAGTTTACA |
| | ACCCGAAGGCCTTCTTCCTTCACGCGGCGTTGCTGGGTCAGGGTTTCCCCCATTGCCCAATATTCCCC |
| | A |
| OTU | >denovo2726 |
| Ref | |
| SEQ ID | CCTGTTTGCTCCCCACGCTTTCGTGCATGAGCGTCAGTACAGGCCCAGGGGATTGCCTTCGCCATCGG |
| NO: 104 | TGTTCCTCCGCATATCTACGCATTTCACTGCTACACGCGGAATTCCATCCCCCTCTGCCGTACTCCAG |
| | CGATGCAGTCACAGATGCAGTTCCCAGGTTGAGCCCGGGGATTTCACAACTGTCTTACATCACCGCC |
| | TGCGCACGCTTTACGCCCAGTAATTCCGATTAACGCTTGCACCCTACGTATTACCGCGGCTGCTGGCA |
| | CGTAGTTAGCCGGTGCTTATTCTTACGGTACCGTCATTAGCCTTCTTTATTAGAAAAGACCGTTTCGT |
| | TCCGTACAAAAGCAGTTTACAACCCGAAGGCCTTCATCCTGCACGCGGCATGGCTGGATCAGGCTTT |
| | CGCCCATTGTCCAAAATTCCCCA |
| OTU | >denovo2768 |
| Ref | |
| SEQ ID | CCTGTTTGCTCCCCACGCTTTCGAGCCTCAGCGTCAGTCATCGTCCAGTAAGCCGCCTTCGCCACTGG |
| NO: 105 | TGTTCCTCCTAATATCTACGCATTTCACCGCTACACTAGGAATTCCACTTACCCCTCCGACACTCTAG |
| | TCCGACAGTTTCCAATGCAGTACCGGGGTTGAGCCCCGGGCTTTCACATCAGACTTGCCGTACCGCC |
| | TGCGCTCCCTTTACACCCAGTAAATCCGGATAACGCTTGCACCATACGTATTACCGCGGCTGCTGGC |
| | ACGTATTTAGCCGGTGCTTCTTAGTCAGGTACCGTCATTTCTTCTTCCCTGCTGATAGAGCTTTACATA |
| | CCGAAATACTTCTTCGCTCACGCGGCGTCGCTGCATCAGGGTTTCCCCCATTGTGCAATATTCCCCA |
| OTU | >denovo2803 |
| Ref | |
| SEQ ID | CCCGTTTGCTCCCCACGCTTTCGTGCCTTAGTGTCAGAACTGTCCCAGTAACCTGCCTACGCTATCGG |
| NO: 106 | TGTTCTTTCTAATATCTACGGATTTCACTCCTACACTAGAAATTCCAGTTACCCCTAACAGTCTCGAG |
| | CTGAGCAGTTTAGATAATAGCCTGAATGGTTGAGCCACCAGATTTCACTATCTACTTACCCTGCCACC |
| | TACGCAACTCTTTACACCCAGTCAATCCGGATAATGCTTGGACCCTACGTATGACCGCGGCTGCTGG |
| | CACGTAGTTAGCCGGTCCTTATTCATGTCTTACCATCATATTTCTCGGACATAAAAGCAGTTTACAAC |
| | CCGAAGGCCGTCATCCTGCACGCGGCGTTGCTCCATCAGGCTTTCGCCCATTGTGAAAGATTCCCTA |
| OTU | >denovo2807 |
| Ref | |
| SEQ ID | CCTGTTCGCTCCCCCAGCTTTCGCGCCTCAGCGTCAGTGACGGCCCAGAGGTCTGCCTTCGCCATCGG |
| NO: 107 | TGTTCTTCCCGATATCTGCGCATTTCACCGCTACACCGGGAATTCCGACCTCCCCTACCGCACTCGAG |
| | ACGCCTAGTTCGGGACCCGGCTTCGGGTTGAGCCCGAAGATTAGAGGTCCCGCTTGGGCATCCGCCT |
| | GCGCGCGCTTTACGCCCAATGAATCCGGATAACGCTCGCCCCCTACGTATTACCGCGGCTGCTGGCA |
| | CGTAGTTAGCCGGGGCTTCTTCTGCAGGTACCGTCATGATCGTTCCTGCTGAAAGCGGTTTACGACCC |
| | GAAGGCCTTCATCCCGCACGCGGCGTTGCTGCGTCAGGGTTTCCCCCATTGCGCAAGATTCCCCA |
| OTU | >denovo2826 |
| Ref | |
| SEQ ID | CCTGTTTGCTCCCCACGCTTTCGTGCATGAGCGTCAGTACAGGTCCAGGGGATTGCCTTCGCCATCGG |
| NO: 108 | TGTTCCTCCGCATATCTACGCATTTCACTGCTACACGCGGAATTCCATCCCCCTCTACCGTACTCTAG |
| | CCATGCAGTCACAAAGGCAGTTCCCAGGTTGAGCCCGGGGATTTCACCTCTGTCTTGCATAACCGCC |
| | TGCGCACGCTTTACGCCCAGTAATTCCGATTAACGCTTGCACCCTACGTATTACCGCGGCTGCTGGCA |
| | CGTAGTTAGCCGGTGCTTATTCTTCAGGTACCGTCATGAGTCCCAGGTATTAGCCAGAACCTTTTCTT |
| | CCCTGACAAAAGCGGTTTACAACCCGAAGGCCTTCTTCCCGCACGCGGCATGGCTGGATCAGGCTTG |
| | CGCCCATTGTCCAAAATTCCCCA |
| OTU | >denovo2828 |
| Ref | |
| SEQ ID | CCTGTTCGCTCCCCACGCTTTCGCGCCTCAGCGTCAGTGTCCGTCCGGCAGGCCGCCTTCGCCTCCGG |
| NO: 109 | TATTCCTCCTGATCTCTGCGCATTTCACCGCTACACCAGGAATTCTGCCTGCCTCTCCGGCACTCCAG |
| | CCGGCCGGTCCGCAGCCCATGGCGGGGTTGGGCCCCACCCTTTGAAGCTGCGCCGCGCCGGCCGCCT |
| | ACGCGCCCTTTACGCCCAGTCATTCCGGACAACGCTCGCCCCCTACGTGTTACCGCGGCTGCTGGCA |
| | CGTAGTTAGCCGGGGCTTTCTCACCGGGTAACGTCAATCTCTTCCCCGGCGACAAAACTTTACGGAC |
| | CGAAGCCCTTCGTCGCTCACGCGGCATCGCTGCGTCAGAGTTTCCTCCATTGCGCAAGATTCCCCA |
| OTU | >denovo283 |
| Ref | |
| SEQ ID | CCTGTTTGCTACCCATGCTTTCGAGCCTCAGCGTCAGTTAGTGCCCAGCAGGCCGCCTTCGCCACTGG |
| NO: 110 | TGTTCCTCCCGATATCTACGCATTCCACCGCTACACCGGGAATTCCGCCTGCCTCTGCACCACTCAAG |
| | ATTTGCAGTTTTGAATGCGAGAAGGGGTTGAGCCCCTCCATTAAACATCCAACTTGCAAACCCGCCT |
| | GCGCTCCCTTTACACCCAGTAATTCCGGACAACGCTTGCCACCTACGTATTACCGCGGCTGCTGGCAC |
| | GTAGTTAGCCGTGGCTTCCTCCTCGGGTACCGTCATACTTCGTCCCCGAAGACAGAGGTTTACAATCC |
| | GAAAATCTTCTTCCCTCACGCGGCGTCGCTGCATCAGAGTTTCCTCCATTGTGCAATATTCCCCA |
| OTU | >denovo2832 |
| Ref | |
| SEQ ID | CCTGTTTGCTCCCCACGCTTTCGTGCCTCAACGTCAGTACAGGCCCAGCAAGCCGCCTTCGCCACCGG |
| NO: 111 | TGTTCCTCCATATATTTACGCATTTTACCGCTACACATGGAATTCCACTTGCCTCTACCGTACTCTAGC |
| | TCAAGAGTTTCTTTTGCGAGATTGGGTTGAGCCCAACAATTTAACAAAAGACTTCTTGTGCCGTCTAC |
| | GCACCCTTTACGCCCAATAAATCCGGATAACGCTCGCCCCCTACGTATTACCGCGGCTGCTGGTACG |
| | TAGTTAGCCGGGGCTTATTCATTAGGTACCGTCAATACGAGTTTTCGTCCCTAATAAAAGAACTTTAC |
| | ATTACGAATAACTTCTTCATTCACGCGGCATTGCTCGGTCAGACTTTCGTCCATTGCCGAAAATTCCC |
| | TA |
| OTU | >denovo2837 |
| Ref | |
| SEQ ID | CCTGTTCGATACCCACGCTTTCGTGCCTGAGCGTCAGTAGGGCGCCGGTGCGCTGCCTTCGCAATCG |
| NO: 112 | GAGTTCTGCGTGATATCTATGCATTTCACCGCTACACCACGCATTCCGCGCACTTCTCGCCCACTCTA |
| | GCCCGGCAGTTCCGACGGCTGGACGGGGTTGAGCCCCGCGATTTGACCGCCGGCTTGTCGGGCCGCC |
| | TGCGCACCCTTTAAACCCAATAAATCCGGATAACGCTCGCATCCTCCGTATTACCGCGGCTGCTGGC |
| | ACGGAGTTAGCCGATGCTTGTTCTACGGGTACTCTCCTTCCGCGGACACGTCCGCGGACTTGCTCCCC |
| | GTCCAAAGAGGTCTGCGACCCGCAGGGCCTTCTTCCCTCACGCGACTTGGCTGGTTCAGGCTTCCGC |
| | CCATTGACCAATATTCCTCA |
| OTU | >denovo2845 |
| Ref | |
| SEQ ID | CCTGTTCGCTCCCCCAGCTTTCGCGCCTCAGCGTCAGTGCCGGCCCAGAGAGCTGCCTTCGCCATCGG |
| NO: 113 | TGTTCTTCCCGATATCTGCGCATTCCACCGCTACACCGGGAATTCCGCTCTCCTCTACCGCACTCGAG |
| | CCGCCCAGTTCGGAACCCGGCCCGAGGTTGAGCCCCGGGGTTAGAGGTCCCGCTTAGGCGGCCGCCT |
| | ACGCGCGCTTTACGCCCAATGAATCCGGATAACGCTCGCTCCCTACGTATTACCGCGGCTGCTGGCA |
| | CGTAGTTAGCCGGAGCTTCTTCTGCAGGTACCGTCAATATCTTCCCTGCTGAAAGCGGTTTACAACCC |
| | GAGGGCCGTCATCCCGCACGCGGCGTTGCTGCGTCAGGGTTCCCCCCATTGCGCAAAATTCCCCA |
| OTU | >denovo2885 |
| Ref | |
| SEQ ID | CCTGTTTGCTCCCCACACTTTCGTGCCTCAGTGTCAGTAACAGTCCAGAAAGCCGCCTTCGCCACTGG |
| NO: 114 | TGTTCCTCCTAATATCTACGCATTTCACCGCTACACTAGGAATTCCGCTTTCCTCTCCTGCACTCAAGT |
| | AGCCCAGTTCAGGGGGCTAACAATGGTTGAGCCATTGCCTTTCACCCCCTGCTTAGGTAACCACCTA |
| | CGCACTCTTTACGCCCAATAATTCCGGATAACGCTTGCCCCCTACGTATTACCGCGGCTGCTGGCACG |
| | TAGTTAGCCGGGGCTTCCTCCAAGGGTACCGTCATTTTTTTCTTCCCCAAGGACAGAGTTTTACAATC |
| | CGAAGACCTTCTTCACTCACGCGGCGTTGCTGCATCAGGCTTTCGCCCATTGTGCAATATTCCCCA |
| OTU | >denovo2920 |
| Ref | |
| SEQ ID | CCTGTTTGCTCCCCATGCTCTCATGCCTCAGCGTCAGTTACAGTCCAGTAAGTCGCCTTCGCCACTGG |
| NO: 115 | TGTTCTTCCTAATCTCTACGCATTTCACCGCTACACTAGGAATTCCACTTACCTCTCCTGCACTCTAGT |
| | TAAACAGTTTTATGTGCTTTCTACAGGTTAAGCCTATATATTTAACACATAACTTACTCAACCGCCTA |
| | CGCATCCTTTACGCCCAGTTATTCCGAACAACGCTTGCCACCTACGTATTACCGCGGCTGCTGGCACG |
| | TAGTTAGCCGTGGCTTCCTCTGCAGGTACCGTCCTTCCTCTTCCCTGCTAACAGAGCTTTACAATCCG |
| | AAAACCTTCTTCACTCACGCGGCGTTGCTGCATCAGGGTTTCCCCCATTGTGCAATATTCCCCA |
| OTU | >denovo2964 |
| Ref | |
| SEQ ID | CCTGTTCGCTCCCCACGCTTTCGTGACTCAGCGTCAGTTTCAGTCCAGTAAGCCGCCTTCGCCACCGG |
| NO: 116 | TGTTCTTCCTAATATCTACGCATTTCACCGCTACACTAGGAATTCCGCTTACCTCTCCTGTACTCTAGT |
| | TACACAGTTTCTTATGCAGCTCTGGAGTTGAGCCCCAGAATTTCACATATGACTTGCATAACCGCCTA |
| | CTCACCCTTTACACCCAGTAAATCCGGATAACGCTTGCACCATACGTATTACCGCGGCTGCTGGCAC |
| | GTATTTAGCCGGTGCTTCTTATTCAGGTACCGTCATTTCTTTCTTCCCTGTCGATAGAAGTTTACATAC |
| | CGAAATACTTCTTCCTTCACGCGGCGTTGCTGCATCAGGGTTCCCCCCATTGTGCAATATTCCCCA |
| OTU | >denovo2989 |
| Ref | |
| SEQ ID | CCTGTTTGCTCCCCACGCTTTCGTGCCTCAGCGTCAGTTACAGTCCAGAGAGCCGCCTTCGCTACTGG |
| NO: 117 | TGTTCCTCCTAATATCTACGCATTTCACCGCTACACTAGGAATTCCACTCTCCTCTCCTGCACTCAAGT |
| | CCTACAGTTCCAAAAGCTTACTACGGTTGAGCCGTAGCCTTTCACTTCTGGCTTGAAAGACCGCCTAC |
| | GCACCCTTTACGCCCAGTAATTCCGGATAACGCTAGCCCCCTACGTATTACCGCGGCTGCTGGCACG |
| | TAGTTAGCCGGGGCTTCCTCCTCAAGTACCGTCATTATCTTCCTTGAGGACAGAGCTTTACGACCCGA |
| | AGGCCTTCATCGCTCACGCGGCGTTGCTGCATCAGGCTTTCGCCCATTGTGCAATATTCCCCA |
| OTU | >denovo3065 |
| Ref | |
| SEQ ID | CCTGTTTGCTCCCCACGCTTTCGAGCCTCAGCGTCAGTTACAGTCCAGAGAGTCGCCTTCGCCACTGG |
| NO: 118 | TGTTCTTCCTAATCTCTACGCATTTCACCGCTACACTAGGAATTCCACTCTCCTCTCCTGCACTCTAGA |
| | TAACCAGTTTGGAATGCAGCACCCAAGTTGAGCCCGGGTATTTCACATCCCACTTAATCATCCGCCT |
| | ACGCTCCCTTTACGCCCAGTAAATCCGGATAACGCTCGCCACCTACGTATTACCGCGGCTGCTGGCA |
| | CGTAGTTAGCCGTGGCTTCCTCCTTGGGTACCGTCATTATCTTCCCCAAAGACAGAGCTTTACGATCC |
| | GAAAACCTTCATCACTCACGCGGCGTTGCTGCATCAGGGTTTCCCCCATTGTGCAATATTCCCCA |
| OTU | >denovo3107 |
| Ref | |
| SEQ ID | CCTGTTTGCTCCCCACGCTTTCGCGCCTCACCGTCAGTTGTCGTCCAGTCATCCGCCTTCGCCACTGGT |
| NO: 119 | GTTCTTCCTTATATCTACGCATTTCACCGCTACACAAGGAATTCCGATGACCTCTCCGATACTCAAGA |
| | AACACAGTTTCAAATGCAGTTTGGAGGTTGAGCCTCCAGATTTCACATCTGACTTGCATTCCCGGCTG |
| | CACGCCCTTTACACCCAGTAAATCCGGACAACGCTTGCCACCTACGTATTACCGCGGCTGCTGGCAC |
| | GTAGTTAGCCGTGGCTTATTCGTCAGGTACCCTCTTCTACTGTTCCCTGACAAAAGAAGTTTACAACC |
| | CGAAAGCCTTCTTCCTTCACGCGGCGTTGCTGGGTCAGACTTGCGTCCATTGCCCAATATTCCCCA |
| OTU | >denovo3110 |
| Ref | |
| SEQ ID | CCTGTTCGATACCCGCACTTTCGAGCTTCAGCGTCAGTTGCGCTCCAGTGAGCTGCCTTCGCAATCGG |
| NO: 120 | AGTTCTTCGTGATATCTAAGCATTTCACCGCTACACCACGAATTCCGCCCACTTTGTGCGTACTCAAG |
| | GAAACCAGTTCGCGCTGCAGTGCAGACGTTGAGCGTCTACATTTCACAACACGCTTAATCTCCGGCC |
| | TACGCTCCCTTTAAACCCAATAAATCCGGATAACGCCCGGACCTTCCGTATTACCGCGGCTGCTGGC |
| | ACGGAATTAGCCGGTCCTTATTCATAAGGTACATGCAAAAAGCCTCACGAGGCTCACTTTATTCCCTT |
| | ATAAAAGCAGTTTACAACCCGGAGGGCCGTCATCCTGCACGCTACTTGGCTGGTTCAGACTTGCGTC |
| | CATTGACCAATATTCCTCA |
| OTU | >denovo3157 |
| Ref | |
| SEQ ID | CCTGTTTGCTCCCCACGCTTTCGCACCTCAGCGTCAGTATTTGCCCAGGAGACCGCCTTCGCCTCTGG |
| NO: 121 | TGTTCTTTCAGATATCTACGCATTCCACCGCTACACCTGAAATTCCATCTCCCTCTGCAATACTCTAG |
| | AAAGGTAGTTCAAAATGACCAACCCAGGTTGAGCCCAGGTCTTTCACATCTTGCTTGCCTTTCCGCCT |
| | ACATGCCCTTTACGCCCAGTTATTCCGATTAACGCTTGCACCCTCCGTATTACCGCGGCTGCTGGCAC |
| | GGAGTTAGCCGGTGCTTCTTCTGTGGGTAACGTCAGTATTTCCTCCCCACTGAAAGTGCTTTACAACC |
| | CGAAGGCCTTCTTCACACACGCGGCATGGCTGCATCAGGGTTTCCCCCATTGTGCAATATTCCCCA |
| OTU | >denovo3177 |
| Ref | |
| SEQ ID | CCTATTTGCTCCCCACGCTTTCGGGACTGAGCGTCAGTTACAGCCCAGATCGTCGCCTTCGCCACTGG |
| NO: 122 | TGTTCCTCCATATATCTACGCATTTCACCGCTACACATGGAATTCCACGATCCTCTACTGCACTCTAG |
| | CTGTGCGGTTTCCACGGCTTACCGAAGTTTAGCTTCGGGCTTTCACCGCAGACCTGCACTGCCGCCTG |
| | CTCCCTCTTTACGCCCAATGATTCCGGATAACGCTCGCCACCTACGTATTACCGCGGCTGCTGGCACG |
| | TAGTTAGCCGTGGCTTCCTCATAAAGTACCGTCACTTACATGGCATTCCCTCCATGTACCGTTCTTCC |
| | TTTATAACAGAGGTTTACATACCGAAGTACTTCCTCCCTCACGCGGCGTTGCTCGGTCAGGCTTTCGC |
| | CCATTGCCGAAAATTCCCTA |
| OTU | >denovo325 |
| Ref | |
| SEQ ID | CCTGTTTGCTCCCCGCGCTTTCGCGCCTCAGCGTCAGTGTGCGTCCAGCAGGCCGCCTTCGCCTCTGG |
| NO: 123 | TATTCCTCCTGATATCTGCGCATTTCACCGCTACACCAGGAATTCTGCCTGCCTCTCCGACACTCTAG |
| | CCACGCGGTTCAGGGCGCTTGCACGGGGTTAGGCCCCGTCTTTTCACACCCTGCCTGCGCGGCCGCC |
| | TGCGCGCCCTTTACGCCCAGTGATTCCGGACAACGCTTGCCCCCCACGTATTGCCGCGGCTGCTGGC |
| | ACGTGGTTGGCCGGGGCTTTCTTACCGGGTACCGTCTGTTTTCTTCCCCGATAACAGAGCTTTACGAG |
| | CCGGAGCCCTTCCTCGCTCACGCGGCATTGCTGCGTCAGAGTTTCCTCCATTGCGCAATATTCCCCA |
| OTU | >denovo3269 |
| Ref | |
| SEQ ID | CCTGTTTGCTACCCACGCTTTCGAGCCTCAGCGTCAGTTAAAGCCCAGCAGGCCGCCTTCGCCACTGG |
| NO: 124 | TGTTCCTCCTAATATCTACGCATTTCACCGCTACACTAGGAATTCCGCCTGCCTCTACTTCACTCAAG |
| | AAAAACAGTTTTGAAAGCAGCTCATGGGTTGAGCCCATGCATTTCACTTCCAACTTGCTTTCCCGCCT |
| | ACGCTCCCTTTACACCCAGTAATTCCGGACAACGCTCGCTCCCTACGTATTACCGCGGCTGCTGGCAC |
| | GTAGTTAGCCGGAGCTTCCTTCTTCGGTACCGTCACTTTCTTCGTCCCGAATGACAGAGGTTTACAAC |
| | CCGAAGGCCGTCTTCCCTCACGCGGCGTCGCTGCATCAGGCTTTCGCCCATTGTGCAATATCCCCCA |
| OTU | >denovo3286 |
| Ref | |
| SEQ ID | CCTATTTGCTCCCCACGCTTTCGTGCCTCAGCGTCAGGTACAGGCCAGGCGGCCGCCTTCGCCGCTGG |
| NO: 125 | TGTTCTTCCACATCTCTACGCATTTTACCGCTACACATGGAGTTCCACCGCCCTCTCCTGTCCTCAAGC |
| | CGTGCAGTTTCCAAAGCCTGAACCGGTTGAGCCGGTCCCTTTTACTTCAGACTTGCACAGCCGCCTGC |
| | GCACCCTTTACGCCCAATCATTCCGGATAACGCTTGCCACCTACGTATTACCGCGGCTGCTGGCACGT |
| | AGTTAGCCGTGGCTTTCTGGCCGGGTACCATCAAACAGTCAGCTTTCCACTCTGGCTGTCCTTTGTCC |
| | CCGGCAACAGGGCTTTACAATCCGAAGACCGTCTTCACCCACGCGGCATTGCTCGTTCAGGCTTGCG |
| | CCCATTGACGAAAATTCCCTA |
| OTU | >denovo3354 |
| Ref | |
| SEQ ID | CCTGTTTGCTCCCCACGCTTTCGAGCCTCAACGTCAGTTACTGTCCAGTAAGCCGCCTTCGCCACTGG |
| NO: 126 | TGTTCCTCCTAATATCTACGCATTTCACCGCTACACTAGGAATTCCACTTACCTCTCCAGCACTCTAG |
| | ATGAACAGTTTCCAATGCAGTCCCGGGGTTGAGCCCCGGGTTTTCACATCAGACTTGCCCATCCGTCT |
| | ACGCTCCCTTTACACCCAGTAAATCCGGATAACGCTTGCACCATACGTATTACCGCGGCTGCTGGCA |
| | CGTATTTAGCCGGTGCTTCTTAGTCAGGTACCGTCATTATCTTCCCTGCTGATAGAGCTTTACATACC |
| | GAAATACTTCATCGCTCACGCGGCGTCGCTGCATCAGGGTTTCCCCCATTGTGCAATATTCCCCA |
| OTU | >denovo3374 |
| Ref | |
| SEQ ID | CCTGTTTGATACCCGCACTTTCGAGCCTCAGCGTCAGTTGCACCCCGGATACCTGCCTTCGCGATCGG |
| NO: 127 | AGTTCTTCATGATATCTGAGCATTTCACCGCTACACCATGAATTCCAGCATCCCTGTGTGCACTCAAG |
| | ACTCCCAGTATCAACTGCAGTCCGACGGTTGAGCCGCCGTATTTCACAACTGACTTAAGAGTCCGCC |
| | TGCGCTCCCTTTAAACCCAATAAATCCGGATAACGCCTGGACCTTCCGTATTACCGCGGCTGCTGGC |
| | ACGGAATTAGCCGGTCCTTTTTCTGATGGTACATACAAAACAGCTCACGAGCTGCACTTTATTCCCAA |
| | CTAAAAGCAGTTTACAACCCGGAGGGCCGTCATCCTGCACGCTACTTGGCTGGTTCAGACTTGCGTC |
| | CATTGACCAATATTCCTCA |
| OTU | >denovo340 |
| Ref | |
| SEQ ID | CCTGTTCGATACCCGCACTTTCGAGCTTCCGCGTCCGTTGCGCTCCAGTGAGCTGCCTTCGCAATCGG |
| NO: 128 | AGTTCTTCGTGATATCTAAGCATTTCACCGCTACACCACGAATTCCGCCCACTTTGTGCGTACTCAAG |
| | GAAACCAGTTCGCGCTGCAGTGCAGACGTTGAGCGTCTACATTTCACAACACGCTTAATCTCCGGCC |
| | TACGCTCCCTTTAAACCCAATAAATCCGGATAACGCCCGGACCTTCCGTATTACCGCGGCTGCTGGC |
| | ACGGAATTAGCCGGTCCTTATTCATAAGATACATGCAAAAAGAGTCACGACTCTCACTTTATTCCCTT |
| | ATAAAAGCAGTTTACAACCCATAGGGCCGTCTTCCTGCACGCTACTTGGCTGGTTCAGACTCTCGTCC |
| | ATTGACCAATATTCCTCA |
| OTU | >denovo3440 |
| Ref | |
| SEQ ID | CCTGTTTGCTCCCCACGCTTTCGTGCCTCAGCGTCAGTTATGGTCCAGAAAGCCGCCTTCGCTACTGG |
| NO: 129 | TGTTCCTCCGAATCTCTACGCATTTCACCGCTACACTCGGAATTCCACTTTCCTCTCCCACACTCTAGC |
| | ACAGCAGTTTCAGAAGCAGCTCTGGGGTTGAGCCCCAAAATTTCACTTCTGACTTACCATGCCGCCT |
| | ACGCACCCTTTACACCCAGTAAATCCGGATAACGCTCGCTCCCTACGTATTACCGCGGCTGCTGGCA |
| | CGTAGTTAGCCGGAGCTTCTTAGTCAGGTACCGTCATTTTTTCTTCCCTGCTGATAGAAGTTTACAAT |
| | CCGAAGACCTTCATCCTTCACGCGGCGTCGCTGCATCAGGCTTGCGCCCATTGTGCAATATTCCCCA |
| OTU | >denovo3487 |
| Ref | |
| SEQ ID | CCTGTTTGATACCCGCACTTTCGAGCATCAGCGTCAGTTACGGTCCAGTAAGCTGCCTTCGCAATCGG |
| NO: 130 | AGTTCTTCGTGATATCTAAGCATTTCACCGCTACACCACGAATTCCGCCTACCTATACCGCACTCAAG |
| | AAATCCAGTATCAACTGCAATTTTACGGTTGAGCCGCAAACTTTCACAACTGACTTAAACTTCCGCCT |
| | ACGCTCCCTTTAAACCCAATAAATCCGGATAACGCTCGGATCCTCCGTATTACCGCGGCTGCTGGCA |
| | CGGAGTTAGCCGATCCTTATTCATACGGTACATACAAAAAAGCACACGTGCTTCACTTTATTCCCGTA |
| | TAAAAGAAGTTTACAACCCATAGGGCAGTCATCCTTCACGCTACTTGGCTGGTTCAGACTCTCGTCC |
| | ATTGACCAATATTCCTCA |
| OTU | >denovo3571 |
| Ref | |
| SEQ ID | CCTGTTTGCTCCCCACGCTTTCGAGCCTCAGCGTCAGTTACAGTCCAGAGAGTCGCCTTCGCCACTGG |
| NO: 131 | TGTTCTTCCTAATCTCTACGCATTTCACCGCTACACTAGGAATTCCACTCTCCTCTCCTGCACTCTAGA |
| | TAATCAGTTTGGAATGCAGCCCCCAGGTTGAGCCTGAGTATTTCACATCCCACTTAATTATCCGCCTA |
| | CGCTCCCTTTACGCCCAATAATTCCGGATAACGCTCGCCACCTACGTATTACCGCGGCTGCTGGCACG |
| | TAGTTAGCCGTGGCTTCCTCCTTGGGTACCGTCATTATCGTCCCCAAAGACAGAGCTTTACAATCCGA |
| | AGACCGTCATCACTCACGCGGCGTTGCTGCATCAGGGTTTCCCCCATTGTGCAATATTCCCCA |
| OTU | >denovo3578 |
| Ref | |
| SEQ ID | CCTGTTTGCTCCCCACGCTTTCGCACCTCAGCGTCAGTATTTGCCCAGGAGACCGCCTTCGCCTCTGG |
| NO: 132 | TGTTCTTTCAGATATCTACGCATTCCACCGCTACACCTGAAATTCCATCTCCCTCTGCAATACTCTAG |
| | AAAAGTAGTTCAAAACGACCAACCCAGGTTGAGCCCGGGTATTTCACGCCTTGCTTGCTTTTCCGCCT |
| | ACATGCCCTTTACGCCCAGTTATTCCGATTAACGCTTGCACCCTCCGTATTACCGCGGCTGCTGGCAC |
| | GGAGTTAGCCGGTGCTTCTTCTGTGGGTAACGTCAGTGTTTCCTCCCCACTGAAAGTGCTTTACAACC |
| | CGAAGGCCTTCTTCACACACGCGGCATGGCTGCATCAGGGTTTCCCCCATTGTGCAATATTCCCTA |
| OTU | >denovo3600 |
| Ref | |
| SEQ ID | CCTGTTTGCTCCCCACGCTTTCGCGCCTCAGCGTCAGTTAATGTCCAGCAGGCCGCCTTCGCCACTGG |
| NO: 133 | TGTTCCTCCCAATATCTACGCATTTCACCGCTACACTGGGAATTCCGCCTGCCTCTCCATCACTCAAG |
| | ATCCGCAGTTTTGAAAGCAGTTTGGGGGTTGAGCCCCCAGATTTCACTCCCAACTTACAGACCCGCC |
| | TACACGCCCTTTACACCCAGTAAATCCGGATAACGCTTGCTCCCTACGTATTACCGCGGCTGCTGGCA |
| | CGTAGTTAGCCGGAGCTTATTCTTTAGGTACCGTCATTTTTTTCGTCCCTAATTAAAGATTTTTACAAT |
| | CCGAAGACCTTCTTCAATCACGCGGCGTTGCTGCGTCAGGGTTGCCCCCATTGCGCAATATTCCCCA |
| OTU | >denovo3643 |
| Ref | |
| SEQ ID | CCCGTTCGCTACCCTGGCTTTCGCATCTCAGCGTCAGACACAGTCCAGAAAGGCGCCTTCGCCACTG |
| NO: 134 | GTGTTCCTCCCAATATCTACGCATTTCACCGCTACACTGGGAATTCCCCTTTCCTCTCCTGCACTCAA |
| | GTCTCCCAGTATCCAGAGCCATACGGGGTTGAGCCCCGCATTTTCACTCCAGACTTAAGAAACCGCC |
| | TACATGCTCTTTACGCCCAATAATTCCGGACAACGCTCGCCACCTACGTATTACCGCGGCTGCTGGCA |
| | CGTAGTTAGCCGTGGCTTATTCGTTTACTACCGTCATTACAAATAATTATTCACAATCTGCACATTCG |
| | TCATAAACAAAAGAGTTTTACGGAACGAATTCCTTCATCACTCACGCGGCATTGCTCCGTCAGGCTTT |
| | CGCCCATTGCGGAAGATTCCCCA |
| OTU | >denovo3652 |
| Ref | |
| SEQ ID | CCTGTTTGCTACCCACGCTTTCGAACCTCAGCGTCAGTTACAGACCAGAGAGCCGCTTTCGCCACTGG |
| NO: 135 | TGTTCTTCCATATATCTACGCATTTCACCGCTACACATGGAGTTCCACTCTCCTCTTCTGCACTCAAGT |
| | CTCCCAGTTTCCAATGCACTACTCCGGTTAAGCCGAAGGCTTTCACATCAGACTTAAAAGACCGCCT |
| | GCGTTCCCTTTACGCCCAATAAATCCGGATAACGCTTGCCACCTACGTATTACCGCGGCTGCTGGCAC |
| | GTAGTTAGCCGTGGCTTTCTGGTTAGATACCGTCGAAACGTGAACAGTTACTCTCACGCACTTTCTTC |
| | TCTAACAACAGGGTTTTACGATCCGAAGACCTTCTTCACCCACGCGGCGTTGCTCCATCAGGCTTTCG |
| | CCCATTGTGGAAGATTCCCTA |
| OTU | >denovo3663 |
| Ref | |
| SEQ ID | CCTGTTCGCTCCCCGCGCTCTCGCGCCTTAGCGTCAGTGTGCGTCCGGCGGGCCGCCTTCGCCTCCGG |
| NO: 136 | TGTTCCCCCTGGCCTCTGCGCATTTCACCGCTACGCCAGGGGTTCCGCCCGCCTCTCCGCCACTCCAG |
| | CCGCCCGGTCCGGGGCGCTTGCCCGGGGTTGGGCCCCGGCTTTTAACGCCCCGCCTAAGCGGCCGCC |
| | TGCGCGCGCTTTACGCCCAGTGATTCCGGACAACGCTCGCCCCCCACGTATTGCCGCGGCTGCTGGC |
| | ACGTGGTTGGCCGGGGCTTTCTTGCCGGGTTGCGTCATTTTTTTCCTCCCCGGCGGCGGGGCTTTACG |
| | GGCCTGAGCCCTTCATCGCCCACGCGGCATCGCTGCGTCAGAGTTCCCTCCATTGCGCAATATTCCCC |
| | A |
| OTU | >denovo3681 |
| Ref | |
| SEQ ID | CCTGTTCGCTCCCCCGGCTTTCGCGCCTCAGCGTCAGTGTCGGCCCAGCAGGCTGCCTTCGCCATCGG |
| NO: 137 | TGTTCCTCCCGATATCTGCGCATTCCACCGCTACACCGGGAATTCCACCTGCCCCTACCGCACTCGAG |
| | CCGCCCAGTTCGGAACCCGGCCGCGGGGTTGAGCCCCGGGATTAGAGGTTCCGCTTAGGCGGCCGCC |
| | TACGCGCGCTTTACGCCCAATGAATCCGGATAACGCTCGCCCCCTACGTATTACCGCGGCTGCTGGC |
| | ACGTAGTTAGCCGGGGCTTCTTCTGCAGGTACCGTCTAGGTTCTTCCCTGCTGAAAGCGGTTTACAAC |
| | CCGAAGGCCTTCGTCCCGCACGCGGCGTTGCTGCGTCAGGGTTCCCCCCATTGCGCAAAATTCCCCA |
| OTU | >denovo3709 |
| Ref | |
| SEQ ID | CCTATTTGCTCCCCACGCTTTCGTGCCTCAGCGTCAGAGTCAGTCCAGGCGGCCGCCTTCGCCACTGG |
| NO: 138 | TGTTCTTCCATATATCTACGCATTTTACCGCTACACATGGAGTTCCACCGCCCTCTCCTGCTCTCCAGC |
| | CGGCCAGTTTCCTCAGCAAACCGAAGTTGAGCTTCGATCTTTCACTGAAGACTTGACCGGCAGCCTG |
| | CGCACCCTTTACGCCCAATCATTCCGGATAACGCTCGCCACCTACGTATTACCGCGGCTGCTGGCAC |
| | GTAGTTAGCCGTGACTTTCTGGCGGGGTACGATCCTGAAGGAAGCTTTCCACTCTTCCTTCCTCTTAT |
| | TCCCCGCAACAGAGCTTTACAGCCCGAAGGCCGTCTTCACTCACGCGGCATTGCTCGTTCAGAGTTTC |
| | CTCCATTGACGAAAATTCCCTA |
| OTU | >denovo3748 |
| Ref | |
| SEQ ID | CCTATTTGCTCCCCACGCTTTCGTGCCTCAGCGTCAGCAACAGGCCAGGCGGCCGCCTTCGCCACTGG |
| NO: 139 | TGTTCTTCCATATATCTACGCATTTTACCGCTACACATGGAGTTCCACCGCCCTCTCCTGCTCTCCAGT |
| | CCTCCAGTTTCCAAAGCCATGCATGAGTTGAGCTCATGCGTTTCACTCCAGACTTGCAGGACCGCCTG |
| | CGCACCCTTTACGCCCAATCATTCCGGATAACGCTCGCCACCTACGTATTACCGCGGCTGCTGGCAC |
| | GTAGTTAGCCGTGACTTTCTGGCGGGGCACCATCAGTCAGCACCCATTTCCTGATGCTGCCTTTTTTC |
| | CCCCGCAACAGAGCTTTACGACCCGAAGGCCTTCTTCACTCACGCGGCATCGCTCGTTCAGGCTTGC |
| | GCCCATTGACGAAAATTCCCTA |
| OTU | >denovo3752 |
| Ref | |
| SEQ ID | CCTGTTCGCTACCCATGCTTTCGAGCCTCAGCGTCAGTTGCAGACCAGAGAGCCGCCTTCGCCACTG |
| NO: 140 | GTGTTCTTCCATATATCTACGCATTCCACCGCTACACATGGAGTTCCACTCTCCTCTTCTGCACTCAA |
| | GAAAAACAGTTTCCGATGCAGTTCCTCGGTTAAGCCGAGGGCTTTCACATCAGACTTATTCTTCCGCC |
| | TGCGCTCGCTTTACGCCCAATAAATCCGGACAACGCTTGCCACCTACGTATTACCGCGGCTGCTGGC |
| | ACGTAGTTAGCCGTGACTTTCTGGTTGATTACCGTCAAATAAAGGCCAGTTACTACCTCTATCCTTCT |
| | TCACCAACAACAGAGCTTTACGATCCGAAAACCTTCTTCACTCACGCGGCGTTGCTCCATCAGACTTT |
| | CGTCCATTGTGGAAGATTCCCTA |
| OTU | >denovo3789 |
| Ref | |
| SEQ ID | CCTGTTTGCTACCCACGCTTTCGTTCCTCAGCGTCAGTTTGTGTCCAGCAAGTCGCCTTCGCCACTGG |
| NO: 141 | TATTCCTCCTAATATCTACGCATTTCACCGCTACACTAGGAATTCCACTTGCCTCTCCACATCTCAAG |
| | AAATACAGTTTCAGATGCTTACAGAGGTTAAGCCTCTGCCTTTCACATCTGACTTGCATTTCCGCCTG |
| | CGAACCCTTTACGCCCAGTAATTCCGGACAACGCTAGCCCCCTACGTATTACCGCGGCTGCTGGCAC |
| | GTAGTTAGCCGGGGCTTCCTCCTATGGTACCGTCATTATCTTCCCATAGGACAGAGCTTTACGATTCG |
| | AAAACCTTCATCGCTCACGCGGCGTCGCTGCATCAGGGTTTCCCCCATTGTGCAATATTCCCCA |
| OTU | >denovo380 |
| Ref | |
| SEQ ID | CCTGTTCGCTCCCCACGCTTTCGAGCCTCAGCGTCAGTTACAGTCCAGAGAGTCGCCTTCGCCACTGG |
| NO: 142 | TGTTCTTCCTAATCTCTACGCATTTCACCGCTACACTAGGAATTCCACTCTCCCCTCCTGCACTCTAGA |
| | TTCCCAGTTTGGAATGCAGCACCCAGGTTGAGCCCGGGTATTTCACATCCCACTTAAAAATCCGCCT |
| | ACGCTCCCTTTACGCCCAGTAAATCCGGACAACGCTCGCCACCTACGTATTACCGCGGCTGCTGGCA |
| | CGTAGTTAGCCGTGGCTTCCTCCTTGGGTACCGTCATTATCGTCCCCAAAGACAGAGCTTTACAATCC |
| | GAAGACCGTCATCACTCACGCGGCGTTGCTGCATCAGGGTTTCCCCCATTGTGCAATATTCCCCA |
| OTU | >denovo3808 |
| Ref | |
| SEQ ID | CCTATTTGCTCCCCACGCTTTCGTGCCTGAGCGTCAGTTACAGACCAGGTAGCCGCTTTCGCCACTGG |
| NO: 143 | TGTTCCTCCATATATCTACGCATTTTACCGCTACACATGGAATTCCACTACCCTCTTCTGCACTCTAGT |
| | CTACCAGTTTTTAAGGCTATATTGGGTTAAGCCCAACGCTTTCACCCTAAACTTAATAGACCGCCTGC |
| | GCACCCTTTACGCCCAATAATTCCGGATAACGCTTGCCACCTACGTATTACCGCGGCTGCTGGCACGT |
| | AGTAAGCCGTGACTTTCTGGTAAAGTACCGTCAAAATATCAGCATTTCCTCAGATATTCATTCTTCCT |
| | TTACAACAGAGCTTTACAACCCAAAGGCCTTCATCACTCACGCGGCGTTGCTCGGTCAGGGTTTCCC |
| | CCATTGCCGAAAATTCCTTA |
| OTU | >denovo3815 |
| Ref | |
| SEQ ID | CCTGTTCGCTCCCCACGCTTTCGCACCTGAGCGTCAGTTGCCAGCCAGCAGGCCGCCTTCGCCTCCGG |
| NO: 144 | TATTCCTCCTGATCTCTGCGCATTTCACCGCTACACCAGGAATTCTGCCTGCCTCTCTGGCACTCGAG |
| | CTCCACGGTTCCGCCGACCGTTACCGGGTTGGGCCCGGTAATTTCATCGGCGGCCCTTGAAGCCGCC |
| | TGCGTGCCCTTTACGCCCAATGATTCCGGACAACGCTCGCCCCCCACGTGTTACCGCAGCTGCTGGC |
| | ACGTAGTTGGCTGGGGCTCCTACCCGGGTACCGTCTTCCTCTTCCCCGGGGACAAAGCTTTACGGGA |
| | CGAGTCCCTTCTTCACTCACGCGGCATCGCTGCGTCAGGGTTTCCCCCATTGCGCAATATTCCCCA |
| OTU | >denovo3832 |
| Ref | |
| SEQ ID | CCTGTTTGCTCCCCACGCTTTCGAGCCTCAACGTCAGTTACTGTCCAGTAAGCCGCCTTCGCCACTGG |
| NO: 145 | TGTTCCTCCTAATATCTACGCATTTCACCGCTACACTAGGAATTCCACTTACCTCTCCAGCACTCTAG |
| | CATCACAGTTTCCAAAGCAGTCCCGGGGTTGAGCCCCGGGCTTTCACTTCAGACTTGCAGCGCCGTC |
| | TACGCTCCCTTTACACCCAGTAAATCCGGATAACGCTTGCCCCCTACGTATTACCGCGGCTGCTGGCA |
| | CGTAGTTAGCCGGGGCTTCTTAGTCAGGTACCGTCATTTTCTTCCCTGCTGATAGAGCTTTACATACC |
| | GAAATACTTCTTCACTCACGCGGCGTTGCTGCATCAGGCTTGCGCCCATTGTGCAATATTCCCCA |
| OTU | >denovo3904 |
| Ref | |
| SEQ ID | CCTGTTTGCTCCCCATGCTTTCGCGCTTCAGCGTCAGTATCTGTCCAGTGAGCTGACTTCTCTATCGGC |
| NO: 146 | ATTCCTACAAATATCTACGAATTTCACCTCTACACTTGTAGTTCCGCCCACCTCTCCAGTACTCTAGT |
| | CTGGCAGTTTCCAACGCAATACGGAGTTGAGCCCCGCATTTCCACATCAGACTTACCAGACCGCCTA |
| | GACGCGCTTTACGCCCAATAAATCCGGATAACGCTTGCGACATACGTATTACCGCGGCTGCTGGCAC |
| | GTATTTAGCCGTCGCTTCTTCTATCGGTACCATCACTTTCTTTTTCCCGATTGAAAGCACTTTACAATC |
| | CTAAGACCGTCATCGTGCACACAGAATTGCTGGATCAGACTTTTGGTCCATTGTCCAATATTCCCCA |
| OTU | >denovo4026 |
| Ref | |
| SEQ ID | CCTGTTTGATACCCGCACTTTCGAGCCTCAGCGTCAGTTACACTCCAGATACCTGCCTTCGCGATCGG |
| NO: 147 | AGTTCCTCATGATATCTGAGCATTTCACCGCTACACCATGAATTCCAGTATCTCTGCGTGTACTCAAG |
| | ACTCCCAGTATCAACTGCAGTCCGACGGTTGAGCCGCCGTATTTCACAACTGACTTAAGAGTCCGCC |
| | TGCGCTCCCTTTAAACCCAATAAATCCGGATAACGCCTGGACCTTCCGTATTACCGCGGCTGCTGGC |
| | ACGGAATTAGCCGGTCCTTTTTCTGATGGTACATACAAAACGGTACACGTACCGCACTTTATTCCCAA |
| | CTAAAAGCAGTTTACAACCCAGAGGGCCGTCATCCTGCACGCTACTTGGCTGGTTCAGACTTCCGTC |
| | CATTGACCAATATTCCTCA |
| OTU | >denovo4099 |
| Ref | |
| SEQ ID | CCTGTTTGCTCCCCACGCTTTCGAGCCTCAACGTCAGTTACAGTCCAGTAAGACGCCTTCGCCACTGG |
| NO: 148 | TGTTCCTCCTAATATCTACGCATTTCACCGCTACACTAGGAATTCCTCTTACCTCTCCTGCACTCCAGT |
| | ATCACAGTTTCCAAAGCAGTCCCGCGGTTGAGCCCCGGGCTTTCACTTCAGACTTGCAGTACCGTCTA |
| | CGCTCCCTTTACACCCAGTAAATCCGGATAACGCTTGCCCCCTACGTATTACCGCGGCTGCTGGCACG |
| | TAGTTAGCCGGGGCTTCTTAGTCAGGTACCGTCATTTTCTTCCCTGCTGATAGAGCTTTACATACCGA |
| | AATACTTCTTCACTCACGCGGCGTCGCTGCATCAGGGTTTCCCCCATTGTGCAATATTCCCCA |
| OTU | >denovo4134 |
| Ref | |
| SEQ ID | GCCTGTTCGCTCCCCACGCTTTCGCTCCTCAGCGTCAGGAAAGGCCCAGAGAACCGCCTTCGCCACT |
| NO: 149 | GGTGTTCCTCCTGATATCTGCGCATTCCACCGCTCCACCAGGAATTCCATTCTCCCCTACCTTCCTCA |
| | AGTCAACCCGTATCGAAAGCACGCTCAGGGTTAAGCCCCAAGATTACACTTCCGACGCGATCAACCA |
| | CCTACGAGCCCTTTACGCCCAATAAATCCGGACAACGCTCGCACCCTACGTATCACCGCGGCTGCTG |
| | GCACGTAGTTAGCCGGTGCTTCTTTACCCATTACCGTCACTCACGCTTCGTCACAGGCGAAAGCGGTT |
| | TACAACCCGAAGGCCGTCATCCCGCACGCGGCGTTGCTGCATCAGGCTTCCGCCCATTGTGCAATAT |
| | TCCCCA |
| OTU | >denovo4154 |
| Ref | |
| SEQ ID | CCTGTTTGCTCCCCACGCTTTCGAGCCTCAACGTCAGTTACCGTCCAGTAAGCCGCCTTCGCCACTGG |
| NO: 150 | TGTTCCTCCTAATATCTACGCATTTCACCGCTACACTAGGAATTCCGCTTACCCCTCCGGTACTCAAG |
| | ATCAACAGTTTCCAATGCAGTCCAGGGGTTGAGCCCCTGCCTTTCACATCAGACTTGCTGCTCCGTCT |
| | ACGCTCCCTTTACACCCAGTAAATCCGGATAACGCTTGCCCCCTACGTATTACCGCGGCTGCTGGCAC |
| | GTAGTTAGCCGGGGCTTCTTAGTCAGGTACCGTCATTATCTTCCCTGCTGATAGAAGTTTACATACCG |
| | AGATACTTCTTCCTTCACGCGGCGTCGCTGCATCAGGGTTTCCCCCATTGTGCAATATTCCCCA |
| OTU | >denovo4182 |
| Ref | |
| SEQ ID | CCTGTTTGCTCCCCACGCTTTCGAGCCTCAACGTCAGTTACCGTCCAGTAAGCCGCCTTCGCCACTGG |
| NO: 151 | TGTTCCTCCTAATATCTACGCATTTCACCGCTACACTAGGAATTCCGCTTACCTCTCCGGCACTCTAG |
| | AAGCACAGTTTCCAATGCAGTCCCGTGGTTGAGCCTCGGGTTTTCACATCAGACTTGCACTTCCGTCT |
| | ACGCTCCCTTTACACCCAGTAAATCCGGATAACGCTTGCCCCCTACGTATTACCGCGGCTGCTGGCAC |
| | GTAGTTAGCCGGGGCTTCTTACTCAGGTACCGTCATTTTCTTCCCTGCTGATAGAAGTTTACATACCG |
| | AAATACTTCATCCTTCACGCGGCGTCGCTGCATCAGGGTTTCCCCCATTGTGCAATATTCCCCA |
| OTU | >denovo4186 |
| Ref | |
| SEQ ID | CCTGTTCGCTCCCCCAGCTTTCGCGCCTCAGCGTCAGTGGCGGCCCAGCAGGCTGCCTTCGCCATCGG |
| NO: 152 | TGTTCTTCCCGATATCTGCGCATTCCACCGCTACACCGGGAATTCCGCCTGCCTCTACCGCACTCGAG |
| | CCGCCCAGTCCGGAACCCGGCCCGAGGTTGAGCCCCGGGATTAGAGGTTCCGCTTAGGCGGCCGCCT |
| | ACGCGCGCTTTACGCCCAATGAATCCGGATAACGCTCGCCCCCTACGTATTACCGCGGCTGCTGGCA |
| | CGTAGTTAGCCGGAGCTTCTTCTGCAGGTACCGTCTATGTCTTCCCTGCTGAAAGCGGTTTACAACCC |
| | GAAGGCCTTCGTCCCGCACGCGGCGTTGCTGCGTCAGGGTTTCCCCCATTGCGCAAAATTCCCCA |
| OTU | >denovo4206 |
| Ref | |
| SEQ ID | CCTGTTTGCTCCCCACGCTTTCGTGCCTCAGTGTCAGTTACAGTCCAGAAAGCCGCCTTCGCTACTGG |
| NO: 153 | TGTTCCTCCTAATATCTACGCATTTCACCGCTACACTAGGAATTCCACTTTCCTCTCCTGCACTCAAGT |
| | TTCCCAGTTTCAAGAGCTTACTACGGTTAAGCCGTAGCCTTTCACTCCTGACTTAAGAAACCACCTAC |
| | GCACCCTTTACGCCCAGTAAATCCGGATAACGCTAGCCCCCTACGTATTACCGCGGCTGCTGGCACG |
| | TAGTTAGCCGGGGCTTCCTCCTCAAGTACCGTCATTATCTTCCTTGAGGACAGAGTTTTACGACCCGA |
| | AGGCCTTCATCACTCACGCGGCGTTGCTGCATCAGGCTTTCGCCCATTGTGCAATATTCCCCA |
| OTU | >denovo4227 |
| Ref | |
| SEQ ID | CCTGTTCGCTCCCCGCGCTCTCGCGCCTCGGCGTCAGTGTCCTTCCGGCGGGCCGCCTTCGCCTCCGG |
| NO: 154 | TGTTCCTCCTGGCCTCTGCGCATTTCACCGCTACGCCAGGAATTCCGCCCGCCCCTCCGGCACTCCAG |
| | CGGCTCGGTTCGGGGCGCTTTCCCGGGGTTGGGCCCCGGATTTTCACACCCCGCCTTAGCCGCCGCCT |
| | GCGCGCGCTTTACGCCCAGTGATTCCGGACAACGCTCGCCCCCCACGTCTTGCCGCGGCTGCTGGCA |
| | CGTGGTTGGCCGGGGCTTTCTTGCCGGGTACCGTCCTGTCTCTTCCCCGGCGGCGGGGCTTTACGGGC |
| | CGAGGCCCTTCATCGCCCACGCGGCATCGCTGCGTCAGAGTTCCCTCCATTGCGCAATATTCCCCA |
| OTU | >denovo4230 |
| Ref | |
| SEQ ID | CCTGTTTGCTCCCCACGCTTTCGAGCCTCAACGTCAGTTACCGTCCAGTAAGCCGCCTTCGCCACTGG |
| NO: 155 | TGTTCCTCCTAATATCTACGCATTTCACCGCTACACTAGGAATTCCGCTTACCTCTCCGGCACTCTAG |
| | AAAAACAGTTTCCAATGCAGTCCTGGGGTTAAGCCCCAGCCTTTCACATCAGACTTGCTCTTCCGTCT |
| | ACGCTCCCTTTACACCCAGTAAATCCGGATAACGCTTGCCCCCTACGTATTACCGCGGCTGCTGGCAC |
| | GTAGTTAGCCGGGGCTTCTTAGTCAGGTACCGTCATTTTCTTCCCTGCTGATAGAAGTTTACATACCG |
| | AGATACTTCTTCCTTCACGCGGCGTCGCTGCATCAGGGTTTCCCCCATTGTGCAATATTCCCCA |
| OTU | >denovo4233 |
| Ref | |
| SEQ ID | CCTGTTTGCTACCCACGCTTTCGCGCTTCAGCGTCAGTATCTGTCCAGTGGGCTGACTTCTCCATCGG |
| NO: 156 | CATTCCTACAAATATCTACGAATTTCACCTCTACACTTGTAGTTCCGCCCACCTCTCCAGTACTCTAG |
| | TTTGACAGTTTCCAACGCAATACGGAGTTGAGCCCCGCATTTTCACATCAGACTTACCAAACCACCT |
| | AGACGCGCTTTACGCCCAATAAATCCGGATAACGCTTGCGACATACGTATTACCGCGGCTGCTGGCA |
| | CGTATTTAGCCGTCGCTTCTTCTGTTGGTACCGTCACGTTCTTCGTCCCAACTGAAAGCACTTTACATT |
| | CCGAAAAACTTCATCGTGCACACAGAATTGCTGGATCAGACTTTTGGTCCATTGTCCAATATTCCCCA |
| OTU | >denovo4282 |
| Ref | |
| SEQ ID | CCTGTTTGCTCCCCACGCTTTCGCACCTCAGCGTCAATACCGGTCCAGGTGGCCGCCTTCGCCACTGA |
| NO: 157 | TGTTCCTCCAGATATCTACGGATTTCACTCCTACACCTGGAATTCCGCCACCCTCTCCCGGATTCAAG |
| | TCGTGCAGTATCAAGGGCAGTTCCACGGTTGAGCCGTGGGATTTCACCCCTGACTTACATAACAGCC |
| | TACGTGCGCTTTACGCCCAGTGATTCCGATTAACGCTTGCACCCTCCGTATTACCGCGGCTGCTGGCA |
| | CGGAGTTAGCCGGTGCTTCCTTTGAAGGTACCGTCAGTAGGATGATGATTAGAGCACCCTAGTTTCTT |
| | CCCTTCTGACAGAGGTTTACGATCCGAAGACCTTCATCCCTCACGCGGCGTCGCTGCGTCAGGCTTTC |
| | GCCCATTGCGCAATATTCCCCA |
| OTU | >denovo4292 |
| Ref | |
| SEQ ID | CCTGTTTGCTCCCCACGCTTTCGAGCCTCAACGTCAGTTACTGTCCAGCAAGCCGCCTTCGCCACTGG |
| NO: 158 | TGTTCCTCCTAATATCTACGCATTTCACCGCTACACTAGGAATTCCGCTTACCTCTCCAGCACTCCAG |
| | CTTAACAGTTTCCAAAGCAGTCCCGGGGTTGAGCCCCGGGCTTTCACTTCAGACTTGCTAAGCCGTCT |
| | ACGCTCCCTTTACACCCAGTAAATCCGGATAACGCTTGCCCCCTACGTATTACCGCGGCTGCTGGCAC |
| | GTAGTTAGCCGGGGCTTCTTAGTCAGGTACCGTCATTCTCTTCCCTGCTGATAGAGCTTTACATACCG |
| | AAATACTTCTTCACTCACGCGGCGTCGCTGCATCAGGGTTTCCCCCATTGTGCAATATTCCCCA |
| OTU | >denovo4298 |
| Ref | |
| SEQ ID | CCTATTTGCTCCCCACGCTTTCGTGCCTGAGCGTCAGTTACAGACCAGGTAGCCGCCTTCGCCACTGG |
| NO: 159 | TGTTCCTCCATATATCTACGCATTTTACCGCTACACATGGAATTCCACTACCCTCTTCTGCACTCTAGT |
| | CTATCAGTTTTTAAGGCTATATTGAGTTGAGCTCAACGCTTTCACCCTAAACTTAATAGACCGCCTGC |
| | GCACCCTTTACGCCCAATAATTCCGGATAACGCTTGCCACCTACGTATTACCGCGGCTGCTGGCACGT |
| | AGTAAGCCGTGACTTTCTGGTAAAGTACCGTCAGTAGTAAATCATTTCCTATTTACTAAGTTCTTCCT |
| | TTACAACAGAGCTTTACAACCCAAAGGCCTTCATCACTCACGCGGCGTTGCTCGGTCAGGGTTTCCC |
| | CCATTGCCGAAAATTCCTTA |
| OTU | >denovo4320 |
| Ref | |
| SEQ ID | CCTGTTTGCTCCCCACGCTTTCGCACCTGAGCGTCAGTGTCTGTCCGGCAGGCCGCCTTCGCCTCTGG |
| NO: 160 | TATTCCTCCTGATATCTGCGCATTTCACCGCTACACCAGGAATTCTGCCTGCCTCTCCAGCACTCCAG |
| | CTTGAGGGTTCGGGATGGCCCTTCAGGGTTGGGCCCTGACTTTTCACATCCCGCCTTCCAAGCCGCCT |
| | GCGTGCCCTTTACGCCCAGTAATTCCGGACAACGCTCGCCCCCTACGTGTTACCGCGGCTGCTGGCA |
| | CGTAGTTAGCCGGGGCTTGCTTGTTAGGTACCGTCTCTCTTCTTCCCTAACGGCAGAGCTTTACGGGA |
| | CGAATCCCTTCTTCACTCACGCGGCATTGCTGCGTCAGAGTTGCCTCCATTGCGCAATATTCCCCA |
| OTU | >denovo4328 |
| Ref | |
| SEQ ID | CCTGTTTGATACCCACACTTTCGAGCATGAACGTCAGTTACAGTTTAGCAAGCTGCCTTCGCAATCGG |
| NO: 161 | GGTTCTTCGTGATATCTAAGCATTTCACCGCTACACCACGAATTCCGCCTGCCTCAACTGCACTCAAG |
| | GAAACCAGTATCAACTGCAATTTTACGGTTGAGCCGCAAACTTTCACAACTGACTTAATCTCCCGTCT |
| | GCGCTCCCTTTAAACCCAATAAATCCGGATAACGCTCGCATCCTCCGTATTACCGCGGCTGCTGGCA |
| | CGGAGTTAGCCGATGCTTATTCATACGGTACATACAAAATCCTACACGTAGGAAACTTTATTCCCGT |
| | ATAAAAGAAGTTTACAATCCGTAGGACCTTCATCCTTCACGCTACTTGGCTGGTTCAGGCTCTCGCCC |
| | ATTGACCAATATTCCTCA |
| OTU | >denovo435 |
| Ref | |
| SEQ ID | CCTGTTCGCTCCCCCAGCTTTCGCGCCTCAGCGTCAGTGCCGGCCCAGCAAGCTGCCTTCGCCGTCGG |
| NO: 162 | TGTTCTTCCCGATATCTGCGCATTCCACCGCTACACCGGGAATTCCACTTGCCTCTACCGCACTCGAG |
| | CCGTTCAGTTCAGAACCCGAACCAAGGTTGAGCCTCGGTCTTAAAGGTTCCGCTTAAGCGGCCGCCT |
| | ACGCGCCCTTTACGCCCAATGAATCCGGATAACGCTCGCCCCCTACGTGTTACCGCGGCTGCTGGCA |
| | CGTAGTTAGCCGGGGCTTCTTCTGTGGGTACCGTCAATTTCTTCCCCACTGAAAGCGGTTTACAACCC |
| | GAAGGCCTTCAGTCCCGCACGCGGCGTTGCTGCGTCAGGGTTGCCCCCATTGCGCAAGATTCCCCA |
| OTU | >denovo4360 |
| Ref | |
| SEQ ID | CCTGTTCGCTCCCCACGCTTTCGCACCTCAGCGTCAGTAAAGGCCCGGCAGACCGCCTTCGCCTCCGG |
| NO: 163 | TATTCCTCCTGATCTCTGCGCATTTCACCGCTACACCAGAAATTCTGTCTGCCTCTACCTCACTCTAGC |
| | TGCCCGGTTTGCAATGCTACCTCAGGGTTAAGCCCCGGGCTTTCACATCACACCTAAACAGCCGCCT |
| | GCGTGCCCTTTACGCCCAATAAATCCGGATAACGCTCGCCCCCTACGTATTACCGCAGCTGCTGGCA |
| | CGTAGTTGGCTGGGGCTTGCTCATCGGGTACCGTCTTCCTCTTCCCCGATAACAGAGCTTTACAGAAC |
| | GAATCCCTTCTTCACTCACGCGGCATTGCTGCGTCAGAGTTGCCTCCATTGCGCAATATTCCCCA |
| OTU | >denovo4364 |
| Ref | |
| SEQ ID | CCTGTTTGCTCCCCACGCTCTCGCGCCTTAGCGTCAGTCTCTTCCCAGCAGGCCGCCTTCGCCTCCGG |
| NO: 164 | TATTCCTCCTGATATCTGCGCATTTCACCGCTACACCAGGAATTCTGCCTGCCTCTAAAGTACTCCAG |
| | CCAGCTGGTTCCCCGTGCTTGACCCGATTGAGTCCGGCCCTTTTACACGAGGCCTCTCTGGCCGCCTA |
| | CGCGCCCTTTACGCCCAGTCATTCCGGACAACGCTTGCCCCCTACGTATTACCGCGGCTGCTGGCACG |
| | TAGTTAGCCGGGGCTTAAAGCAGGATAATGTCTCTCTCTTTTCCTGCTACTGAGCTCTGCGGGACGAA |
| | TCCCTTCCTCACTCACGCGGCATCGCTGCGTCAGAGTTGCCTCCATTGCGCAATATTCCCCA |
| OTU | >denovo443 |
| Ref | |
| SEQ ID | CCTGTTTGCTCCCCACGCTTTCGAGCCTCAACGTCAGTTACCGTCCAGTAAGCCGCCTTCGCCACTGG |
| NO: 165 | TGTTCCTCCTAATATCTACGCATTTCACCGCTACACTAGGAATTCCGCTTACCTCTCCGGCACTCTAG |
| | ATGGACAGTTTCCAAAGCAGTCCAGGGGTTGAGCCCCTGCCTTTCACTTCAGACTTGCCCGTCCGTCT |
| | ACGCTCCCTTTACACCCAGTAAATCCGGATAACGCTTGCCCCCTACGTATTACCGCGGCTGCTGGCAC |
| | GTAGTTAGCCGGGGCTTCTTAGTCAGGTACCGTCATTGTCTTCCCTGCTGATAGAAGTTTACATACCG |
| | AGATACTTCTTCCTTCACGCGGCGTCGCTGCATCAGGGTTTCCCCCATTGTGCAATATTCCCTA |
| OTU | >denovo4472 |
| Ref | |
| SEQ ID | CCTGTTTGCTCCCCACGCTTTCGTGCCTCAGCGTCAGTTACAGTCCAGAGAGCCGCCTTCGCAACTGG |
| NO: 166 | TATTCCTCCTAATATCTACGCATTTCACCGCTACACTAGGAATTCTACTCTCCTCTCCTGCACTCAAGT |
| | CTTACAGTTTCAAGAGCTTACTACGGTTGAGCCGTAGCCTTTCACTCCTGACTTGAAAGACCGCCTAC |
| | GCACCCTTTACGCCCAGTAATTCCGGATAACGCTAGCCCCCTACGTATTACCGCGGCTGCTGGCACG |
| | TAGTTAGCCGGGGCTTCCTCCTCAAGTACCGTCATTATCTTCCTTGAGGACAGAGCTTTACGACCCGA |
| | AGGCCTTCATCACTCACGCGGCGTTGCTGCATCAGGCTTTCGCCCATTGTGCAATATTCCCCA |
| OTU | >denovo4475 |
| Ref | |
| SEQ ID | CCTGTTTGCTCCCCACGCTTTCGCGCCTCAGCGTCAGTTGCTGTCCAGTTGACCGCCTTCGCCACTGG |
| NO: 167 | TGTTCCTCCTAATATCTACGCATTTCACCGCTACACTAGGAATTCCGTCAACCTCTCCAGTACTCAAG |
| | AACAACAGTTTCAAATGCAGTTTCAGGGTTGAGCCCTGAGATTTCACATCTGACTTGCCATCCCGCCT |
| | ACGCGCCCTTTACACCCAGTAAATCCGGATAACGCTTGCTCCCTACGTATTACCGCGGCTGCTGGCA |
| | CGTAGTTAGCCGGAGCTTATTCATCAGGTACCGTCACATCCTTCGTCCCTGATAAAAGAAGTTTACA |
| | ACCCGAAGGCCTTCTTCCTTCACGCGGCATTGCTGGGTCAGGCTTTCGCCCATTGCCCAATATTCCCC |
| | A |
| OTU | >denovo4477 |
| Ref | |
| SEQ ID | CCTGTTCGATACCCGCACTTTCGTGCCTGAGCGTCAGTTGAGCGCCGGTAAGCTGCCTTCGCAATCGG |
| NO: 168 | CGTTCTGCGTGATATCTATGCATTTCACCGCTACACCACGCATTCCGCTTACTTCTCGCTCACTCTAGT |
| | GCGGAAGTTTCAACGGCTCGGCGGAGTTGAGCTCCGTAATTTTACCGCTGACTTTCCGTACCGCCTGC |
| | GCACCCTTTAAACCCAATAAATCCGGATAACGCTCGCATCCTCCGTATTACCGCGGCTGCTGGCACG |
| | GAGTTAGCCGATGCTTTTTCTTCGGGTACTCTCCCTGATGACTCGCAAGTCACCCATTGCTCCCCGAC |
| | AAAAGAGGTTTACGACCCGTAGGGCCTTAGTCCCTCACGCGACTTGGCTGGTTCAGCCTTCCGGCCA |
| | TTGACCAATATTCCTCA |
| OTU | >denovo4485 |
| Ref | |
| SEQ ID | CCTGTTTGCTCCCCACGCTCTCGCGCCTTAGCGTCAGTCTCTTCCCGGCAGGCCGCCTTCGCCTCCGG |
| NO: 169 | TATTCCTCCTGATATCTGCGCATTTCACCGCTACACCAGGAATTCTGCCTGCCTCTAAAGGACTCCAG |
| | CCTCTCGGTTCCTGTCGCCTCCCCGGGTTGGGCCCGGGACTTTTACAACAGGCCTTCTAGGCCGCCTA |
| | CGCGCGCTTTACGCCCAGTAATTCCGGACAACGCTCGCCCCCTACGTATTACCGCGGCTGCTGGCAC |
| | GTAGTTAGCCGGGGCTTTAAAGACGGGTGACATCTCTCTTCTCCCCGTCGACTGAGCTCTGCGGGAC |
| | GAATCCCTTCCTCACTCACGCGGCATCGCTGCGTCAGAGTTTCCTCCATTGCGCAATATTCCCCA |
| OTU | >denovo4547 |
| Ref | |
| SEQ ID | CCTGTTTGCTCCCCACGCTCTCGCACCTGAGCGTCAGTGTCTGTCCGGCAGGCCGCCTTCGCCTCTGG |
| NO: 170 | TATTCCTCCTGATATCTGCGCATTTCACCGCTACACCAGGAATTCTGCCTGCCTCTCCAGCACTCGAG |
| | CTGCATGGTTCGGGATGCACCTCCAGGGTTGGGCCCTGGCTTTTCACATCCCGCCTACACAGCCGCCT |
| | GCGTGCCCTTTACGCCCAGTCATTCCGGACAACGCTCGCCCCCTACGTGTTACCGCGGCTGCTGGCAC |
| | GTAGTTAGCCGGGGCTTGCTTGTTAGGTACCGTCTGCTCTCTTCCCTAACGGCAGAGCTTTACGGAAC |
| | GAATTCCTTCTTCACTCACGCGGCATTGCTGCGTCAGAGTTGCCTCCATTGCGCAATATTCCCCA |
| OTU | >denovo4582 |
| Ref | |
| SEQ ID | CCTGTTTGCTCCCCACGCTTTCGTGCCTGAGCGTCAGTTATGTCCCAGGAGGCTGCCTTCGCCATCGG |
| NO: 171 | TGTTCTTCCACATATCTACGCATTTCACTGCTACACGTGGAATTCCACCTCCCTCTGACACACTCTAG |
| | TCGAGCAGTCACATATGCCATTCCCGAGTTAAGCTCGGGGATTTCACATCTGTCTTACTCAACCGCCT |
| | GCGCACCCTTTACGCCCAGTAATTCCGATTAACGCTCGCACCCTACGTATTACCGCGGCTGCTGGCAC |
| | GTAGTTAGCCGGTGCTTATTCTTAGAGTACCGTCAGCAACATCCTTTATTCGAAGATGTCTTTTCGTT |
| | CTCTACAAAAGTGGTTTACAACCCGAGGGCCTTCATCCCACACGCGGAATAGCTGGATCAGGCTTGC |
| | GCCCATTGTCCAAAATTCCCCA |
| OTU | >denovo4659 |
| Ref | |
| SEQ ID | CCTGTTTGATACCCGCACTTTCGAGCATCAGCGTCAGTTACGGTCCAGCAAGCTGCCTTCGCAATCGG |
| NO: 172 | AGTTCTTCGTGATATCTAAGCATTTCACCGCTACACCACGAATTCCGCCTGCCTTTACCGCACTCAAG |
| | AACTCCAGTATCAACTGCAATTTTACGGTTGAGCCGCAAACTTTCACAACTGACTTAAAATTCCGCCT |
| | ACGCTCCCTTTAAACCCAATAAATCCGGATAACGCTCGGATCCTCCGTATTACCGCGGCTGCTGGCA |
| | CGGAGTTAGCCGATCCTTATTCATACGGTACATACAAAAAACCACACGTGGCTAACTTTATTCCCGT |
| | ATAAAAGAAGTTTACAACCCATAGGGCAGTCATCCTTCACGCTACTTGGCTGGTTCAGACTCTCGTC |
| | CATTGACCAATATTCCTCA |
| OTU | >denovo4676 |
| Ref | |
| SEQ ID | CCTATTTGCTCCCCACGCTTTCGTGCTTCAGTGTCAGTTACAGGCCAGGCAACCGCCTTCGCCACTGG |
| NO: 173 | TGTTCTTCCATATATCTACGCATTTTACCGCTACACATGGAGTTCCATTGCCCTCTCCTGCACTCTAGC |
| | TTGCCAGTTTCCAAAGCGTACGACAGTTGAGCTGCCGGCTTTAACTCCAGACTTAACAAGCCACCTA |
| | CGCACCCTTTACGCCCAATCATTCCGGATAACGCTCGCCACCTACGTATTACCGCGGCTGCTGGCAC |
| | GTAGTTAGCCGTGACTTTCTGGTAAGCTGATCATTTATGGATCATTCCCTATCCATATCGTTTCTAACT |
| | TACAACAGAGCTTTACAATCCGAAGACCGTCTTCACTCACGCGGCATTGCTCGTTCAGGGTTCCCCCC |
| | ATTGACGAAAATTCCCTA |
| OTU | >denovo4681 |
| Ref | |
| SEQ ID | CCTATTTGCTCCCCACGCTTTCGTGCCTGAGCGTCAGTTACAGACTAGCAAGCCGCCTTCGCCACCGG |
| NO: 174 | TGTTCCTCCATATATCTATGCATTTTACCGCTACACATGGAATTCCACTTGCCCCTTCTGCACTCTAGT |
| | TCACCAGTTTCTAAGCCTGGATGGGGTTGAGCCCCACAATTTAAGCTTAAACTTAATAAACCGCCTG |
| | CGCACCCTTTACGCCCAATAATTCCGGATAACGCTCGTCACCTTCGTATTACCGCGGCTGCTGGCACG |
| | AAGTTAGCCGTGACTTTCTGGTAAAGTACCGTCACTTACGTAGTTGATACGTAACATTCTTCCTTTAC |
| | AACAGAGCTTTACAAGCCGAAGACCTTCATCACTCACGCGGCATTGCTCGGTCAGGCTTGCGCCCAT |
| | TGCCGAAAATTCCCTA |
| OTU | >denovo4699 |
| Ref | |
| SEQ ID | CCTGTTTGATCCCCGCACTTTCGTGCCTGAGCGTCAGTTGAGCGCCGGTATGCTGCCTTCGCAATCGG |
| NO: 175 | GGTTCTGCGTGATATCTATGCATTTCACCGCTACACCACGCATTCCGCATACTTCTCGCTCACTCTAG |
| | CATGTCAGTTTCAACGGCTATACGGCGTTGAGCACCGCATTTTTACCGCTGACTTGGCACGCCGCCTG |
| | CGCACCCTTTAAACCCAATAAATCCGGATAACGCTCGCATCCTCCGTATTACCGCGGCTGCTGGCAC |
| | GGAGTTAGCCGATGCTTTTTCTTCAGGTACTCTCGTCAGTCCACACGTGGACCTCATTGCTCCCTGAC |
| | AAAAGAGGTTTACGACCCATAGGGCCTTATTCCCTCACGCGACTTGGCTGGTTCAGCCTTCCGGCCA |
| | TTGACCAATATTCCTCA |
| OTU | >denovo4702 |
| Ref | |
| SEQ ID | CCTGTTTGCTCCCCACGCTTTCGCGCCTCAGCGTCAGTTACGGCCCAGCAAGTCGCCTTCGCCACTGG |
| NO: 176 | TGTTCCTCCTAATATCTACGCATTTCACCGCTACACTAGGAATTCCACTTGCCTCTTCCGCACTCAAG |
| | TCTGCCAGTATCGGATGCACGTTAAGAGTTGAGCCCTTACATTTCACATCCGACTTAACAGACCGCCT |
| | ACGCGCCCTTTACGCCCAGTGATTCCGGACAACGCTTGCCCCCTATGTTTTACCGCGGCTGCTGGCAC |
| | GTAGTTAGCCGGGGCTTCCTCCTCAAGTACCGTCATTATCTTCCTTGAGGACAGAGCTTTACGACCCG |
| | AAGGCCTTCATCGCTCACGCGGCGTTGCTGCATCAGGCTTTCGCCCATTGTGCAATATTCCCCA |
| OTU | >denovo4744 |
| Ref | |
| SEQ ID | CCTGTTTGCTCCCCACGCTTTCGTGCATGAGCGTCAGTGACATCCCAGGAGGCTGCCTTCGCCATTGG |
| NO: 177 | TGTTCTTCCAAATATCTACGCATTTCACTGCTACACTTGGAATTCCACCTCCCTCTGATGGACTCTAGT |
| | CCTGCAGTCACGAATGCAATTCCCAGGTTGAGCCCGGGGATTTCACATCCGTCTTACAGAACCGCCT |
| | GCGCACGCTTTACGCCCAGTAATTCCGATTAACGCTTGCACCCTACGTATTACCGCGGCTGCTGGCAC |
| | GTAGTTAGCCGGTGCTTATTCTTCAGGTACCGTCAGCAGATCCCGGTATTAGCGGAATCCTTTTCGTC |
| | CCTGACAAAAGCAGTTTACAATCCGAAGACCTTCATCCTGCACGCGGCATGGCTGGATCAGGGTTGC |
| | CCCCATTGTCCAAAATTCCCCA |
| OTU | >denovo477 |
| Ref | |
| SEQ ID | CCTGTTCGCTCCCCACGCTTTCGCGCCTGAGCGTCAGTATCCGGCTAGCAGACCGCCTTCGCCTCTGG |
| NO: 178 | TATTCCTCCTGATCTCTGCGCATTTCACCGCTACACCAGGAATTCTGTCTGCCCCGCCGGCACTCTAG |
| | TTGCCCGGTCCGCGATGACCGGCCGGGGTTGAGCCCCGGACTTTCACACCGCGCCTAAACAACCGCC |
| | TGCGCGCCCTTTACGCCCAATCAATCCGGATAACGCTCGCCCCCTACGTATTACCGCAGCTGCTGGC |
| | ACGTAGTTGGCTGGGGCTTGCTCGCCGGGTACCGTCTTAACTCTTTCCCGGCAACAGGGCTTTACAG |
| | GACGAATCCCGTCTTCACCCACGCGGCATTGCTGCGTCAGAGTTTCCTCCATTGCGCAATATTCCCCA |
| OTU | >denovo4780 |
| Ref | |
| SEQ ID | CCTGTTTGATCCCCACGCTTTCGTGCATCAGCGTCAGTCATGGCTTGGCAGGCTGCCTTCGCAATCGG |
| NO: 179 | GGTTCTGCGTGATATCTAAGCATTTCACCGCTACACCACGCATTCCGCCTGCCTCAAACATACTCAAG |
| | CCTCCCAGTTTCAACGGCAATTCTATGGTTGAGCCACAGACTTTCACCGCTGACTTAAAAGGCCGCCT |
| | ACGCACCCTTTAAACCCAATAAATCCGGATAACGCTCGGATCCTCCGTATTACCGCGGCTGCTGGCA |
| | CGGAGTTAGCCGATCCTTATTCATAAGGTACATACAAAACGGGACACGTCCCGCACTTTATTCCCTT |
| | ATAAAAGAGGTTTACGATCCATAGAACCTTCATCCCTCACGCGACTTGGCTGGTTCAGGCTCGCGCC |
| | CATTGACCAATATTCCTCA |
| OTU | >denovo4815 |
| Ref | |
| SEQ ID | CCTGTTTGCTACCCACGCTTTCGCGCTTCAGCGTCAGTATCTGTCCAGTGGGCTGACTTCTCCATCGG |
| NO: 180 | CATTCCTACAAATATCTACGAATTTCACCTCTACACTTGTAGTTCCGCCCACCTCTCCAGTACTCTAG |
| | TTTGACAGTTTCCAACGCAATACGGAGTTGAGCCCCGCATTTTCACATCAGACTTACCAAACCACCT |
| | AGACGCGCTTTACGCCCAATAAATCCGGATAACGCTTGCGACATACGTATTACCGCGGCTGCTGGCA |
| | CGTATTTAGCCGTCGCTTCTTCTGTTGGTACCGTCACGTTCTTCGTCCCAACTGAAAGCACTTTACATT |
| | CCGAAGACCTTCATCCTGCACGCGGCATGGCTGGATCAGGGTTGCCCCCATTGTCCAAAATTCCCCA |
| OTU | >denovo4819 |
| Ref | |
| SEQ ID | CCTGTTCGATCCCCGCACTTTCGAGCTTCAGCGTCAGTTGCGCTCCAGTGAGCTGCCTTCGCAATCGG |
| NO: 181 | AGTTCTTCGTGATATCTAAGCATTTCACCGCTACACCACGAATTCCGCCCACTTTGTGCGTACTCAAG |
| | GAAACCAGTTCGCGCTGCAGTGCAGATGTTGAGCATCTACATTTCACAACACGCTTAATCTCCGGCC |
| | TACGCTCCCTTTAAACCCAATAAATCCGGATAACGCCCGGACCTTCCGTATTACCGCGGCTGCTGGC |
| | ACGGAATTAGCCGGTCCTTATTCATAAGGTACATACAAAAAGAGTCACGACTCTCACTTTATTCCCTT |
| | ATAAAAGCAGTTTACAACCCATAGGGCCGTCTTCCTGCACGCTACTTGGCTGGTTCAGGCTCTCGCCC |
| | ATTGACCAATATTCCTCA |
| OTU | >denovo4833 |
| Ref | |
| SEQ ID | CCTGTTCGCTCCCCACGCTTTCGCTCCTCAGCGTCAGTGACGGCCCAGAGACCTGCCTTCGCCATTGG |
| NO: 182 | TGTTCTTCCCGATATCTACACATTCCACCGTTACACCGGGAATTCCAGTCTCCCCTACCGCACTCCAG |
| | CCCGCCCGTACCCGGCGCAGATCCACCGTTAGGCGATGGACTTTCACACCGGACGCGACGAACCGCC |
| | TACGAGCCCTTTACGCCCAATAAATCCGGATAACGCTCGCACCCTACGTATTACCGCGGCTGCTGGC |
| | ACGTAGTTAGCCGGTGCTTATTCGAACAATCCACTCAACACGGCCCGAAGCCGTGCCTTGCCCTTGA |
| | ACAAAAGCGGTTTACAACCCGAAGGCCTCCATCCCGCACGCGGCGTCGCTGCATCAGGCTTGCGCCC |
| | ATTGTGCAATATTCCCCA |
| OTU | >denovo4838 |
| Ref | |
| SEQ ID | CCTGTTCGCTCCCCCAGCTTTCGCGCCTCAGCGTCAGTGCCGGCCCAGCAGGCTGCCTTCGCCGTCGG |
| NO: 183 | TGTTCTTCCCGATATCTGCGCATTTCACCGCTACACCGGGAATTCCGCCTGCCCCTACCGCACTCGAT |
| | CCCCCCAGTTCGGCCCGCTTCCGGGGGTTGAGCCCCCGGCTTCGACGGTGCCGCTTGAGGAGACGCC |
| | TACGCGCGCTTTACGCCCAATGAATCCGGATAACGCTCGCCCCCTACGTATTACCGCGGCTGCTGGC |
| | ACGTAGTTAGCCGGGGCTTCTTCTGCAGGTACCGTCAAAATTCTTCCCTGCCGAAAGCGGTTTACGA |
| | CCCGAAGGCCTTCGTCCCGCACGCGGCGTTGCTGCGTCAGGGTTTCCCCCATTGCGCAAAATTCCCC |
| | A |
| OTU | >denovo4853 |
| Ref | |
| SEQ ID | CCTGTTCGCTCCCCACGCTTTCGCACCTCAGCGTCAGTATAAGCCCAGCAGGCCGCCTCCGCCACCG |
| NO: 184 | GTATTCCTCCTGATCTCTGCGCATTTCACCGCTACACCAGGAATTCTGCCTGCCTCTGCTTCACTCCA |
| | GCAGCACGGTTTGCCGTGCAACCCGGGGTTGGGCCCCGGGTTTTAACACCGCACCTTCGCTGCCGCC |
| | TGCGTGCCCTTTACGCCCAATAAATCCGGATAACGCTCGCCCCCCACGTGTTACCGCAGCTGCTGGC |
| | ACGTGGTTGGCTGGGGCTCATACGCCGGGTACCGTCTTAACTCTTCCCCGACAACAGGGCTTTACAG |
| | GACGAATCCCTTCTTCACCCACGCGGCATTGCTGCGTCAGAGTTGCCTCCATTGCGCAATATTCCCCA |
| OTU | >denovo4867 |
| Ref | |
| SEQ ID | CCTGTTTGATACCCACACTTTCGAGCATCAGCGTCAGTTACAATCCAGTAAGCTGCCTTCGCAATCGG |
| NO: 185 | AGTTCTTCGTGATATCTAAGCATTTCACCGCTACACCACGAATTCCGCCTACCTCTGTTGCACTCAAG |
| | GTCGCCAGTATCAACTGCAATTTTACGGTTGAGCCGCAAACTTTCACAACTGACTTAACAACCCGCC |
| | TACGCTCCCTTTAAACCCAATAAATCCGGATAACGCTCGGATCCTCCGTATTACCGCGGCTGCTGGC |
| | ACGGAGTTAGCCGATCCTTATTCATACGGTACATACAAAAAGCCACACGTGGCTAACTTTATTCCCG |
| | TATAAAAGAAGTTTACAACCCATAGGGCAGTCATCCTTCACGCTACTTGGCTGGTTCAGACTCTCGTC |
| | CATTGACCAATATTCCTCA |
| OTU | >denovo4876 |
| Ref | |
| SEQ ID | CCTGTTTGCTCCCCACGCTTTCGAGCCTCAGCGTCAGTTACAGTCCAGAGAGTCGCCTTCGCCACTGG |
| NO: 186 | TGTTCTTCCTAATCTCTACGCATTTCACCGCTACACTAGGAATTCCACTCTCCCCTCCTGTACTCTAGA |
| | TCCCCAGTTTGGAATGCAGCCCCCAGGTTGAGCCCGAGTATTTCACATCCCACTTAAAGATCCGCCT |
| | ACGCTCCCTTTACGCCCAGTAAATCCGGACAACGCTTGCCACCTACGTATTACCGCGGCTGCTGGCA |
| | CGTAGTTAGCCGTGGCTTCCTCCTTAGGTACCGTCATTATCGTCCCTAAAGACAGAGCTTTACAATCC |
| | GAAGACCGTCATCACTCACGCGGCGTTGCTGCATCAGGGTTTCCCCCATTGTGCAATATTCCCCA |
| OTU | >denovo4881 |
| Ref | |
| SEQ ID | CCTGTTTGCTCCCCACGCTCTCGCGCCTGAGCGTCAGTGTCTGTCCGGCAGGCCGCCTTCGCCTCTGG |
| NO: 187 | TATTCCTCCTGATCTCTGCGCATTTCACCGCTACACCAGGAATTCTGCCTGCCTCTCCAGCACTCGAG |
| | CCGCGCGGTTCGGGATGCCCGAACAGGGTTGGGCCCTGATCTTTCACATCCCGCCTTCGCGGCCGCC |
| | TGCGCGCCCTTTACGCCCAGTCATTCCGGACAACGCTCGCCCCCTACGTATTACCGCGGCTGCTGGCA |
| | CGTAGTTAGCCGGGGCTTGCTTGTCAGGTACCGTCTCTCTCTTCCCTGACAACAGAGCTTTACGGGAC |
| | GAATCCCTTCTTCGCTCACGCGGCATTGCTGCGTCAGAGTTGCCTCCATTGCGCAATATTCCCCA |
| OTU | >denovo4895 |
| Ref | |
| SEQ ID | CCTGTTTGCTCCCCACGCTTTCGAGCCTCAACGTCAGTTACCGTCCAGTAAGCCGCCTTCGCCACTGG |
| NO: 188 | TGTTCCTCCTAATATCTACGCATTTCACCGCTACACTAGGAATTCCGCTTACCCCTCCGGTACTCAAG |
| | ACTGACAGTTTCCAATGCAGTCCAGGGGTTGAGCCCCTGCCTTTCACATCAGACTTGCCATTCCGTCT |
| | ACGCTCCCTTTACACCCAGTAAATCCGGATAACGCTTGCCCCCTACGTATTACCGCGGCTGCTGGCAC |
| | GTAGTTAGCCGGGGCTTCTTAGTCAGGTACCGTCATTTTCTTCCCTGCTGATAGAAGTTTACATACCG |
| | AAATACTTCTTCGCTCACGCGGCGTCGCTGCATCAGGGTTTCCCCCATTGTGCAATATTCCCCA |
| OTU | >denovo4914 |
| Ref | |
| SEQ ID | CCTGTTTGATACCCACACTTTCGAGCATGAACGTCAGTTACACTTTAGCAAGCTGCCTTCGCAATCGG |
| NO: 189 | AGTTCTTCGTGATATCTAAGCATTTCACCGCTACACCACGAATTCCGCCTGCCTCAACTGCACTCAAG |
| | GTCTCCAGTATCAACTGCAATTTTAAGGTTGAGCCCCAAACTTTCACAGCTGACTTAAAAACCCGTCT |
| | GCGCTCCCTTTAAACCCAATAAATCCGGATAACGCTCGCATCCTCCGTATTACCGCGGCTGCTGGCA |
| | CGGAGTTAGCCGATGCTTATTCGTACCGGTACATACAAGCGCCTACACGTAGGCGGTTTTATTCCCG |
| | GACAAAAGAAGTTTACAACCCATAGGGCAGTCATCCTTCACGCTACTTGGCTGGTTCAGGCTCGCGC |
| | CCATTGACCAATATTCCTCA |
| OTU | >denovo4917 |
| Ref | |
| SEQ ID | CCTGTTTGCTACCCATGCTTTCGAGCCTCAGCGTCAGTTAGTGCCCAGTAGGCCGCCTTCGCCACTGG |
| NO: 190 | TGTTCCTCCCGATATCTACGCATTCCACCGCTACACCGGGAATTCCGCCTACCTCTACACCACTCAAG |
| | ATCCGCAGTTTTGAAAGCAGTTCATGGGTTGAGCCCATGAGTTTCACTTCCAACTTGCAGACCCGCCT |
| | GCGCTCCCTTTACACCCAGTAATTCCGGACAACGCTTGCCACCTACGTTTTACCGCGGCTGCTGGCAC |
| | GTAGTTAGCCGTGGCTTTCTTGTAAGGTACCGTCACTATCGTCCCTTACGACAGGAGTTTACAACCCG |
| | AAGGCCTTCTTCCTCCACGCGGCGTCGCTGCATCAGGGTTTCCCCCATTGTGCAATATTCCCCA |
| OTU | >denovo498 |
| Ref | |
| SEQ ID | CCTGTTTGATACCCACACTTTCGAGCCTCAATGTCAGTTGCAGCTTAGCAGGCTGCCTTCGCAATCGG |
| NO: 191 | AGTTCTTCGTGATATCTAAGCATTTCACCGCTACACCACGAATTCCGCCTGCCTCAACTGCACTCAAG |
| | ATATCCAGTATCAACTGCAATTTTACGGTTGAGCCGCAAACTTTCACAACTGACTTAAACATCCATCT |
| | ACGCTCCCTTTAAACCCAATAAATCCGGATAACGCTCGGATCCTCCGTATTACCGCGGCTGCTGGCA |
| | CGGAGTTAGCCGATCCTTATTCATAAAGTACATGCAAACGGGTATGCATACCCGACTTTATTCCTTTA |
| | TAAAAGAAGTTTACAACCCATAGGGCAGTCATCCTTCACGCTACTTGGCTGGTTCAGGCTCTCGCCC |
| | ATTGACCAATATTCCTCA |
| OTU | >denovo5016 |
| Ref | |
| SEQ ID | CCTGTTTGCTCCCCACGCTTTCGAGCCTCAACGTCAGTCATCGTCCAGCAAGCCGCCTTCGCCACTGG |
| NO: 192 | TGTTCCTCCTAATATCTACGCATTTCACCGCTACACTAGGAATTCCGCTTGCCTCTCCGACACTCTAG |
| | CTGCACAGTTTCCAAAGCAGTCCCGGGGTTGAGCCCCGGGCTTTCACTTCAGACTTGCACTGCCGTCT |
| | ACGCTCCCTTTACACCCAGTAAATCCGGATAACGCTTGCCCCCTACGTATTACCGCGGCTGCTGGCAC |
| | GTAGTTAGCCGGGGCTTCTTAGTCAGGTACCGTCATTTTCTTCCCTGCTGATAGAAGTTTACATACCG |
| | AAATACTTCATCCTTCACGCGGCGTCGCTGCATCAGGGTTTCCCCCATTGTGCAATATTCCCCA |
| OTU | >denovo5045 |
| Ref | |
| SEQ ID | CCTGTTTGCTCCCCACGCTTTCGCGCCTCAGCGTCAGTTATCGTCCAGCAATCCGCCTTCGCCACTGG |
| NO: 193 | TGTTCCTCCGTATATCTACGCATTTCACCGCTACACACGGAATTCCGATTGCCTCTCCGACACTCAAG |
| | AACTACAGTTTCAAATGCAGGCTATGGGTTGAGCCCATAGTTTTCACACCTGACTTGCAGTCCCGCCT |
| | ACACGCCCTTTACACCCAGTAAATCCGGATAACGCTCGCCACCTACGTATTACCGCGGCTGCTGGCA |
| | CGTAGTTAGCCGTGGCTTATTCAACAGGTACCGTCATTTGTTTCGTCCCTGTTAAAAGAAGTTTACAA |
| | CCCGAAAGCCTTCTTCCTTCACGCGGCGTTGCTGGGTCAGGCTTGCGCCCATTGCCCAATATTCCCCA |
| OTU | >denovo509 |
| Ref | |
| SEQ ID | CCTATTTGCTCCCCACGCTTTCGTGCCTCAGCGTCAGGTACAGGCCAGGCGGCCGCCTTCGCCGCTGG |
| NO: 194 | TGTTCTTCCACATCTCTACGCATTTTACCGCTACACATGGAGTTCCACCGCCCTCTCCTGTCCTCAAGC |
| | CGTGCAGTTTCCAAAGCCTGAACCGGTTGAGCCGGTCCCTTTTACTTCAGACTTGCACAGCCGCCTGC |
| | GCACCCTTTACGCCCAATCATTCCGGATAACGCTTGCCACCTACGTATTACCGCGGCTGCTGGCACGT |
| | AGTTAGCCGTGGCTTTCTGGCCGGGTACCATCAAACAGTCAGCTTTCCACTCTGGCTGTCCTTTGTCC |
| | CCGGCAACAGGGCTTTACAACCCGAAGGCCGTCATCCCGCACGCGGCGTCGCTGCGTCAGGCTTTCG |
| | CCCATTGCGCAAGATTCCCCA |
| OTU | >denovo5115 |
| Ref | |
| SEQ ID | CCTGTTTGCTACCCACGCTTTCGTGCCTCAACGTCAGTACAGGCCCAGCAAGCCGCCTTCGCCACTGG |
| NO: 195 | TGTTCCTCCATATATTTACGCATTTTACCGCTACACATGGAATTCCACTTGCCTCTACCGTACTCTAGC |
| | TTAAGAGTTTCTTTTGCTCGATTGGGTTGAGCCCAACAATTTTACAAAAGACTTCTTATGCCGTCTAC |
| | GCACCCTTTACGCCCAATAAATCCGGATAACGCTCGCCCCCTACGTATTACCGCGGCTGCTGGCACG |
| | TAGTTAGCCGGGGCTTATTCATTAGGTACCGTCAATACGAGTTTTCGTCCCTAATAAAAGAACTTTAC |
| | ATTACGAATAACTTCTTCGTTCACGCGGCATTGCTCGGTCAGACTTTCGTCCATTGCCGAAAATTCCC |
| | TA |
| OTU | >denovo5129 |
| Ref | |
| SEQ ID | CCTGTTTGCTCCCCACGCTTTCGTGCCTCAGCGTCAGTATCGGGCCAGTGAGCCGCCTTCGCCACTGG |
| NO: 196 | TGTTCTTGCGAATATCTACGAATTTCACCTCTACACTCGCAGTTCCACTCACCTCTCCCGAACTCAAG |
| | ATCTTCAGTATCAAAGGCAGTTCTGGAGTTGAGCTCCAGGATTTCACCCCTGACTTAAAGACCCGCC |
| | TACGCACCCTTTACGCCCAGTGATTCCGAGCAACGCTAGCCCCCTTCGTATTACCGCGGCTGCTGGCA |
| | CGAAGTTAGCCGGGGCTTATTCTTGCGGTACCGTCATTATCTTCCCGCACAAAAGAGCTTTACAACCC |
| | TAGGGCCTTCATCACTCACGCGGCATGGCTGGATCAGGCTTGCGCCCATTGTCCAATATTCCCCA |
| OTU | >denovo5135 |
| Ref | |
| SEQ ID | CCTGTTCGCTCCCCACGCTTTCGCACCTGAGCGTCAGTCACTGGCCGGCAGACCGCCTTCGCCTCCGG |
| NO: 197 | TATTCCTCCTGATCTCTGCGCATTTCACCGCTACACCAGGAATTCTGTCTGCCTCTCCAGCACTCGAG |
| | ACCTCCGGTTCGCCGTGGCCTCCCGGGGTTGAGCCCCGGTCTTTAACACGGCGCCTGAAAGTCCGCC |
| | TGCGTGCCCTTTACGCCCAATAAATCCGGATAACGCTCGCCCCCCACGTATTACCGCAGCTGCTGGC |
| | ACGTGGTTGGCTGGGGCTTGCTCGTCAGGTACCGTCTTCTTCTTCCCTGACAACAGAGCTTTACGGAA |
| | CGAATCCCTTCTTCACTCACGCGGCATTGCTGCGTCAGAGTTTCCTCCATTGCGCAATATTCCCCA |
| OTU | >denovo5161 |
| Ref | |
| SEQ ID | CCTGTTTGATACCCACGCTTTCGTGCCTCAGCGTCAATCGCGGATTCGTCAGATGCCTTCGCAATCGG |
| NO: 198 | TGTTCTGTGTGATATCTATGCATTTCACCGCTACACCACACATTCCTCCAACGACATCCGCATTCAAG |
| | CCTACCAGTATCAACGGCAGTTTCCTCGTTGAGCGAGGACATTTCACCGCTGACTTAACAGGCCGCC |
| | TACGCACCCTTTAAACCCAATAAATCCGGATAACGCTTGCATCCCCCGTATTACCGCGGCTGCTGGC |
| | ACGGAGTTAGCCGATGCTTATTCGTGAGATACTCTCATCAATCCACTCGTGGACCTTATTGCTCTCTC |
| | ACAAAAGAAGTTTAACGCCCATAGGGCACTGACCTTCACGCGGCATGGCTGGTTCAGCCTTTCGGCC |
| | ATTGACCAATATTCCTCA |
| OTU | >denovo5209 |
| Ref | |
| SEQ ID | CCTGTTTGCTCCCCACGCTTTCGAGCCTCAGCGTCAGTAAAAGCCCAGCAGGCCGCCTTCGCCACTG |
| NO: 199 | GTGTTCCTCCTAATATCTACGCATTTCACCGCTACACTAGGAATTCCGCCTGCCTCTACTTCACTCAA |
| | GAGCCCCAGTTTCGAACGCAGCTACCGGTTGAGCCGGTAGATTTAACATTCGACTTAAGACCCCGCC |
| | TACGCTCCCTTTACACCCAGTAATTCCGGACAACGCTCGCTCCCTACGTATTACCGCGGCTGCTGGCA |
| | CGTAGTTAGCCGGAGCTTCCTCCTTGGGTACCGTCATTATCGTCCCCAAAGACAGAGGTTTACAATCC |
| | GAAGACCGTCTTCCCTCACGCGGCGTCGCTGCATCAGAGTTTCCTCCATTGTGCAATATCCCCCA |
| OTU | >denovo5302 |
| Ref | |
| SEQ ID | CCTGTTTGCTCCCCCAGCTTTCGCGCCTCAGCGTCCGTCTCGGCCCAGAGGGCCGCCTTCGCCACCGG |
| NO: 200 | TGTTCCACCCGATATCTGCGCATTCCACCGCTACACCGGGTGTTCCACCCTCCCCTACCGGACCCGAG |
| | CCGCGGGGTTCGGGGGGCGGACCGGGGTTGAGCCCCGGGATTTGACCCCCCGCCTACGCGGCCGCCT |
| | ACGCGCGCTTTACGCCCAATGAATCCGGATAACGCGCGCCCCCTACGTATTACCGCGGCTGCTGGCA |
| | CGTAGTTAGCTGGGGCTTCTTCTGCAGGTACCGTCTTGTCTCGTCCCTGCTGAAAGCGGTTTACGACC |
| | CGAAGGCCTTCGTCCCGCACGCGGCGTCGCTGCGTCAGGGTTTCCCCCATTGCGCAAGATTCCCCA |
| OTU | >denovo5334 |
| Ref | |
| SEQ ID | CCTATTTGCTCCCCACGCTTTCGTGCCTGAGCGTCAGTTACAGTCCAGGCGGCCGCCTTCGCCACTGG |
| NO: 201 | TGTTCTTCCATATATCTACGCATTTTACCGCTACACATGGAGTTCCACCGCCCTCTCCTGTCCTCTAGC |
| | GTGACAGTTTCCAAAGCCTGTACAGGTTGAGCCCGTACCTTTCACTTCAGACTTGTCACGCCGCCTGC |
| | GCACCCTTTACGCCCAATCATTCCGGATAACGCTCGCCACCTACGTATTACCGCGGCTGCTGGCACGT |
| | AGTTAGCCGTGACTTTCTGGCAGGCTACCATCAAAAGACAAGCATTTCCTCTTGCCTTCCTTTTTGAC |
| | CTGCAACAGAGCTTTACGATCCGAAGACCTTCCTCACTCACGCGGCATTGCTCGTTCAGGCTTGCGCC |
| | CATTGACGAAAATTCCCTA |
| OTU | >denovo5338 |
| Ref | |
| SEQ ID | CCTGTTTGCTCCCCACGCTTTCGAGCCTCAACGTCAGTTACTGTCCAGTAAGCCGCCTTCGCCACTGG |
| NO: 202 | TGTTCCTCCTAATATCTACGCATTTCACCGCTACACTAGGAATTCCACTTACCTCTCCAGCACTCTAG |
| | CAGAACAGTTTCCAAAGCAGTCCCGGGGTTGAGCCCCGGGCTTTCACTTCAGACTTGCTCCGCCGTC |
| | TACGCTCCCTTTACACCCAGTAAATCCGGATAACGCTTGCCCCCTACGTATTACCGCGGCTGCTGGCA |
| | CGTAGTTAGCCGGGGCTTCTTAGTCAGGTACCGTCATTTTCTTCCCTGCTGATAGAGCTTTACATACC |
| | GAGATACTTCTTCACTCACGCGGCGTCGCTGCATCAGGGTTTCCCCCATTGTGCAATATTCCCCA |
| OTU | >denovo5385 |
| Ref | |
| SEQ ID | CCTGTTTGCTCCCCACGCTTTCGAGCCTCAGTGTCAGTTACAGTCCAGTAAGCCGCCTTCGCCGCTGG |
| NO: 203 | TGTTCCTCCTAATATCTACGCATTTCACCGCTACACTAGGAATTCCACTTACCTCTCCTGCACTCCAG |
| | CCCAGCAGTTTCAAAAGCAGTCCCGGGGTTGAGCCCCGGGCTTTCACTTCTGACTTGCTGTGCCACCT |
| | ACACTCCCTTTACACCCAGTAAATCCGGATAACGCTTGCTCCATACGTATTACCGCGGCTGCTGGCAC |
| | GTATTTAGCCGGAGCTTCTTAGTCAGGTACCGTCATTATCTTCCCTGCTGATAGAGCTTTACATACCG |
| | AAATACTTCTTCACTCACGCGGCGTCGCTGCATCAGAGTTTCCTCCATTGTGCAATATTCCCCA |
| OTU | >denovo5399 |
| Ref | |
| SEQ ID | CCTGTTTGCTCCCCACGCTTTCGCGCATCAGCGTCAGTAATGTTCCAGCAGGTCGCCTTCGCAATGAG |
| NO: 204 | TATTCCTCTTGATCTCTACGGATTTTACCCCTACACCAAGAATTCCACCTACCTCTCCCATACTCTAGA |
| | AAAGTAGTTTCAAATGCAGTTCTGTAGTTAAGCCACAGGATTTCACATCTGACTTACCTTCCCGCCTA |
| | CGCGCTCTTTACGCCCAGTGATTCCGAGTAACGCTTGCACCCTCCGTATTACCGCGGCTGCTGGCACG |
| | GAGTTAGCCGGTGCTTATTCGTTAGATACCGTCATAATCTTCTCTAACAAAAGGAGTTTACAATCCTA |
| | AAACCTTCATCCTCCACGCGGCGTTGCTGCTTCAGGGTTTCCCCCATTGAGCAATATTCCCTA |
| OTU | >denovo5427 |
| Ref | |
| SEQ ID | CCTATTTGCTCCCCACGCTTTCGTGCCTCAGTGTCAGTTACAGGCCAGGCGACCGCCTTCGCCACTGG |
| NO: 205 | TGTTCCTCCATATATCTACGCATTTTACCGCTACACATGGAATTCCATCGCCCTCTCCTGCACTCTAGT |
| | TCACCAGTTTCCAAAGCGAACAGCAGTTGAGCTGCTGCCTTTTACTTCAGACTTAATGTACCACCTAC |
| | GCACCCTTTACGCCCAATCATTCCGGATAACGCTTGCCACCTACGTATTACCGCGGCTGCTGGCACGT |
| | AGTTAGCCGTGGCTTTCTGGTAAGATACCATCACTATCATGTCATTCCCTACATGATCCGTTTTTCTCT |
| | TACAACAGAGCTTTACGATCCGAAGACCTTCTTCACTCACGCGGCATTGCTCGTTCAGGGTTCCCCCC |
| | ATTGACGAAAATTCCCTA |
| OTU | >denovo5455 |
| Ref | |
| SEQ ID | CCTGTTTGCTCCCCACGCTTTCGCACCTGAGCGTCAGTCTTCGTCCAGGGGGCCGCCTTCGCCACCGG |
| NO: 206 | TATTCCTCCAGATCTCTACGCATTTCACCGCTACACCTGGAATTCTACCCCCCTCTACGAGACTCAAG |
| | CTTGCCAGTATCAGATGCAGTTCCCAGGTTGAGCCCGGGGATTTCACATCTGACTTAACAAACCGCC |
| | TGCGTGCGCTTTACGCCCAGTAATTCCGATTAACGCTTGCACCCTCCGTATTACCGCGGCTGCTGGCA |
| | CGGAGTTAGCCGGTGCTTCTTCTGCGGGTAACGTCAATGAGCAAAGGTATTAACTTTACTCCCTTCCT |
| | CCCCGCTGAAAGTACTTTACAACCCGAAGGCCTTCTTCATACACGCGGCATGGCTGCATCAGGCTTG |
| | CGCCCATTGTGCAATATTCCCCA |
| OTU | >denovo546 |
| Ref | |
| SEQ ID | CCTGTTCGCTCCCCACGCTTTCGTGCCTCAGCGTCAGTTTCCAGTCCAGAAAGCCGCCTTCGCCACTG |
| NO: 207 | GTGTTCTTCCTAATATCTACGCATTTCACCGCTACACTAGGAATTCCGCTTTCCTCTCCTGTACTCTAG |
| | CTTAGCAGTTTAAAATGCAATCCCGAGGTTAAGCCTCGGGCTTTCACATCTTACTTGCCATGCCGCCT |
| | ACTCACCCTTTACACCCAGTAATTCCGGATAACGCTTGCCCCCTACGTATTACCGCGGCTGCTGGCAC |
| | GTAGTTAGCCGGGGCTTCTTATTTAGGTACAGTCACTTTCTTCGTCCCTATTGATAGAAGTTTACAAT |
| | CCGAAAACCGTCTTCCTTCACGCGGCGTTGCTGCATCAGGGTTTCCCCCATTGTGCAATATTCCCCA |
| OTU | >denovo5466 |
| Ref | |
| SEQ ID | CCTGTTTGATACCCACACTTTCGAGCCTCAATGTCAGTTACAGCTTAGCAGGCTGCCTTCGCAATCGG |
| NO: 208 | AGTTCTTCGTGATATCTAAGCATTTCACCGCTACACCACGAATTCCGCCTGCCTCAACTGCACTCAAG |
| | GCGTCCAGTATCAACTGCAATTTTACGGTTGAGCCGCAAACTTTCACAACTGACTTAAACGCCCATCT |
| | ACGCTCCCTTTAAACCCAATAAATCCGGATAACGCTCGGATCCTCCGTATTACCGCGGCTGCTGGCA |
| | CGGAGTTAGCCGATCCTTATTCATACGGTACATACAAAAAGGTACGCGTACCTCACTTTATTCCCGTA |
| | TAAAAGAAGTTTACAACCCATAGGGCAGTCATCCTTCACGCTACTTGGCTGGTTCAGACTCTCGTCC |
| | ATTGACCAATATTCCTCA |
| OTU | >denovo5487 |
| Ref | |
| SEQ ID | CCCGTTCGCTACCCTAGCTTTCGAGCCTCAGCGTCAGTTACAGTCCAGAAAGCCGCCTTCGCCACTGG |
| NO: 209 | TGTTCTTCCCAATATCTACGCATTTCACCGCTACACTGGGAATTCCGCTTTCCTCTCCTGCACTCAAG |
| | AAAATCAGTTCGGACCCCATCACGAGGTTGAGCCCCGCACTTTTAAGATCCGCTTAATTTCCCGCCTG |
| | CGCTCCCTTTACGCCCAATAATTCCGGACAACGCTTGCCACCTACGTATTACCGCGGCTGCTGGCACG |
| | TAGTTAGCCGTGGCTTCCTCGTTAAGTACCGTCAAATACTTACTGTATTATAATAAGCATCCTTCGCC |
| | CTTAACAACAGAACTTTACGATCCGAAGACCTTCTTCGTTCACGCGGCGTTGCTCCGTCAGGCTTTCG |
| | CCCATTGCGGAAGATTCCCCA |
| OTU | >denovo5517 |
| Ref | |
| SEQ ID | CCTGTTCGCTCCCCCAGCTTTCGCGCCTCAGCGTCGGTCTCGGCCCAGAGGGCCGCCTTCGCCACCGG |
| NO: 210 | TGTTCCACCCGATATCTGCGCATTCCACCGCTACACCGGGTGTTCCACCCTCCCCTACCGGACCCGAG |
| | CCCGGCGGTTCAGGGGGCGGGACGGGGTTGAGCCCCGCCATTTGACCCCCTGCCTGCCGGGCCGCCT |
| | ACGCGCGCTTTACGCCCAATGAATCCGGATAACGCTCGCCCCCTACGTATTACCGCGGCTGCTGGCA |
| | CGTAGTTAGCCGGGGCTTCTTCTGCAGGTACCGTCTTGTCTCATCCCTGCTGAAAGCGGTTTACGACC |
| | CGAGGGCCTTCGTCCCGCACGCGGCGTCGCTGCGTCAGGGTTCCCCCCATTGCGCAAGATTCCCCA |
| OTU | >denovo5529 |
| Ref | |
| SEQ ID | CCCGTTCGCTACCCTAGCTTTCGAGCCTCAGCGTCAGTTACAGTCCAGAAAGCCGCCTTCGCCACTGG |
| NO: 211 | TGTTCTTCCCAATATCTACGCATTTCACCGCTACACTGGGAATTCCGCTTTCCTCTCCTGCACTCAAG |
| | AAAACCAGTTCGGACCCCATCACGGGGTTGAGCCCCGCACTTTTAAGATCCGCTTAGTTTCCCGCCT |
| | GCGCTCCCTTTACGCCCAATAATTCCGGACAACGCTTGCCGCCTACGTATTACCGCGGCTGCTGGCAC |
| | GTAGTTAGCCGTGGCTTCCTCGTTAGGTACCGTCAAATAAAAACCTTATTATGATTCTTACCCTTCGT |
| | CCCTAACAACAGAACTTTACGATCCTAAGACCTTCATCGTTCACGCGGCGTTGCTCCGTCAGGCTTTC |
| | GCCCATTGCGGAAGATTCCCCA |
| OTU | >denovo5549 |
| Ref | |
| SEQ ID | CCTGTTTGCTACCCATGCTTTCGAGCCTCAGCGTCAGTTAAAGCCCAGTAGGCCGCCTTCGCCACTGG |
| NO: 212 | TGTTCCTTCCGATATCTACGCATTCCACCGCTACACCGGAAATTCCGCCTACCTCTACTTCACTCAAG |
| | TTCCACAGTTTTGAACGCTGATACGGGTTGAGCCCGTAGATTAAACGTCCAACTTGCAGAACCGCCT |
| | GCGCTCCCTTTACACCCAGTAATTCCGGACAACGCTCGCACCCTACGTTTTACCGCGGCTGCTGGCAC |
| | GTAGTTAGCCGGTGCTTCCTCCTCGGGTACCGTCATTATCGTCCCCGAAGACAGAGGTTTACAATAC |
| | GAATACCTTCTTCCCTCACGCGGCGTCGCTGCATCAGAGTTTCCTCCATTGTGCAATATTCCCCA |
| OTU | >denovo5571 |
| Ref | |
| SEQ ID | CCTGTTTGCTCCCCACGCTTTCGTGCCTCAGCGTCAGCTCCAGCCCAGACGGCCGCCTTCGCCGCTGG |
| NO: 213 | TGTTCTTCCATATCTCTATGCATTTTACCGCTACACATGGAGTTCCGCCGTCCTCTGCTGGCCTCCAGC |
| | CTGCCAGTTTCCACAGCCTGGCAAAGTTGAGCTTCGCCCTTTTACTGCAGACTTAACAGGCCGCCTGC |
| | GCACCCTTTACGCCCAATCATTCCGGATAACGCTCGCCACCTACGTATTACCGCGGCTGCTGGCACGT |
| | AGTTAGCCGTGACTTCCTCGTGGACTTCCATCAAACTAAAGCCATTTCCTGCTCTTGTCCTTTTTCATC |
| | CACAACAGGGCTTTACACTCCGAAAAGCTCCTTCACCCACGCGGCATCGCTCGTTCAGGCTTGCGCC |
| | CATTGACGAAGATTCCCTA |
| OTU | >denovo5573 |
| Ref | |
| SEQ ID | CCTATTTGCTCCCCACGCTTTCGTGCCTGAGCGTCAGTTACAGGCCAGGCGGCCGCCTTCGCCACTGG |
| NO: 214 | TGTTCTTCCACATCTCTACGCATTTTACCGCTACACGTGGAGTTCCACCGCCCTCTCCTGTCCTCGAGC |
| | ATACCAGTTTCCAAAGCCTGTACAGGTTGAGCCCGTACCTTTCACTTCAGACTTGATATGCCGCCTGC |
| | GCACCCTTTACGCCCAATCATTCCGGATAACGCTCGCCACCTACGTATTACCGCGGCTGCTGGCACGT |
| | AGTTAGCCGTGACTTTCTGGCGAGGTACCATCAAAAGAGAATCATTCCCTCTTCTCTTCCTTTTTCCC |
| | TCGCAACAGAGCTTTACGATCCGAAGACCTTCTTCACCCACGCGGCATTGCTCGTTCAGGCTTGCGCC |
| | CATTGACGAAAATTCCCTA |
| OTU | >denovo5581 |
| Ref | |
| SEQ ID | CCTATTTGCTCCCCACGCTTTCGTGCCTGAGCGTCAGTTACAGACTAGCAAGCCGCCTTCGCCACCGG |
| NO: 215 | TGTTCCTCCATATATCTATGCATTTTACCGCTACACATGGAATTCCACTTGCCCCTTCTGCACTCTAGT |
| | TCACCAGTTTCTAAGCCTGGATGGGGTTGAGCCCCACAATTTAAGCTTAAACTTAGCAAACCGCCTG |
| | CGCACCCTTTACGCCCAATAATTCCGGATAACGCTTGTCACCTTCGTATTACCGCGGCTGCTGGCACG |
| | AAGTTAGCCGTGACTTTCTGGTAAAGTACCGTCAGTTACATAGTTGATATGTAATATTCTTCCTTTAC |
| | AACAGAGCTTTACAATCCGAAGACCTTCATCACTCACGCGGCATTGCTCGGTCAGGCTTTCGCCCATT |
| | GCCGAAAATTCCCTA |
| OTU | >denovo5585 |
| Ref | |
| SEQ ID | CCTGTTTGCTCCCCACGCTTTCGTGCCTCAGCGTCAGCTCCAGCCCAGACGGCCGCCTTCGCCGCTGG |
| NO: 216 | TGTTCTTCCATATCTCTATGCATTTTACCGCTACACATGGAGTTCCGCCGTCCTCTGCTGGCCTCCAGC |
| | CTGCCAGTTTCCACAGCCTGGCAAAGTTGAGCTTCGCCCTTTTACTGCAGACTTAACAGGCCGCCTGC |
| | GCACCCTTTACGCCCAATCATTCCGGATAACGCTCGCCACCTACGTATTACCGCGGCTGCTGGCACGT |
| | AGTTAGCCGTGACTTCCTCGTGGACTTCCATCAAAAGCAGACCATTTCCTGTCTCCTCCCTTTTTCATC |
| | CACAACAGGGCTTTACACTCCGAAAAGCTCCTTCACCCACGCGGCATCGCTCGTTCAGGCTTGCGCC |
| | CATTGACGAAGATTCCCTA |
| OTU | >denovo5632 |
| Ref | |
| SEQ ID | CCTATTTGCTCCCCACGCTTTCGTGCCTCAGCGTCAGGTACAGGCCAGGCGGCCGCCTTCGCCGCTGG |
| NO: 217 | TGTTCTTCCACATCTCTACGCATTTTACCGCTACACATGGAGTTCCACCGCCCTCTCCTGTCCTCAAGC |
| | CGTGCAGTTTCCAAAGCCTGAACCGGTTGAGCCGGTCCCTTTTACTTCAGACTTGCACAGCCGCCTGC |
| | GCACCCTTTACGCCCAATCATTCCGGATAACGCTTGCCACCTACGTATTACCGCGGCTGCTGGCACGT |
| | AGTTAGCCGTGGCTTTCTGGCCGGGTACCATCAAACAGTCAGCTTTCCACTCTGGCTATCCTTTGTCC |
| | CCGGCAACAGGGCTTTACAGCCCGAAGGCCGTCTTCACTCACGCGGCATTGCTCGTTCAGAGTTTCC |
| | TCCATTGACGAAAATTCCCTA |
| OTU | >denovo5653 |
| Ref | |
| SEQ ID | CCTGTTCGATACCCACGCTTTCGTGCTTCAGCGTCAGTAGGGCGCCGGTATGCTGCCTTCGCAATCGG |
| NO: 218 | AGTTCTGCGTGATATCTATGCATTTCACCGCTACACCACGCATTCCGCATACTTCTCGCCCACTCAAG |
| | ATCTCCAGTTTCAACGGCGGGATGAGGTTGAGCCTCACTATTTTACCGCTGACTTAAAAATCCGCCTA |
| | CGCACCCTTTAAACCCAATAAATCCGGATAACGCTCGCATCCTCCGTATTACCGCGGCTGCTGGCAC |
| | GGAGTTAGCCGATGCTTTTTCTTCGGGTAATCTCGCTTCAGGCACACGTGCCTTCGTTGCTCCCCGAC |
| | AAAAGAGGTTTACAATCCATAGGACCGTCTTCCCTCACGCGACTTGGCTGGTTCAGGCTCTCGCCCA |
| | TTGACCAATATTCCTCA |
| OTU | >denovo5657 |
| Ref | |
| SEQ ID | CCTGTTTGCTCCCCACGCTTTCGAGCCTCAGCGTCATTTACAGTCCAGTAAGCCGCCTTCGCCACTGG |
| NO: 219 | TGTTCCTCCTAATATCTACGCATTTCACCGCTACACTAGGAATTCCACTTACCTCTCCTGCAATCTAG |
| | CAATACAGTTTCAAAAGCAGTCCCGGAGTTGAGCCCCGGGCTTTCACTTCTGACTTGCATCGCCGCCT |
| | ACGCTCCCTTTACACCCAGTAAATCCGGATAACGCTTGCCCCCTACGTATTACCGCGGCTGCTGGCAC |
| | GTAGTTAGCCGGGGCTTCTTAGTCAGGTACCGTCATTTTCTTCCCTGCTGATAGAAGTTTACATACCG |
| | AAATACTTCTTCCTTCACGCGGCGTCGCTGCATCAGGGTTTCCCCCATTGTGCAATATTCCCCA |
| OTU | >denovo5659 |
| Ref | |
| SEQ ID | CCTGTTTGCTACCCACGCTTTCGCACCTCAGCGTCAGTTACCGACCAGATTGCCGCCTTCGCTACCGG |
| NO: 220 | TATTCCTCCTGATATCTACGCATTTCACCGCTACACCAGGAATTCTGCAATCCTCTCCGATACTCTAG |
| | TATTATAGTATCACTAGCCTTACTAAAGTTGAGCTCTAGCCTTTGACTAATGACTTACAACACCGCCT |
| | ACGTGCTCTTTACGCCCAGTCACTCCGAACAACGCTCGCCCCCTCCGTATTACCGCGGCTGCTGGCAC |
| | GGAGTTAGCCGGGGCTGCTTATACAGGTACCGTCACTATATATGCAGTTACTCATATATATTTTCTTC |
| | CCTGTCGAAAGAACTTTACGACCCGAAGGCCTTCTTCATTCACGCGGCGTCGCTGCGTCACTCTATCG |
| | AGCATTGCGCAAAATTCCCCA |
| OTU | >denovo5686 |
| Ref | |
| SEQ ID | CCTGTTTGCTCCCCACGCTCTCGCACCTGAGCGTCAGTATCTGTCCGGCAGGCCGCCTTCGCCTCTGG |
| NO: 221 | TATTCCTCCTGATATCTGCGCATTTCACCGCTACACCAGGAATTCTGCCTGCCTCTCCAGTACTCTAG |
| | CCGCATGGTTCGGGATGCCCTTCCAGGGTTGGGCCCTGATCTTTCACATCCCGCCTACACGGCCGCCT |
| | GCGTGCCCTTTACGCCCAGTCATTCCGGACAACGCTCGCCCCCTACGTGTTACCGCGGCTGCTGGCAC |
| | GTAGTTAGCCGGGGCTTGCTTGTTAGGTACCGTCTTCTCTCTTCCCTAACGGCAGAGCTTTACGGAAC |
| | GAATTCCTTCTTCACTCACGCGGCATTGCTGCGTCAGAGTTGCCTCCATTGCGCAATATTCCCCA |
| OTU | >denovo5704 |
| Ref | |
| SEQ ID | CCTGTTTGCTCCCCACGCTTTCGCACCTCAGCGTCAGTATCTGCCCAGGAAATCGCCTTCGCCTCTGG |
| NO: 222 | TGTTCTTCCAGATATCTACGCATTTCACCGCTACACCTGGAATTCCATTTCCCTCTGCAATACTCTAG |
| | ATAGTTAGTTTAAAGTGCAGTTCCCAGGTTGAGCCCGGGGCTTTCACACCTTACTTGACTATCCGCCT |
| | ACATGCCCTTTACGCCCAGTTATTCCGATTAACGCTTGCACCCTCCGTATTACCGCGGCTGCTGGCAC |
| | GGAGTTAGCCGGTGCTTCTTCTGTGGGTAACGTCACTCTTGCGATTTCCTCCCCACTGAAAGTGCTTT |
| | ACAACCCGAAGGCCTTCTTCACACACGCGGCATGGCTGCATCAGGGTTCCCCCCATTGTGCAATATT |
| | CCCTA |
| OTU | >denovo5727 |
| Ref | |
| SEQ ID | CCTGTTTGCTCCCCACGCTCTCGCGCCTGAGCGTCAGTCTCTCTCCAGCAGGCCGCCTTCGCCTCCGG |
| NO: 223 | TATTCCTCCTGATCTCTGCGTATTTCACCACTACACCAGGAATTCTGCCTGCCTCTAGAGTACTCTAG |
| | CTAAGCTGGTTCTTTCTGCTTCCCCGGGGTGGGCCCGGGGCTTTTACAAAAAGCCTCTCTTGCCGCCT |
| | GCGCGCCCTTTACGCCCAATAATTCCGGACAACGCTCGCCCCCTACGTATTGCCGCGGCTGCTGGCA |
| | CGTAGTTAGCCGGGGCTCAAAACCGGCTGGTCTTCTCTCCTAACCGGTGACTGAGCTTTACGGGACG |
| | AATCCCTTCCTCACTCACGCGGCATCGCTGCGTCAGAGTTTCCTCCATTGCGCAATATTCCCCA |
| OTU | >denovo5753 |
| Ref | |
| SEQ ID | CCTGTTTGCTCCCCACGCTTTCGAGCCTCAACGTCAGTTACCGTCCAGTAAGCCGCCTTCGCCACTGG |
| NO: 224 | TGTTCCTCCTAATATCTACGCATTTCACCGCTACACTAGGAATTCCGCTTACCTCTCCGGCACTCGAG |
| | TATCACAGTTTCCAATGCAGTCCAGGGGTTGAGCCCCCGCCTTTCACATCAGACTTGCAACACCGTCT |
| | ACGCTCCCTTTACACCCAGTAAATCCGGATAACGCTTGCCCCCTACGTATTACCGCGGCTGCTGGCAC |
| | GTAGTTAGCCGGGGCTTCTTAGTCAGGTACCGTCATTTTCTTCCCTGCTGATAGAAGTTTACATACCG |
| | AGATACTTCTTCCTTCACGCGGCGTCGCTGCATCAGGGTTTCCCCCATTGTGCAATATTCCCCA |
| OTU | >denovo588 |
| Ref | |
| SEQ ID | CCTGTTTGCTCCCCACGCTTTCGCGCCTCAGCGTCAGTTAATGTCCAGCAGGCCGCCTTCGCCACTGG |
| NO: 225 | TGTTCCTCCGTATATCTACGCATTTCACCGCTACACACGGAATTCCGCCTGCCTCTCCATCACTCAAG |
| | AAAAACAGTTTCAAATGCAGGCCAGTGGTTGAGCCACTGGGTTTCACATCTGACTTGCTTTCCCGCCT |
| | ACACGCCCTTTACACCCAGTAAATCCGGATAACGCTTGCTCCCTACGTATTACCGCGGCTGCTGGCA |
| | CGTAGTTAGCCGGAGCTTATTCATTAAGTACCGTCATTTGTTTCGTCCTTAATAAAAGAAGTTTACAA |
| | CCCGAAAGCCTTCTTCCTTCACGCGGCGTTGCTGGGTCAGGCTTGCGCCCATTGCCCAATATTCCCCA |
| OTU | >denovo5917 |
| Ref | |
| SEQ ID | CCTGTTCGCTCCCCACGCTTTCGCGCCTGAGCGTCAGTCGCCAGCCAGCAGGCCGCCTTCGCCTCCGG |
| NO: 226 | TGTTCCTCCTGATCTCTGCGCATTTCACCGCTACACCAGGAATTCCGCCTGCCTCTCTGGCACTCGAG |
| | CCGCCCGGTTCCGCCGGCAGCCGCCGGGTTGGGCCCGGCGGTTTCACCGGCGGCCTAAACGGCCGCC |
| | TGCGCGCCCTTTACGCCCAATGATTCCGGACAACGCTTGCCCCCCACGTCTTACCGCAGCTGCTGGCA |
| | CGTGGTTGGCTGGGGCTCCTGGCCGGGTACCGTCATTGTCTTCCCCGGCGACAGAGCTTTACGGAAC |
| | GAGTCCCTTCTTCGCTCACGCGGCATTGCTGCGTCAGGGTCTCCCCCATTGCGCAATATTCCCCA |
| OTU | >denovo5959 |
| Ref | |
| SEQ ID | CCTGTTTGCTCCCCACGCTTTCGCGCCTCAGCGTCAGTTGCTGTCCAGTTGGCCGCCTTCGCCACTGG |
| NO: 227 | TGTTCCTCCTAATATCTACGCATTTCACCGCTACACTAGGAATTCCGCCAACCTCTCCAGTACTCAAG |
| | AACCACAGTTTCAAACGCAGGCCAGAGGTTGAGCCCCTGGTTTTCACGCCTGACTTGCAGTCCCGCC |
| | TACACGCCCTTTACACCCAGTAAATCCGGACAACGCTTGCCACCTACGTATTACCGCGGCTGCTGGC |
| | ACGTAGTTAGCCGTGGCTTATTCATCAGGTACAGTCTCATTCATCTTCCCTGATAAAAGAAGTTTACA |
| | ACCCGAAAGCCTTCTTCCTTCACGCGGCGTTGCTGGGTCAGGCTTGCGCCCATTGCCCAATATTCCCC |
| | A |
| OTU | >denovo6010 |
| Ref | |
| SEQ ID | CCTGTTTGCTACCCACGCTTTCGTGCCTCAGTGTCAGTTGCAGTCCAGAAAGCCGCCTTCGCTACTGG |
| NO: 228 | TGTTCTTCCCAATATCTACGCATTTCACCGCTACACTGAGAATTCCACTTTCCTCTCCTGCACTCAAGT |
| | CTTCCAGTTTCGGCGGCTTAATACGGTTGAGCCGTAGCCTTTAACCACCGACTTGAAAGACCACCTA |
| | CGCACCCTTTACGCCCAGTAAATCCGGATAACGCTAGCACCCTACGTATTACCGCGGCTGCTGGCAC |
| | GTAGTTAGCCGGTGCTTCCTCACAGGGTACCGTCATTATCTTCCCCTGCAACAGAACTTTACGATCCG |
| | AAGACCTTCATCGTTCACGCGGCGTTGCTGCATCAGGCTTTCGCCCATTGTGCAATATCCCCCA |
| OTU | >denovo6025 |
| Ref | |
| SEQ ID | CCTGTTTGCTCCCCTAGCTTTCGCGCCTCAGCGTCAGTGATGGCCCAGAAGGCCGCCTTCGCCACCGG |
| NO: 229 | TGTTCTTCCCGATATCTGCGCATTCCACCGCTACACCGGGAATTCCGCCTTCCCCTACCACACTCAAG |
| | CCAGCCGGTATCGGGAGCGAGACGGGGTTGAGCCCCGAAATTTGACTCCCGACCTAACTGGCCGCCT |
| | ACGCGCGCTTTACGCCCAATGAATCCGGATAACGCTCGCCCCCTACGTATTACCGCGGCTGCTGGCA |
| | CGTAGTTAGCCGGGGCTTCTTCTGCAGGTACCGTCACCTTGCGGCCTCGTCCCTGCTGAAAGCGGTTT |
| | ACAACCCGAAGGCCGTCATCCCGCACGCGGCGTCGCTGCGTCAGGCTTTCGCCCATTGCGCAAGATT |
| | CCCCA |
| OTU | >denovo6038 |
| Ref | |
| SEQ ID | CCTGTTCGATCCCCACGCTTTCGTGCCTCAGCGTCAGACCGGCGCCGGTACGCTGCCTTCGCAATCGG |
| NO: 230 | AGTTCTGCGCGATATCTATGCATTTCACCGCTACACCGCGCATTCCGCGTACTTCTCGCCGTCTCAAG |
| | TCCCCCAGTCTAAACGGCTCGGCGGGGTTGAGCCCCGCGATTTTACCGCTTACTTAAGAGACCGCCT |
| | ACGCACCCTTTAAACCCAATAAATCCGGATAACGCTCGCATCCTCCGTATTACCGCGGCTGCTGGCA |
| | CGGAGTTAGCCGATGCTTTTTCTCCGGGTACACTCGGATGAGGACGCGTCCTCACTTTTGTTCCCCGG |
| | CAAAAGAGGTTTACAACACGTAGTGCCGTCTTCCCTCACGCGGCTTGGCTGGTTCAGGATCTCTCCC |
| | ATTGACCAATATTCCTCA |
| OTU | >denovo6039 |
| Ref | |
| SEQ ID | CCTGTTTGCTCCCCACGCTTTCGCGCCTCAGCGTCAGTTACGGCCCAGCAAGTCGCCTTCGCCACTGG |
| NO: 231 | TGTTCCTCCTAATATCTACGCATTTCACCGCTACACTAGGAATTCCACTTGCCTCTTCCGCACTCAAG |
| | TCTGCCAGTATCGGATGCACGTTAAGGGTTGAGCCCTTACATTTCACATCCGACTTAACAGACCGCCT |
| | ACGCGCCCTTTACGCCCAGTGATTCCGGACAACGCTTGCCCCCTATGTTTTACCGCGGCTGCTGGCAC |
| | ATAGTTAGCCGGGGCTTCCTTCTTGTCTACCGTCCTCAGACTGTCAGTTACTACAGTCTGCTCTCTTG |
| | GACAAGGATAGAGCTTTACAACCCGAAGGCCTTCTTCACTCACGCGGCATTGCTCCGTCAGGCTTTC |
| | GCCCATTGCGGAAGATTCCCCA |
| OTU | >denovo621 |
| Ref | |
| SEQ ID | CCTATTTGCTCCCCACGCTTTCGGGACTCAGCGTCTGTTTCAGCCCAGATCGTCGCCTTCGCCACTGG |
| NO: 232 | TGTTCCTCCATATATCTACGCATTTCACCGCTACACATGGAATTCCACGATCCTCTGCTGTACTCTAG |
| | CCACACGGTTTCTTTGCCTTCCCGATGTTGAGCATCGGGTTTTTAACACTAGACCCTCATGGCCGCCT |
| | GCTCCCTCTTTACGCCCAATAATTCCGGATAACGCTCGCCACCTACGTATTACCGCGGCTGCTGGCAC |
| | GTAGTTAGCCGTGGCTTTCTAACCAGGTACCGTCCTATATGAATCATTTCCTATTCATATCTCTCTTCC |
| | CTGATAACAGAACTTTACAATCCGAAGACCTTCTTCGTTCACGCGGCGTTGCTCGGTCAGACTTTCGT |
| | CCATTGCCGAAAATTCCCTA |
| OTU | >denovo6236 |
| Ref | |
| SEQ ID | CCTGTTCGATACCCACGCCTTCGTGCTTCAGCGTCAGTTGGGCGCCGGTACGCTGCCTTCGCAATCGG |
| NO: 233 | AGTTCTGCGTGATATCTATGCATTTCACCGCTACACCACGCATTCCGCGTACTTCTCGCCCACTCAAG |
| | GCACCCAGTTTCAACGGCTCGACGAGGTTGAGCCCCGCAATTTTACCGCTGACTTAAGCGTCCGCCT |
| | ACGCACCCTTTAAACCCAATAAATCCGGATAACGCTCGCATCCTCCGTATTACCGCGGCTGCTGGCA |
| | CGGAGTTAGCCGATGCTTTTTCTCCGGGTACTCTCCAAACCGCACACGTGCGGCACTTTGCTCCCCGG |
| | CAAAAGAGGTTTACAACCCATAGGGCCGTCATCCCTCACGCGACTTGGCTGGTTCAGGCTCTCGCCC |
| | ATTGACCAATATTCCTCA |
| OTU | >denovo6253 |
| Ref | |
| SEQ ID | CCTGTTTGCTCCCCACGCTTTCGAGCCTCAACGTCAGTTACCGTCCAGTAAGCCGCCTTCGCCACTGG |
| NO: 234 | TGTTCTTCCTAATATCTACGCATTTCACCGCTACACTAGGAATTCCGCTTACCTCTCCGGCACTCTAG |
| | CTAAACAGTTTCCAAAGCAGTCCCGGCGTTGAGCACCGGGCTTTCACTTCAGACTTGCCTTGCCGTCT |
| | ACACTCCCTTTACACCCAGTAAATCCGGATAACGCTTGCCCCCTACGTATTACCGCGGCTGCTGGCAC |
| | GTAGTTAGCCGGGGCTTCTTATTCAGGTACCGTCACTTTCTTCCCTGCTGATAGAGCTTTACATACCG |
| | AAATACTTCTTCACTCACGCGGCGTCGCTGCATCAGGGTTTCCCCCATTGTGCAATATTCCCCA |
| OTU | >denovo6261 |
| Ref | |
| SEQ ID | CCTGTTTGCTCCCCACGCTTTCGCGCCTCACCGTCAGTTGTCGTCCAGTAATCCGCCTTCGCCACTGG |
| NO: 235 | TGTTCTTCCTTATATCTACGCATTTCACCGCTACACAAGGAATTCCGATTACCTCTCCGATACTCAAG |
| | ATGCACAGTTTCAAATGCAGTTTGGAGGTTGAGCCTCCAGATTTCACATCTGACTTGCGCACCCGGCT |
| | ACACGCCCTTTACACCCAGTAAATCCGGACAACGCTTGCCACCTACGTATTACCGCGGCTGCTGGCA |
| | CGTAGTTAGCCGTGGCTTATTCGTCAGGTACCGTCTTCTACTCTTCCCTGACAAAAGAAGTTTACAAC |
| | CCGAAAGCCTTCTTCCTTCACGCGGCGTTGCTGGGTCAGACTTGCGTCCATTGCCCAATATTCCCCA |
| OTU | >denovo6274 |
| Ref | |
| SEQ ID | CCTGTTTGCTCCCCACGCTTTCGCGCCTCAGTGTCAGTTGCAGACCAGGAAGCCGCCTTCGCCACTGG |
| NO: 236 | TGTTCCTCCATATCTCTACGCATTTCACCGCTACACATGGAATTCCACTTCCCTCTTCTGCACTCAAGT |
| | CAACCAGTTTCCAATGACCCTCCACGGTTAAGCCGTGGGCTTTCACATCAGACTTAATTAACCACCTG |
| | CGCGCTCTTTACGCCCAATAATTCCGGATAACGCTTGCCACCTACGTATTACCGCGGCTGCTGGCACG |
| | TAGTTAGCCGTGGCTTTCTCATAAGGTACCGTCAATTGATAGTCATTTCCTCCTATCACCTTTCTTCCC |
| | TTATAACAGAATTTTACAACCCGAAGGCCTTCTTCATTCACGCGGCGTTGCTCGGTCAGGCTTTCGCC |
| | CATTGCCGAAGATTCCCTA |
| OTU | >denovo6339 |
| Ref | |
| SEQ ID | CCTGTTTGCTCCCCACGCTTTCGAGCCTCAACGTCAGTTACCGTCCAGTAAGCCGCCTTCGCCACTGG |
| NO: 237 | TGTTCCTCCCAATATCTACGCATTTCACCGCTACACTGGGAATTCCGCTTACCTCTCCGGCACTCTAG |
| | ATACACAGTTTCCAAAGCAGTCCCGGGGTTGAGCCCCGGGCTTTCACTTCAGACTTGCACATCCGTCT |
| | ACGCTCCCTTTACACCCAGTAAATCCGGATAACGCTTGCCCCCTACGTATTACCGCGGCTGCTGGCAC |
| | GTAGTTAGCCGGGGCTTCTTAGTCAGGTACCGTCATTTTCTTCCCTGCTGATAGAGCTTTACATACCG |
| | AAATACTTCTTCGCTCACGCGGCGTCGCTGCATCAGGGTTTCCCCCATTGTGCAATATTCCCCA |
| OTU | >denovo6368 |
| Ref | |
| SEQ ID | CCTGTTTGCTCCCCACGCTCTCGCGCCTGAGCGTCAGTCTCTCTCCAGCAGGCCGCCTTCGCCTCCGG |
| NO: 238 | TATTCCTCCTGATCTCTGCGTATTTCACCACTACACCAGGAATTCTGCCTGCCTCTAGAGCACTCTAG |
| | TCAGCTGGTTCTTCCTGCTTACCCGGGGTGGGCCCGGGCCTTTTACAAAAAGCCTCTCTGACCGCCTG |
| | CGCGCGCTTTACGCCCAATGATTCCGGACAACGCTCGCCCCCTACGTATTGCCGCGGCTGCTGGCAC |
| | GTAGTTAGCCGGGGCTTAAAGTCAGGTAGTCTCTCTCTTGCCTGACGACTGAGCTCTGCGGGACGAA |
| | TCCCTTCCTCACTCACGCGGCATCGCTGCGTCAGAGTTGCCTCCATTGCGCAATATTCCCCA |
| OTU | >denovo638 |
| Ref | |
| SEQ ID | CCTGTTCGCTCCCCACGCTTTCGCACCTCAGCGTCAGTCTCCGGCCGGCAGACCGCCTTCGCCTCCGG |
| NO: 239 | TATTCCTCCTGATCTCTGCGCATTTCACCGCTACACCAGGAATTCTGTCTGCCTCTCCGGTACTCGAG |
| | TTAAACGGTTCGCGCTGACCTTCCGGGGTTGAGCCCCGGGCTTTGACAACGCGCCTGCCTAACCGCC |
| | TGCGTGCCCTTTACGCCCAATAAATCCGGATAACGCTCGCCCCCTACGTATTACCGCAGCTGCTGGC |
| | ACGTAGTTGGCTGGGGCTTGCTCGCCGGGTACCGTCTTAACTCTTCCCCGACAACAGGGCTTTACGG |
| | AACGAATCCCTTCTTCACCCACGCGGCATTGCTGCGTCAGAGTTGCCTCCATTGCGCAATATTCCCCA |
| OTU | >denovo6392 |
| Ref | |
| SEQ ID | CCTGTTTGCTCCCCACGCTTTCGAGCCTCAACGTCAGTTACCGTCCAGTAAGCCGCCTTCGCCACTGG |
| NO: 240 | TGTTCCTCCTAATATCTACGCATTTCACCGCTACACTAGGAATTCCGCTTACCTCTCCGGCACTCGAG |
| | TATCACAGTTTCCAATGCAGTCCAGGGGTTGAGCCCCCGCCTTTCACATCAGACTTGCAACACCGTCT |
| | ACGCTCCCTTTACACCCAGTAAATCCGGATAACGCTTGCCCCCTACGTATTACCGCGGCTGCTGGCAC |
| | GTAGTTAGCCGGGGCTTCTTAGTCAGGTACCGTCATTTTCTTCCCTGCTGATAGAGCTTTACATACCG |
| | AAATACTTCATCGCTCACGCGGCGTTGCTGCATCAGGCTTGCGCCCATTGTGCAATATTCCCCA |
| OTU | >denovo641 |
| Ref | |
| SEQ ID | CCTGTTTGCTCCCCACGCTTTCGAGCCTCAACGTCAGTTACTGTCCAGTAAGCCGCCTTCGCCACTGG |
| NO: 241 | TGTTCCTCCTAATATCTACGCATTTCACCGCTACACTAGGAATTCCACTTACCTCTCCAGCACTCTAG |
| | CAACACAGTTTCCAAAGCAGTCCCAGGGTTGAGCCCTGGGTTTTCACTTCAGACTTGCATCGCCGTCT |
| | ACGCTCCCTTTACACCCAGTAAATCCGGATAACGCTTGCCCCCTACGTATTACCGCGGCTGCTGGCAC |
| | GTAGTTAGCCGGGGCTTCTTAGTCAGGTACCGTCTTGTTTCTTCCCTGCTGATAGAGCTTTACATACC |
| | GAAATACTTCTTCACTCACGCGGCGTCGCTGCATCAGGGTTTCCCCCATTGTGCAATATTCCCCA |
| OTU | >denovo6415 |
| Ref | |
| SEQ ID | CCTATTTGCTCCCCACGCTTTCGTGCATGAGCGTCAGTAACAGTCCAGGCGGCCGCCTTCGCAACTGG |
| NO: 242 | TGTTCTTCCATATATCTACGCATTTTACCGCTACACATGGAGTTCCACCGCCCTCTCCTGTCCTCTAGC |
| | CTGCCAGTTTCCGAAGCATATACAGGTTAAGCCCGTACCTTTCACTTCAGACTTGGCATGCAGCCTGC |
| | GCACCCTTTACGCCCAATCATTCCGGATAACGCTTGCCACCTACGTATTACCGCGGCTGCTGGCACGT |
| | AGTTAGCCGTGACTTTCTGGCGGGGTACCATCAAAAGAAGAACATTTCCTTTCCTCTTCCTTTTTCCC |
| | CCGCAACAGAGCTTTACGATCCGAAGACCTTCCTCACTCACGCGGCATTGCTCGTTCAGGCTTGCGC |
| | CCATTGACGAAAATTCCCTA |
| OTU | >denovo6444 |
| Ref | |
| SEQ ID | CCTGTTCGATACCCACGCTTTCGTGCCTCAGCGTCCGTAGGCGCCCGGTATGCTGCCTTCGCAATTGG |
| NO: 243 | AGTTCTGCGTCATATCTATGCATTTCACCGCTACACAACGCATTCCGCATACCTCGACGCCACGCTAG |
| | ATAATCAGTTTCAACGGCTGAGCGGGGTTGAGCCCCGCGATTTTACCGCTGACTTGACTGTCCGCCT |
| | GCGCACCCTTTAAACCCAATAAATCCGGATAACGCTCGCATCCTCCGTATTACCGCGGCTGCTGGCA |
| | CGGAGTTAGCCGATGCTTATTCTTGCGGTACACTCATAAGGGGACACGTCCCCTCCTTTGTTCCCGCA |
| | CTAAAGAAGTTTACAACCCGTAGGGCCGTCTTCCTTCACGCGACTTGGCTGGTTCAGACTTCCGTCCA |
| | TTGACCAATATTCCTCA |
| OTU | >denovo6453 |
| Ref | |
| SEQ ID | CCTGTTTGCTCCCCACGCTTTCGTGCATGAGCGTCAGTGTTATCCCAGGGGGCTGCCTTCGCCATCGG |
| NO: 244 | TATTCCTCCACATCTCTACGCATTTCACTGCTACACGTGGAATTCTACCCCCCTCTGACACACTCTAG |
| | CCGTGCAGTCACCAATGCAATTCCCAAGTTAAGCTCGGGGATTTCACATCGGTCTTGCACAACCGCC |
| | TGCGCACGCTTTACGCCCAGTAATTCCGATTAACGCTTGGACCCTACGTATTACCGCGGCTGCTGGCA |
| | CGTAGTTAGCCGGTCCTTATTCTTCCGGTACCGTCATCGACCCCAGGTATTAACCAGAGCCATTTCTT |
| | TCCGGACAAAAGTGCTTTACAACCCGAAGGCCTTCTTCACACACGCGGCATTGCTGGATCAGGCTTT |
| | CGCCCATTGTCCAAAATTCCCCA |
| OTU | >denovo6458 |
| Ref | |
| SEQ ID | CCTGTTTGCTCCCCACGCTCTCGCGCCTTAGCGTCAGTCTCTCTCCAGCAGGCCGCCTTCGCCTCCGG |
| NO: 245 | TATTCCTCCTGATCTCTGCGCATTTCACCGCTACACCAGGAATTCTGCCTGCCTCTCAAGTACTCTAG |
| | CTGGCCCGGTTCCCGTCGCCTCCCCGGGTTGAGCCCGGGAATTTTACAACAGGCCTCTGCCGCCGCCT |
| | ACGCGCCCTTTACGCCCAGTAATTCCGGACAACGCTCGCCCCCTACGTATTACCGCGGCTGCTGGCA |
| | CGTAGTTAGCCGGGGCTTTAAAATGGGGTAGTATCTCTCTTCTCCCCCACAACTGAGCTCTGCGGGA |
| | CGAATCCCTTCCTCACTCACGCGGCATCGCTGCGTCAGAGTTTCCTCCATTGCGCAATATTCCCCA |
| OTU | >denovo6463 |
| Ref | |
| SEQ ID | CCTGTTTGCTCCCCACGCTTTCGCGCCTTAGCGTCAGTTGAGTTCCAGCAGATCGCCTTCGCAATGGG |
| NO: 246 | TATTCTTGGTGATATCTACGGATTTTACCCCTACACCACCAATTCCATCTGCCTCTCCCTCACTCTAGA |
| | CTATCAGTTTCCCAAGCAGTTTAATGGTTAAGCCATTAGATTTCACAAGAGACTTGATAATCCGCCTA |
| | CGCGCCCTTTACGCCCAGTGATTCCGAGTAACGCTTGCACCCTCCGTATTACCGCGGCTGCTGGCACG |
| | GAGTTAGCCGGTGCTTATTCCTTAGGTACCGTCAGAATTCTTCCCTAAGAAAAGGAGTTTACGCTCCG |
| | AAAAGTGTCATCCTCCACGCGGCGTTGCTGCGTCAGGGTTTCCCCCATTGCGCAATATTCCCTA |
| OTU | >denovo6468 |
| Ref | |
| SEQ ID | CCTGTTCGCTCCCCACGCTTTCGTGCCTCAGCGTCAGTTACAGTCCAGAAAGCCGCCTTCGCTACTGG |
| NO: 247 | TGTTCCTCCGAATCTCTACGCATTTCACCGCTACACTCGGAATTCCGCTTTCCTCTCCTGCACTCTAGC |
| | AACTCAGTTTCAGATGCAGTCTCAAGGTTGAGCCCTGAGTTTTCACATCTGACTTAAATCGCCGCCTA |
| | CGCACCCTTTACACCCAGTAATTCCGGATAACGCTTGCCCCCTACGTATTACCGCGGCTGCTGGCACG |
| | TAGTTAGCCGGGGCTTCTTAGTCAGGTACCGTCATGGTTTCTTCCCTGCTGATAGAAGTTTACGATCC |
| | GAAGACCTTCATCCTTCACGCGGCGTCGCTGCATCAGGCTTGCGCCCATTGTGCAATATTCCCCA |
| OTU | >denovo6501 |
| Ref | |
| SEQ ID | CCTGTTTGCTCCCCACGCTTTCGAGCCTCAACGTCAGTTACAGTCCAGTAAGCCGCCTTCGCCACTGG |
| NO: 248 | TGTTCCTCCTAATATCTACGCATTTCACCGCTACACTAGGAATTCCGCTTACCTCTCCTGCACTCTAGC |
| | TTAACAGTTTCCAATGCAGTCTTGGGGTTGAGCCCCAAGTTTTCACATCAGACTTGCCATGCCGTCTA |
| | CGCTCCCTTTACACCCAGTAAATCCGGATAACGCTTGCCCCCTACGTATTACCGCGGCTGCTGGCACG |
| | TAGTTAGCCGGGGCTTCTTAGTCAGGTACCGTCATTTTCTTCCCTGCTGATAGAAGTTTACATACCGA |
| | AATACTTCTTCCTTCACGCGGCGTCGCTGCATCAGGGTTTCCCCCATTGTGCAATATTCCCCA |
| OTU | >denovo6525 |
| Ref | |
| SEQ ID | CCTGTTCGATCCCCACGCTTTCGTGCCTCAGCGTCAGTGACGACTTCGCCAGATGCCTCCGCAATCGG |
| NO: 249 | TGTTCTGTGTGATATCTATGCATTTCACCGCTACACCACACATTCCTCCAGCGGCAGTCGTACTCGAG |
| | CAAAGCAGTATCAACGGCACGGTCCCTGTTGAGCAGGAAGTTTTCACCGCTGACTTACCTCGCAGCC |
| | TACGCACCCTTTAAACCCAATAAATCCGGATAACGCTTGCATCCTCCGTATTACCGCGGCTGCTGGC |
| | ACGGAGTTAGCCGATGCTTATTCGTTGCATACTCTCATCAAACCTCACGAGGTCCTTATTGCTCTGCA |
| | ACAAAAGAAGTTTACAACCCATAGGGCCTTAATCCTTCACGCGGCATGGCTGGTTCAGACTTCCGTC |
| | CATTGACCAATATTCCTCA |
| OTU | >denovo6564 |
| Ref | |
| SEQ ID | CCTGTTTGCTCCCCACGCTTTCGCGCCTCAGCGTCAGTTGTCGTCCAGCAAGCCGCCTTCGCCACTGG |
| NO: 250 | TGTTCCTCCTAATATCTACGCATTTCACCGCTACACTAGGAATTCCGCTTGCCTCTCCGATACTCAAG |
| | GGCTACAGTTTCAAATGCAGTTCCGGGGTTGAGCCCCGGGATTTCACATCTGACTTGCAGCTCCGCCT |
| | ACACGCCCTTTACACCCAGTAATTCCGGACAACGCTTGCAACCTACGTATTACCGCGGCTGCTGGCA |
| | CGTAGTTAGCCGTTGCTTTTTCTGTAGGTACCGTCACGTTCTTCGTCCCTACTAAAAGAAGTTTACAA |
| | CCCGAAGGCCTTCTTCCTTCACGCGGCGTTGCTGGGTCAGGGTTTCCCCCATTGCCCAATATTCCCCA |
| OTU | >denovo6593 |
| Ref | |
| SEQ ID | CCTGTTTGCTCCCCTAGCTTTCGCGCCTCAGCGTCAGTGACGGCCCAGAGGACTGCCTTCGCCATCGG |
| NO: 251 | TGTTCTTCCCGATATCTGCGCATTCCACCGCTACACCGGGAATTCCATCCTCCTCTACCGCACTCGAG |
| | GCGTGCAGTTCGCGACCCGGCTTGGGGTTGAGCCCCAAGATTAGAGGTCGCGCATACACACCCGCCT |
| | ACGCGCTCTTTACGCCCAATGAATCCGGATAACGCTCGCTCCCTACGTATTACCGCGGCTGCTGGCA |
| | CGTAGTTAGCCGGAGCTTCTTCTGCAGGTACTGTCGAAGTCATCCCTGCTGAAAGCGGTTTACAACC |
| | CGAAGGCCGTCATCCCGCACGCGGCGTTGCTGCGTCAGGGTTTCCCCCATTGCGCAAAATTCCCCA |
| OTU | >denovo660 |
| Ref | |
| SEQ ID | CCTGTTCGCTCCCCTAGCTTTCGCGCCTCAGCGTCAGTCGTGGCCCAGAAGGCCGCCTTCGCCGCCGG |
| NO: 252 | TGTTCTTCCCAATATCTGCGCATTCCACCGCTACACTGGGAATTCCGCCTTCCCCTACCAGACTCAAG |
| | CCTGCCGGTATCGGGGGCGGACCGGGGTTGAGCCCCGGGATTTGACCCCCGACCTAACAGGCCGCCT |
| | ACGCGCGCTTTACGCCCAATGAATCCGGATAACGCTCGCCCCCTACGTATTACCGCGGCTGCTGGCA |
| | CGTAGTTAGCCGGGGCTTCTTCTGCAGGTACCGTCACCCAGAGGGCCTCGTCCCTGCTGAAAGCGGT |
| | TTACGACCCGAAGGCCTTCGTCCCGCACGCGGCGTCGCTGCGTCAGGCTTTCGCCCATTGCGCAAGA |
| | TTCCCCA |
| OTU | >denovo6639 |
| Ref | |
| SEQ ID | CCTGTTTGCTCCCCACGCTTTCGCGCATCAGCGTCAGTAATGTTCCAGCAGGTCGCCTTCGCAATGAG |
| NO: 253 | TATTCCTCTTGATCTCTACGGATTTTACCCCTACACCAAGAATTCCACCTACCTCTCCCATACTCTAGA |
| | TGGTCAGTTTCAAATGCAGTTCTGTAGTTAAGCTACAGGATTTCACATCTGACTTGACCACCCGCCTA |
| | CGCGCTCTTTACGCCCAGTGATTCCGAGTAACGCTTGCACCCTCCGTATTACCGCGGCTGCTGGCACG |
| | GAGTTAGCCGGTGCTTATTCGTAAGATACCGTCATAATCTTCTCTTACAAAAGGAGTTTACAATCCTA |
| | AAACCTTCATCCTCCACGCGGCGTTGCTGCTTCAGGGTTTCCCCCATTGAGCAATATTCCCTA |
| OTU | >denovo6671 |
| Ref | |
| SEQ ID | CCTATTTGCTCCCCACGCTTTCGTGCCTCAGCGTCAGTAACAGTCCAGGCGGCCGCCTTCGCAACTGG |
| NO: 254 | TGTTCTTCCATATATCTACGCATTTTACCGCTACACATGGAGTTCCACCGCCCTCTCCTGTCCTCGAGC |
| | GCGCCAGTTTCCAAAGCCTGCACAGGTTGAGCCTGTACCTTTTACTTCAGACTTGACGCGCCGCCTGC |
| | GCACCCTTTACGCCCAATCATTCCGGATAACGCTCGCCACCTACGTATTACCGCGGCTGCTGGCACGT |
| | AGTTAGCCGTGACTTTCTGGCGGGGTACCATCAAAAGCAGACCATTTCCTTTCTGCTTCCTTTTTCCC |
| | CCGCAACAGAGCTTTACGATCCGAAGACCTTCCTCACTCACGCGGCATTGCTCGTTCAGGCTTGCGC |
| | CCATTGACGAAAATTCCCTA |
| OTU | >denovo6675 |
| Ref | |
| SEQ ID | CCTATTTGCTCCCCACGCTTTCGGGAATGAGCGTCAGTTACAGGCCAGACCGTCGCCTTCGCCACTGG |
| NO: 255 | TGTTCCTCCATATATCTACGCATTTCACCGCTACACATGGAATTCCACGATCCTCTCCTGCACTCTAG |
| | CCGCCCGGTTTCTATGGCTTACTGAAGTTAAGCTTCAGTCTTTCACCACAGACCTTTGCCGCCGCCTG |
| | CTCCCTCTTTACGCCCAATAATTCCGGATAACGCTTGCCACCTACGTATTACCGCGGCTGCTGGCACG |
| | TAGTTAGCCGTGGCTTTCTCATAAAGTACCGTCACTCGGATATCATTCCCTATATCCGCCGTTCTTCCT |
| | TTATAACAGAAGTTTACAGAACGAATTCCTTCTTCCTTCACGCGGCGTTGCTCGGTCAGGGTTCCCCC |
| | CATTGCCGAAAATTCCCTA |
| OTU | >denovo6680 |
| Ref | |
| SEQ ID | CCTGTTTGCTCCCCACGCTTTCGAGCCTCAGCGTCAGTTACAGTCCAGAGAATCGCCTTCGCCACTGG |
| NO: 256 | TGTTCTTCCTAATCTCTACGCATTTCACCGCTACACTAGGAATTCCATTCTCCTCTCCTGCACTCTAGA |
| | TACCCAGTTTGGAATGCAGCTCCCAGGTTAAGCCCAGGTATTTCACATCCCACTTAAGTATCCGCCTA |
| | CGCTCCCTTTACGCCCAGTAAATCCGGATAACGCTTGCCACCTACGTATTACCGCGGCTGCTGGCAC |
| | GTAGTTAGCCGTGGCTTCCTCCTTAGGTACCGTCATTATCGTCCCTAAAGACAGAGCTTTACAATCCG |
| | AAGACCTTCATCACTCACGCGGCGTTGCTGCATCAGGGTTTCCCCCATTGTGCAATATTCCCCA |
| OTU | >denovo6693 |
| Ref | |
| SEQ ID | CCTGTTTGCTCCCCACGCTTTCGCGCCTCAGCGTCAGTTACGGCCCAGCAAGTCGCCTTCGCCACTGG |
| NO: 257 | TGTTCCTCCTAATATCTACGCATTTCACCGCTACACTAGGAATTCCACTTGCCTCTTCCGCACTCAAG |
| | TCTGCCAGTATCGGATGCACGTTAAGGGTTGAGCCCTTACATTTCACATCCGACTTAACAGACCGCCT |
| | ACGCGCCCTTTACGCCCAGTGATTCCGGACAACGCTTGCCCCCTATGTTTTACCGCGGCTGCTGGCAC |
| | ATAGTTAGCCGGGGCTTCCTTCTTGTCTACCGTCCTCGAGTAGTCAGTTACTACCACTCGCTCTCTTG |
| | GACAAGGATAGAGCTTTACAACCCGAAGGCCTTCTTCACTCACGCGGCATTGCTCCGTCAGGCTTTC |
| | GCCCATTGCGGAAGATTCCCCA |
| OTU | >denovo6696 |
| Ref | |
| SEQ ID | CCTGTTCGATACCCACGCTTTCGTGCCTCAGCGTCAGTTGGGCGCCGGTATGCTGCCTTCGCAATCGG |
| NO: 258 | GGTTCTGCGTGATATCTATGCATTTCACCGCTACACCACGCATTCCGCATACTTCTCGCCCACTCAAG |
| | TCCGCCAGTTTCAACGGCAGGCCGGCGTTGAGCGCCGACATTTGACCACTGACTTAACAAACCGCCT |
| | ACGCACCCTTTAAACCCAATAAATCCGGATAACGCTCGCATCCTCCGTATTACCGCGGCTGCTGGCA |
| | CGGAGTTAGCCGATGCTTTTTCTCCGGGTACTCTCCTTCCCAGCTCACGAGCTGGGCATTGCTCCCCG |
| | GCAAAAGAGGTTTACAACCCGTAGGGCCGTCTTCCCTCACGCGACTTGGCTGGTTCAGGCTTCCGCC |
| | CATTGACCAATATTCCTCA |
| OTU | >denovo6735 |
| Ref | |
| SEQ ID | CCTGTTTGCTCCCCACGCTTTCGAGCCTCAGCGTCAGTTACAATCCAGAGAAGCGCCTTCGCCACTGG |
| NO: 259 | TGTTCTTCCTAATCTCTACGCATTTCACCGCTACACTAGGAATTCCCTTCTCCTCTCCTGCACTCTAGA |
| | CTTCCAGTTTGAAATGCAGCACCCAAGTTAAGCCCGGGTATTTCACATCTCACTTAAAAATCCGCCTA |
| | CGCTCCCTTTACGCCCAGTAAATCCGGACAACGCTTGCCACCTACGTATTACCGCGGCTGCTGGCAC |
| | GTAGTTAGCCGTGGCTTCCTCCTCGGGTACCGTCATTATCGTCCCCGAAGACAGAGCTTTACAACCCG |
| | AAGGCCGTCATCACTCACGCGGCATTGCTGCATCAGGGTTTCCCCCATTGTGCAATATTCCCCA |
| OTU | >denovo6746 |
| Ref | |
| SEQ ID | CCTATTTGCTCCCCACGCTTTCGGGACTGAGCGTCAGTTATGCGCCAGATCGTCGCCTTCGCCACTGG |
| NO: 260 | TGTTCCTCCATATATCTACGCATTTCACCGCTACACATGGAATTCCACGATCCTCTCACACACTCTAG |
| | CTCTACGGTTTCCATGGCTTACCGAAGTTAAGCTTCGATCTTTCACCACAGACCCTTAGTGCCGCCTG |
| | CTCCCTCTTTACGCCCAATAATTCCGGATAACGCTCGCCACCTACGTATTACCGCGGCTGCTGGCACG |
| | TAGTTAGCCGTGGCTTTCTTATAGAGTACCGTCACTTGGATATCATTCCCTATATCCACCGTTCTTCCT |
| | CTATGACAGAAGTTTACATAACGAATTACTTCTTCCTTCACGCGGCGTTGCTCGGTCAGGGTTTCCCC |
| | CATTGCCGAAAATTCCCTA |
| OTU | >denovo6791 |
| Ref | |
| SEQ ID | CCTGTTTGATACCCGCACTTTCGAGCCTCAGCGTCAGTAACACCCCAGAAGACTGCCTTCGCAATCG |
| NO: 261 | GAGTTCTTCGTGATATCTAAGCATTTCACCGCTACACCACGAATTCCATCTTCCTTGTGTGAACTCAA |
| | GACAACCAGTATCAACGGCGGAGAATGGTTGAGCCACTCTATTTTACCGCTGACTTAATCGTCCGCC |
| | TACGCTCCCTTTAAACCCAATAAATCCGGATAACGCTCGCATCTTCCGTATTACCGCGGCTGCTGGCA |
| | CGGAATTAGCCGATGCTTATTCATAAGGTACATACAATAGAGCACACGTGCTCTTTTTTATTCCCATA |
| | TAAAAGAAGTTTACAACCCATAGGGCAGTCATCCTTCACGCTACTTGGCTGGTTCAGACTCTCGTCC |
| | ATTGACCAATATTCCTCA |
| OTU | >denovo6829 |
| Ref | |
| SEQ ID | CCTGTTTGCTCCCCACGCTTTCGTGCATGAGCGTCAGTCGCATCCCAGGGGGCTGCCTTCGCCATTGG |
| NO: 262 | TGTTCTTCCAAATATCTACGCATTTCACTGCTACACTTGGAATTCCACCCCCCTCTGATGAACTCTAG |
| | TCCTGCAGTCACAAATGCAATTCCCAGGTTGAGCCCGGGGATTTCACATCTGTCTTACAGAACCGCC |
| | TGCGCACGCTTTACGCCCAGTAATTCCGATTAACGCTTGCACCCTACGTATTACCGCGGCTGCTGGCA |
| | CGTAGTTAGCCGGTGCTTATTCTTCAGGTACCGTCAGCAGATCATGGTATTAGCACAATCCTTTTCGT |
| | CCCTGACAAAAGCAGTTTACAATCCGAAGACCTTCATCCTGCACGCGGCATGGCTGGATCAGGGTTG |
| | CCCCCATTGTCCAAAATTCCCCA |
| OTU | >denovo6843 |
| Ref | |
| SEQ ID | CCTGTTTGCTCCCCATGCTTTCGCGCTTCAGTGTCAGTATTCGTCCAGTGAGCTGACTTCTCTATCGGC |
| NO: 263 | ATTCCTACAAATATCTACGAATTTCACCTCTACACTTGTAGTTCCGCCCACCTCTCCGATACTCTAGTT |
| | AGGCAGTTTCCAACGCAATACGGAGTTGAGCCCCGCATTTTCACATCAGACTTACCAAACCACCTAG |
| | ACGCGCTTTACGCCCAATAAATCCGGATAACGCTTGCGACATACGTATTACCGCGGCTGCTGGCACG |
| | TATTTAGCCGTCGCTTCTTCTATTGGTACCGTCATTTTTTTCTTCCCAATTGAAAGCACTTTACAATCC |
| | TAAGACCGTCATCGTGCACACAGAATTGCTGGATCAGACTTTTGGTCCATTGTCCAATATTCCCCA |
| OTU | >denovo6855 |
| Ref | |
| SEQ ID | CCTGTTTGCTCCCCACGCTTTCGCACCTGAGCGTCAGTAAGTGCCTGGCAGACCGCCTTCGCCTCCGG |
| NO: 264 | TATTCCTCCTGATCTCTGCGCATTTCACCGCTACACCAGGAATTCTGTCTGCCCCGACATTACTCGAG |
| | TTATCCGGTTTGCCATGACCTGCGGGGTTGAGCCCCACCCTTGAACATCACACCTGGATAACCGCCT |
| | GCGTGCCCTTTACGCCCAATAAATCCGGATAACGCTCGCCCCCTACGTATTACCGCAGCTGCTGGCA |
| | CGTAGTTGGCTGGGGCTTGCTCACCGGGTACCGTCAGGCTTCTTCCCCGGTAACAGAGCTTTACGGA |
| | ACGAATCCCTTCTTCACTCACGCGGCATTGCTGCGTCAGAGTTGCCTCCATTGCGCAATATTCCCCA |
| OTU | >denovo6868 |
| Ref | |
| SEQ ID | CCTGTTTGATCCCCACGCTTTCGTGCTTCAGTGTCAGTTATGGCTTAGTAAGCTGCCTTCGCAATCGG |
| NO: 265 | AGTTCTGCGTGATATCTATGCATTTCACCGCTACACCACGCATTCCGCCTACCTCAAACACACTCAAG |
| | TCTCCCAGTTTCAACGGCAATTTTATGGTTAAGCCACAAACTTTCACCGCTGACTTAAAAAACAACCT |
| | ACGCACCCTTTAAACCCAATAAATCCGGATAACGCTCGCATCCTCCGTATTACCGCGGCTGCTGGCA |
| | CGGAGTTAGCCGATGCTTATTCATAGGGTACATACAAAACAGGACACGTCCCGCACTTTATTCCCCT |
| | ATAAAAGAAGTTTACAAACCATAGATCCTTCTTCCTTCACGCGACTTGGCTGGTTCAGGCTCGCGCCC |
| | ATTGACCAATATTCCTCA |
| OTU | >denovo6872 |
| Ref | |
| SEQ ID | CCTATTTGCTCCCCACGCTTTCGTGCCTCAGCGTCAGTAACAGGCCAGGCGGCCGCCTTCGCCACTGG |
| NO: 266 | TGTTCTTCCATATATCTACGCATTTTACCGCTACACATGGAGTTCCACCGCCCTCTCCTGTCCTCCAGC |
| | CTGCCAGTTTCCAAAGCCTGTACCGGTTGAGCCGGTACCTTTCACTTCAGACTTAACAGGCCGCCTAC |
| | GCACCCTTTACGCCCAATCATTCCGGATAACGCTCGCCACCTACGTATTACCGCGGCTGCTGGCACGT |
| | AGTTAGCCGTGACTTCCTCGAAAGGTAATATCACTTCACCAGCATTTCCTCTGGTGTTCCTTTTTCCCT |
| | CTCAACAGAACTTTACGATCCGAAGACCTTCCTCGTTCACGCGGCATTGCTCGTTCAGGCTTGCGCCC |
| | ATTGACGAAAATTCCCTA |
| OTU | >denovo6894 |
| Ref | |
| SEQ ID | CCTATTTGCTCCCCACGCTTTCGTGCCTCAGCGTCAGTCACAGGCCAGGCGGCCGCCTTCGCCACTGG |
| NO: 267 | TGTTCTTCCATATCTCTGCGCATTTTACCGCTACACATGGAGTTCCACCGCCCTCTCCTGTCCTCAAGT |
| | CTGCCAGTTTCTGAATCATGAATGAGTTGAGCTCATCCCTTTGCCTTCAGACTTAACAGACCGCCTGC |
| | GCACCCTTTACGCCCAATCATTCCGGATAACGCTCGCCACCTACGTATTACCGCGGCTGCTGGCACGT |
| | AGTTAGCCGTGGCTTTCTGGCCGGGTACCATCCATGAAATGCCATTTCCTGCATTCCCTCTTTTTCCCC |
| | GGCAACAGAGCTTTACAATCCGAAGACCTTCTTCACTCACGCGGCATTGCTCGTTCAGGCTTGCGCC |
| | CATTGACGAAAATTCCCTA |
| OTU | >denovo6902 |
| Ref | |
| SEQ ID | CCTGTTTGCTCCCCACGCTTTCGCGCCTCAGCGTCAGTTGTCGTCCAGCAGGCCGCCTTCGCCACTGG |
| NO: 268 | TGTTCCTCCTAATATCTACGCATTTCACCGCTACACTAGGAATTCCGCCTGCCTCTCCGATACTCAAG |
| | AAGAACAGTTTCAAATGCAATTTATGGGTTAAGCCCATAGATTTCACATCTGACTTGCTCTCCCGCCT |
| | ACACGCCCTTTACACCCAGTAAATCCGGACAACGCTTGCCACCTACGTATTACCGCGGCTGCTGGCA |
| | CGTAGTTAGCCGTGGCTTATTCGTCAGGTACCGTCTTCTGCTCTTCCCTGACAAAAGAAGTTTACAAC |
| | CCGAAAGCCTTCTTCCTTCACGCGGCGTTGCTGGGTCAGGCTTGCGCCCATTGCCCAATATTCCCCA |
| OTU | >denovo6969 |
| Ref | |
| SEQ ID | CCTGTTTGCTCCCCACGCTTTCGTGCCTGAGCGTCAGTTATGGTCCAGAAAGCCGCCTTCGCTACTGG |
| NO: 269 | TATTCCTTTGAATCTCTACGCATTTCACCGCTACACTCAAAGTTCTACTTTCCTCTCCCACACTCTAGC |
| | CTAGCAGTTTCAAAAGCAGCTCCGAAGTTGAGCCTCGGAATTTCACTTCTGACTTGCTTGGCCGCCTG |
| | CGCACCCTTTACACCCAGTAAATCCGGATAACGCTTGCTCCCTACGTATTACCGCGGCTGCTGGCAC |
| | GTAGTTAGCCGGAGCTTCTTAGTCAGGTACCGTCATTTTTTCTTCCCTGCTGATAGAAGTTTACGATC |
| | CGAAAACCTTCTTCCTTCACGCGGCGTTGCTGCATCAGGCTTGCGCCCATTGTGCAATATTCCCCA |
| OTU | >denovo6971 |
| Ref | |
| SEQ ID | CCTGTTTGCTACCCACGCTTTCGAGCCTCAGCGTCAGTTAAAGCCCAGTAGGCCGCCTTCGCCACTGG |
| NO: 270 | TGTTCCTTCCGATATCTACGCATTCCACCGCTACACCGGAAATTCCGCCTACCTCTACTTCACTCAAG |
| | CCCTGCAGTTTTGAAAGCGACTATGGGTTGAGCCCATAGATTATACTCCCAACTTGCTGAGCCGCCT |
| | ACGCTCCCTTTACACCCAGTAATTCCGGACAACGCTCGCACCCTACGTTTTACCGCGGCTGCTGGCAC |
| | GTAGTTAGCCGGTGCTTCCTCCTCGGGTACCGTCATTATCGTTCCCGAAGACAGAGGTTTACAATACG |
| | AATACCTTCTTCCCTCACGCGGCGTCGCTGCATCAGAGTTTCCTCCATTGTGCAATATTCCCCA |
| OTU | >denovo6995 |
| Ref | |
| SEQ ID | CCTGTTCGCTCCCCACGCTTTCGAGTCTCAGCGTCAGTTATAGTCCAGCAAGCCGCCTTCGCCACCGG |
| NO: 271 | TGTTCTTCCTGATATCTACGCATTTCACCGCTACACCAGGAATTCCGCTTGCCCTTCCTACACTCTAGC |
| | TGTACAGTTTCAAAAGCAGTCTTGGGGTTGAGCCCCAAGTTTTCACTCCTGACTTGTACTGCCGCCTA |
| | CACTCCCTTTACACCCAGTAAATCCGGATAACGCTTGCCCCATACGTATTACCGCGGCTGCTGGCAC |
| | GTATTTAGCCGGGGCTTCTTCTGTAGGTACCGTCATTATCTTCCCTACTGATAGAGCTTTACATACCG |
| | AAATACTTCTTCACTCACGCGGCGTCGCTGCATCAGAGTTGCCTCCATTGTGCAATATTCCCCA |
| OTU | >denovo7009 |
| Ref | |
| SEQ ID | CCTGTTCGATACCCACGCTTTCGTGCTTCAGCGTCAGTTGGGCGCCGGTATGCTGCCTTCGCAATCGG |
| NO: 272 | AGTTCTGCGTGATATCTATGCATTTCACCGCTACACCACGCATTCCGCATACTTCTCGCCCACTCAAG |
| | AACACCAGTTTCAACGGCGAAGCGAGGTTGAGCCCCGCGATTTGACCGCTGACTTGACATTCCGCCT |
| | ACGCACCCTTTAAACCCAATAAATCCGGATAACGCTCGCATCCTCCGTATTACCGCGGCTGCTGGCA |
| | CGGAGTTAGCCGATGCTTTTTCTTCAGGTACTCTCGTAACACCTACTCGTAGATGTTGTTGCTCCCTG |
| | ACAAAAGAGGTTTACAACCCATAGGGCAGTCTTCCCTCACGCGACTTGGCTGGTTCAGGCGTAAGCC |
| | CATTGACCAATATTCCTCA |
| OTU | >denovo7024 |
| Ref | |
| SEQ ID | CCTGTTTGCTCCCCACGCTTTCGCACCTCAGCGTCAGTATGTGCCCAGGAGACCGCCTTCGCCTCTGG |
| NO: 273 | TGTTCTTTCAGATATCTACGCATTCCACCGCTACACCTGAAATTCCATCTCCCTCTGCACTACTCTAG |
| | ACAGGCAGTTCAGAATGACCAACCCAGGTTGAGCCCGGGTATTTCACATCCTGCTTGCCAATCCGCC |
| | TACATGCCCTTTACGCCCAGTTATTCCGATTAACGCTTGCACCCTCCGTATTACCGCGGCTGCTGGCA |
| | CGGAGTTAGCCGGTGCTTCTTCTGTGGGTAACGTCAGTATTTCCTCCCCACTGAAAGTGCTTTACAAC |
| | CCGAAGGCCTTCTTCACACACGCGGCATGGCTGCATCAGGGTTTCCCCCATTGTGCAATATTCCCTA |
| OTU | >denovo7070 |
| Ref | |
| SEQ ID | CCTGTTTGCTCCCCACGCTTTCGTGCATGAGCGTCAGTATTATCCCAGGAGGCTGCCTTCGCCATCGG |
| NO: 274 | TGTTCTTCCAAATATCTACGCATTTCACTGCTACACTTGGAATTCCACCTCCCTCTGACATACTCTAGC |
| | CTGACAGTCACAAGTGCTATTCCCAAGTTGAGCTCGGGGATTTCACACCTGTCTTATCAAGCCGCCTG |
| | CGCACGCTTTACGCCCAGTAATTCCGATTAACGCTTGCACCCTACGTATTACCGCGGCTGCTGGCACG |
| | TAGTTAGCCGGTGCTTATTCTTTAGGTACCGTCATTAGGTAGCATTATTAGTACTACCCGTTTCGTTCC |
| | TAACAAAAGCAGTTTACAACCCGAAGGCCTTCTTCCTGCACGCGGCATGGCTGGATCAGGGTTGCCC |
| | CCATTGTCCAAAATTCCCCA |
| OTU | >denovo7107 |
| Ref | |
| SEQ ID | CCTGTTCGCTCCCCACGCTTTCGCTCCTCAGCGTCAGTAACGGCCCAGAGACCTGCCTTCGCCATTGG |
| NO: 275 | TGTTCTTCCCGATATCTACACATTCCACCGTTACACCGGGAATTCCAGTCTCCCCTACCGCACTCAAG |
| | CCCGCCCGTACCCGGCGCGGATCCACCGTTAAGCGATGGACTTTCACACCGGACGCGACGAACCGCC |
| | TACGAGCCCTTTACGCCCAATAAATCCGGATAACGCTTGCACCCTACGTATTACCGCGGCTGCTGGC |
| | ACGTAGTTAGCCGGTGCTTATTCGAACAATCCACTCAACACGGCCAAAGACCGTGCCTTGCCCTTGA |
| | ACAAAAGCGGTTTACAACCCGAAGGCCTCCATCCCGCACGCGGCGTCGCTGCATCAGGCTTGCGCCC |
| | ATTGTGCAATATTCCCCA |
| OTU | >denovo7110 |
| Ref | |
| SEQ ID | CCTGTTTGCTCCCCACGCTTTCGCGCCTCAGCGTCAGTTGCTGTCCAGCAGACCGCCTTCGCCACTGG |
| NO: 276 | TGTTCCTCCTAATATCTACGCATTTCACCGCTACACTAGGAATTCCGTCTGCCTCTCCAGTACTCAAG |
| | AACCACAGTTTCAAATGCAGGCTGGAGGTTGAGCCTCCAGTTTTCACATCTGACTTGCGATCCCGCCT |
| | ACACGCCCTTTACACCCAGTAAATCCGGATAACGCTTGCCACCTACGTATTACCGCGGCTGCTGGCA |
| | CGTAGTTAGCCGTGGCTTATTCCTCAGGTACCGTCACTTGCTTCGTCCCTGAGAAAAGAAGTTTACAA |
| | CCCGAAAGCCTTCTTCCTTCACGCGGCGTTGCTGGGTCAGGCTTGCGCCCATTGCCCAATATTCCCCA |
| OTU | >denovo7111 |
| Ref | |
| SEQ ID | CCTGTTCGCTCCCCGCGCTTTCGCACCTGAGCGTCAGTGCCCGGCCGGCAGGCCGCCTTCGCCTCCGG |
| NO: 277 | TGTTCCTCCTGATCTCTGCGCATTTCACCGCTACACCAGGAGTTCCGCCTGCCTCTCCGGCACTCGAG |
| | CCGCGCGGTTCGCGCCGCCGTTCCGGGGTTGGGCCCCGGCCTTTCACGGCGCGCCCTCGCGGCCGCC |
| | TGCGTGCCCTTTACGCCCAATGAATCCGGACAACGCCCGCCCCCCACGTGTTACCGCAGCTGCTGGC |
| | ACGTGGTTGGCTGGGGCTTCCTCGCCGGGTACCGTCTCTCTCTTCCCCGGCAACAGGGCTTTACGAAC |
| | CGAAGTCCTTCATCGCCCACGCGGCATCGCTGCGTCAGAGTTGCCTCCATTGCGCAATATTCCCCA |
| OTU | >denovo7117 |
| Ref | |
| SEQ ID | CCTGTTTGCTCCCCACGCTTTCGTGCCTCAGTGTCAGTTACAGTCCAGAGAGCCGCCTTCGCAACTGG |
| NO: 278 | TATTCCTCCTAATATCTACGCATTTCACCGCTACACTAGGAATTCTACTCTCCTCTCCTGCACTCAAGT |
| | TTCTCAGTTTCAAAGGCTTACTACGGTTGAGCCGTAGCCTTTCACCTCTGACTTAAGAAACCACCTAC |
| | GCACCCTTTACGCCCAGTAATTCCGGATAACGCTAGCCCCCTACGTATTACCGCGGCTGCTGGCACG |
| | TAGTTAGCCGGGGCTTCCTCCTCAAGTACCGTCATTATCTTCCTTGAGGACAGAGCTTTACGACCCGA |
| | AGGCCTTCATCGCTCACGCGGCGTTGCTGCATCAGGCTTTCGCCCATTGTGCAATATTCCCCA |
| OTU | >denovo7198 |
| Ref | |
| SEQ ID | CCTGTTTGCTCCCCACGCTTTCGAGCCTCAGCGTCAGTTTCAGTCCAGAAAGCCGCCTTCGCCACTGG |
| NO: 279 | TGTTCTTCCTAATATCTACGCATTTCACCGCTACACTAGGAATTCCGCTTTCCTCTCCTGTACTCTAGC |
| | TCGCCAGTTCCAAATGCAATCCTCGGGGTTAAGCCCCGGGCTTTCACATCTGGCTTGACGTGCCGCCT |
| | ACGCTCCCTTTACACCCAGTAATTCCGGATAACGCTTGCCCCCTACGTATTACCGCGGCTGCTGGCAC |
| | GTAGTTAGCCGGGGCTTCTTATTCAGGTACCGTCACTTTCTTCGTCCCTGTTGATAGAAGTTTACGAT |
| | CCGAAAACCTTCTTCCTTCACGCGGCGTTGCTGCATCAGAGTTTCCTCCATTGTGCAATATTCCCCA |
| OTU | >denovo7224 |
| Ref | |
| SEQ ID | CCTGTTCGCTCCCCCAGCTTTCGCGCCTCAGCGTCAGTGGCGGCCCAGCAGGCTGCCTTCGCCATCGG |
| NO: 280 | TGTTCTTCCCGATATCTGCGCATTCCACCGCTACACCGGGAATTCCGCCTGCCTCTACCGCACTCGAG |
| | CCGCCCAGTTCGGAACCCGGCCCGAGGTTGAGCCCCGGGGTTAGAGGTTCCGCTTAGGCAGCCGCCT |
| | ACGCGCGCTTTACGCCCAATGAATCCGGATAACGCTCGCTCCCTACGTATTACCGCGGCTGCTGGCA |
| | CGTAGTTAGCCGGAGCTTCTTCTGTGGGTACCGTCATCGTCGTCCCCACTGAAAGCGGTTTACAACCC |
| | GAGGGCCGTCGTCCCGCACGCGGCGTTGCTGCGTCAGGGTTTCCCCCATTGCGCAAAATTCCCCA |
| OTU | >denovo7232 |
| Ref | |
| SEQ ID | CCTGTTTGCTCCCCACGCTTTCGCGCCTCAGCGTCAGTTTCAGTCCAGAAAGCCGCCTTCGCCACTGG |
| NO: 281 | TGTTCCTACCGATCTCTACGCATTTCACCGCTACACCGGTAATTCCGCTTTCCTCTCCTGCACTCCAGC |
| | CCACCAGTATCCAAAGCAATTCCCACCTTGAAAGCAGGACTTTCACTCCAGACTTAGCAGGCCGCCT |
| | ACGCGCCCTTTACGCCCAATCATTCCGGACAACGCTCGCCCCCTACGTATTACCGCGGCTGCTGGCA |
| | CGTAGTTAGCCGGGGCTTCCTCCTATGGTACCGTCACTTCCTTCGTCCCATAGGACAGAGGTTTACGA |
| | GCCGAAACCCTTCTTCCCTCACGCGGCGTTGCTGGGTCAGGCTTTCGCCCATTGCCCAATATTCCCCA |
| OTU | >denovo7247 |
| Ref | |
| SEQ ID | CCTGTTTGCTCCCCACGCTCTCGCACCTGAGCGTCAGTATCTGTCCGGCAGGCCGCCTTCGCCTCTGG |
| NO: 282 | TATTCCTCCTGATCTCTGCGCATTTCACCGCTACACCAGGAATTCTGCCTGCCTCTCCAGTACTCGAG |
| | CTGCATGGTTCGGGATGCACCTCCAGGGTTGGGCCCTGGCTTTTCACATCCCGCCTACACAGCCGCCT |
| | GCGTGCCCTTTACGCCCAGTCATTCCGGACAACGCTCGCCCCCTACGTGTTACCGCGGCTGCTGGCAC |
| | GTAGTTAGCCGGGGCTTGCTTGTTAGGTACCGTCATTCTTCTTCCCTAACGGCAGAGCTTTACGGAAC |
| | GAATTCCTTCTTCACTCACGCGGCATTGCTGCGTCAGAGTTTCCTCCATTGCGCAATATTCCCCA |
| OTU | >denovo7260 |
| Ref | |
| SEQ ID | CCTGTTCGATACCCGCACCTTCGAGCTTCAGCGTCAGTCGCACTCCCGCAAGCTGCCTTCGCAATCGG |
| NO: 283 | AGTTCTTCGTGATATCTAAGCATTTCACCGCTACACCACGAATTCCGCCTGCGTTGCGTGCACTCAAG |
| | CCCCCCAGTTCGCGCTGCAGTGCCACGGTTGAGCCGCGGCATTTCACAACACGCTTAAGGGGCGGCC |
| | TACGCTCCCTTTAAACCCAATAAATCCGGATAACGCCTGGACCTTCCGTATTACCGCGGCTGCTGGC |
| | ACGGAATTAGCCGGTCCTTATTCATATGGTACCTGCAATAAGCTACACGTAGCTCACTTTATCCCCAT |
| | ATAAAAGCAGTTTACAACCCATAGGGCCGTCATCCTGCACGCTACTTGGCTGGTTCAGGCTCTCGCC |
| | CATTGACCAATATTCCTCA |
| OTU | >denovo7275 |
| Ref | |
| SEQ ID | CCTGTTCGCTCCCCACGCTTTCGCACCTGAGCGTCAGTATCTGGCCGGCAGACCGCCTTCGCCTCCGG |
| NO: 284 | TATTCCTCCTGATCTCTGCGCATTTCACCGCTACACCAGGAATTCTGTCTGCCTCTCCAGCACTCGAG |
| | CTGCCCGGTTCACAGTGGCTCTCCGGGGTTGAGCCCCGGTCTTTCACACTGCGCCTAAACAGCCGCCT |
| | GCGTGCCCTTTACGCCCAATAAATCCGGATAACGCTCGCCCCCCACGTATTACCGCAGCTGCTGGCA |
| | CGTGGTTGGCTGGGGCTTGCTCATCGGGTACCGTCTCCCTCTTCCCCGATAACAGAGCTTTACGGAAC |
| | GAATCCCTTCTTCACTCACGCGGCATTGCTGCGTCAGAGTTGCCTCCATTGCGCAATATTCCCCA |
| OTU | >denovo7324 |
| Ref | |
| SEQ ID | CCTGTTTGATCCCCGCACTTTCGTGCCTCAGCGTCAGTTGAGCGCCGGTATGCTGCCTTCGCAATCGG |
| NO: 285 | GGTTCTGCGTGATATCTATGCATTTCACCGCTACACCACGCATTCCGCATACTTCTCGCTCACTCAAG |
| | ACCGACAGTTTCAACGGCTCGGCGGGGTTGAGCCCCGCAATTTTACCGCTGACTTATCAGTCCGCCT |
| | ACGCACCCTTTAAACCCAATAAATCCGGATAACGCTCGCATCCTCCGTATTACCGCGGCTGCTGGCA |
| | CGGAGTTAGCCGATGCTTTTTCTTCAGGTACTCTCCTTCTGGCTACGCGTAGCCAGCTTTGCTCCCTG |
| | ACAAAAGAGGTTTACGACCCGTAGGGCCTTATTCCCTCACGCGACTTGGCTGGTTCAGCCTTCCGGC |
| | CATTGACCAATATTCCTCA |
| OTU | >denovo7330 |
| Ref | |
| SEQ ID | CCTGTTTGCTCCCCATGCTCTCATGCCTCAGCGTCAGTTACAGTCCAGTAAGTCGCCTTCGCCACTGG |
| NO: 286 | TGTTCTTCCTAATCTCTACGCATTTCACCGCTACACTAGGAATTCCACTTACCTCTCCTGCACTCTAGT |
| | TACACAGTTTTATATGCCTTCTACAGGTTAAGCCTATATATTTGACACATAACTTATCTAACCGCCTA |
| | CGCATCCTTTACGCCCAGTTATTCCGAACAACGCTTGCCACCTACGTATTACCGCGGCTGCTGGCACG |
| | TAGTTAGCCGTGGCTTCCTCTTCAGGTACCGTCCTTCCTCTTCCCTGCTAACAGAGCTTTACAATCCG |
| | AAAACCTTCTTCACTCACGCGGCGTTGCTGCATCAGGGTTCCCCCCATTGTGCAATATCCCCCA |
| OTU | >denovo7334 |
| Ref | |
| SEQ ID | CCTGTTCGCTCCCCGCGCTCTCGCGCCTCGGCGTCAGTGTCCTTCCGGCGGGCCGCCTTCGCCTCTGG |
| NO: 287 | TGTTCCTCCTGGCATCTGCGCATTTCACCGCTACGCCAGGAATTCCGCCCGCCCCTCCGGCACTCCAG |
| | CGACCCGGTTCGGGGCGCTTTCCCGGGGTTGGGCCCCGGATTTTCACACCCCGCCTAAGCCGCCGCC |
| | TGCGCGCGCTTTACGCCCAGTGATTCCGGACAACGCTCGCCCCCCACGTATTGCCGCGGCTGCTGGC |
| | ACGTGGTTGGCCGGGGCTTTCTTGCCGGGTACCGTCATCTGTTCTTCCCCGGCGGCGGGGCTTTACGG |
| | GCCGAGGCCCTTCATCGCCCACGCGGCATCGCTGCGTCAGAGTTCCCTCCATTGCGCAATATTCCCCA |
| OTU | >denovo769 |
| Ref | |
| SEQ ID | CCTGTTTGCTCCCCACGCTTTCGAGCCTCAACGTCAGTAATCGTCCAGTAAGCCGCCTTCGCCACTGG |
| NO: 288 | TGTTCCTCCTAATATCTACGCATTTCACCGCTACACTAGGAATTCCGCTTACCTCTCCGACACTCTAG |
| | TTGCATAGTTTCCAATGCAGTCCCGGGGTTGAGCCCCGGGTTTTCACATCAGACTTACACAACCGTCT |
| | ACGCTCCCTTTACACCCAGTAAATCCGGATAACGCTTGCACCATACGTATTACCGCGGCTGCTGGCA |
| | CGTATTTAGCCGGTGCTTCTTAGTCAGGTACCGTCATTTTCTTCCCTGCTGATAGAGCTTTACATACC |
| | GAAATACTTCTTCGCTCACGCGGCGTTGCTGCATCAGGGTTTCCCCCATTGTGCAATATTCCCCA |
| OTU | >denovo812 |
| Ref | |
| SEQ ID | CCTGTTTGCTCCCCGCGCTTTCGCGCCTAAGCGTCAGTGTCTGTCCGGCAGGCCGCCTTCGCCTCTGG |
| NO: 289 | TATTCCTCCTGATCTCTGCGCATTTCACCGCTACACCAGGAATTCTGCCTGCCCCTCCAGCACCCGAG |
| | CCGCGCGGTTTGGAAGGCCTCTCCGGGGTTGGGCCCCGGGCTTTGACATCCCACCCGCGCAGCCGCC |
| | TGCGCGCCCTTTACGCCCAGTGATTCCGGACAACGCTCGCCCCTCACGTCTTACCGCGGCTGCTGGCA |
| | CGTGATTGGCCGGGGCTTGCTTGCCGGGTACCGTCTTCTCTCTTCCCCGGCGGCAGGGCTTTACGGGC |
| | CGGAGCCCTTCTTCGCCCACGCGGCATCGCTGCGTCAGAGTTTCCTCCATTGCGCAATATTCCCCA |
| OTU | >denovo857 |
| Ref | |
| SEQ ID | CCTGTTTGCTCCCCACGCTCTCGCGCCTTAGCGTCAGTTGCTGTCCAGCAGGCCGCCTTCGCCTCCGG |
| NO: 290 | TATTCCTCCTGATCTCTGCGCATTTCACCGCTACACCAGGAATTCTGCCTGCCTCTCCAGCACTCCAG |
| | CTGACTCGGTTCCTGTCGCCTCTCCGGGTTGAGCCCGGATATTTTACAACAGGCCTTCGCCGCCGCCT |
| | ACGCGCCCTTTACGCCCAGTAATTCCGGACAACGCTCGCCCCCTACGTATTACCGCGGCTGCTGGCA |
| | CGTAGTTAGCCGGGGCTTGAAAAAAGGGTTGAATCTCTCTTTTCCCCTTCGACTGAGCTCTGCGGGA |
| | CGAATCCCTTCCTCACTCACGCGGCATCGCTGCGTCAGAGTTGCCTCCATTGCGCAATATTCCCCA |
| OTU | >denovo867 |
| Ref | |
| SEQ ID | CCTGTTCGATACCCACGCTTTCGTGCTTCAGCGTCAGTTGGGCGCCGGTATGCTGCCTTCGCAATCGG |
| NO: 291 | AGTTCTGCGTGATATCTATGCATTTCACCGCTACACCACGCATTCCGCATACTTCTCGCCCACTCAAG |
| | ACCGTCAGTTTCAACGGCGGGCGGAGGTTGAGCCCCCGTATTTTACCGCTGACTTGACAGTCCGCCT |
| | ACGCACCCTTTAAACCCAATAAATCCGGATAACGCTCGCATCCTCCGTATTACCGCGGCTGCTGGCA |
| | CGGAGTTAGCCGATGCTTTTTCTTCGGGTACTCTCGACTCCGCCACACGTGGCGGAATTTGCTCCCCG |
| | ACAAAAGAGGTTTACAACCCGTAGGGCCGTCATCCCTCACGCGACTTGGCTGGTTCAGGCTCGCGCC |
| | CATTGACCAATATTCCTCA |
| OTU | >denovo894 |
| Ref | |
| SEQ ID | CCTATTTGCTCCCCACGCTTTCGTGCCTGAGCGTCAGCAACAGTCCAGGCGGTCGCCTTCGCAACTGG |
| NO: 292 | TGTTCTTCCATATATCTACGCATTTTACCGCTACACATGGAGTTCCACCGCCCTCTCCTGTCCTCTAGC |
| | GTGCCAGTTTCCAAAGCCTGTACCGGTTAAGCCCGTACCTTTCACTTCAGACTTGACGCGCAGCCTGC |
| | GCACCCTTTACGCCCAATCATTCCGGATAACGCTTGCCACCTACGTATTACCGCGGCTGCTGGCACGT |
| | AGTTAGCCGTGACTTTCTGGCGGGGTACCATCAAAAGAAAAGCATTTCCTCTTTCCTTCCTTTTTCCC |
| | CCGCAACAGAGTTTTACGATCCGAAGACCTTCTTCGCTCACGCGGCATTGCTCGTTCAGGCTTGCGCC |
| | CATTGACGAAAATTCCCTA |
| OTU | >denovo943 |
| Ref | |
| SEQ ID | CCTGTTTGATACCCACACTTTCGAGCCTCAATGTCAGTTGCAGCTTGGCGGACTGCTTTCGCAATCGG |
| NO: 293 | GGTTCTTCGTGATATCTAAGCATTTCACCGCTACACCACGAATTCCATCCGCCTCAAATGCACTCAAG |
| | AAAACCAGTATCAACTGCAATTTTACGGTTGAGCCGCAAACTTTCACAGCTGACTTAATCTCCCATCT |
| | ACGCTCCCTTTAAACCCAATAAATCCGGATAACGCTCGCATCCTCCGTATTACCGCGGCTGCTGGCA |
| | CGGAGTTAGCCGATGCTTATTCATAAGGTACATACAAACAGGTACACGTACCTGACTTTATTCCCTTA |
| | TAAAAGAAGTTTACAACCCATAGGGCAGTCTTCCTTCACGCTACTTGGCTGGTTCAGACTCTCGTCCA |
| | TTGACCAATATTCCTCA |

### REFERENCES

1. WAKSHLAG J J, SIMPSON K W, STRUBLE A M, DOWD S E. Negative Fecal Characteristics Are Associated with pH and Fecal Flora Alterations During Dietary Change in Dog. International Journal of Applied Research in Veterinary Medicine. 2011; 9(3):278.
2. G. M. The WALTHAM Faeces Scoring System—a tool for veterinarians and pet owners: how does your pet rate? Waltham Focus. 2001; 11(2):24-5.
3. ANTHARAM, V. C., LI, E. C., ISHMAEL, A., SHARMA, A., MAI, V., RAND, K. H. & WANG, G. P. 2013. Intestinal dysbiosis and depletion of butyrogenic bacteria in Clostridium difficile infection and nosocomial diarrhea. J Clin Microbiol, 51, 2884-92.
4. ANTHARAM, V. C., MCEWEN, D. C., GARRETT, T. J., DOSSEY, A. T., LI, E. C., KOZLOV, A. N., MESBAH, Z. & WANG, G. P. 2016. An Integrated Metabolomic and Microbiome Analysis Identified Specific Gut Microbiota Associated with Fecal Cholesterol and Coprostanol in Clostridium difficile Infection. PLoS One, 11, e0148824.
5. BARRY K A, HERNOT D C, VAN LOO J, FAHEY G C JR, D E GODOY MR. Fructan supplementation of senior Beagle dogs affects stool metabolite concentrations and fecal microbiota concentrations, but not nitrogen partitioning in excreta. J Anim Sci. 2014 November; 92(11):4964-71.
6. BELL E T, SUCHODOLSKI J S, ISAIAH A, FLEEMAN L M, COOK A K, STEINER J M, MANSFIELD CS. Fecal microbiota of Beagle dogs with insulin-treated diabetes mellitus. PLoS One. 2014 Oct. 3; 9(10):e108729.
7. BELOSHAPKA, A. N., DOWD, S. E., SUCHODOLSKI, J. S., STEINER, J. M., DUCLOS, L. & SWANSON, K. S. 2013. Fecal microbial communities of healthy adult dogs fed raw meat-based diets with or without inulin or yeast cell wall extracts as assessed by 454 pyrosequencing. FEMS Microbiol Ecol, 84, 532-41.
8. BERMINGHAM, E. N., YOUNG, W., KITTELMANN, S., KERR, K. R., SWANSON, K. S., ROY, N. C. & THOMAS, D. G. 2013. Dietary format alters fecal bacterial populations in the domestic cat (Felis catus). Microbiologyopen, 2, 173-81.
9. DAVID, L. A., MAURICE, C. F., CARMODY, R. N., GOOTENBERG, D. B., BUTTON, J. E., WOLFE, B. E., LING, A. V., DEVLIN, A. S., VARMA, Y., FISCHBACH, M. A. AND BIDDINGER, S. B., 2014. Diet rapidly and reproducibly alters the human gut microbiome. Nature, 505(7484), p. 559.
10. D E GODOY, M. R., KERR, K. R. & FAHEY, G. C., JR. 2013. Alternative dietary fiber sources in companion animal nutrition. Nutrients, 5, 3099-117.
11. DEUSCH, O., O'FLYNN, C., COLYER, A., MORRIS, P., ALLAWAY, D., JONES, P. G. & SWANSON, K. S. 2014. Deep Illumina-based shotgun sequencing reveals dietary effects on the structure and function of the fecal microbiome of growing kittens. PLoS One, 9, e101021.
12. FADROSH, D. W., MA, B., GAJER, P., SENGAMALAY, N., OTT, S., BROTMAN, R. M., & RAVEL, J. (2014). An improved dual-indexing approach for multiplexed 16S rRNA gene sequencing on the Illumina MiSeq platform. Microbiome, 2(1), 6.
13. FERRARIO C, STATELLO R, CARNEVALI L, et al. How to Feed the Mammalian Gut Microbiota: Bacterial and Metabolic Modulation by Dietary Fibers. Front Microbiol. 2017; 8:1749. Published 2017 Sep. 12. doi:10.3389/fmicb.2017.01749
14. HOODA, S., VESTER BOLER, B. M., KERR, K. R., DOWD, S. E. & SWANSON, K. S. 2013. The gut microbiome of kittens is affected by dietary protein:carbohydrate ratio and associated with blood metabolite and hormone concentrations. Br J Nutr, 109, 1637-46.
15. MARCHESI, J. R., ADAMS, D. H., FAVA, F., HERMES, G. D., HIRSCHFIELD, G. M., HOLD, G., QURAISHI, M. N., KINROSS, J., SMIDT, H., TUOHY, K. M., THOMAS, L. V., ZOETENDAL, E. G. & HART, A. 2016. The gut microbiota and host health: a new clinical frontier. Gut, 65, 330-9.
16. MCDONALD D, PRICE M N, GOODRICH J, NAWROCKI E P, DESANTIS T Z, PROBST A, ANDERSEN G L, KNIGHT R, HUGENHOLTZ P. An improved greengenes taxonomy with explicit ranks for ecological and evolutionary analyses of bacteria and archaea. ISME J. 2012; 6(3):610-8.
17. MIDDELBOS, I. S., VESTER BOLER, B. M., QU, A., WHITE, B. A., SWANSON, K. S. & FAHEY, G. C., JR. 2010. Phylogenetic characterization of fecal microbial communities of dogs fed diets with or without supplemental dietary fiber using 454 pyrosequencing. PLoS One, 5, e9768.
18. MINAMOTO, Y., DHANANI, N., MARKEL, M. E., STEINER, J. M. & SUCHODOLSKI, J. S. 2014. Prevalence of Clostridium perfringens, Clostridium perfringens enterotoxin and dysbiosis in fecal samples of dogs with diarrhea. Vet Microbiol, 174, 463-73.
19. MINAMOTO, Y., OTONI, C. C., STEELMAN, S. M., BUYUKLEBLEBICI, O., STEINER, J. M., JERGENS, A. E. & SUCHODOLSKI, J. S. 2015. Alteration of the fecal microbiota and serum metabolite profiles in dogs with idiopathic inflammatory bowel disease. Gut Microbes, 6, 33-47.
20. MOXHAM, G The WALTHAM Faeces Scoring System—a tool for veterinarians and pet owners: how does your pet rate? Waltham Focus. 2001; 11(2):24-5.
21. PANASEVICH, M. R., KERR, K. R., DILGER, R. N., FAHEY, G. C., JR., GUERIN-DEREMAUX, L., LYNCH, G. L., WILS, D., SUCHODOLSKI, J. S., STEER, J. M., DOWD, S. E. & SWANSON, K. S. 2015. Modulation of the fecal microbiome of healthy adult dogs by inclusion of potato fibre in the diet. Br J Nutr, 113, 125-33.
22. SIMPSON J M, MARTINEAU B, JONES W E, BALLAM J M, MACKIE R I. Characterization of fecal bacterial populations in canines: effects of age, breed and dietary fiber. Microb Ecol. 2002; 44(2):186-97.
23. SUCHODOLSKI J S, CAMACHO J, STEINER J M. Analysis of bacterial diversity in the canine duodenum, jejunum, ileum, and colon by comparative 16S rRNA gene analysis. FEMS Microbiol Ecol. 2008 December; 66(3):567-78.
24. SUCHODOLSKI J S, MARKEL M E, GARCIA-MAZCORRO J F, UNTERER S, HEILMANN R M, DOWD S E, KACHROO P, IVANOV I, MINAMOTO Y, DILLMAN E M, et al. The fecal microbiome in dogs with acute diarrhea and idiopathic inflammatory bowel disease. PLoS One. 2012; 7:e51907.
25. SUCHODOLSKI, J. S., DOWD, S. E., WILKE, V., STEINER, J. M. & JERGENS, A. E. 2012a. 16S rRNA gene pyrosequencing reveals bacterial dysbiosis in the duodenum of dogs with idiopathic inflammatory bowel disease. PLoS One, 7, e39333.
26. SUCHODOLSKI, J. S., MARKEL, M. E., GARCIA-MAZCORRO, J. F., UNTERER, S., HEILMANN, R. M., DOWD, S. E., KACHROO, P., IVANOV, I., MINAMOTO, Y., DILLMAN, E. M., STEINER, J. M., COOK, A. K. & TORESSON, L. 2012b. The fecal microbiome in dogs with acute diarrhea and idiopathic inflammatory bowel disease. PLoS One, 7, e51907.
27. SUCHODOLSKI, J. S., 2016. Diagnosis and interpretation of intestinal dysbiosis in dogs and cats. The Veterinary Journal, 215, pp. 30-37.
28. SUNVOLD G D, FAHEY G C, MERCHEN N R, TITGEMEYER E C, BOURQUIN L D, BAUER L L et al. Dietary fiber for dogs: IV. In vitro fermentation of selected fiber sources by dog fecal inoculum and in vivo digestion and metabolism of fiber-supplemented diets. Journal of Animal Science. 1995; 73(4):1099.
29. VANDEPUTTE D, FALONY G, VIEIRA-SILVA S, et al. Stool consistency is strongly associated with gut microbiota richness and composition, enterotypes and bacterial growth rates. Gut 2016; 65:57-62.
30. VAZQUEZ-BAEZA, Y., HYDE, E. R., SUCHODOLSKI, J. S. & KNIGHT, R. 2016. Dog and human inflammatory bowel disease rely on overlapping yet distinct dysbiosis networks. Nat Microbiol, 1, 16177.
31. VÁZQUEZ-BAEZA, Y., HYDE, E. R., SUCHODOLSKI, J. S. AND KNIGHT, R., 2016. Dog and human inflammatory bowel disease rely on overlapping yet distinct dysbiosis networks. Nature microbiology, 1(12), p. 16177.
32. VICKERS, R. J., SUNVOLD, G. D., KELLEY, R. L. & REINHART, G. A. 2001. Comparison of fermentation of selected fructooligosaccharides and other fiber substrates by canine colonic microflora. American Journal of Veterinary Research, 62, 609-615.
33. WAKSHLAG, J. J., SIMPSON, K. W., STRUBLE, A. M. & DOWD, S. E. 2011. Negative Fecal Characteristics Are Associated with pH and Fecal Flora Alterations During Dietary Change in Dog. International Journal of Applied Research inVeterinary Medicine, 9, 278.
34. YILMAZ P, PARFREY L W, YARZA P, GERKEN J, PRUESSE E, QUAST C, SCHWEER T, PEPLIES J, LUDWIG W, GLÖCKNER F O. The SILVA and “All-species Living Tree Project (LTP)” taxonomic frameworks. Nucleic Acids Res. 2014; 42(Database issue):643-8.
35.

Although the presently disclosed subject matter and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the presently disclosed subject matter, processes, machines, manufacture, compositions of matter, means, methods, or steps, presently existing or later to be developed that perform substantially the same function or achieve substantially the same result as the corresponding embodiments described herein can be utilized according to the presently disclosed subject matter. Accordingly, the appended claims are intended to include within their scope such processes, machines, manufacture, compositions of matter, means, methods, or steps.

Patents, patent applications, publications, product descriptions and protocols are cited throughout this application the disclosures of which are incorporated herein by reference in their entireties for all purposes.

1. A pet food product comprising sugar beet pulp at a concentration between about 0.5% w/w and about 6% w/w.

2. (canceled)

3. (canceled)

4. The pet food product of claim 1, wherein the pet food product is a topper.

5. The pet food product of claim 4, wherein the topper is fed to a companion animal at a dosage of between about 0.5 g/day to about 90 g/day of the sugar beet pulp.

6. The pet food product of claim 1, further comprising additional fiber, a probiotic, and/or a prebiotic.

7. A pet food product comprising a bacterium selected from the group consisting of Lachnospiraceae sp., Faecalibacterium prausnitzii, Bacteroides plebeius, Holdemania [Eubacterium] biforme, Dorea sp., Ruminococcaceae sp., Bacteroides sp., Blautia sp., Erysipelotrichaceae sp., Lachnospiraceae sp. and any combination thereof in an amount effective to improve intestinal health and/or fecal quality in a companion animal.

8. The pet food product of claim 7, wherein the bacterium is selected from the group consisting of Faecalibacterium prausnitzii, Bacteroides plebeius, Holdemania [Eubacterium] biforme and any combination thereof.

9. The pet food product of claim 8, wherein the bacterium comprises a 16S ribosomal RNA (rRNA) comprising a nucleotide sequence having at least about 95% sequence identity or identical to the nucleotide sequence of any one of SEQ ID NOs: 1-14.

10. The pet food product of claim 9, wherein the bacterium is selected from the group consisting of denovo1184, denovo1244, denovo1696, denovo2407, denovo2451, denovo283, denovo3487, denovo4154, denovo4328, denovo4681, denovo498, denovo5338, denovo6995, denovo943, and any combination thereof.

11. The pet food product of claim 7, wherein the effective amount of the bacterium is between about 10 thousand CFU and about 100 trillion CFU.

12. The pet food product of claim 7, further comprising sugar beet pulp.

13. The pet food product of claim 12, wherein the pet food product comprises sugar beet pulp in an amount effective to improve intestinal health and/or fecal quality in the companion animal.

14. The pet food product of claim 12, wherein the sugar beet pulp is present in the pet food product at a concentration between about 0.5% w/w and about 5% w/w.

15. (canceled)

16. (canceled)

17. The pet food product of claim 7, further comprising a probiotic in addition to the bacterium.

18. (canceled)

19. The pet food product of claim 1, wherein the pet food product improves intestinal health and/or fecal quality in a companion animal within about 14 days after administering the pet food product to the companion animal.

20. (canceled)

21. The pet food product of claim 7, wherein the pet food product is a topper.

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. A pet food product comprising sugar beet pulp for use in treating an intestinal dysbiosis in a companion animal, wherein the sugar beet pulp is at a concentration between about 0.1% w/w and about 10% w/w in the pet food product.

50. The pet food product of claim 49, wherein the pet food product is a dietary supplement or a functional food.

51. The pet food product of claim 49, wherein the pet food product is a topper.

52. The pet food product of claim 49, wherein the topper is fed to the companion animal at a dosage of between about 0.5 g/day to about 90 g/day of the sugar beet pulp.

53. The pet food product of claim 49, wherein the sugar beet pulp is at a concentration between about 0.5% w/w and about 6% w/w in the pet food product.

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. (canceled)

74. (canceled)

75. (canceled)


SEQUENCE LISTING (293 sequences)

SEQ ID NO: 1 - DNA, 404 bp, Unknown
  source: /note=“Description of Unknown 16S rRNA sequence”
cctgtttgctccccacgctttcgagcctcaacgtcagtctctgtccagtaagccgccttcgccactggtgttcctcctaatatctacgcatttcaccgctacactaggaattccacttacctctccagtactctagctcaacagtttccaaagcagtcccgtggttaagcctcgggctttcacttcagacttgccgagccgtctacgctccctttacacccagtaaatccggataacgctcgccccctacgtattaccgcggctgctggcacgtagttagccggggcttcttagtcaggtaccgtcattctcttccctgctgatagagctttacgtaccgaagtacttcttcactcacgcggcgtcgctgcatcagggtttcccccattgtgcaatatccccca

SEQ ID NO: 2 - DNA, 404 bp, Unknown
  source: /note=“Description of Unknown 16S rRNA sequence”
cctgtttgctacccacactttcgagcctcagcgtcagttggtgcccagtaggccgccttcgccactggtgttcctcccgatatctacgcattccaccgctacaccgggaattccgcctacctctgcactactcaagaaaaacagttttgaaagcagtttatgggttgagcccatagatttcacttccaacttgtcttcccgcctgcgctccctttacacccagtaattccggacaacgcttgtgacctacgttttaccgcggctgctggcacgtagttagccgtcacttccttgttgggtaccgtcattatcttccccaacaacaggagtttacaatccgaagaccttcttcctccacgcggcgtcgctgcatcagggtttcccccattgtgcaatattcccca

SEQ ID NO: 3 - DNA, 424 bp, Unknown
  source: /note=“Description of Unknown 16S rRNA sequence”
cctgtttgatacccacactttcgagcatgaacgtcagttacggcttagtgtgctgccttcgcaatcggagttcttcgtgatatctaagcatttcaccgctacaccacgaattccgcacacctcaaccgcactcaaggacgccagtatcaactgcaattttaaggttgagccccaaactttcacagctgacttaacgacccgtctgcgctccctttaaacccaataaatccggataacgctcgcatcctccgtattaccgcggctgctggcacggagttagccgatgcttattcataaggtacatacaagctcccacacgtgggaggttttattcccttataaaagaagtttacaatccgtaggaccttcatccttcacgctacttggctggttcagactctcgtccattgaccaatattcctca

SEQ ID NO: 4 - DNA, 429 bp, Unknown
  source: /note=“Description of Unknown 16S rRNA sequence”
cctatttgctccccacgctttcgtgcttcagtgtcagaatccagaccagacggccgccttcgccaccggtgttcttccatatatctacgcattttaccgctacacatggagttccgccgtcctcttctgttctctagctgatcagtttccagagcaagtacgggttgagcccataccttttactccagacttgatctgccacctacgcaccctttacgcccaatcattccggataacgctcgccacctacgtattaccgcggctgctggcacgtagttagccgtgactttctggtaagataccatcactcactcatcattccctatgagtgccgtttttctcttacaacagagctttacgatccgaagaccttcctcactcacgcggcattgctcgttcagggttccccccattgacgaaaattcccta

SEQ ID NO: 5 - DNA, 404 bp, Unknown
  source: /note=“Description of Unknown 16S rRNA sequence”
cctgtttgctccccacgctttcgagcctcaacgtcagttatcgtccagtaagccgccttcgccactgatgttcctcctaatatctacgcatttcaccgctacactaggaattccgcttacctctccgacactctagaagcacagtttccaaagcagtcacggggttgagccccgggctttcacttcagacttgcacttccgtctacgctccctttacacccagtaaatccggataacgcttgccccctacgtattaccgcggctgctggcacgtagttagccggggcttcttagtcaggtaccgtcattttcttccctgctgatagaagtttacataccgaaatacttcttccttcacgcggcgtcgctgcatcagggtttcccccattgtgcaatattcccca

SEQ ID NO: 6 - DNA, 404 bp, Unknown
  source: /note=“Description of Unknown 16S rRNA sequence”
cctgtttgctacccatgctttcgagcctcagcgtcagttagtgcccagcaggccgccttcgccactggtgttcctcccgatatctacgcattccaccgctacaccgggaattccgcctgcctctgcaccactcaagatttgcagttttgaatgcgagaaggggttgagcccctccattaaacatccaacttgcaaacccgcctgcgctccctttacacccagtaattccggacaacgcttgccacctacgtattaccgcggctgctggcacgtagttagccgtggcttcctcctcgggtaccgtcatacttcgtccccgaagacagaggtttacaatccgaaaatcttcttccctcacgcggcgtcgctgcatcagagtttcctccattgtgcaatattcccca

SEQ ID NO: 7 - DNA, 424 bp, Unknown
  source: /note=“Description of Unknown 16S rRNA sequence”
cctgtttgatacccgcactttcgagcatcagcgtcagttacggtccagtaagctgccttcgcaatcggagttcttcgtgatatctaagcatttcaccgctacaccacgaattccgcctacctataccgcactcaagaaatccagtatcaactgcaattttacggttgagccgcaaactttcacaactgacttaaacttccgcctacgctccctttaaacccaataaatccggataacgctcggatcctccgtattaccgcggctgctggcacggagttagccgatccttattcatacggtacatacaaaaaagcacacgtgcttcactttattcccgtataaaagaagtttacaacccatagggcagtcatccttcacgctacttggctggttcagactctcgtccattgaccaatattcctca

SEQ ID NO: 8 - DNA, 404 bp, Unknown
  source: /note=“Description of Unknown 16S rRNA sequence”
cctgtttgctccccacgctttcgagcctcaacgtcagttaccgtccagtaagccgccttcgccactggtgttcctcctaatatctacgcatttcaccgctacactaggaattccgcttacccctccggtactcaagatcaacagtttccaatgcagtccaggggttgagcccctgcctttcacatcagacttgctgctccgtctacgctccctttacacccagtaaatccggataacgcttgccccctacgtattaccgcggctgctggcacgtagttagccggggcttcttagtcaggtaccgtcattatcttccctgctgatagaagtttacataccgagatacttcttccttcacgcggcgtcgctgcatcagggtttcccccattgtgcaatattcccca

SEQ ID NO: 9 - DNA, 424 bp, Unknown
  source: /note=“Description of Unknown 16S rRNA sequence”
cctgtttgatacccacactttcgagcatgaacgtcagttacagtttagcaagctgccttcgcaatcggggttcttcgtgatatctaagcatttcaccgctacaccacgaattccgcctgcctcaactgcactcaaggaaaccagtatcaactgcaattttacggttgagccgcaaactttcacaactgacttaatctcccgtctgcgctccctttaaacccaataaatccggataacgctcgcatcctccgtattaccgcggctgctggcacggagttagccgatgcttattcatacggtacatacaaaatcctacacgtaggaaactttattcccgtataaaagaagtttacaatccgtaggaccttcatccttcacgctacttggctggttcaggctctcgcccattgaccaatattcctca

SEQ ID NO: 10 - DNA, 423 bp, Unknown
  source: /note=“Description of Unknown 16S rRNA sequence”
cctatttgctccccacgctttcgtgcctgagcgtcagttacagactagcaagccgccttcgccaccggtgttcctccatatatctatgcattttaccgctacacatggaattccacttgccccttctgcactctagttcaccagtttctaagcctggatggggttgagccccacaatttaagcttaaacttaataaaccgcctgcgcaccctttacgcccaataattccggataacgctcgtcaccttcgtattaccgcggctgctggcacgaagttagccgtgactttctggtaaagtaccgtcacttacgtagttgatacgtaacattcttcctttacaacagagctttacaagccgaagaccttcatcactcacgcggcattgctcggtcaggcttgcgcccattgccgaaaattcccta

SEQ ID NO: 11 - DNA, 424 bp, Unknown
  source: /note=“Description of Unknown 16S rRNA sequence”
cctgtttgatacccacactttcgagcctcaatgtcagttgcagcttagcaggctgccttcgcaatcggagttcttcgtgatatctaagcatttcaccgctacaccacgaattccgcctgcctcaactgcactcaagatatccagtatcaactgcaattttacggttgagccgcaaactttcacaactgacttaaacatccatctacgctccctttaaacccaataaatccggataacgctcggatcctccgtattaccgcggctgctggcacggagttagccgatccttattcataaagtacatgcaaacgggtatgcatacccgactttattcctttataaaagaagtttacaacccatagggcagtcatccttcacgctacttggctggttcaggctctcgcccattgaccaatattcctca

SEQ ID NO: 12 - DNA, 404 bp, Unknown
  source: /note=“Description of Unknown 16S rRNA sequence”
cctgtttgctccccacgctttcgagcctcaacgtcagttactgtccagtaagccgccttcgccactggtgttcctcctaatatctacgcatttcaccgctacactaggaattccacttacctctccagcactctagcagaacagtttccaaagcagtcccggggttgagccccgggctttcacttcagacttgctccgccgtctacgctccctttacacccagtaaatccggataacgcttgccccctacgtattaccgcggctgctggcacgtagttagccggggcttcttagtcaggtaccgtcattttcttccctgctgatagagctttacataccgagatacttcttcactcacgcggcgtcgctgcatcagggtttcccccattgtgcaatattcccca

SEQ ID NO: 13 - DNA, 404 bp, Unknown
  source: /note=“Description of Unknown 16S rRNA sequence”
cctgttcgctccccacgctttcgagtctcagcgtcagttatagtccagcaagccgccttcgccaccggtgttcttcctgatatctacgcatttcaccgctacaccaggaattccgcttgcccttcctacactctagctgtacagtttcaaaagcagtcttggggttgagccccaagttttcactcctgacttgtactgccgcctacactccctttacacccagtaaatccggataacgcttgccccatacgtattaccgcggctgctggcacgtatttagccggggcttcttctgtaggtaccgtcattatcttccctactgatagagctttacataccgaaatacttcttcactcacgcggcgtcgctgcatcagagttgcctccattgtgcaatattcccca

SEQ ID NO: 14 - DNA, 424 bp, Unknown
  source: /note=“Description of Unknown 16S rRNA sequence”
cctgtttgatacccacactttcgagcctcaatgtcagttgcagcttggcggactgctttcgcaatcggggttcttcgtgatatctaagcatttcaccgctacaccacgaattccatccgcctcaaatgcactcaagaaaaccagtatcaactgcaattttacggttgagccgcaaactttcacagctgacttaatctcccatctacgctccctttaaacccaataaatccggataacgctcgcatcctccgtattaccgcggctgctggcacggagttagccgatgcttattcataaggtacatacaaacaggtacacgtacctgactttattcccttataaaagaagtttacaacccatagggcagtcttccttcacgctacttggctggttcagactctcgtccattgaccaatattcctca

SEQ ID NO: 15 - DNA, 406 bp, Unknown
  source: /note=“Description of Unknown 16S rRNA sequence”
cctgtttgctccccacgctttcgtacctcagtgtcagttacagtccagaaagccgccttcgccactggtgttcctcctaatatctacgcatttcaccgctacactaggaattccgctttcctctcctgcactcaagtttgccagttcgcagggcgaacaatggttgagccattgccttaaaccctgcgcttggtaaaccacctacgtaccctttacgcccaataattccggataacgcttgccccctccgtattaccgcggctgctggcacggagttagccggggcttcctccaagggtaccgtcatttgtttcttccccaaggacagagctttacgacccaaaggccttcatcgctcacgcggcgttgctgcatcaggcttgcgcccattgtgcaatattcccca

SEQ ID NO: 16 - DNA, 404 bp, Unknown
  source: /note=“Description of Unknown 16S rRNA sequence”
cctgttcgctccccacgctttcgcacctcagtgtcagtataagcctggcagaccgccttcgcctccggtattcctcctgatctctgcgcatttcaccgctacaccaggaattctgcctgcctcgacaatactccagctgcccggtttgcggtgacctcccagggttgagccctggacttttacaccgcacctaaacaaccacctgcgtgccctttacgcccaataattccggataacgctcgccccctacgtattaccgcagctgctggcacgtagttggctggggcttgcttaccgggtaccgtcatcgtcttccccggtaacagagctttacagaacgaatcccttcttcactcacgcggcatcgctgcgtcagagttgcctccattgcgcaatattcccca

SEQ ID NO: 17 - DNA, 404 bp, Unknown
  source: /note=“Description of Unknown 16S rRNA sequence”
cctgtttgctccccacgctttcgagcctcaacgtcagttaccgtccagtaagccgccttcgccactggtgttcctcctaatatctacgcatttcaccgctacactaggaattccgcttacctctccggcactcaagtcccacagtttccaatgcaatccagaagttgagcctctgcctttcacatcagacttgcagaaccgtctacgctccctttacacccagtaaatccggataacgcttgccccctacgtattaccgcggctgctggcacgtagttagccggggcttcttagtcaggtaccgtcattttcttccctgctgatagagctttacatcacgaatgacttcttcactcacgcggcgtcgctgcatcagggtttcccccattgtgcaatattcccca

SEQ ID NO: 18 - DNA, 406 bp, Unknown
  source: /note=“Description of Unknown 16S rRNA sequence”
cctgttcgctccccgcgctctcgcgcctcagcgtcagtgtccgtccggcgggccgccttcgcctccggtgttcctcctggcctctgcgcatttcaccgctacgccaggagttccgcccgcccctccggcactccagccgcccggtccggggcgcttgcccggggttgggccccggcttttcacaccccgcctaagcggccgcctgcgcgcgctttacgcccagtgattccggacaacgctcgccccccacgtattgccgcggctgctggcacgtggttggccggggctttcgcgccgggttcagtcatttcttcctccccggctacggggctttacgggccgaggcccttcgtcgcccacgcggcgtcgctgcgtcagagttccctccattgcgcaatattcccca

SEQ ID NO: 19 - DNA, 404 bp, Unknown
  source: /note=“Description of Unknown 16S rRNA sequence”
cctgtttgctccccacgctttcgagcctcagcgtcagttacagtccagagaatcgccttcgccactggtgttcttcctaatctctacgcatttcaccgctacactaggaattccattctcctctcctgcactctagacttccagtttgaaatgcagcacccaagttgagcccgggtatttcacatctcacttaaaagtccgcctacgctccctttacgcccagtaaatccggacaacgctcgccacctacgtattaccgcggctgctggcacgtagttagccgtggcttcctcctcaggtaccgtcattatcgtccctgaagacagagctttacaacccgaaggccgtcatcactcacgcggcgttgctgcatcagggtttcccccattgtgcaatattcccca

SEQ ID NO: 20 - DNA, 407 bp, Unknown
  source: /note=“Description of Unknown 16S rRNA sequence”
cctgttcgctccccgcgctctcgcgcctcagcgtcagtgtccgtccggcgggccgccttcgcctccggtgttcctcccggcctctgcgcatttcaccgctacgccgggagttccgcccgcccctccggcgctccagccgcccggtccggggcgcttgcccggggttgggccccggcttttcacaccccgcctaagcggccgcctgcgcgcgctttacgcccagtgattccggacaacgctcgccccccacgtattgccgcggctgctggcacgtggttggccggggctttcgtgccgggttcagtcatttccttcctccccggcggcggggctttacgggcctgggcccttcatcgcccacgcggcgtcgctgcgtcagagttgcctccattgcgcaatattcccca

SEQ ID NO: 21 - DNA, 407 bp, Unknown
  source: /note=“Description of Unknown 16S rRNA sequence”
cctgttcgctccccgcgctctcgcgccttagcgtcagtgtgcgtccggcgggccgccttcgcctccggtgttccccctggcctctgcgcatttcaccgctacgccaggggttccgcccgcctctccgccactccagccgcccggtccggggcgcttgcccggggttgggccccggcttttaacgccccgcctaagcggccgcctgcgcgcgctttacgcccagtgattccggacaacgctcgccccccacgtattgccgcggctgctggcacgtggttggccggggctttcttgccgggttgcgtcatttttttcctccccggcggcggggctttacgggcctgagcccttcatcgcccacgcggcatcgctgcgtcagagttccctccattgcgcaatattcccca

SEQ ID NO: 22 - DNA, 403 bp, Unknown
  source: /note=“Description of Unknown 16S rRNA sequence”
cctgtttgctccccacgctttcgtgcctcagtgtcagttacagtccagaaagccgccttcgctactggtgttcctcctaatatctacgcatttcaccgctacactaggaattccactttcctctcctgcactcaagtttcccagtttcaagagcttactacggttaagccgtagcctttcactcctgacttaagaaaccacctacgcaccctttacgcccagtaaatccggataacgctagccccctacgtattaccgcggctgctggcacgtagttagccggggcttcctcctcaagtaccgtcattatcttccttgaggacagagttttacgacccgaaggccttcatcactcacgcggcgttgctgcatcaggctttcgcccattgtgcaatattcccca

SEQ ID NO: 23 - DNA, 403 bp, Unknown
  source: /note=“Description of Unknown 16S rRNA sequence”
cctgtttgctccccacgctctcgcgccttagcgtcagtctcttcccggcaggccgccttcgcctccggtattcctcctgatatctgcgcatttcaccgctacaccaggaattctgcctgcctctaaaggactccagcctctcggttcctgtcgcctccccgggttgggcccgggacttttacaacaggccttctaggccgcctacgcgcgctttacgcccagtaattccggacaacgctcgccccctacgtattaccgcggctgctggcacgtagttagccggggctttaaagacgggtgacatctctcttctccccgtcgactgagctctgcgggacgaatcccttcctcactcacgcggcatcgctgcgtcagagtttcctccattgcgcaatattcccca

SEQ ID NO: 24 - DNA, 400 bp, Unknown
  source: /note=“Description of Unknown 16S rRNA sequence”
cctgtttgctccccacgctctcgcgcctgagcgtcagtctctctccagcaggccgccttcgcctccggtattcctcctgatctctgcgtatttcaccactacaccaggaattctgcctgcctctagagcactctagtcagctggttcttcctgcttacccggggtgggcccgggccttttacaaaaagcctctctgaccgcctgcgcgcgctttacgcccaatgattccggacaacgctcgccccctacgtattgccgcggctgctggcacgtagttagccggggcttaaagtcaggtagtctctctcttgcctgacgactgagctctgcgggacgaatcccttcctcactcacgcggcatcgctgcgtcagagttgcctccattgcgcaatattcccca

SEQ ID NO: 25 - DNA, 403 bp, Unknown
  source: /note=“Description of Unknown 16S rRNA sequence”
cctgtttgctccccacgctttcgtgcctcagtgtcagttacagtccagagagccgccttcgcaactggtattcctcctaatatctacgcatttcaccgctacactaggaattctactctcctctcctgcactcaagtttctcagtttcaaaggcttactacggttgagccgtagcctttcacctctgacttaagaaaccacctacgcaccctttacgcccagtaattccggataacgctagccccctacgtattaccgcggctgctggcacgtagttagccggggcttcctcctcaagtaccgtcattatcttccttgaggacagagctttacgacccgaaggccttcatcgctcacgcggcgttgctgcatcaggctttcgcccattgtgcaatattcccca

SEQ ID NO: 26 - DNA, 404 bp, Unknown
  source: /note=“Description of Unknown 16S rRNA sequence”
cctgtttgctccccacgctctcgcgcctgagcgtcagtgtctgtccggcaggccgccttcgcctctggtattcctcctgatctctgcgcatttcaccgctacaccaggaattctgcctgcctctccagcactcgagccgcgcggttcgggatgcccgaacagggttgggccctgatctttcacatcccgccttcgcggccgcctgcgcgccctttacgcccagtcattccggacaacgctcgccccctacgtattaccgcggctgctggcacgtagttagccggggcttgcttgtcaggtaccgtctctctcttccctgacaacagagctttacgggacgaatcccttcttcgctcacgcggcattgctgcgtcagagttgcctccattgcgcaatattcccca

SEQ ID NO: 27 - DNA, 404 bp, Unknown
  source: /note=“Description of Unknown 16S rRNA sequence”
cctgttcgctcccccagctttcgcgcctccgcgtcagtggcggcccagaaggctgccttcgccgtcggtgttcttcccgatatctgcgcatttcaccgctacaccgggaattccgccttcccctaccgcactcgatccccccagttcggcccgcttccgggggttgagcccccggcttcgacggtgccgcttgaagggacgcctacgcgcgctttacgcccaatgaatccggataacgctcgccccctacgtattaccgcggctgctggcacgtagttagccggggcttcttctgcaggtaccgtcaacttcttccctgctgaaagcggtttacgacccgaaggccttcgtcccgcacgcggcgttgctgcgtcagggttgcccccattgcgcaaaattcccca

SEQ ID NO: 28 - DNA, 406 bp, Unknown
  source: /note=“Description of Unknown 16S rRNA sequence”
cctgtttgctccccacgctttcgtgcctcagcgtcagttatggtccagaaagccgccttcgctactggtgttcctttgaatctctacgcatttcaccgctacactcaaagttccactttcctctcccacactctagctcggcagtttccgaagcagctccggggttgagccccgaaatttcacttcagacttaccaagccgcctacgcaccctttacacccagtaaatccggataacgcttgctccctacgtattaccgcggctgctggcacgtagttagccggagcttcttagttaggtaccgtcattttttcttccctactgatagaagtttacaatccgaagaccttcttccttcacgcggcgttgctgcatcaggcttgcgcccattgtgcaatattcccca

SEQ ID NO: 29 - DNA, 423 bp, Unknown
  source: /note=“Description of Unknown 16S rRNA sequence”
cctgttcgatccccacgctttcgtgcctcagcgtcagtcaggcgccagtacgctgccttcgcaatcggagttctgcgcaatatctaagcatttcaccgctacactgcgcattccgcgtacctctcgccttctctagcactgcagtttcaacggctcgccggggttgagccccgggcttttaccgctgacttgcatcgccgcctacgcaccctttaaacccaataaatccggataacgctcgcatcctccgtattaccgcggctgctggcacggagttagccgatgctttttcttcaggtacactcagactagtatacatactcgcttttgttccctgacaaaagaggtttacgacccatagggccttcctccctcacgcggcttggctggttcaggctcccgcccattgaccaatattcctca

SEQ ID NO: 30 - DNA, 407 bp, Unknown
  source: /note=“Description of Unknown 16S rRNA sequence”
cctgtttgctacccacgctttcgcgctttagcgtcagtatctgtccagtaggctggcttccccatcggcattcctacaaatatctacgaatttcacctctacacttgtagttccgcctacctctccagtactctagtttggcagtttccaacgcaatacggagttgagccccgcattttcacatcagacttaccaaaccgcctagacgcgctttacgcccaataaatccggataacgcttgcgacatacgtattaccgcggctgctggcacgtatttagccgtcgcttcttctgttggtaccgtcactttcttcttcccaactgaaagcactttacattccgaaaaacttcatcgtgcacacagaattgctggatcagacttttggtccattgtccaatattcccca

SEQ ID NO: 31 - DNA, 406 bp, Unknown
  source: /note=“Description of Unknown 16S rRNA sequence”
cctgttcgctccccgcgctctcgcgcctcagcgtcagtgtccgtccggcgggccgccttcgcctccggtgttcctcctggcctctgcgcatttcaccgctacgccaggagttccgcccgcccctccggcactccagccgcccggtccggggcgcttgcccggggttgggccccggcttttcacaccccgcccaggccgccgcctgcgcgcgctttacgcccagtgattccggacaacgctcgccccccacgtgttgccgcggctgctggcacgtggttggccggggctttcgtgccgggttcagtcataccttcctccccggcggcggggctttacgggcctgggcccttcatcgcccacgcggcatcgctgcgtcagagttgcctccattgcgcaatattcccca

SEQ ID NO: 32 - DNA, 404 bp, Unknown
  source: /note=“Description of Unknown 16S rRNA sequence”
cctgtttgctccccacgctttcgagcctcagtgtcagttacagtccagtaagccgccttcgccactggtgttcctcctaatatctacgcatttcaccgctacactaggaattccacttacctctcctgcactctagtctgacagtttcaaaggcagtcccggggttgagccccgggctttcacctctgacttgtcagaccacctacactccctttacacccagtaaatccggataacgcttgcaccatacgtattaccgcggctgctggcacgtatttagccggtgcttcttagtcaggtaccgtcactatcttccctgctgatagagctttacataccgaaatacttcttcactcacgcggcgtcgctgcatcagagtttcctccattgtgcaatattcccca

SEQ ID NO: 33 - DNA, 424 bp, Unknown
  source: /note=“Description of Unknown 16S rRNA sequence”
cctgttcgatacccgcactttcgagcttccgcgtccgttgcgctccagtgagctgccttcgcaatcggagttcttcgtgatatctaagcatttcaccgctacaccacgaattccgcccactttgtgcgtactcaaggaaaccagttcgcgctgcagtgcagacgttgagcgtctacatttcacaacacgcttaatctccggcctacgctccctttaaacccaataaatccggataacgcccggaccttccgtattaccgcggctgctggcacggaattagccggtccttattcataaggtacatgcaaaaagtctcacgagactaactttattcccttataaaagcagtttacaacccatagggccgtcatcctgcacgctacttggctggttcaggctctcgcccattgaccaatattcctca

SEQ ID NO: 34 - DNA, 424 bp, Unknown
  source: /note=“Description of Unknown 16S rRNA sequence”
cctgtttgatccccacgctttcgtgcttcagtgtcagttatggtttagtaagctgccttcgcaatcggagttctgcgtgatatctatgcatttcaccgctacaccacgcattccgcctacctcaaacacactcaagtaacccagtttcaacggcaattttatggttgagccacaaactttcaccgctgacttaaatcaccacctacgcaccctttaaacccaataaatccggataacgctcgcatcctccgtattaccgcggctgctggcacggagttagccgatgcttattcatagggtacatacaaaaaaggacacgtcctccactttattcccctataaaagaagtttacaaaccatagatccttcttccttcacgcgacttggctggttcagcctctcggccattgaccaatattcctca

SEQ ID NO: 35 - DNA, 424 bp, Unknown
  source: /note=“Description of Unknown 16S rRNA sequence”
cctgtttgatccccgcactttcgagcctcagcgtcagtagcaccccggatacctgccttcgcgatcggagttcttcatgatatctgagcatttcaccgctacaccatgaattccagtatccctgtgtgcactcaagacttccagtatcaactgcagtccgacggttgagccgccgcatttcacaactgacttaaaaatccgcctgcgctccctttaaacccaataaatccggataacgcccggaccttccgtattaccgcggctgctggcacggaattagccggtcctttttctgatggtacatacaaaacaactcacgagctgcactttattcccaactaaaagcagtttacaacccggagggccgtcatcctgcacgctacttggctggttcagacttgcgtccattgaccaatattcctca

SEQ ID NO: 36 - DNA, 404 bp, Unknown; identical to SEQ ID NO: 1
  source: /note=“Description of Unknown 16S rRNA sequence”

SEQ ID NO: 37 - DNA, 405 bp, Unknown
  source: /note=“Description of Unknown 16S rRNA sequence”
cctgtttgctccccacgctttcgcgcctgagcgtcagtgtctgtccggcaggccgccttcgcctccggtattcctcctgatatctgcgcatttcaccgctacaccaggaattctgcctgcctctccagcactcgagccctccggtttgagatgctctcccggggttgggccccggtctttaacacctcacctaaaaagccgcctgcgcgccctttacgcccagtaattccggacaacgctcgcccctcacgtcttaccgcggctgctggcacgtgattggccggggctttcttgccgggtactgtcttctctcttccccggcggcagagctttacgggacgaatcccttcctcactcacgcggcatcgctgcgtcagagttgcctccattgcgcaatattcccca

SEQ ID NO: 38 - DNA, 406 bp, Unknown
  source: /note=“Description of Unknown 16S rRNA sequence”
cctgtttgctccccacgctttcgcacctgagcgtcagtgtctgtccggcaggccgccttcgcctccggtattcctcctgatatctgcgcatttcaccgctacaccaggaattctgcctgcctctccagcactctagctgtgcggttcggggtgtcctctaagggttgggccctcatcttttacatccccgcctgctacagccgcctgcgtgccctttacgcccagtgattccggacaacgctcgccccctacgtgttaccgcggctgctggcacgtagttagccggggctttctcgccaggtactgtcttactcatccctggcaacagagctttacgggacgaatcccttcttcactcacgcggcatcgctgcgtcagagttccctccattgcgcaatattcccca

SEQ ID NO: 39 - DNA, 406 bp, Unknown; identical to SEQ ID NO: 15
  source: /note=“Description of Unknown 16S rRNA sequence”

SEQ ID NO: 40 - DNA, 428 bp, Unknown
  source: /note=“Description of Unknown 16S rRNA sequence”
cctatttgctccccacgctttcgtgcctgagcgtcagcaacagtccaggcggccgccttcgccactggtgttcttccatatatctacgcattttaccgctacacatggagttccaccgccctctcctgtcctcgagcgcgccagtttccaaagcctgtacaggttgagcctgtacctttcacttcagacttgacgcgccgcctgcgcaccctttacgcccaatcattccggataacgcttgccacctacgtattaccgcggctgctggcacgtagttagccgtgactttctggcggggtaccatcaagaagaaatcatttcctcttccttccctttttcccccgcaacagagctttacgatccgaagaccttcctcactcacgcggcattgctcgttcaggcttgcgcccattgacgaaaattcccta

SEQ ID NO: 41 - DNA, 424 bp, Unknown
  source: /note=“Description of Unknown 16S rRNA sequence”
cctgtttgatacccacactttcgagcatgaacgtcagttacggcttagtgtgctgccttcgcaatcggggttcttcgtgatatctaagcatttcaccgctacaccacgaattccgcacacctcaaccgcactcaaggacgccagtatcaactgcaattttacggttgagccgcaaactttcacagctgacttaacatcccgtctgcgctccctttaaacccaataaatccggataacgctcgcatcctccgtattaccgcggctgctggcacggagttagccgatgcttattcatacggtacatacaagctcccacacgtgggaggttttattcccgtataaaagaagtttacaatccatagaaccttcatccttcacgctacttggctggttcaggctctcgcccattgaccaatattcctca

SEQ ID NO: 42 - DNA, 404 bp, Unknown; identical to SEQ ID NO: 2
  source: /note=“Description of Unknown 16S rRNA sequence”

SEQ ID NO: 43 - DNA, 404 bp, Unknown
  source: /note=“Description of Unknown 16S rRNA sequence”
cctgttcgctcccccagctttcgcgcctcagcgtcggtctcggcccagagggccgccttcgccaccggtgttcctcccgatatctgcgcattccaccgctacaccgggaattccaccctcccctaccggacccgagccgcggggttcggggggcggaccggggttgagccccgggatttgaccccccgcctacgcggccgcctacgcgcgctttacgcccaatgaatccggataacgctcgccccctacgtattaccgcggctgctggcacgtagttagccggggcttcttctgcaggtaccgtcttgactcgtccctgctgaaagcggtttacgacccgaaggccttcgtcccgcacgcggcgtcgctgcgtcagggttgcccccattgcgcaagattcccca

SEQ ID NO: 44 - DNA, 425 bp, Unknown
  source: /note=“Description of Unknown 16S rRNA sequence”
cctgtttgatacccacactttcgagcatcagtgtcagttacagtccagtgagctgccttcgcaatcggagttcttcgtgatatctaagcatttcaccgctacaccacgaattccgcccacctctactgcactcaagaaaaccagtatcaactgcaattttacggttgagccgcaaactttcacaggctgacttaatcttccacctacgctccctttaaacccaataaatccggataacgctcggatcctccgtattaccgcggctgctggcacggagttagccgatccttattcatacggtacatacaaaattccacacgtggaacactttattcccgtataaaagaagtttacaacccatagggcagtcatccttcacgctacttggctggttcagactctcgtccattgaccaatattcctca

SEQ ID NO: 45 - DNA, 429 bp, Unknown
  source: /note=“Description of Unknown 16S rRNA sequence”
cctgtttgctccccacgctttcgagcctcagcgtcagttacagaccagagagccgctttcgccaccggtgttcctccatatatctacgcatttcaccgctacacatggaattccactctccccttctgcactcaagtttaacagtttccaaagcgaacaatggttaagccactgcctttaacttcagacttattaaaccgcctgcgctcgctttacgcccaataaatccggacaacgctcgggacctacgtattaccgcggctgctggcacgtagttagccgtccctttctggtaagttaccgtcactgtgtgaactttccactctcacacacattcttctcttacaacagagctttacgatccgaaaaccttcttcactcacgcggcgttgctcggtcagggttgcccccattgccgaagattcccta

SEQ ID NO: 46 - DNA, 405 bp, Unknown
  source: /note=“Description of Unknown 16S rRNA sequence”
cctgttcgctccccacgctttcgcacctcagcgtcagtatttgtccagcaagccgccttcgccactggtgttcctcctaatatctacgcatttcaccgctacactaggaattccacttgcctctccaatactcaagtctaccagtttccaatgcacttcaccggttgagccggtacctttcacatcagacttaataaaccgcctacgcgccctttacgcccagtcattccggacaacgctcgtcccctacgtattaccgcggctgctggcacgtagttagccgggacttcctcattaggtaccgtcattattcttccctaataacagagctttacgatccgaaaaccttcttcactcacgcggtattgctgcgtcagggtttcccccattgcgcaatattcccca

SEQ ID NO: 47 - DNA, 404 bp, Unknown
  source: /note=“Description of Unknown 16S rRNA sequence”
cctgtttgctccccacgctttcgagcctcaacgtcagttaccgtccagtaagccgccttcgccactggtgttcctcctaatatctacgcatttcaccgctacactaggaattccgcttacctctccggcactctagactgacagtttccaatgcagtccaggggttaagcccctgcctttcacatcagacttgccattccgtctacgctccctttacacccagtaaatccggataacgcttgccccttacgtattaccgcggctgctggcacgtaattagccggggcttcttagtcaggtaccgtcattatcttccctgctgatagaagtttacataccgagatacttcttccttcacgcggcgtcgctgcatcagggtttcccccattgtgcaatattcccca

SEQ ID NO: 48 - DNA, 424 bp, Unknown
  source: /note=“Description of Unknown 16S rRNA sequence”
cctgtttgatacccacactttcgtgcctcagcgtcagtcgcagtctggcaagctgcctacgcaatcggagttcttcgtgatatctaagcatttcaccgctacaccacgaattccgcctgcctcgtctgaactcaaggactccagtatcaactgcaattttacggttgagccgcaaactttcacaactgacttaaaatcccgcctacgcaccctttaaacccaataaatccggataacgctcggatcctccgtattaccgcggctgctggcacggagttagccgatccttattcatacggtacatacaggcacccacacgtggatgatgttattcccgtataaaagaagtttacaacccatagggcagtcatccttcacgctacttggctggttcaggctcgcgcccattgaccaatattcctca

SEQ ID NO: 49 - DNA, 424 bp, Unknown
  source: /note=“Description of Unknown 16S rRNA sequence”
cctgttcgatccccgcactttcgtgcctcagcgtcagttgagcgccggcaggctgccttcgcaatcggggttctgcgtgatatctatgcatttcaccgctacaccacgcattccgcctgcttctcgcccactcaaggctgccagtttcaacggccggacggggttgagccccgaatttttaccgctgacttaacagtccgcctacgcaccctttaaacccaataaatccggataacgctcgcatcctccgtattaccgcggctgctggcacggagttagccgatgctttttctccgggtactctcctaatcaggacacgtcccgattcttgctccccggcaaaagaggtttacgacccgtagggccttattccctcacgcgacttggctggttcaggcttccgcccattgaccaatattcctca

SEQ ID NO: 50 - DNA, 404 bp, Unknown; identical to SEQ ID NO: 16
  source: /note=“Description of Unknown 16S rRNA sequence”

SEQ ID NO: 51 - DNA, 404 bp, Unknown
  source: /note=“Description of Unknown 16S rRNA sequence”
cctgtttgctacccatgctttcgagcctcagcgtcagttagtgcccagtaggccgccttcgccactggtgttcctcccgatatctacgcattccaccgctacaccgggaattccgcctacctctacaccactcaagatctgcagttttgaaagcagtccataggttgagcccatggttttcacttccaacttgcaaacccgcctgcgctccctttacacccagtaattccggacaacgcttgccacctacgttttaccgcggctgctggcacgtagttagccgtggctttcttgtaaggtaccgtcattatcgtcccttacgacagaagtttacaacccgaaggccttcttccttcacgcggcgtcgctgcatcagggtttcccccattgtgcaatattcccca

SEQ ID NO: 52 - DNA, 428 bp, Unknown
  source: /note=“Description of Unknown 16S rRNA sequence”
cccgttcgctcccctggctttcgagcctcagtgtcagttacagtccagaaagccgccttcgccactggtgttcctcctaatatctacgcatttcaccgctacactaggaattccgctttcctctcctgcactcaagaaaagtagtttcaatcccctcacggggttgagccccgcacttttaagactgacttactttcccacctgcgctccctttacgcccaataattccggacaacgctcgccacctacgtattaccgcggctgctggcacgtagttagccgtggcttcctcgaaaggtaccgtcctacaaagctcatttcctagccttgcctctcgtccctctcaacagagctttacgatccgaaaaccttcttcactcacgcggcgttgctccgtcaggcttgcgcccattgcggaagattcccca

SEQ ID NO: 53 - DNA, 425 bp, Unknown
  source: /note=“Description of Unknown 16S rRNA sequence”
gcctgttcgatccccgcactttcgtgcctcagcgtcagttgagcgccggtaagctgccttcgcaatcggagttctgcgtgatatctatgcatttcaccgctacaccacgcattccgcctacttctcgctcactcaaggctcccagtttcaacggctctacggcgttgagcaccgcaattttaccgctgacttaagagcccgcctacgcaccctttaaacccaataaatccggataacgctcgcatcctccgtattaccgcggctgctggcacggagttagccgatgctttttctccgggtactctccaatctgggacacgtcccagatcttgctccccggcaaaagaggtttacgacccatagggccttattccctcacgcgacttggctggttcaggctttcgcccattgaccaatattcctca

SEQ ID NO: 54 - DNA, 423 bp, Unknown
  source: /note=“Description of Unknown 16S rRNA sequence”
cctgttcgctacccacgctctcgtgcttcagcgtcagtaacagtctggtaagctgccttcgctatcggagttctaagttatatctaagcatttcaccgctacttaacttattccgcctacctcgtctgtactcaagctgaccagtattaatggctggccaaggtttagccatgacattttaccactaacttaatcaaccgcctacgcaccctttaaacccaataaatccggataacgctcgcatcctccgtattaccgcggctgctggcacggagttagccgatgcttattcattcggtacaggcatcgttccacgcgtgaaacttattcttcccgaataaaagaggtttacaacccatagggcagtcttccctcacgcgacttggctggttcagccttccggccattgaccaatattcctca

SEQ ID NO: 55 - DNA, 411 bp, Unknown
  source: /note=“Description of Unknown 16S rRNA sequence”
cctgttcgctcccccagctttcgcgcctcagcgtcagtggtggcccagaaggccgccttcgccaccggtgttcttcccgatatctgcgcattccaccgctacaccgggaattccgccttcccctaccacactcgagcccgccggtatcggaggctcgccggggttgagccccgggcttttacctccgacctaacgggccgcctacgcgcgctttacgcccaatgaatccggataacgctcgccccatacgtattaccgcggctgctggcacgtatttagccggggcttcttctgcaggtaccgtcaccgtagcggcctcgtccctgctgaaagcggtttacgacccgaaggccttcgtcccgcacgcggcgtcgctgcgtcagggttgcccccattgcgcaagattcccca

SEQ ID NO: 56 - DNA, 404 bp, Unknown
  source: /note=“Description of Unknown 16S rRNA sequence”
cctgtttgctccccacgctttcgagcctcaacgtcagttactgtccagcaagccgccttcgccactggtgttcctcctaatatctacgcatttcaccgctacactaggaattccgcttgcctctccagcactctagcccaacagtttccaaagcagttcccgggttgagcccggggatttcacttcagacttgctgtgccgtctacgctccctttacacccagtaaatccggataacgcttgccccctacgtattaccgcggctgctggcacgtagttagccggggcttcttagtcaggtaccgtcactttcttccctgctgatagagctttacataccgaaatacttcttcactcacgcggcgttgctgcatcagggtttcccccattgtgcaatattcccca

SEQ ID NO: 57 - DNA, 424 bp, Unknown
  source: /note=“Description of Unknown 16S rRNA sequence”
cctgtttgatacccacactttcgagcatcaacgtcagttacgctccagcaagctgccttcgcaatcggagttcttcgtgatatctaagcatttcaccgctacaccacgaattccgcctgcctctgccgcactcaagaatgccagtttcaactgcaattttaaggttgagccccaaactttcacagctgacttaacatcccgtctacgctccctttaaacccaataaatccggataacgctcgcatcctccgtattaccgcggctgctggcacggagttagccgatgcttattcataaggtacatgcaaaacactacacgtaatgtctgttattcccttataaaagaagtttacaatccatagaaccttcatccttcacgctacttggctggttcaggctctcgcccattgaccaatattcctca

SEQ ID NO: 58 - DNA, 405 bp, Unknown
  source: /note=“Description of Unknown 16S rRNA sequence”
cctgtttgctccccacgctttcgtgcctcagtgtcagtttcagtccagaaagccgccttcgccactggtgttcctcctaatatctacgcatttcaccgctacactaggaattccgctttcctctcctgcactcaagtagcccagttcgcagggcatacaatggttgggccattgccttaaaccctgcgcttaggttaccacctacgcactctttacgcccaataattccggataacgcttgccccctacgtattaccgcggctgctggcacgtagttagccggggcttcctccttaggtaccgtcagttcttcttccctaaggacagaagtttacgacccgaaggccttcttccttcacgcggcgttgctgcatcaggctttcgcccattgtgcaatattcccca

SEQ ID NO: 59 - DNA, 405 bp, Unknown
  source: /note=“Description of Unknown 16S rRNA sequence”
cctgttcgctccccacgctctcgcgcctcagcgtcagtgtccgtccggcgggccgccttcgcctctggtgttcctcccggcctctgcgcatttcaccgctacgccgggagttccgcccgcccctccggcactccagccgctcggttcggggcgctttcccggggttgggccccggattttcacaccccgccctagcggccgcctgcgcgcgctttacgcccagtgattccggacaacgctcgccccccacgtattgccgcggctgctggcacgtggttggccggggctttcgcgccaggtaccgtctctgctcttccctggctacggggctttacgggccgaggcccttcctcgcccacgcggcatcgctgcgtcagagtttcctccattgcgcaatattcccca

SEQ ID NO: 60 - DNA, 429 bp, Unknown
  source: /note=“Description of Unknown 16S rRNA sequence”
cctgtttgctccccacgctttcgcgcctcagtgtcagttacagaccaggaagccgccttcgccactggtgttcctccatatctctacgcatttcaccgctacacatggaattccacttccctcttctgcactcaagtcgaccagtttccaatgaccctccacggttaagccgtgggctttcacatcagacttaatcaaccacctgcgcgctctttacgcccaataattccggataacgctcgccacctacgtattaccgcggctgctggcacgtagttagccgtggctttctcataaggtaccgtcacactctagccatttcctactaaagtcgttcttcccttataacagaattttacaacccgaaggccttcatcattcacgcggcgttgctcggtcaggctttcgcccattgccgaagattcccta

SEQ ID NO: 61 - DNA, 424 bp, Unknown; identical to SEQ ID NO: 3
  source: /note=“Description of Unknown 16S rRNA sequence”

SEQ ID NO: 62 - DNA, 404 bp, Unknown
  source: /note=“Description of Unknown 16S rRNA sequence”
cctgtttgctacccacactttcgagcctcagcgtcagttagtgcccagtaggccgccttcgccactggtgttcctcccgatatctacgcattccaccgctacaccgggaattccgcctacctctgcaccactcaagaaaagcagttttgaaagcaatttacgggttgagcccgtagatttcacttccaacttaccttcccgcctgcgctccctttacacccagtaattccggacaacgcttgtgacctacgttttaccgcggctgctggcacgtagttagccgtcactttcttgtaaggtaccgtcattatcgtcccttacaacaggagtttacaatccgaagaccttcttcctccacgcggcgtcgctgcatcagggtttcccccattgtgcaatattcccca

SEQ ID NO: 63 - DNA, 404 bp, Unknown; identical to SEQ ID NO: 17
  source: /note=“Description of Unknown 16S rRNA sequence”

SEQ ID NO: 64 - DNA, 404 bp, Unknown
  source: /note=“Description of Unknown 16S rRNA sequence”
cctgtttgctccccacgctttcgagcctcaacgtcagtcatcgtccagtaagccgccttcgccactggtgttcctcctaatatctacgcatttcaccgctacactaggaattccacttacctctccgacactctagaaaaacagtttccaatgcagtcccggggttgagccccgggttttcacatcagacttgcctctccgtctacgctccctttacacccagtaaatccggataacgcttgcaccatacgtattaccgcggctgctggcacgtatttagccggtgcttcttagtcaggtaccgtcattttcttccctgctgatagagctttacataccgaaatacttcatcgctcacgcggcgtcgctgcatcagggtttcccccattgtgcaatattcccca

SEQ ID NO: 65 - DNA, 410 bp, Unknown
  source: /note=“Description of Unknown 16S rRNA sequence”
cctgtttgctcccctagctttcgcgcctgagcgtcagtcatggcccagaaggccgccttcgccaccggtgttcctcccgatatctgcgcattccaccgctacaccgggagttccgccttcccctaccagactcaagcctgccggtatcgagagcggcccggggttgagccccgggattagaccctcgacctaacaggccgcctacgcgcgctttacgcccaatgaatccggataacgctcgccccctacgtgttaccgcggctgctggcacgtagttagccggagcttcttctgcaggtaccgtcagctttcgccctcgtccctgctgaaagcggtttacgatccgaagaccttcatcccgcacgcggcgtcgctgcgtcagggttgcccccattgcgcaagattcccca

SEQ ID NO: 66 - DNA, 428 bp, Unknown
  source: /note=“Description of Unknown 16S rRNA sequence”
cctatttgctccccacgctttcgtgcctcagcgtcagctgcaggccagacggccgccttcgccactggtgttcctccatatatctacgcattttaccgctacacatggaattccgccgtcctctcctgtgctccagctccccagtttccagggctcgccatggttgagccatgggcttttaccccggacttagggagccgcctgcgcaccctttacgcccaatgattccggataacgctcgccacctacgtattaccgcggctgctggcacgtagttagccgtggctttctggcgggctaccgtcctgctccgggcatttcctcccgcagcctctcttcgcccgcaacagagctttacagcccgaaggccgtcttcgctcacgcggcattgctcggtcagggtttcccccattgccgaaaattcccta

SEQ ID NO: 67 - DNA, 407 bp, Unknown
  source: /note=“Description of Unknown 16S rRNA sequence”
cctgtttgctccccacgctttcgagcctcagcgtcagtttcggtccagcaagccgccttcgccactggtgttcctcctaatatctatgcatttcaccgctacactaggaattccgcttgcctctcccgtactcaagctatgcagttttccatgcaacccccaggttaagcccgggtatttcacatgaaacttgcattgccgcctacgctctctttacgcccagtaattccggacaacgctcgccccctacgtattaccgcggctgctggcacgtagttagccggggctttcttctatggtaccgtcacttccttcttcccatagaacagaattttacaatcctaagaccttcttcattcacgcggcattgctgggtcaggctttcgcccattgcccaatattcccca

SEQ ID NO: 68 - DNA, 422 bp, Unknown
  source: /note=“Description of Unknown 16S rRNA sequence”
cctgttcgatacccacgctttcgtgcctcagcgtcagtaagaggttggtacgctgccttcgcaatcggagttctgcgtgatatctatgcatttcaccgctacaccacgcattccgcatacctcacttccactctagcccgacagtttcaacggcaggccggggttgagccccgacatttgaccgctgacttgccagacagcctacgcaccctttaaacccaataaatccggataacgctcgcatcctccgtattaccgcggctgctggcacggagttagccgatgcttattcttgcggtacgctcaactggccacacgtggccgatttgttcccgcactaaagaagtttacaatccatagaaccttcttccttcacgcgacttggctggttcaggctcgcgcccattgaccaatattcctca

SEQ ID NO: 69 - DNA, 406 bp, Unknown
  source: /note=“Description of Unknown 16S rRNA sequence”
cctgtttgctccccacgctttcgtgcctcagcgtcagttatggtccagaaagccgccttcgctactggtgttcctttgaatctctacgcatttcaccgctacactcaaagttccactttcctctcccacactctagcctctcagtttcggtagcagctccggggttgagccccgaaatttcacttccgacttaaaaggccgcctacgcaccctttacacccagtaaatccggataacgcttgctccctacgtattaccgcggctgctggcacgtagttagccggagcttcttagtcaggtaccgtcatttcttcttccctgctgatagaagtttacaatccgaagaccttcttccttcacgcggcgttgctgcatcaggctttcgcccattgtgcaatattcccca

SEQ ID NO: 70 - DNA, 406 bp, Unknown
  source: /note=“Description of Unknown 16S rRNA sequence”
cctgtttgctccccacgctttcgcacctcagcgtcagtatttgcccaggagaccgccttcgcctctggtgttctttcagatatctacgcattccaccgctacacctgaaattccatctccctctgcaatactctagaaaggcagtttagaatgaccaacccaggttgagcccgggtatttcacatcctacttacctttccgcctacatgccctttacgcccagttattccgattaacgcttgcaccctccgtattaccgcggctgctggcacggagttagccggtgcttcttctgtgggtaacgtcagtgtttcctccccactgaaagtgctttacaacccgaaggccttcttcacacacgcggcatggctgcatcagggtttcccccattgtgcaatattcccta

SEQ ID NO: 71 - DNA, 404 bp, Unknown
  source: /note=“Description of Unknown 16S rRNA sequence”
cctgtttgctccccacgctttcgagcctcagtgtcagttacagtccagtgagccgccttcgcctccggtgttcctcctaatatctacgcatttcaccgctacactaggaattccgctcacccctcctgcactctagcatgacagttccaaaagcaatcccgcggttgagccccgggttttcacttctggcttgccttgccacctacgctccctttacacccagtaaatccggataacgcttgcaccatacgtattaccgcggctgctggcacgtatttagccggtgcttcttagtcaggtaccgtcattttcttccctgctgatagagctttacataccgaaatacttcttcactcacgcggcgtcgctgcatcagagtttcctccattgtgcaatattcccca

SEQ ID NO: 72 - DNA, 405 bp, Unknown
  source: /note=“Description of Unknown 16S rRNA sequence”
cctgtttgctacccacgctttcgagcctcagcgtcagttacagcccagtaggccgccttcgccactggtgttcctcctaatatctacgcatttcaccgctacactaggaattccgcctacctctactgcactcaagagtgatagttttgaacgcgactatcagttaagccgatagtttaaacattcaacttgtctccccgcctacgctccctttacacccagtaattccggacaacgcttgccacctacgtattaccgcggctgctggcacgtagttagccgtggcttcctccttgggtaccgtcattttttcgtccccaaagacagaggtttacaatccgaaaaccgtcttccctcacgcggcgtcgctgcatcagggtttcccccattgtgcaatatccccca

SEQ ID NO: 73 - DNA, 406 bp, Unknown; identical to SEQ ID NO: 18
  source: /note=“Description of Unknown 16S rRNA sequence”

SEQ ID NO: 74 - DNA, 429 bp, Unknown
  source: /note=“Description of Unknown 16S rRNA sequence”
cctatttgctccccacgctttcgtgcctcagcgtcagatgcaggccagacggccgccttcgccaccggtgttcttccatatatctacgcattttaccgctacacatggagttccaccgtcctctcctgctctccagccggacagtttccgcagcccctgaaggttgagcctccagtttttactgcggacttgcccggccgcctgcgcaccctttacgcccaatcattccggataacgctcgccacctacgtattaccgcggctgctggcacgtagttagccgtgacttcctcgccgggtaccatcactcagaaagctttccactctttctgccttttgtccccggcaacagagctttacgatccgaagaccttcctcgctcacgcggcattgctcgttcagggttccccccattgacgaaaattcccta

SEQ ID NO: 75 - DNA, 407 bp, Unknown
  source: /note=“Description of Unknown 16S rRNA sequence”
cctgtttgctccccacgctttcgcgcttcagcgtcagtatctgtccagtgagctgacttctccatcggcattcctacaaatatctacgaatttcacctctacacttgtagttccgcccacctctccagtactctagaaagacagtttccaacgcaatacggagttgagccccgcattttcacatcagacttatctttccgcctagacgcgctttacgcccaataaatccggataacgcttgcgacatacgtattaccgcggctgctggcacgtatttagccgtcgcttcttctgttggtaccgtcactttcttcttcccaactgaaagcactttacaatccgaagaccttcatcgtgcacacagaattgctggatcaggcttgtggcccattgtccaatattcccca

SEQ ID NO: 76 - DNA, 428 bp, Unknown
  source: /note=“Description of Unknown 16S rRNA sequence”
cctatttgctccccacgctttcgtgcctgagcgtcagtaacagtccaggcggccgccttcgccactggtgttcttccatatatctacgcattttaccgctacacatggagttccaccgccctctcctgtcctcgagcgtgacagtttccaaagcctgtacaggttgagcccgtacctttcacttcagacttgccacgccgcctgcgcaccctttacgcccaatcattccggataacgcttgccacctacgtattaccgcggctgctggcacgtagttagccgtgactttctggcaggttactatcaaaagaaaagcatttcctcttcccttcttttctgacctgcaacagagctttacgatccgaagaccttcctcactcacgcggcattgctcgttcaggcttgcgcccattgacgaaaattcccta

SEQ ID NO: 77 - DNA, 424 bp, Unknown
  source: /note=“Description of Unknown 16S rRNA sequence”
cctgttcgatacccgcactttcgagcttcagcgtcagttgcgctccagtgagctgccttcgcaatcggagttcttcgtgatatctaagcatttcaccgctacaccacgaattccgcccactttgtgcgtactcaaggaaaccagttcgcgctgcagttcagatgttgagcatctacatttcacaacacgcttaatctccggcctacgctccctttaaacccaataaatccggataacgcccggaccttccgtattaccgcggctgctggcacggaattagccggtccttattcataaggtacatgcaaaaagtctcacgagactcactttattcccttataaaagcagtttacaacccatagggccgtcttcctgcacgctacttggctggttcagactctcgtccattgaccaatattcctca

SEQ ID NO: 78 - DNA, 404 bp, Unknown
  source: /note=“Description of Unknown 16S rRNA sequence”
cctgtttgctacccatgctttcgagcctcagcgtcagttagtgcccagcaggccgccttcgccactggtgttcctcccgatatctacgcattccaccgctacaccgggaattccgcctgcctctgcaccactcaagatctgcagttttgaatgcacctatgggttgagcccatagtttaaacatccaacttgcaaacccgcctgcgctccctttacacccagtaattccggacaacgcttgccacctacgtattaccgcggctgctggcacgtagttagccgtggcttcctcctcgggtaccgtcatacttcgtccccgaagacagaggtttacaatccgaaaatcttcttccctcacgcggcgtcgctgcatcagagtttcctccattgtgcaatattcccca

SEQ ID NO: 79 - DNA, 429 bp, Unknown
  source: /note=“Description of Unknown 16S rRNA sequence”
cctgttcgctacccatgctttcgagcctcagcgtcagttgcagaccagagagccgccttcgccactggtgttcttccatatatctacgcattccaccgctacacatggagttccactctcctcttctgcactcaagttcaacagtttctgatgcaattctccggttgagccgaaggctttcacatcagacttattgaaccgcctgcactcgctttacgcccaataaatccggacaacgcttgccacctacgtattaccgcggctgctggcacgtagttagccgtgactttctaagtaattaccgtcaaataaaggccagttactacctctatctttcttcactaccaacagagctttacgagccgaaacccttcttcactcacgcggcgttgctccatcagactttcgtccattgtggaagattcccta

SEQ ID NO: 80 - DNA, 406 bp, Unknown
  source: /note=“Description of Unknown 16S rRNA sequence”
cctgtttgctccccacgctttcgcacctcagcgtcagtatctttccagcaggccgccttcgcctctggtattcctcctgaactctacgcatttcaccgctactccaggaattctgcctgcctctccaatactctagtctaccagttccgggtgctcacagtggttgagccactgcctttcacatccagcttgataaaccgcctacgtgccctttacgcccagtcattccggacaacgcttgtcccctacgtattaccgcggctgctggcacgtagttagccgggacttcatcttagggtaccgtcacttgcttcttcccctatgatagagctttacaacccgaaggccttcttcactcacgcggtattgctgcgtcagagttccctccattgcgcaatattcccca

SEQ ID NO: 81 - DNA, 404 bp, Unknown; identical to SEQ ID NO: 19
  source: /note=“Description of Unknown 16S rRNA sequence”

SEQ ID NO: 82 - DNA, 404 bp, Unknown
  source: /note=“Description of Unknown 16S rRNA sequence”
cctgtttgctccccacgctttcgagcctcaacgtcagttaccgtccagtaagccgccttcgccactggtgttcctcctaatatctacgcatttcaccgctacactaggaattccgcttacccctccggcactcaagcctggcagtttccaatgcagtccaggagttgagcccctgcctttcacatcagacttgccatgccgtctacgctccctttacacccagtaaatccggataacgcttgccccctacgtattaccgcggctgctggcacgtagttagccggggcttcttagtcaggtaccgtcattatcttccctgctgatagaagtttacataccgagatacttcttccttcacgcggcgtcgctgcatcagggtttcccccattgtgcaatattcccca

SEQ ID NO: 83 - DNA, 403 bp, Unknown
  source: /note=“Description of Unknown 16S rRNA sequence”
cctgtttgctccccacgctttcgtgcctcagcgtcagttacagtccagaaagccgccttcgctactggtgttcctcccaatatctacgcatttcaccgctacactgggaattccgctttcctctcctgcactcaagtcagacagtatcaggagcttactacggttgagccgtagcctttaactcctgacttgaaagaccgcctacgcaccctttacgcccagtaaatccggataacgctagccccctacgtattaccgcggctgctggcacgtagttagccggggcttcctcctcaagtaccgtcattatcttccttgaggacagagctttacgacccgaaggccttcatcgctcacgcggcgttgctgcatcaggcttgcgcccattgtgcaatattcccca

SEQ ID NO: 84 - DNA, 407 bp, Unknown
  source: /note=“Description of Unknown 16S rRNA sequence”
cctgtttgctccccacgctttcgcgcctcagcgtcagttaatgtccagcaggccgccttcgccactggtgttcctcccaatatctacgcatttcaccgctacactgggaattccgcctgcctctccatcactcaagactcgcagttttgaaagcagtttcggggttaagccccgagatttcacttccaacttgcaagcccgcctacacgccctttacacccagtaaatccggataacgcttgctccctacgtattaccgcggctgctggcacgtagttagccggagcttattcttcaggtaccgtcattttcttcgtccctgattaaagatttttacaatccgaagaccttcatcaatcacgcggcgttgctgcgtcagggttgcccccattgcgcaatattcccca

SEQ ID NO: 85 - DNA, 406 bp, Unknown
  source: /note=“Description of Unknown 16S rRNA sequence”
cctgtttgctccccacgctttcgctcctcagcgtcagttaatgtccagcaggccgccttcgccactggtgttcctccgtatatctacgcatttcaccgctacacacggaattccgcctgcctctccatcactcaagaccagcagttttgaaagcagtttgggggttgagcccccagatttcacttccaacttaccagcccgcctactcgccctttacacccagtaaatccggataacgcttgctccctacgtattaccgcggctgctggcacgtagttagccggagcttattctttaggtaccgtcaaattttcgtccctaattaaagatctttacaacccgaaggccttcttcaatcacgcggcgttgctgcgtcagggtttcccccattgcgcaatattcccca

SEQ ID NO: 86 - DNA, 404 bp, Unknown
  source: /note=“Description of Unknown 16S rRNA sequence”
cctgtttgctccccacgctttcgagcctcagcgtcattgtcagtccagtaagccgccttcgccactggtgttcctcctaatatctacgcatttcaccgctacactaggaattccgcttacctctcctgaaatctagccgcacagtttcaaaagcagtcccggagttaagccccgggctttcacttctgacttgcgctgccgcctacgctccctttacacccagtaaatccggataacgcttgccccctacgtattaccgcggctgctggcacgtagttagccggggcttcttagtcaggtaccgtcgttatcttccctgctgatagaagtttacataccgaaatacttcatccttcacgcggcgttgctgcatcaggctttcgcccattgtgcaatattcccca

SEQ ID NO: 87 - DNA, 404 bp, Unknown
  source: /note=“Description of Unknown 16S rRNA sequence”
cctgtttgctccccacgctttcgagcctcaacgtcagttatcgtccagtaagccgccttcgccactggtgttcctcctaatatctacgcatttcaccgctacactaggaattccacttacccctccgacactctagactgacagtttccaatgcagtcccggggttgagccccgggttttcacatcagacttgccagtccgtctacgctccctttacacccagtaaatccggataacgcttgccccctacgtattaccgcggctgctggcacgtagttagccggggcttcttagtcaggtaccgtcattttcttccctgctgatagagctttacataccgaaatacttcttcgctcacgcggcgtcgctgcatcagggtttcccccattgtgcaatattcccca

SEQ ID NO: 88 - DNA, 429 bp, Unknown
  source: /note=“Description of Unknown 16S rRNA sequence”
cctgttcgctacccatgctttcgagcctcagcgtcagttgcagaccagacagccgccttcgccactggtgttcttccatatatctacgcattccaccgctacacatggagttccactgtcctcttctgcactcaagtcgcccggtttccgatgcacttcttcggttaagccgaaggctttcacatcagacctaagcaaccgcctgcgctcgctttacgcccaataaatccggataacgcttgccacctacgtattaccgcggctgctggcacgtagttagccgtgactttctggttggataccgtcactgcgtgaacagttactctcacgcacgttcttctccaacaacagagctttacgagccgaaacccttcttcactcacgcggtgttgctccatcaggcttgcgcccattgtggaagattcccta

SEQ ID NO: 89 - DNA, 404 bp, Unknown
  source: /note=“Description of Unknown 16S rRNA sequence”
cctgtttgctccccacgctttcgagcctcaacgtcagtcatcgtccagtaagccgccttcgccactggtgttcctcctaatatctacgcatttcaccgctacactaggaattccgcttacctctccgacactctagcctgacagttccaaatgcagtcccggggttgagccccgggctttcacatctggcttgccatgccgtctacgctccctttacacccagtaaatccggataacgcttgccccctacgtattaccgcggctgctggcacgtagttagccggggcttcttagtcaggtaccgtcattttcttccctgctgatagagctttacataccgaaatacttcatcgctcacgcggcgtcgctgcatcagggtttcccccattgtgcaatattcccca

SEQ ID NO: 90 - DNA, 407 bp, Unknown; identical to SEQ ID NO: 20
  source: /note=“Description of Unknown 16S rRNA sequence”

SEQ ID NO: 91 - DNA, 423 bp, Unknown
  source: /note=“Description of Unknown 16S rRNA sequence”
cctgtttgatacccgcaccttcgagcctcagcgtcagtaatgccccggtacaatgcctgcgcgatcggagttcctcgtgatatctaagcatttcaccgctacaccacgaattccatgtaccctgtgcatactcaaggctgccagtttcaacggcggaacgcggttgagccgcgccatttgaccgctgacttgactgcccgcctacgctccctttaaacccaataaatccggataacgctcgcatcttccgtattaccgcggctgctggcacggaattagccgatgcttattcataaggtacctgcaaatggccatacatggccaactttatccccttataaaaggagtttacaacccgtagggccgtcgtcctccacgctacttggctggttcaggctcgcgcccattgaccaatattcctca

SEQ ID NO: 92 - DNA, 407 bp, Unknown
  source: /note=“Description of Unknown 16S rRNA sequence”
cctgtttgctccccacgctttcgcgcttcagcgtcagtatctgtccagtgagctgacttctccatcggcattcctacaaatatctacgaatttcacctctacacttgtagttccgcccacctctccagtactctagaaagacagtttccaacgcaatacggagttgagccccgcattttcacatcagacttatcaaaccgcctagacgcgctttacgcccaataaatccggataacgcttgcgacatacgtattaccgcggctgctggcacgtatttagccgtcgcttcttctgttggtaccgtcactttcttcttcccaactgaaagcactttacattccgaaaaacttcatcgtgcacacagaattgctggatcagacttttggtccattgtccaatattcccca

SEQ ID NO: 93 - DNA, 429 bp, Unknown
  source: /note=“Description of Unknown 16S rRNA sequence”
cctgtttgctccccacgctttcgtgcatgagcgtcagtgttatcccagggggctgccttcgccatcggtattcctccacatctctacgcatttcactgctacacgtggaattctacccccctctgacacactctagccgtgcagtcaccaatgcaattcccaggttaagcccggggatttcacatcggtcttgcacaaccgcctgcgcacgctttacgcccagtaattccgattaacgcttggaccctacgtattaccgcggctgctggcacgtagttagccggtccttattcttccggtaccgtcatccacacctaatattagtaagtgcgatttctttccggacaaaagtgctttacaacccgaaggccttcttcacacacgcggcattgctggatcagggttgcccccattgtccaaaattcccca

SEQ ID NO: 94 - DNA, 429 bp, Unknown; identical to SEQ ID NO: 4
  source: /note=“Description of Unknown 16S rRNA sequence”

SEQ ID NO: 95 - DNA, 405 bp, Unknown
  source: /note=“Description of Unknown 16S rRNA sequence”
cctgtttgctacccacgctttcgagcctcagcgtcagttacagcccagtaggccgccttcgccactggtgttcctcctaatatctacgcatttcaccgctacactaggaattccgcctacctctactgcactcaagagtggcagttttgaacgcgactatcagttgagccgatagtttagacattcaacttgcctccccgcctacgctccctttacacccagtaattccggacaacgcttgccacctacgtattaccgcggctgctggcacgtagttagccgtggcttcctcctcgggtaccgtcatttattcgtccccgaagacagaggtttacaacccgaaggccgtcttccctcacgcggcgtcgctgcatcagggtttcccccattgtgcaatatccccca

SEQ ID NO: 96 - DNA, 423 bp, Unknown
  source: /note=“Description of Unknown 16S rRNA sequence”
cctgttcgatacccacgctttcgtgcatgagcgtcagttgagcgccggtatgctgccttcgcaatcggagttctgcgtgatatctatgcatttcaccgctacaccacgcattccgcatacttctcgctcactcaagaacgccagtttcaacggcacgacggggttgagccccgaaattttaccgctgacttgacattccgcctgcgcaccctttaaacccaataaatccggataacgctcgcatcctccgtattaccgcggctgctggcacggagttagccgatgctttttcttcaggtactcgcaaaacgccacacgtggcgccctttgctccctgacaaaagaggtttacaatccataggaccgtcatccctcacgcgacttggctggttcagccctgcggccattgaccaatattcctca

SEQ ID NO: 97 - DNA, 404 bp, Unknown; identical to SEQ ID NO: 5
  source: /note=“Description of Unknown 16S rRNA sequence”

SEQ ID NO: 98 - DNA, 428 bp, Unknown
  source: /note=“Description of Unknown 16S rRNA sequence”
cctatttgctccccacgctttcgggactgagcgtcagctgcagaccagatcgccgccttcgccactggtgttcctccatatatctacgcatttcaccgctacacatggaattccgcgatcctcttctgctctctagctatctggtttccatggcttactgaagttgagcttcagtctttcaccacagacctttattgccgcctgctccctctttacgcccaataattccggataacgcttgccacctacgtattaccgcggctgctggcacgtagttagccgtggctttctcataaagtaccgtcacttatatatcattccctatatataccgttcttcctttataacagaagtttacaatccgaagaccttcttccttcacgcggcgttgctcggtcagggttccccccattgccgaaaattcccta

SEQ ID NO: 99 - DNA, 403 bp, Unknown
  source: /note=“Description of Unknown 16S rRNA sequence”
cctgttcgctccccacgctttcgtcctttagcgtccgagtccgcccagcaggccgccttcgcctctggtattcctcctgatctctgcgcatttcaccgctacaccaggaattctgcctgcctcttcggctctctagctagggggtcctgggcaccgccctgatgtttcaccagtcttttaatgcccactccccttagccgccttcagacgctttaagcccagtgattccggacaacgctcgccccctacgtattaccgcggctgctggcacgtagttggccggggcttgcttgccgggtaccgtctctctcttccccggctacagcgctttacggtacgaataccttcttcgcgcacgcggcatcgctgcgtcagagttgcctccattgcgcaagattcccca

SEQ ID NO: 100 - DNA, 404 bp, Unknown
  source: /note=“Description of Unknown 16S rRNA sequence”
cctgtttgctccccacgctttcgagcctcaacgtcagttaccgtccagtaggccgccttcgccaccggtgttcttcctgatatctacgcatttcaccgctacaccaggaattccgcctacccctccggtactcaagatcaacagtttccaaagcagttacagggttgagccctatgatttcacttcagacttgctgttccgtctacgctccctttacacccagtaaatccggataacgcttgccccctacgtattaccgcggctgctggcacgtagttagccggggcttcttagtcaggtaccgtcattttcttccctgctgatagggctttacattccgaaaaacttcttcacccacgcggcgtcgctgcatcaggctttcgcccattgtgcaatattcccca

SEQ ID NO: 101 - DNA, 404 bp, Unknown
  source: /note=“Description of Unknown 16S rRNA sequence”
cctgtttgctccccacgctttcgagcctcaacgtcagttacagtccagtaagccgccttcgccactggtgttcctcctaatatctacgcatttcaccgctacactaggaattccacttacctctcctgcactctagtcctacagtttccaaagcagtcccggggttaagccccgggctttcacttcagacttgcaaaaccgtctacgctccctttacacccagtaaatccggataacgcttgccccctacgtattaccgcggctgctggcacgtagttagccggggcttcttagtcaggtaccgtcattttctttcctgctgatagagctttacataccgaaatacttcttcactcacgcggcgttgctgcatcagggtttcccccattgtgcaatattcccca

SEQ ID NO: 102 - DNA, 408 bp, Unknown
  source: /note=“Description of Unknown 16S rRNA sequence”
cctgtttgctccccacgctttcgagcctcagcgtcagtttcagtccagaaagccgccttcgccactggtgttcttcctaatatctacgcatttcaccgctacactaggaattccgctttcctctcctgtactctagcttgatagttccaaatgcaatcctcgaagttaagcttcgggctttcacatctggcttaccatgccgcctacgctccctttacacccagtaattccggataacgcttgccccctacgtattaccgcggctgctggcacgtagttagccggggcttcttatttaggtaccgtcatttttttcgtccctattgatagaagtttacgatccgaaaaccttcatccttcacgcggcgttgctgcatcagggtttcccccattgtgcaatattcccca

SEQ ID NO: 103 - DNA, 407 bp, Unknown
  source: /note=“Description of Unknown 16S rRNA sequence”
cctgtttgctccccacgctttcgcgcctcagcgtcagttatcgtccagcaagccgccttcgccactggtgttcctcctaatatctacgcatttcaccgctacactaggaattccgcttgcctctccgacactcaaggggtacagttttgaaagcagttccggggttgagccccgggatttcactcccaacttgcacctccgcctacacgccctttacacccagtaaatccggacaacgcttgcaacctacgtattaccgcggctgctggcacgtagttagccgttgctttttctccaggtaccgtcacgttcttcgtccctggtaaaagaagtttacaacccgaaggccttcttccttcacgcggcgttgctgggtcagggtttcccccattgcccaatattcccca

SEQ ID NO: 104 - DNA, 429 bp, Unknown
  source: /note=“Description of Unknown 16S rRNA sequence”
cctgtttgctccccacgctttcgtgcatgagcgtcagtacaggcccaggggattgccttcgccatcggtgttcctccgcatatctacgcatttcactgctacacgcggaattccatccccctctgccgtactccagcgatgcagtcacagatgcagttcccaggttgagcccggggatttcacaactgtcttacatcaccgcctgcgcacgctttacgcccagtaattccgattaacgcttgcaccctacgtattaccgcggctgctggcacgtagttagccggtgcttattcttacggtaccgtcattagccttctttattagaaaagaccgtttcgttccgtacaaaagcagtttacaacccgaaggccttcatcctgcacgcggcatggctggatcaggctttcgcccattgtccaaaattcccca

SEQ ID NO: 105 - DNA, 406 bp, Unknown
  source: /note=“Description of Unknown 16S rRNA sequence”
cctgtttgctccccacgctttcgagcctcagcgtcagtcatcgtccagtaagccgccttcgccactggtgttcctcctaatatctacgcatttcaccgctacactaggaattccacttacccctccgacactctagtccgacagtttccaatgcagtaccggggttgagccccgggctttcacatcagacttgccgtaccgcctgcgctccctttacacccagtaaatccggataacgcttgcaccatacgtattaccgcggctgctggcacgtatttagccggtgcttcttagtcaggtaccgtcatttcttcttccctgctgatagagctttacataccgaaatacttcttcgctcacgcggcgtcgctgcatcagggtttcccccattgtgcaatattcccca

SEQ ID NO: 106 - DNA, 406 bp, Unknown
  source: /note=“Description of Unknown 16S rRNA sequence”
cccgtttgctccccacgctttcgtgccttagtgtcagaactgtcccagtaacctgcctacgctatcggtgttctttctaatatctacggatttcactcctacactagaaattccagttacccctaacagtctcgagctgagcagtttagataatagcctgaatggttgagccaccagatttcactatctacttaccctgccacctacgcaactctttacacccagtcaatccggataatgcttggaccctacgtatgaccgcggctgctggcacgtagttagccggtccttattcatgtcttaccatcatatttctcggacataaaagcagtttacaacccgaaggccgtcatcctgcacgcggcgttgctccatcaggctttcgcccattgtgaaagattcccta

SEQ ID NO: 107 - DNA, 403 bp, Unknown
  source: /note=“Description of Unknown 16S rRNA sequence”
cctgttcgctcccccagctttcgcgcctcagcgtcagtgacggcccagaggtctgccttcgccatcggtgttcttcccgatatctgcgcatttcaccgctacaccgggaattccgacctcccctaccgcactcgagacgcctagttcgggacccggcttcgggttgagcccgaagattagaggtcccgcttgggcatccgcctgcgcgcgctttacgcccaatgaatccggataacgctcgccccctacgtattaccgcggctgctggcacgtagttagccggggcttcttctgcaggtaccgtcatgatcgttcctgctgaaagcggtttacgacccgaaggccttcatcccgcacgcggcgttgctgcgtcagggtttcccccattgcgcaagattcccca

SEQ ID NO: 108 - DNA, 429 bp, Unknown
  source: /note=“Description of Unknown 16S rRNA sequence”
cctgtttgctccccacgctttcgtgcatgagcgtcagtacaggtccaggggattgccttcgccatcggtgttcctccgcatatctacgcatttcactgctacacgcggaattccatccccctctaccgtactctagccatgcagtcacaaaggcagttcccaggttgagcccggggatttcacctctgtcttgcataaccgcctgcgcacgctttacgcccagtaattccgattaacgcttgcaccctacgtattaccgcggctgctggcacgtagttagccggtgcttattcttcaggtaccgtcatgagtcccaggtattagccagaaccttttcttccctgacaaaagcggtttacaacccgaaggccttcttcccgcacgcggcatggctggatcaggcttgcgcccattgtccaaaattcccca

SEQ ID NO: 109 - DNA, 403 bp, Unknown
  source: /note=“Description of Unknown 16S rRNA sequence”
cctgttcgctccccacgctttcgcgcctcagcgtcagtgtccgtccggcaggccgccttcgcctccggtattcctcctgatctctgcgcatttcaccgctacaccaggaattctgcctgcctctccggcactccagccggccggtccgcagcccatggcggggttgggccccaccctttgaagctgcgccgcgccggccgcctacgcgccctttacgcccagtcattccggacaacgctcgccccctacgtgttaccgcggctgctggcacgtagttagccggggctttctcaccgggtaacgtcaatctcttccccggcgacaaaactttacggaccgaagcccttcgtcgctcacgcggcatcgctgcgtcagagtttcctccattgcgcaagattcccca

SEQ ID NO: 110 - DNA, 404 bp, Unknown; identical to SEQ ID NO: 6
  source: /note=“Description of Unknown 16S rRNA sequence”

SEQ ID NO: 111 - DNA, 410 bp, Unknown
  source: /note=“Description of Unknown 16S rRNA sequence”
cctgtttgctccccacgctttcgtgcctcaacgtcagtacaggcccagcaagccgccttcgccaccggtgttcctccatatatttacgcattttaccgctacacatggaattccacttgcctctaccgtactctagctcaagagtttcttttgcgagattgggttgagcccaacaatttaacaaaagacttcttgtgccgtctacgcaccctttacgcccaataaatccggataacgctcgccccctacgtattaccgcggctgctggtacgtagttagccggggcttattcattaggtaccgtcaatacgagttttcgtccctaataaaagaactttacattacgaataacttcttcattcacgcggcattgctcggtcagactttcgtccattgccgaaaattcccta

SEQ ID NO: 112 - DNA, 424 bp, Unknown
  source: /note=“Description of Unknown 16S rRNA sequence”
cctgttcgatacccacgctttcgtgcctgagcgtcagtagggcgccggtgcgctgccttcgcaatcggagttctgcgtgatatctatgcatttcaccgctacaccacgcattccgcgcacttctcgcccactctagcccggcagttccgacggctggacggggttgagccccgcgatttgaccgccggcttgtcgggccgcctgcgcaccctttaaacccaataaatccggataacgctcgcatcctccgtattaccgcggctgctggcacggagttagccgatgcttgttctacgggtactctccttccgcggacacgtccgcggacttgctccccgtccaaagaggtctgcgacccgcagggccttcttccctcacgcgacttggctggttcaggcttccgcccattgaccaatattcctca

SEQ ID NO: 113 - DNA, 403 bp, Unknown
  source: /note=“Description of Unknown 16S rRNA sequence”
cctgttcgctcccccagctttcgcgcctcagcgtcagtgccggcccagagagctgccttcgccatcggtgttcttcccgatatctgcgcattccaccgctacaccgggaattccgctctcctctaccgcactcgagccgcccagttcggaacccggcccgaggttgagccccggggttagaggtcccgcttaggcggccgcctacgcgcgctttacgcccaatgaatccggataacgctcgctccctacgtattaccgcggctgctggcacgtagttagccggagcttcttctgcaggtaccgtcaatatcttccctgctgaaagcggtttacaacccgagggccgtcatcccgcacgcggcgttgctgcgtcagggttccccccattgcgcaaaattcccca

SEQ ID NO: 114 - DNA, 406 bp, Unknown
  source: /note=“Description of Unknown 16S rRNA sequence”
cctgtttgctccccacactttcgtgcctcagtgtcagtaacagtccagaaagccgccttcgccactggtgttcctcctaatatctacgcatttcaccgctacactaggaattccgctttcctctcctgcactcaagtagcccagttcagggggctaacaatggttgagccattgcctttcaccccctgcttaggtaaccacctacgcactctttacgcccaataattccggataacgcttgccccctacgtattaccgcggctgctggcacgtagttagccggggcttcctccaagggtaccgtcatttttttcttccccaaggacagagttttacaatccgaagaccttcttcactcacgcggcgttgctgcatcaggctttcgcccattgtgcaatattcccca

SEQ ID NO: 115 - DNA, 405 bp, Unknown
  source: /note=“Description of Unknown 16S rRNA sequence”
cctgtttgctccccatgctctcatgcctcagcgtcagttacagtccagtaagtcgccttcgccactggtgttcttcctaatctctacgcatttcaccgctacactaggaattccacttacctctcctgcactctagttaaacagttttatgtgctttctacaggttaagcctatatatttaacacataacttactcaaccgcctacgcatcctttacgcccagttattccgaacaacgcttgccacctacgtattaccgcggctgctggcacgtagttagccgtggcttcctctgcaggtaccgtccttcctcttccctgctaacagagctttacaatccgaaaaccttcttcactcacgcggcgttgctgcatcagggtttcccccattgtgcaatattcccca

SEQ ID NO: 116 - DNA, 407 bp, Unknown
  source: /note=“Description of Unknown 16S rRNA sequence”
cctgttcgctccccacgctttcgtgactcagcgtcagtttcagtccagtaagccgccttcgccaccggtgttcttcctaatatctacgcatttcaccgctacactaggaattccgcttacctctcctgtactctagttacacagtttcttatgcagctctggagttgagccccagaatttcacatatgacttgcataaccgcctactcaccctttacacccagtaaatccggataacgcttgcaccatacgtattaccgcggctgctggcacgtatttagccggtgcttcttattcaggtaccgtcatttctttcttccctgtcgatagaagtttacataccgaaatacttcttccttcacgcggcgttgctgcatcagggttccccccattgtgcaatattcccca

SEQ ID NO: 117 - DNA, 403 bp, Unknown
  source: /note=“Description of Unknown 16S rRNA sequence”
cctgtttgctccccacgctttcgtgcctcagcgtcagttacagtccagagagccgccttcgctactggtgttcctcctaatatctacgcatttcaccgctacactaggaattccactctcctctcctgcactcaagtcctacagttccaaaagcttactacggttgagccgtagcctttcacttctggcttgaaagaccgcctacgcaccctttacgcccagtaattccggataacgctagccccctacgtattaccgcggctgctggcacgtagttagccggggcttcctcctcaagtaccgtcattatcttccttgaggacagagctttacgacccgaaggccttcatcgctcacgcggcgttgctgcatcaggctttcgcccattgtgcaatattcccca

SEQ ID NO: 118 - DNA, 404 bp, Unknown
  source: /note=“Description of Unknown 16S rRNA sequence”
cctgtttgctccccacgctttcgagcctcagcgtcagttacagtccagagagtcgccttcgccactggtgttcttcctaatctctacgcatttcaccgctacactaggaattccactctcctctcctgcactctagataaccagtttggaatgcagcacccaagttgagcccgggtatttcacatcccacttaatcatccgcctacgctccctttacgcccagtaaatccggataacgctcgccacctacgtattaccgcggctgctggcacgtagttagccgtggcttcctccttgggtaccgtcattatcttccccaaagacagagctttacgatccgaaaaccttcatcactcacgcggcgttgctgcatcagggtttcccccattgtgcaatattcccca

SEQ ID NO: 119 - DNA, 406 bp, Unknown
  source: /note=“Description of Unknown 16S rRNA sequence”
cctgtttgctccccacgctttcgcgcctcaccgtcagttgtcgtccagtcatccgccttcgccactggtgttcttccttatatctacgcatttcaccgctacacaaggaattccgatgacctctccgatactcaagaaacacagtttcaaatgcagtttggaggttgagcctccagatttcacatctgacttgcattcccggctgcacgccctttacacccagtaaatccggacaacgcttgccacctacgtattaccgcggctgctggcacgtagttagccgtggcttattcgtcaggtaccctcttctactgttccctgacaaaagaagtttacaacccgaaagccttcttccttcacgcggcgttgctgggtcagacttgcgtccattgcccaatattcccca

SEQ ID NO: 120 - DNA, 424 bp, Unknown
  source: /note=“Description of Unknown 16S rRNA sequence”
cctgttcgatacccgcactttcgagcttcagcgtcagttgcgctccagtgagctgccttcgcaatcggagttcttcgtgatatctaagcatttcaccgctacaccacgaattccgcccactttgtgcgtactcaaggaaaccagttcgcgctgcagtgcagacgttgagcgtctacatttcacaacacgcttaatctccggcctacgctccctttaaacccaataaatccggataacgcccggaccttccgtattaccgcggctgctggcacggaattagccggtccttattcataaggtacatgcaaaaagcctcacgaggctcactttattcccttataaaagcagtttacaacccggagggccgtcatcctgcacgctacttggctggttcagacttgcgtccattgaccaatattcctca

SEQ ID NO: 121 - DNA, 406 bp, Unknown
  source: /note=“Description of Unknown 16S rRNA sequence”
cctgtttgctccccacgctttcgcacctcagcgtcagtatttgcccaggagaccgccttcgcctctggtgttctttcagatatctacgcattccaccgctacacctgaaattccatctccctctgcaatactctagaaaggtagttcaaaatgaccaacccaggttgagcccaggtctttcacatcttgcttgcctttccgcctacatgccctttacgcccagttattccgattaacgcttgcaccctccgtattaccgcggctgctggcacggagttagccggtgcttcttctgtgggtaacgtcagtatttcctccccactgaaagtgctttacaacccgaaggccttcttcacacacgcggcatggctgcatcagggtttcccccattgtgcaatattcccca

SEQ ID NO: 122 - DNA, 428 bp, Unknown
  source: /note=“Description of Unknown 16S rRNA sequence”
cctatttgctccccacgctttcgggactgagcgtcagttacagcccagatcgtcgccttcgccactggtgttcctccatatatctacgcatttcaccgctacacatggaattccacgatcctctactgcactctagctgtgcggtttccacggcttaccgaagtttagcttcgggctttcaccgcagacctgcactgccgcctgctccctctttacgcccaatgattccggataacgctcgccacctacgtattaccgcggctgctggcacgtagttagccgtggcttcctcataaagtaccgtcacttacatggcattccctccatgtaccgttcttcctttataacagaggtttacataccgaagtacttcctccctcacgcggcgttgctcggtcaggctttcgcccattgccgaaaattcccta

SEQ ID NO: 123 - DNA, 405 bp, Unknown
  source: /note=“Description of Unknown 16S rRNA sequence”
cctgtttgctccccgcgctttcgcgcctcagcgtcagtgtgcgtccagcaggccgccttcgcctctggtattcctcctgatatctgcgcatttcaccgctacaccaggaattctgcctgcctctccgacactctagccacgcggttcagggcgcttgcacggggttaggccccgtcttttcacaccctgcctgcgcggccgcctgcgcgccctttacgcccagtgattccggacaacgcttgccccccacgtattgccgcggctgctggcacgtggttggccggggctttcttaccgggtaccgtctgttttcttccccgataacagagctttacgagccggagcccttcctcgctcacgcggcattgctgcgtcagagtttcctccattgcgcaatattcccca

SEQ ID NO: 124 - DNA, 407 bp, Unknown
  source: /note=“Description of Unknown 16S rRNA sequence”
cctgtttgctacccacgctttcgagcctcagcgtcagttaaagcccagcaggccgccttcgccactggtgttcctcctaatatctacgcatttcaccgctacactaggaattccgcctgcctctacttcactcaagaaaaacagttttgaaagcagctcatgggttgagcccatgcatttcacttccaacttgctttcccgcctacgctccctttacacccagtaattccggacaacgctcgctccctacgtattaccgcggctgctggcacgtagttagccggagcttccttcttcggtaccgtcactttcttcgtcccgaatgacagaggtttacaacccgaaggccgtcttccctcacgcggcgtcgctgcatcaggctttcgcccattgtgcaatatccccca

SEQ ID NO: 125 - DNA, 429 bp, Unknown
  source: /note=“Description of Unknown 16S rRNA sequence”
cctatttgctccccacgctttcgtgcctcagcgtcaggtacaggccaggcggccgccttcgccgctggtgttcttccacatctctacgcattttaccgctacacatggagttccaccgccctctcctgtcctcaagccgtgcagtttccaaagcctgaaccggttgagccggtcccttttacttcagacttgcacagccgcctgcgcaccctttacgcccaatcattccggataacgcttgccacctacgtattaccgcggctgctggcacgtagttagccgtggctttctggccgggtaccatcaaacagtcagctttccactctggctgtcctttgtccccggcaacagggctttacaatccgaagaccgtcttcacccacgcggcattgctcgttcaggcttgcgcccattgacgaaaattcccta

SEQ ID NO: 126 - DNA, 404 bp, Unknown
  source: /note=“Description of Unknown 16S rRNA sequence”
cctgtttgctccccacgctttcgagcctcaacgtcagttactgtccagtaagccgccttcgccactggtgttcctcctaatatctacgcatttcaccgctacactaggaattccacttacctctccagcactctagatgaacagtttccaatgcagtcccggggttgagccccgggttttcacatcagacttgcccatccgtctacgctccctttacacccagtaaatccggataacgcttgcaccatacgtattaccgcggctgctggcacgtatttagccggtgcttcttagtcaggtaccgtcattatcttccctgctgatagagctttacataccgaaatacttcatcgctcacgcggcgtcgctgcatcagggtttcccccattgtgcaatattcccca

SEQ ID NO: 127 - DNA, 424 bp, Unknown
  source: /note=“Description of Unknown 16S rRNA sequence”
cctgtttgatacccgcactttcgagcctcagcgtcagttgcaccccggatacctgccttcgcgatcggagttcttcatgatatctgagcatttcaccgctacaccatgaattccagcatccctgtgtgcactcaagactcccagtatcaactgcagtccgacggttgagccgccgtatttcacaactgacttaagagtccgcctgcgctccctttaaacccaataaatccggataacgcctggaccttccgtattaccgcggctgctggcacggaattagccggtcctttttctgatggtacatacaaaacagctcacgagctgcactttattcccaactaaaagcagtttacaacccggagggccgtcatcctgcacgctacttggctggttcagacttgcgtccattgaccaatattcctca

SEQ ID NO: 128 - DNA, 424 bp, Unknown
  source: /note=“Description of Unknown 16S rRNA sequence”
cctgttcgatacccgcactttcgagcttccgcgtccgttgcgctccagtgagctgccttcgcaatcggagttcttcgtgatatctaagcatttcaccgctacaccacgaattccgcccactttgtgcgtactcaaggaaaccagttcgcgctgcagtgcagacgttgagcgtctacatttcacaacacgcttaatctccggcctacgctccctttaaacccaataaatccggataacgcccggaccttccgtattaccgcggctgctggcacggaattagccggtccttattcataagatacatgcaaaaagagtcacgactctcactttattcccttataaaagcagtttacaacccatagggccgtcttcctgcacgctacttggctggttcagactctcgtccattgaccaatattcctca

SEQ ID NO: 129 - DNA, 406 bp, Unknown
  source: /note=“Description of Unknown 16S rRNA sequence”
cctgtttgctccccacgctttcgtgcctcagcgtcagttatggtccagaaagccgccttcgctactggtgttcctccgaatctctacgcatttcaccgctacactcggaattccactttcctctcccacactctagcacagcagtttcagaagcagctctggggttgagccccaaaatttcacttctgacttaccatgccgcctacgcaccctttacacccagtaaatccggataacgctcgctccctacgtattaccgcggctgctggcacgtagttagccggagcttcttagtcaggtaccgtcattttttcttccctgctgatagaagtttacaatccgaagaccttcatccttcacgcggcgtcgctgcatcaggcttgcgcccattgtgcaatattcccca

SEQ ID NO: 130 - DNA, 424 bp, Unknown; identical to SEQ ID NO: 7
  source: /note=“Description of Unknown 16S rRNA sequence”

SEQ ID NO: 131 - DNA, 404 bp, Unknown
  source: /note=“Description of Unknown 16S rRNA sequence”
cctgtttgctccccacgctttcgagcctcagcgtcagttacagtccagagagtcgccttcgccactggtgttcttcctaatctctacgcatttcaccgctacactaggaattccactctcctctcctgcactctagataatcagtttggaatgcagcccccaggttgagcctgagtatttcacatcccacttaattatccgcctacgctccctttacgcccaataattccggataacgctcgccacctacgtattaccgcggctgctggcacgtagttagccgtggcttcctccttgggtaccgtcattatcgtccccaaagacagagctttacaatccgaagaccgtcatcactcacgcggcgttgctgcatcagggtttcccccattgtgcaatattcccca

SEQ ID NO: 132 - DNA, 406 bp, Unknown
  source: /note=“Description of Unknown 16S rRNA sequence”
cctgtttgctccccacgctttcgcacctcagcgtcagtatttgcccaggagaccgccttcgcctctggtgttctttcagatatctacgcattccaccgctacacctgaaattccatctccctctgcaatactctagaaaagtagttcaaaacgaccaacccaggttgagcccgggtatttcacgccttgcttgcttttccgcctacatgccctttacgcccagttattccgattaacgcttgcaccctccgtattaccgcggctgctggcacggagttagccggtgcttcttctgtgggtaacgtcagtgtttcctccccactgaaagtgctttacaacccgaaggccttcttcacacacgcggcatggctgcatcagggtttcccccattgtgcaatattcccta

SEQ ID NO: 133 - DNA, 407 bp, Unknown
  source: /note=“Description of Unknown 16S rRNA sequence”
cctgtttgctccccacgctttcgcgcctcagcgtcagttaatgtccagcaggccgccttcgccactggtgttcctcccaatatctacgcatttcaccgctacactgggaattccgcctgcctctccatcactcaagatccgcagttttgaaagcagtttgggggttgagcccccagatttcactcccaacttacagacccgcctacacgccctttacacccagtaaatccggataacgcttgctccctacgtattaccgcggctgctggcacgtagttagccggagcttattctttaggtaccgtcatttttttcgtccctaattaaagatttttacaatccgaagaccttcttcaatcacgcggcgttgctgcgtcagggttgcccccattgcgcaatattcccca

SEQ ID NO: 134 - DNA, 429 bp, Unknown
  source: /note=“Description of Unknown 16S rRNA sequence”
cccgttcgctaccctggctttcgcatctcagcgtcagacacagtccagaaaggcgccttcgccactggtgttcctcccaatatctacgcatttcaccgctacactgggaattcccctttcctctcctgcactcaagtctcccagtatccagagccatacggggttgagccccgcattttcactccagacttaagaaaccgcctacatgctctttacgcccaataattccggacaacgctcgccacctacgtattaccgcggctgctggcacgtagttagccgtggcttattcgtttactaccgtcattacaaataattattcacaatctgcacattcgtcataaacaaaagagttttacggaacgaattccttcatcactcacgcggcattgctccgtcaggctttcgcccattgcggaagattcccca

SEQ ID NO: 135 - DNA, 429 bp, Unknown
  source: /note=“Description of Unknown 16S rRNA sequence”
cctgtttgctacccacgctttcgaacctcagcgtcagttacagaccagagagccgctttcgccactggtgttcttccatatatctacgcatttcaccgctacacatggagttccactctcctcttctgcactcaagtctcccagtttccaatgcactactccggttaagccgaaggctttcacatcagacttaaaagaccgcctgcgttccctttacgcccaataaatccggataacgcttgccacctacgtattaccgcggctgctggcacgtagttagccgtggctttctggttagataccgtcgaaacgtgaacagttactctcacgcactttcttctctaacaacagggttttacgatccgaagaccttcttcacccacgcggcgttgctccatcaggctttcgcccattgtggaagattcccta

SEQ ID NO: 136 - DNA, 407 bp, Unknown; identical to SEQ ID NO: 21
  source: /note=“Description of Unknown 16S rRNA sequence”

SEQ ID NO: 137 - DNA, 405 bp, Unknown
  source: /note=“Description of Unknown 16S rRNA sequence”
cctgttcgctcccccggctttcgcgcctcagcgtcagtgtcggcccagcaggctgccttcgccatcggtgttcctcccgatatctgcgcattccaccgctacaccgggaattccacctgcccctaccgcactcgagccgcccagttcggaacccggccgcggggttgagccccgggattagaggttccgcttaggcggccgcctacgcgcgctttacgcccaatgaatccggataacgctcgccccctacgtattaccgcggctgctggcacgtagttagccggggcttcttctgcaggtaccgtctaggttcttccctgctgaaagcggtttacaacccgaaggccttcgtcccgcacgcggcgttgctgcgtcagggttccccccattgcgcaaaattcccca

SEQ ID NO: 138 - DNA, 429 bp, Unknown
  source: /note=“Description of Unknown 16S rRNA sequence”
cctatttgctccccacgctttcgtgcctcagcgtcagagtcagtccaggcggccgccttcgccactggtgttcttccatatatctacgcattttaccgctacacatggagttccaccgccctctcctgctctccagccggccagtttcctcagcaaaccgaagttgagcttcgatctttcactgaagacttgaccggcagcctgcgcaccctttacgcccaatcattccggataacgctcgccacctacgtattaccgcggctgctggcacgtagttagccgtgactttctggcggggtacgatcctgaaggaagctttccactcttccttcctcttattccccgcaacagagctttacagcccgaaggccgtcttcactcacgcggcattgctcgttcagagtttcctccattgacgaaaattcccta

SEQ ID NO: 139 - DNA, 429 bp, Unknown
  source: /note=“Description of Unknown 16S rRNA sequence”
cctatttgctccccacgctttcgtgcctcagcgtcagcaacaggccaggcggccgccttcgccactggtgttcttccatatatctacgcattttaccgctacacatggagttccaccgccctctcctgctctccagtcctccagtttccaaagccatgcatgagttgagctcatgcgtttcactccagacttgcaggaccgcctgcgcaccctttacgcccaatcattccggataacgctcgccacctacgtattaccgcggctgctggcacgtagttagccgtgactttctggcggggcaccatcagtcagcacccatttcctgatgctgccttttttcccccgcaacagagctttacgacccgaaggccttcttcactcacgcggcatcgctcgttcaggcttgcgcccattgacgaaaattcccta

SEQ ID NO: 140 - DNA, 429 bp, Unknown
  source: /note=“Description of Unknown 16S rRNA sequence”
cctgttcgctacccatgctttcgagcctcagcgtcagttgcagaccagagagccgccttcgccactggtgttcttccatatatctacgcattccaccgctacacatggagttccactctcctcttctgcactcaagaaaaacagtttccgatgcagttcctcggttaagccgagggctttcacatcagacttattcttccgcctgcgctcgctttacgcccaataaatccggacaacgcttgccacctacgtattaccgcggctgctggcacgtagttagccgtgactttctggttgattaccgtcaaataaaggccagttactacctctatccttcttcaccaacaacagagctttacgatccgaaaaccttcttcactcacgcggcgttgctccatcagactttcgtccattgtggaagattcccta

SEQ ID NO: 141 - DNA, 403 bp, Unknown
  source: /note=“Description of Unknown 16S rRNA sequence”
cctgtttgctacccacgctttcgttcctcagcgtcagtttgtgtccagcaagtcgccttcgccactggtattcctcctaatatctacgcatttcaccgctacactaggaattccacttgcctctccacatctcaagaaatacagtttcagatgcttacagaggttaagcctctgcctttcacatctgacttgcatttccgcctgcgaaccctttacgcccagtaattccggacaacgctagccccctacgtattaccgcggctgctggcacgtagttagccggggcttcctcctatggtaccgtcattatcttcccataggacagagctttacgattcgaaaaccttcatcgctcacgcggcgtcgctgcatcagggtttcccccattgtgcaatattcccca

SEQ ID NO: 142 - DNA, 404 bp, Unknown
  source: /note=“Description of Unknown 16S rRNA sequence”
cctgttcgctccccacgctttcgagcctcagcgtcagttacagtccagagagtcgccttcgccactggtgttcttcctaatctctacgcatttcaccgctacactaggaattccactctcccctcctgcactctagattcccagtttggaatgcagcacccaggttgagcccgggtatttcacatcccacttaaaaatccgcctacgctccctttacgcccagtaaatccggacaacgctcgccacctacgtattaccgcggctgctggcacgtagttagccgtggcttcctccttgggtaccgtcattatcgtccccaaagacagagctttacaatccgaagaccgtcatcactcacgcggcgttgctgcatcagggtttcccccattgtgcaatattcccca

SEQ ID NO: 143 - DNA, 428 bp, Unknown
  source: /note=“Description of Unknown 16S rRNA sequence”
cctatttgctccccacgctttcgtgcctgagcgtcagttacagaccaggtagccgctttcgccactggtgttcctccatatatctacgcattttaccgctacacatggaattccactaccctcttctgcactctagtctaccagtttttaaggctatattgggttaagcccaacgctttcaccctaaacttaatagaccgcctgcgcaccctttacgcccaataattccggataacgcttgccacctacgtattaccgcggctgctggcacgtagtaagccgtgactttctggtaaagtaccgtcaaaatatcagcatttcctcagatattcattcttcctttacaacagagctttacaacccaaaggccttcatcactcacgcggcgttgctcggtcagggtttcccccattgccgaaaattcctta

SEQ ID NO: 144 - DNA, 403 bp, Unknown
  source: /note=“Description of Unknown 16S rRNA sequence”
cctgttcgctccccacgctttcgcacctgagcgtcagttgccagccagcaggccgccttcgcctccggtattcctcctgatctctgcgcatttcaccgctacaccaggaattctgcctgcctctctggcactcgagctccacggttccgccgaccgttaccgggttgggcccggtaatttcatcggcggcccttgaagccgcctgcgtgccctttacgcccaatgattccggacaacgctcgccccccacgtgttaccgcagctgctggcacgtagttggctggggctcctacccgggtaccgtcttcctcttccccggggacaaagctttacgggacgagtcccttcttcactcacgcggcatcgctgcgtcagggtttcccccattgcgcaatattcccca

SEQ ID NO: 145 - DNA, 404 bp, Unknown
  source: /note=“Description of Unknown 16S rRNA sequence”
cctgtttgctccccacgctttcgagcctcaacgtcagttactgtccagtaagccgccttcgccactggtgttcctcctaatatctacgcatttcaccgctacactaggaattccacttacctctccagcactctagcatcacagtttccaaagcagtcccggggttgagccccgggctttcacttcagacttgcagcgccgtctacgctccctttacacccagtaaatccggataacgcttgccccctacgtattaccgcggctgctggcacgtagttagccggggcttcttagtcaggtaccgtcattttcttccctgctgatagagctttacataccgaaatacttcttcactcacgcggcgttgctgcatcaggcttgcgcccattgtgcaatattcccca

SEQ ID NO: 146 - DNA, 407 bp, Unknown
  source: /note=“Description of Unknown 16S rRNA sequence”
cctgtttgctccccatgctttcgcgcttcagcgtcagtatctgtccagtgagctgacttctctatcggcattcctacaaatatctacgaatttcacctctacacttgtagttccgcccacctctccagtactctagtctggcagtttccaacgcaatacggagttgagccccgcatttccacatcagacttaccagaccgcctagacgcgctttacgcccaataaatccggataacgcttgcgacatacgtattaccgcggctgctggcacgtatttagccgtcgcttcttctatcggtaccatcactttctttttcccgattgaaagcactttacaatcctaagaccgtcatcgtgcacacagaattgctggatcagacttttggtccattgtccaatattcccca

SEQ ID NO: 147 - DNA, 424 bp, Unknown
  source: /note=“Description of Unknown 16S rRNA sequence”
cctgtttgatacccgcactttcgagcctcagcgtcagttacactccagatacctgccttcgcgatcggagttcctcatgatatctgagcatttcaccgctacaccatgaattccagtatctctgcgtgtactcaagactcccagtatcaactgcagtccgacggttgagccgccgtatttcacaactgacttaagagtccgcctgcgctccctttaaacccaataaatccggataacgcctggaccttccgtattaccgcggctgctggcacggaattagccggtcctttttctgatggtacatacaaaacggtacacgtaccgcactttattcccaactaaaagcagtttacaacccagagggccgtcatcctgcacgctacttggctggttcagacttccgtccattgaccaatattcctca

SEQ ID NO: 148 - DNA, 404 bp, Unknown
  source: /note=“Description of Unknown 16S rRNA sequence”
cctgtttgctccccacgctttcgagcctcaacgtcagttacagtccagtaagacgccttcgccactggtgttcctcctaatatctacgcatttcaccgctacactaggaattcctcttacctctcctgcactccagtatcacagtttccaaagcagtcccgcggttgagccccgggctttcacttcagacttgcagtaccgtctacgctccctttacacccagtaaatccggataacgcttgccccctacgtattaccgcggctgctggcacgtagttagccggggcttcttagtcaggtaccgtcattttcttccctgctgatagagctttacataccgaaatacttcttcactcacgcggcgtcgctgcatcagggtttcccccattgtgcaatattcccca

SEQ ID NO: 149 - DNA, 410 bp, Unknown
  source: /note=“Description of Unknown 16S rRNA sequence”
gcctgttcgctccccacgctttcgctcctcagcgtcaggaaaggcccagagaaccgccttcgccactggtgttcctcctgatatctgcgcattccaccgctccaccaggaattccattctcccctaccttcctcaagtcaacccgtatcgaaagcacgctcagggttaagccccaagattacacttccgacgcgatcaaccacctacgagccctttacgcccaataaatccggacaacgctcgcaccctacgtatcaccgcggctgctggcacgtagttagccggtgcttctttacccattaccgtcactcacgcttcgtcacaggcgaaagcggtttacaacccgaaggccgtcatcccgcacgcggcgttgctgcatcaggcttccgcccattgtgcaatattcccca

SEQ ID NO: 150 - DNA, 404 bp, Unknown; identical to SEQ ID NO: 8
  source: /note=“Description of Unknown 16S rRNA sequence”

SEQ ID NO: 151 - DNA, 404 bp, Unknown
  source: /note=“Description of Unknown 16S rRNA sequence”
cctgtttgctccccacgctttcgagcctcaacgtcagttaccgtccagtaagccgccttcgccactggtgttcctcctaatatctacgcatttcaccgctacactaggaattccgcttacctctccggcactctagaagcacagtttccaatgcagtcccgtggttgagcctcgggttttcacatcagacttgcacttccgtctacgctccctttacacccagtaaatccggataacgcttgccccctacgtattaccgcggctgctggcacgtagttagccggggcttcttactcaggtaccgtcattttcttccctgctgatagaagtttacataccgaaatacttcatccttcacgcggcgtcgctgcatcagggtttcccccattgtgcaatattcccca

SEQ ID NO: 152 - DNA, 403 bp, Unknown
  source: /note=“Description of Unknown 16S rRNA sequence”
cctgttcgctcccccagctttcgcgcctcagcgtcagtggcggcccagcaggctgccttcgccatcggtgttcttcccgatatctgcgcattccaccgctacaccgggaattccgcctgcctctaccgcactcgagccgcccagtccggaacccggcccgaggttgagccccgggattagaggttccgcttaggcggccgcctacgcgcgctttacgcccaatgaatccggataacgctcgccccctacgtattaccgcggctgctggcacgtagttagccggagcttcttctgcaggtaccgtctatgtcttccctgctgaaagcggtttacaacccgaaggccttcgtcccgcacgcggcgttgctgcgtcagggtttcccccattgcgcaaaattcccca

SEQ ID NO: 153 - DNA, 403 bp, Unknown; identical to SEQ ID NO: 22
  source: /note=“Description of Unknown 16S rRNA sequence”

SEQ ID NO: 154 - DNA, 405 bp, Unknown
  source: /note=“Description of Unknown 16S rRNA sequence”
cctgttcgctccccgcgctctcgcgcctcggcgtcagtgtccttccggcgggccgccttcgcctccggtgttcctcctggcctctgcgcatttcaccgctacgccaggaattccgcccgcccctccggcactccagcggctcggttcggggcgctttcccggggttgggccccggattttcacaccccgccttagccgccgcctgcgcgcgctttacgcccagtgattccggacaacgctcgccccccacgtcttgccgcggctgctggcacgtggttggccggggctttcttgccgggtaccgtcctgtctcttccccggcggcggggctttacgggccgaggcccttcatcgcccacgcggcatcgctgcgtcagagttccctccattgcgcaatattcccca

SEQ ID NO: 155 - DNA, 404 bp, Unknown
  source: /note=“Description of Unknown 16S rRNA sequence”
cctgtttgctccccacgctttcgagcctcaacgtcagttaccgtccagtaagccgccttcgccactggtgttcctcctaatatctacgcatttcaccgctacactaggaattccgcttacctctccggcactctagaaaaacagtttccaatgcagtcctggggttaagccccagcctttcacatcagacttgctcttccgtctacgctccctttacacccagtaaatccggataacgcttgccccctacgtattaccgcggctgctggcacgtagttagccggggcttcttagtcaggtaccgtcattttcttccctgctgatagaagtttacataccgagatacttcttccttcacgcggcgtcgctgcatcagggtttcccccattgtgcaatattcccca

SEQ ID NO: 156 - DNA, 407 bp, Unknown
  source: /note=“Description of Unknown 16S rRNA sequence”
cctgtttgctacccacgctttcgcgcttcagcgtcagtatctgtccagtgggctgacttctccatcggcattcctacaaatatctacgaatttcacctctacacttgtagttccgcccacctctccagtactctagtttgacagtttccaacgcaatacggagttgagccccgcattttcacatcagacttaccaaaccacctagacgcgctttacgcccaataaatccggataacgcttgcgacatacgtattaccgcggctgctggcacgtatttagccgtcgcttcttctgttggtaccgtcacgttcttcgtcccaactgaaagcactttacattccgaaaaacttcatcgtgcacacagaattgctggatcagacttttggtccattgtccaatattcccca

SEQ ID NO: 157 - DNA, 429 bp, Unknown
  source: /note=“Description of Unknown 16S rRNA sequence”
cctgtttgctccccacgctttcgcacctcagcgtcaataccggtccaggtggccgccttcgccactgatgttcctccagatatctacggatttcactcctacacctggaattccgccaccctctcccggattcaagtcgtgcagtatcaagggcagttccacggttgagccgtgggatttcacccctgacttacataacagcctacgtgcgctttacgcccagtgattccgattaacgcttgcaccctccgtattaccgcggctgctggcacggagttagccggtgcttcctttgaaggtaccgtcagtaggatgatgattagagcaccctagtttcttcccttctgacagaggtttacgatccgaagaccttcatccctcacgcggcgtcgctgcgtcaggctttcgcccattgcgcaatattcccca

SEQ ID NO: 158 - DNA, 404 bp, Unknown
  source: /note=“Description of Unknown 16S rRNA sequence”
cctgtttgctccccacgctttcgagcctcaacgtcagttactgtccagcaagccgccttcgccactggtgttcctcctaatatctacgcatttcaccgctacactaggaattccgcttacctctccagcactccagcttaacagtttccaaagcagtcccggggttgagccccgggctttcacttcagacttgctaagccgtctacgctccctttacacccagtaaatccggataacgcttgccccctacgtattaccgcggctgctggcacgtagttagccggggcttcttagtcaggtaccgtcattctcttccctgctgatagagctttacataccgaaatacttcttcactcacgcggcgtcgctgcatcagggtttcccccattgtgcaatattcccca

SEQ ID NO: 159 - DNA, 428 bp, Unknown
  source: /note=“Description of Unknown 16S rRNA sequence”
cctatttgctccccacgctttcgtgcctgagcgtcagttacagaccaggtagccgccttcgccactggtgttcctccatatatctacgcattttaccgctacacatggaattccactaccctcttctgcactctagtctatcagtttttaaggctatattgagttgagctcaacgctttcaccctaaacttaatagaccgcctgcgcaccctttacgcccaataattccggataacgcttgccacctacgtattaccgcggctgctggcacgtagtaagccgtgactttctggtaaagtaccgtcagtagtaaatcatttcctatttactaagttcttcctttacaacagagctttacaacccaaaggccttcatcactcacgcggcgttgctcggtcagggtttcccccattgccgaaaattcctta

SEQ ID NO: 160 - DNA, 405 bp, Unknown
  source: /note=“Description of Unknown 16S rRNA sequence”
cctgtttgctccccacgctttcgcacctgagcgtcagtgtctgtccggcaggccgccttcgcctctggtattcctcctgatatctgcgcatttcaccgctacaccaggaattctgcctgcctctccagcactccagcttgagggttcgggatggcccttcagggttgggccctgacttttcacatcccgccttccaagccgcctgcgtgccctttacgcccagtaattccggacaacgctcgccccctacgtgttaccgcggctgctggcacgtagttagccggggcttgcttgttaggtaccgtctctcttcttccctaacggcagagctttacgggacgaatcccttcttcactcacgcggcattgctgcgtcagagttgcctccattgcgcaatattcccca

SEQ ID NO: 161 - DNA, 424 bp, Unknown; identical to SEQ ID NO: 9
  source: /note=“Description of Unknown 16S rRNA sequence”

SEQ ID NO: 162 - DNA, 404 bp, Unknown
  source: /note=“Description of Unknown 16S rRNA sequence”
cctgttcgctcccccagctttcgcgcctcagcgtcagtgccggcccagcaagctgccttcgccgtcggtgttcttcccgatatctgcgcattccaccgctacaccgggaattccacttgcctctaccgcactcgagccgttcagttcagaacccgaaccaaggttgagcctcggtcttaaaggttccgcttaagcggccgcctacgcgccctttacgcccaatgaatccggataacgctcgccccctacgtgttaccgcggctgctggcacgtagttagccggggcttcttctgtgggtaccgtcaatttcttccccactgaaagcggtttacaacccgaaggccttcagtcccgcacgcggcgttgctgcgtcagggttgcccccattgcgcaagattcccca

SEQ ID NO: 163 - DNA, 404 bp, Unknown
  source: /note=“Description of Unknown 16S rRNA sequence”
cctgttcgctccccacgctttcgcacctcagcgtcagtaaaggcccggcagaccgccttcgcctccggtattcctcctgatctctgcgcatttcaccgctacaccagaaattctgtctgcctctacctcactctagctgcccggtttgcaatgctacctcagggttaagccccgggctttcacatcacacctaaacagccgcctgcgtgccctttacgcccaataaatccggataacgctcgccccctacgtattaccgcagctgctggcacgtagttggctggggcttgctcatcgggtaccgtcttcctcttccccgataacagagctttacagaacgaatcccttcttcactcacgcggcattgctgcgtcagagttgcctccattgcgcaatattcccca

SEQ ID NO: 164 - DNA, 402 bp, Unknown
  source: /note=“Description of Unknown 16S rRNA sequence”
cctgtttgctccccacgctctcgcgccttagcgtcagtctcttcccagcaggccgccttcgcctccggtattcctcctgatatctgcgcatttcaccgctacaccaggaattctgcctgcctctaaagtactccagccagctggttccccgtgcttgacccgattgagtccggcccttttacacgaggcctctctggccgcctacgcgccctttacgcccagtcattccggacaacgcttgccccctacgtattaccgcggctgctggcacgtagttagccggggcttaaagcaggataatgtctctctcttttcctgctactgagctctgcgggacgaatcccttcctcactcacgcggcatcgctgcgtcagagttgcctccattgcgcaatattcccca

SEQ ID NO: 165 - DNA, 404 bp, Unknown
  source: /note=“Description of Unknown 16S rRNA sequence”
cctgtttgctccccacgctttcgagcctcaacgtcagttaccgtccagtaagccgccttcgccactggtgttcctcctaatatctacgcatttcaccgctacactaggaattccgcttacctctccggcactctagatggacagtttccaaagcagtccaggggttgagcccctgcctttcacttcagacttgcccgtccgtctacgctccctttacacccagtaaatccggataacgcttgccccctacgtattaccgcggctgctggcacgtagttagccggggcttcttagtcaggtaccgtcattgtcttccctgctgatagaagtttacataccgagatacttcttccttcacgcggcgtcgctgcatcagggtttcccccattgtgcaatattcccta

SEQ ID NO: 166 - DNA, 403 bp, Unknown
  source: /note=“Description of Unknown 16S rRNA sequence”
cctgtttgctccccacgctttcgtgcctcagcgtcagttacagtccagagagccgccttcgcaactggtattcctcctaatatctacgcatttcaccgctacactaggaattctactctcctctcctgcactcaagtcttacagtttcaagagcttactacggttgagccgtagcctttcactcctgacttgaaagaccgcctacgcaccctttacgcccagtaattccggataacgctagccccctacgtattaccgcggctgctggcacgtagttagccggggcttcctcctcaagtaccgtcattatcttccttgaggacagagctttacgacccgaaggccttcatcactcacgcggcgttgctgcatcaggctttcgcccattgtgcaatattcccca

SEQ ID NO: 167 - DNA, 407 bp, Unknown
  source: /note=“Description of Unknown 16S rRNA sequence”
cctgtttgctccccacgctttcgcgcctcagcgtcagttgctgtccagttgaccgccttcgccactggtgttcctcctaatatctacgcatttcaccgctacactaggaattccgtcaacctctccagtactcaagaacaacagtttcaaatgcagtttcagggttgagccctgagatttcacatctgacttgccatcccgcctacgcgccctttacacccagtaaatccggataacgcttgctccctacgtattaccgcggctgctggcacgtagttagccggagcttattcatcaggtaccgtcacatccttcgtccctgataaaagaagtttacaacccgaaggccttcttccttcacgcggcattgctgggtcaggctttcgcccattgcccaatattcccca

SEQ ID NO: 168 - DNA, 424 bp, Unknown
  source: /note=“Description of Unknown 16S rRNA sequence”
cctgttcgatacccgcactttcgtgcctgagcgtcagttgagcgccggtaagctgccttcgcaatcggcgttctgcgtgatatctatgcatttcaccgctacaccacgcattccgcttacttctcgctcactctagtgcggaagtttcaacggctcggcggagttgagctccgtaattttaccgctgactttccgtaccgcctgcgcaccctttaaacccaataaatccggataacgctcgcatcctccgtattaccgcggctgctggcacggagttagccgatgctttttcttcgggtactctccctgatgactcgcaagtcacccattgctccccgacaaaagaggtttacgacccgtagggccttagtccctcacgcgacttggctggttcagccttccggccattgaccaatattcctca

SEQ ID NO: 169 - DNA, 403 bp, Unknown; identical to SEQ ID NO: 23
  source: /note=“Description of Unknown 16S rRNA sequence”

SEQ ID NO: 170 - DNA, 405 bp, Unknown
  source: /note=“Description of Unknown 16S rRNA sequence”
cctgtttgctccccacgctctcgcacctgagcgtcagtgtctgtccggcaggccgccttcgcctctggtattcctcctgatatctgcgcatttcaccgctacaccaggaattctgcctgcctctccagcactcgagctgcatggttcgggatgcacctccagggttgggccctggcttttcacatcccgcctacacagccgcctgcgtgccctttacgcccagtcattccggacaacgctcgccccctacgtgttaccgcggctgctggcacgtagttagccggggcttgcttgttaggtaccgtctgctctcttccctaacggcagagctttacggaacgaattccttcttcactcacgcggcattgctgcgtcagagttgcctccattgcgcaatattcccca

SEQ ID NO: 171 - DNA, 429 bp, Unknown
  source: /note=“Description of Unknown 16S rRNA sequence”
cctgtttgctccccacgctttcgtgcctgagcgtcagttatgtcccaggaggctgccttcgccatcggtgttcttccacatatctacgcatttcactgctacacgtggaattccacctccctctgacacactctagtcgagcagtcacatatgccattcccgagttaagctcggggatttcacatctgtcttactcaaccgcctgcgcaccctttacgcccagtaattccgattaacgctcgcaccctacgtattaccgcggctgctggcacgtagttagccggtgcttattcttagagtaccgtcagcaacatcctttattcgaagatgtcttttcgttctctacaaaagtggtttacaacccgagggccttcatcccacacgcggaatagctggatcaggcttgcgcccattgtccaaaattcccca

SEQ ID NO: 172 - DNA, 424 bp, Unknown
  source: /note=“Description of Unknown 16S rRNA sequence”
cctgtttgatacccgcactttcgagcatcagcgtcagttacggtccagcaagctgccttcgcaatcggagttcttcgtgatatctaagcatttcaccgctacaccacgaattccgcctgcctttaccgcactcaagaactccagtatcaactgcaattttacggttgagccgcaaactttcacaactgacttaaaattccgcctacgctccctttaaacccaataaatccggataacgctcggatcctccgtattaccgcggctgctggcacggagttagccgatccttattcatacggtacatacaaaaaaccacacgtggctaactttattcccgtataaaagaagtttacaacccatagggcagtcatccttcacgctacttggctggttcagactctcgtccattgaccaatattcctca

SEQ ID NO: 173 - DNA, 426 bp, Unknown
  source: /note=“Description of Unknown 16S rRNA sequence”
cctatttgctccccacgctttcgtgcttcagtgtcagttacaggccaggcaaccgccttcgccactggtgttcttccatatatctacgcattttaccgctacacatggagttccattgccctctcctgcactctagcttgccagtttccaaagcgtacgacagttgagctgccggctttaactccagacttaacaagccacctacgcaccctttacgcccaatcattccggataacgctcgccacctacgtattaccgcggctgctggcacgtagttagccgtgactttctggtaagctgatcatttatggatcattccctatccatatcgtttctaacttacaacagagctttacaatccgaagaccgtcttcactcacgcggcattgctcgttcagggttccccccattgacgaaaattcccta

SEQ ID NO: 174 - DNA, 423 bp, Unknown; identical to SEQ ID NO: 10
  source: /note=“Description of Unknown 16S rRNA sequence”

SEQ ID NO: 175 - DNA, 423 bp, Unknown
  source: /note=“Description of Unknown 16S rRNA sequence”
cctgtttgatccccgcactttcgtgcctgagcgtcagttgagcgccggtatgctgccttcgcaatcggggttctgcgtgatatctatgcatttcaccgctacaccacgcattccgcatacttctcgctcactctagcatgtcagtttcaacggctatacggcgttgagcaccgcatttttaccgctgacttggcacgccgcctgcgcaccctttaaacccaataaatccggataacgctcgcatcctccgtattaccgcggctgctggcacggagttagccgatgctttttcttcaggtactctcgtcagtccacacgtggacctcattgctccctgacaaaagaggtttacgacccatagggccttattccctcacgcgacttggctggttcagccttccggccattgaccaatattcctca

SEQ ID NO: 176 - DNA, 404 bp, Unknown
  source: /note=“Description of Unknown 16S rRNA sequence”
cctgtttgctccccacgctttcgcgcctcagcgtcagttacggcccagcaagtcgccttcgccactggtgttcctcctaatatctacgcatttcaccgctacactaggaattccacttgcctcttccgcactcaagtctgccagtatcggatgcacgttaagagttgagcccttacatttcacatccgacttaacagaccgcctacgcgccctttacgcccagtgattccggacaacgcttgccccctatgttttaccgcggctgctggcacgtagttagccggggcttcctcctcaagtaccgtcattatcttccttgaggacagagctttacgacccgaaggccttcatcgctcacgcggcgttgctgcatcaggctttcgcccattgtgcaatattcccca

SEQ ID NO: 177 - DNA, 429 bp, Unknown
  source: /note=“Description of Unknown 16S rRNA sequence”
cctgtttgctccccacgctttcgtgcatgagcgtcagtgacatcccaggaggctgccttcgccattggtgttcttccaaatatctacgcatttcactgctacacttggaattccacctccctctgatggactctagtcctgcagtcacgaatgcaattcccaggttgagcccggggatttcacatccgtcttacagaaccgcctgcgcacgctttacgcccagtaattccgattaacgcttgcaccctacgtattaccgcggctgctggcacgtagttagccggtgcttattcttcaggtaccgtcagcagatcccggtattagcggaatccttttcgtccctgacaaaagcagtttacaatccgaagaccttcatcctgcacgcggcatggctggatcagggttgcccccattgtccaaaattcccca

SEQ ID NO: 178 - DNA, 405 bp, Unknown
  source: /note=“Description of Unknown 16S rRNA sequence”
cctgttcgctccccacgctttcgcgcctgagcgtcagtatccggctagcagaccgccttcgcctctggtattcctcctgatctctgcgcatttcaccgctacaccaggaattctgtctgccccgccggcactctagttgcccggtccgcgatgaccggccggggttgagccccggactttcacaccgcgcctaaacaaccgcctgcgcgccctttacgcccaatcaatccggataacgctcgccccctacgtattaccgcagctgctggcacgtagttggctggggcttgctcgccgggtaccgtcttaactctttcccggcaacagggctttacaggacgaatcccgtcttcacccacgcggcattgctgcgtcagagtttcctccattgcgcaatattcccca

SEQ ID NO: 179 - DNA, 424 bp, Unknown
  source: /note=“Description of Unknown 16S rRNA sequence”
cctgtttgatccccacgctttcgtgcatcagcgtcagtcatggcttggcaggctgccttcgcaatcggggttctgcgtgatatctaagcatttcaccgctacaccacgcattccgcctgcctcaaacatactcaagcctcccagtttcaacggcaattctatggttgagccacagactttcaccgctgacttaaaaggccgcctacgcaccctttaaacccaataaatccggataacgctcggatcctccgtattaccgcggctgctggcacggagttagccgatccttattcataaggtacatacaaaacgggacacgtcccgcactttattcccttataaaagaggtttacgatccatagaaccttcatccctcacgcgacttggctggttcaggctcgcgcccattgaccaatattcctca

SEQ ID NO: 180 - DNA, 406 bp, Unknown
  source: /note=“Description of Unknown 16S rRNA sequence”
cctgtttgctacccacgctttcgcgcttcagcgtcagtatctgtccagtgggctgacttctccatcggcattcctacaaatatctacgaatttcacctctacacttgtagttccgcccacctctccagtactctagtttgacagtttccaacgcaatacggagttgagccccgcattttcacatcagacttaccaaaccacctagacgcgctttacgcccaataaatccggataacgcttgcgacatacgtattaccgcggctgctggcacgtatttagccgtcgcttcttctgttggtaccgtcacgttcttcgtcccaactgaaagcactttacattccgaagaccttcatcctgcacgcggcatggctggatcagggttgcccccattgtccaaaattcccca

SEQ ID NO: 181 - DNA, 424 bp, Unknown
  source: /note=“Description of Unknown 16S rRNA sequence”
cctgttcgatccccgcactttcgagcttcagcgtcagttgcgctccagtgagctgccttcgcaatcggagttcttcgtgatatctaagcatttcaccgctacaccacgaattccgcccactttgtgcgtactcaaggaaaccagttcgcgctgcagtgcagatgttgagcatctacatttcacaacacgcttaatctccggcctacgctccctttaaacccaataaatccggataacgcccggaccttccgtattaccgcggctgctggcacggaattagccggtccttattcataaggtacatacaaaaagagtcacgactctcactttattcccttataaaagcagtttacaacccatagggccgtcttcctgcacgctacttggctggttcaggctctcgcccattgaccaatattcctca

SEQ ID NO: 182 - DNA, 422 bp, Unknown
  source: /note=“Description of Unknown 16S rRNA sequence”
cctgttcgctccccacgctttcgctcctcagcgtcagtgacggcccagagacctgccttcgccattggtgttcttcccgatatctacacattccaccgttacaccgggaattccagtctcccctaccgcactccagcccgcccgtacccggcgcagatccaccgttaggcgatggactttcacaccggacgcgacgaaccgcctacgagccctttacgcccaataaatccggataacgctcgcaccctacgtattaccgcggctgctggcacgtagttagccggtgcttattcgaacaatccactcaacacggcccgaagccgtgccttgcccttgaacaaaagcggtttacaacccgaaggcctccatcccgcacgcggcgtcgctgcatcaggcttgcgcccattgtgcaatattcccca

SEQ ID NO: 183 - DNA, 405 bp, Unknown
  source: /note=“Description of Unknown 16S rRNA sequence”
cctgttcgctcccccagctttcgcgcctcagcgtcagtgccggcccagcaggctgccttcgccgtcggtgttcttcccgatatctgcgcatttcaccgctacaccgggaattccgcctgcccctaccgcactcgatccccccagttcggcccgcttccgggggttgagcccccggcttcgacggtgccgcttgaggagacgcctacgcgcgctttacgcccaatgaatccggataacgctcgccccctacgtattaccgcggctgctggcacgtagttagccggggcttcttctgcaggtaccgtcaaaattcttccctgccgaaagcggtttacgacccgaaggccttcgtcccgcacgcggcgttgctgcgtcagggtttcccccattgcgcaaaattcccca

SEQ ID NO: 184 - DNA, 404 bp, Unknown
  source: /note=“Description of Unknown 16S rRNA sequence”
cctgttcgctccccacgctttcgcacctcagcgtcagtataagcccagcaggccgcctccgccaccggtattcctcctgatctctgcgcatttcaccgctacaccaggaattctgcctgcctctgcttcactccagcagcacggtttgccgtgcaacccggggttgggccccgggttttaacaccgcaccttcgctgccgcctgcgtgccctttacgcccaataaatccggataacgctcgccccccacgtgttaccgcagctgctggcacgtggttggctggggctcatacgccgggtaccgtcttaactcttccccgacaacagggctttacaggacgaatcccttcttcacccacgcggcattgctgcgtcagagttgcctccattgcgcaatattcccca

SEQ ID NO: 185 - DNA, 424 bp, Unknown
  source: /note=“Description of Unknown 16S rRNA sequence”
cctgtttgatacccacactttcgagcatcagcgtcagttacaatccagtaagctgccttcgcaatcggagttcttcgtgatatctaagcatttcaccgctacaccacgaattccgcctacctctgttgcactcaaggtcgccagtatcaactgcaattttacggttgagccgcaaactttcacaactgacttaacaacccgcctacgctccctttaaacccaataaatccggataacgctcggatcctccgtattaccgcggctgctggcacggagttagccgatccttattcatacggtacatacaaaaagccacacgtggctaactttattcccgtataaaagaagtttacaacccatagggcagtcatccttcacgctacttggctggttcagactctcgtccattgaccaatattcctca

SEQ ID NO: 186 - DNA, 404 bp, Unknown
  source: /note=“Description of Unknown 16S rRNA sequence”
cctgtttgctccccacgctttcgagcctcagcgtcagttacagtccagagagtcgccttcgccactggtgttcttcctaatctctacgcatttcaccgctacactaggaattccactctcccctcctgtactctagatccccagtttggaatgcagcccccaggttgagcccgagtatttcacatcccacttaaagatccgcctacgctccctttacgcccagtaaatccggacaacgcttgccacctacgtattaccgcggctgctggcacgtagttagccgtggcttcctccttaggtaccgtcattatcgtccctaaagacagagctttacaatccgaagaccgtcatcactcacgcggcgttgctgcatcagggtttcccccattgtgcaatattcccca

SEQ ID NO: 187 - DNA, 404 bp, Unknown; identical to SEQ ID NO: 26
  source: /note=“Description of Unknown 16S rRNA sequence”

SEQ ID NO: 188 - DNA, 404 bp, Unknown
  source: /note=“Description of Unknown 16S rRNA sequence”
cctgtttgctccccacgctttcgagcctcaacgtcagttaccgtccagtaagccgccttcgccactggtgttcctcctaatatctacgcatttcaccgctacactaggaattccgcttacccctccggtactcaagactgacagtttccaatgcagtccaggggttgagcccctgcctttcacatcagacttgccattccgtctacgctccctttacacccagtaaatccggataacgcttgccccctacgtattaccgcggctgctggcacgtagttagccggggcttcttagtcaggtaccgtcattttcttccctgctgatagaagtttacataccgaaatacttcttcgctcacgcggcgtcgctgcatcagggtttcccccattgtgcaatattcccca

SEQ ID NO: 189 - DNA, 425 bp, Unknown
  source: /note=“Description of Unknown 16S rRNA sequence”
cctgtttgatacccacactttcgagcatgaacgtcagttacactttagcaagctgccttcgcaatcggagttcttcgtgatatctaagcatttcaccgctacaccacgaattccgcctgcctcaactgcactcaaggtctccagtatcaactgcaattttaaggttgagccccaaactttcacagctgacttaaaaacccgtctgcgctccctttaaacccaataaatccggataacgctcgcatcctccgtattaccgcggctgctggcacggagttagccgatgcttattcgtaccggtacatacaagcgcctacacgtaggcggttttattcccggacaaaagaagtttacaacccatagggcagtcatccttcacgctacttggctggttcaggctcgcgcccattgaccaatattcctca

SEQ ID NO: 190 - DNA, 404 bp, Unknown
  source: /note=“Description of Unknown 16S rRNA sequence”
cctgtttgctacccatgctttcgagcctcagcgtcagttagtgcccagtaggccgccttcgccactggtgttcctcccgatatctacgcattccaccgctacaccgggaattccgcctacctctacaccactcaagatccgcagttttgaaagcagttcatgggttgagcccatgagtttcacttccaacttgcagacccgcctgcgctccctttacacccagtaattccggacaacgcttgccacctacgttttaccgcggctgctggcacgtagttagccgtggctttcttgtaaggtaccgtcactatcgtcccttacgacaggagtttacaacccgaaggccttcttcctccacgcggcgtcgctgcatcagggtttcccccattgtgcaatattcccca

SEQ ID NO: 191 - DNA, 424 bp, Unknown; identical to SEQ ID NO: 11
  source: /note=“Description of Unknown 16S rRNA sequence”

SEQ ID NO: 192 - DNA, 404 bp, Unknown
  source: /note=“Description of Unknown 16S rRNA sequence”
cctgtttgctccccacgctttcgagcctcaacgtcagtcatcgtccagcaagccgccttcgccactggtgttcctcctaatatctacgcatttcaccgctacactaggaattccgcttgcctctccgacactctagctgcacagtttccaaagcagtcccggggttgagccccgggctttcacttcagacttgcactgccgtctacgctccctttacacccagtaaatccggataacgcttgccccctacgtattaccgcggctgctggcacgtagttagccggggcttcttagtcaggtaccgtcattttcttccctgctgatagaagtttacataccgaaatacttcatccttcacgcggcgtcgctgcatcagggtttcccccattgtgcaatattcccca

SEQ ID NO: 193 - DNA, 407 bp, Unknown
  source: /note=“Description of Unknown 16S rRNA sequence”
cctgtttgctccccacgctttcgcgcctcagcgtcagttatcgtccagcaatccgccttcgccactggtgttcctccgtatatctacgcatttcaccgctacacacggaattccgattgcctctccgacactcaagaactacagtttcaaatgcaggctatgggttgagcccatagttttcacacctgacttgcagtcccgcctacacgccctttacacccagtaaatccggataacgctcgccacctacgtattaccgcggctgctggcacgtagttagccgtggcttattcaacaggtaccgtcatttgtttcgtccctgttaaaagaagtttacaacccgaaagccttcttccttcacgcggcgttgctgggtcaggcttgcgcccattgcccaatattcccca

SEQ ID NO: 194 - DNA, 429 bp, Unknown
  source: /note=“Description of Unknown 16S rRNA sequence”
cctatttgctccccacgctttcgtgcctcagcgtcaggtacaggccaggcggccgccttcgccgctggtgttcttccacatctctacgcattttaccgctacacatggagttccaccgccctctcctgtcctcaagccgtgcagtttccaaagcctgaaccggttgagccggtcccttttacttcagacttgcacagccgcctgcgcaccctttacgcccaatcattccggataacgcttgccacctacgtattaccgcggctgctggcacgtagttagccgtggctttctggccgggtaccatcaaacagtcagctttccactctggctgtcctttgtccccggcaacagggctttacaacccgaaggccgtcatcccgcacgcggcgtcgctgcgtcaggctttcgcccattgcgcaagattcccca

SEQ ID NO: 195 - DNA, 410 bp, Unknown
  source: /note=“Description of Unknown 16S rRNA sequence”
cctgtttgctacccacgctttcgtgcctcaacgtcagtacaggcccagcaagccgccttcgccactggtgttcctccatatatttacgcattttaccgctacacatggaattccacttgcctctaccgtactctagcttaagagtttcttttgctcgattgggttgagcccaacaattttacaaaagacttcttatgccgtctacgcaccctttacgcccaataaatccggataacgctcgccccctacgtattaccgcggctgctggcacgtagttagccggggcttattcattaggtaccgtcaatacgagttttcgtccctaataaaagaactttacattacgaataacttcttcgttcacgcggcattgctcggtcagactttcgtccattgccgaaaattcccta

SEQ ID NO: 196 - DNA, 404 bp, Unknown
  source: /note=“Description of Unknown 16S rRNA sequence”
cctgtttgctccccacgctttcgtgcctcagcgtcagtatcgggccagtgagccgccttcgccactggtgttcttgcgaatatctacgaatttcacctctacactcgcagttccactcacctctcccgaactcaagatcttcagtatcaaaggcagttctggagttgagctccaggatttcacccctgacttaaagacccgcctacgcaccctttacgcccagtgattccgagcaacgctagcccccttcgtattaccgcggctgctggcacgaagttagccggggcttattcttgcggtaccgtcattatcttcccgcacaaaagagctttacaaccctagggccttcatcactcacgcggcatggctggatcaggcttgcgcccattgtccaatattcccca

SEQ ID NO: 197 - DNA, 404 bp, Unknown
  source: /note=“Description of Unknown 16S rRNA sequence”
cctgttcgctccccacgctttcgcacctgagcgtcagtcactggccggcagaccgccttcgcctccggtattcctcctgatctctgcgcatttcaccgctacaccaggaattctgtctgcctctccagcactcgagacctccggttcgccgtggcctcccggggttgagccccggtctttaacacggcgcctgaaagtccgcctgcgtgccctttacgcccaataaatccggataacgctcgccccccacgtattaccgcagctgctggcacgtggttggctggggcttgctcgtcaggtaccgtcttcttcttccctgacaacagagctttacggaacgaatcccttcttcactcacgcggcattgctgcgtcagagtttcctccattgcgcaatattcccca

SEQ ID NO: 198 - DNA, 423 bp, Unknown
  source: /note=“Description of Unknown 16S rRNA sequence”
cctgtttgatacccacgctttcgtgcctcagcgtcaatcgcggattcgtcagatgccttcgcaatcggtgttctgtgtgatatctatgcatttcaccgctacaccacacattcctccaacgacatccgcattcaagcctaccagtatcaacggcagtttcctcgttgagcgaggacatttcaccgctgacttaacaggccgcctacgcaccctttaaacccaataaatccggataacgcttgcatcccccgtattaccgcggctgctggcacggagttagccgatgcttattcgtgagatactctcatcaatccactcgtggaccttattgctctctcacaaaagaagtttaacgcccatagggcactgaccttcacgcggcatggctggttcagcctttcggccattgaccaatattcctca

SEQ ID NO: 199 - DNA, 403 bp, Unknown
  source: /note=“Description of Unknown 16S rRNA sequence”
cctgtttgctccccacgctttcgagcctcagcgtcagtaaaagcccagcaggccgccttcgccactggtgttcctcctaatatctacgcatttcaccgctacactaggaattccgcctgcctctacttcactcaagagccccagtttcgaacgcagctaccggttgagccggtagatttaacattcgacttaagaccccgcctacgctccctttacacccagtaattccggacaacgctcgctccctacgtattaccgcggctgctggcacgtagttagccggagcttcctccttgggtaccgtcattatcgtccccaaagacagaggtttacaatccgaagaccgtcttccctcacgcggcgtcgctgcatcagagtttcctccattgtgcaatatccccca

SEQ ID NO: 200 - DNA, 404 bp, Unknown
  source: /note=“Description of Unknown 16S rRNA sequence”
cctgtttgctcccccagctttcgcgcctcagcgtccgtctcggcccagagggccgccttcgccaccggtgttccacccgatatctgcgcattccaccgctacaccgggtgttccaccctcccctaccggacccgagccgcggggttcggggggcggaccggggttgagccccgggatttgaccccccgcctacgcggccgcctacgcgcgctttacgcccaatgaatccggataacgcgcgccccctacgtattaccgcggctgctggcacgtagttagctggggcttcttctgcaggtaccgtcttgtctcgtccctgctgaaagcggtttacgacccgaaggccttcgtcccgcacgcggcgtcgctgcgtcagggtttcccccattgcgcaagattcccca

SEQ ID NO: 201 - DNA, 428 bp, Unknown
  source: /note=“Description of Unknown 16S rRNA sequence”
cctatttgctccccacgctttcgtgcctgagcgtcagttacagtccaggcggccgccttcgccactggtgttcttccatatatctacgcattttaccgctacacatggagttccaccgccctctcctgtcctctagcgtgacagtttccaaagcctgtacaggttgagcccgtacctttcacttcagacttgtcacgccgcctgcgcaccctttacgcccaatcattccggataacgctcgccacctacgtattaccgcggctgctggcacgtagttagccgtgactttctggcaggctaccatcaaaagacaagcatttcctcttgccttcctttttgacctgcaacagagctttacgatccgaagaccttcctcactcacgcggcattgctcgttcaggcttgcgcccattgacgaaaattcccta

SEQ ID NO: 202 - DNA, 404 bp, Unknown; identical to SEQ ID NO: 12
  source: /note=“Description of Unknown 16S rRNA sequence”

SEQ ID NO: 203 - DNA, 404 bp, Unknown
  source: /note=“Description of Unknown 16S rRNA sequence”
cctgtttgctccccacgctttcgagcctcagtgtcagttacagtccagtaagccgccttcgccgctggtgttcctcctaatatctacgcatttcaccgctacactaggaattccacttacctctcctgcactccagcccagcagtttcaaaagcagtcccggggttgagccccgggctttcacttctgacttgctgtgccacctacactccctttacacccagtaaatccggataacgcttgctccatacgtattaccgcggctgctggcacgtatttagccggagcttcttagtcaggtaccgtcattatcttccctgctgatagagctttacataccgaaatacttcttcactcacgcggcgtcgctgcatcagagtttcctccattgtgcaatattcccca

SEQ ID NO: 204 - DNA, 404 bp, Unknown
  source: /note=“Description of Unknown 16S rRNA sequence”
cctgtttgctccccacgctttcgcgcatcagcgtcagtaatgttccagcaggtcgccttcgcaatgagtattcctcttgatctctacggattttacccctacaccaagaattccacctacctctcccatactctagaaaagtagtttcaaatgcagttctgtagttaagccacaggatttcacatctgacttaccttcccgcctacgcgctctttacgcccagtgattccgagtaacgcttgcaccctccgtattaccgcggctgctggcacggagttagccggtgcttattcgttagataccgtcataatcttctctaacaaaaggagtttacaatcctaaaaccttcatcctccacgcggcgttgctgcttcagggtttcccccattgagcaatattcccta

SEQ ID NO: 205 - DNA, 428 bp, Unknown
  source: /note=“Description of Unknown 16S rRNA sequence”
cctatttgctccccacgctttcgtgcctcagtgtcagttacaggccaggcgaccgccttcgccactggtgttcctccatatatctacgcattttaccgctacacatggaattccatcgccctctcctgcactctagttcaccagtttccaaagcgaacagcagttgagctgctgccttttacttcagacttaatgtaccacctacgcaccctttacgcccaatcattccggataacgcttgccacctacgtattaccgcggctgctggcacgtagttagccgtggctttctggtaagataccatcactatcatgtcattccctacatgatccgtttttctcttacaacagagctttacgatccgaagaccttcttcactcacgcggcattgctcgttcagggttccccccattgacgaaaattcccta

SEQ ID NO: 206 - DNA, 429 bp, Unknown
  source: /note=“Description of Unknown 16S rRNA sequence”
cctgtttgctccccacgctttcgcacctgagcgtcagtcttcgtccagggggccgccttcgccaccggtattcctccagatctctacgcatttcaccgctacacctggaattctacccccctctacgagactcaagcttgccagtatcagatgcagttcccaggttgagcccggggatttcacatctgacttaacaaaccgcctgcgtgcgctttacgcccagtaattccgattaacgcttgcaccctccgtattaccgcggctgctggcacggagttagccggtgcttcttctgcgggtaacgtcaatgagcaaaggtattaactttactcccttcctccccgctgaaagtactttacaacccgaaggccttcttcatacacgcggcatggctgcatcaggcttgcgcccattgtgcaatattcccca

SEQ ID NO: 207 - DNA, 408 bp, Unknown
  source: /note=“Description of Unknown 16S rRNA sequence”
cctgttcgctccccacgctttcgtgcctcagcgtcagtttccagtccagaaagccgccttcgccactggtgttcttcctaatatctacgcatttcaccgctacactaggaattccgctttcctctcctgtactctagcttagcagtttaaaatgcaatcccgaggttaagcctcgggctttcacatcttacttgccatgccgcctactcaccctttacacccagtaattccggataacgcttgccccctacgtattaccgcggctgctggcacgtagttagccggggcttcttatttaggtacagtcactttcttcgtccctattgatagaagtttacaatccgaaaaccgtcttccttcacgcggcgttgctgcatcagggtttcccccattgtgcaatattcccca

SEQ ID NO: 208 - DNA, 424 bp, Unknown
  source: /note=“Description of Unknown 16S rRNA sequence”
cctgtttgatacccacactttcgagcctcaatgtcagttacagcttagcaggctgccttcgcaatcggagttcttcgtgatatctaagcatttcaccgctacaccacgaattccgcctgcctcaactgcactcaaggcgtccagtatcaactgcaattttacggttgagccgcaaactttcacaactgacttaaacgcccatctacgctccctttaaacccaataaatccggataacgctcggatcctccgtattaccgcggctgctggcacggagttagccgatccttattcatacggtacatacaaaaaggtacgcgtacctcactttattcccgtataaaagaagtttacaacccatagggcagtcatccttcacgctacttggctggttcagactctcgtccattgaccaatattcctca

SEQ ID NO: 209 - DNA, 429 bp, Unknown
  source: /note=“Description of Unknown 16S rRNA sequence”
cccgttcgctaccctagctttcgagcctcagcgtcagttacagtccagaaagccgccttcgccactggtgttcttcccaatatctacgcatttcaccgctacactgggaattccgctttcctctcctgcactcaagaaaatcagttcggaccccatcacgaggttgagccccgcacttttaagatccgcttaatttcccgcctgcgctccctttacgcccaataattccggacaacgcttgccacctacgtattaccgcggctgctggcacgtagttagccgtggcttcctcgttaagtaccgtcaaatacttactgtattataataagcatccttcgcccttaacaacagaactttacgatccgaagaccttcttcgttcacgcggcgttgctccgtcaggctttcgcccattgcggaagattcccca

SEQ ID NO: 210 - DNA, 404 bp, Unknown
  source: /note=“Description of Unknown 16S rRNA sequence”
cctgttcgctcccccagctttcgcgcctcagcgtcggtctcggcccagagggccgccttcgccaccggtgttccacccgatatctgcgcattccaccgctacaccgggtgttccaccctcccctaccggacccgagcccggcggttcagggggcgggacggggttgagccccgccatttgaccccctgcctgccgggccgcctacgcgcgctttacgcccaatgaatccggataacgctcgccccctacgtattaccgcggctgctggcacgtagttagccggggcttcttctgcaggtaccgtcttgtctcatccctgctgaaagcggtttacgacccgagggccttcgtcccgcacgcggcgtcgctgcgtcagggttccccccattgcgcaagattcccca

SEQ ID NO: 211 - DNA, 429 bp, Unknown
  source: /note=“Description of Unknown 16S rRNA sequence”
cccgttcgctaccctagctttcgagcctcagcgtcagttacagtccagaaagccgccttcgccactggtgttcttcccaatatctacgcatttcaccgctacactgggaattccgctttcctctcctgcactcaagaaaaccagttcggaccccatcacggggttgagccccgcacttttaagatccgcttagtttcccgcctgcgctccctttacgcccaataattccggacaacgcttgccgcctacgtattaccgcggctgctggcacgtagttagccgtggcttcctcgttaggtaccgtcaaataaaaaccttattatgattcttacccttcgtccctaacaacagaactttacgatcctaagaccttcatcgttcacgcggcgttgctccgtcaggctttcgcccattgcggaagattcccca

SEQ ID NO: 212 - DNA, 403 bp, Unknown
  source: /note=“Description of Unknown 16S rRNA sequence”
cctgtttgctacccatgctttcgagcctcagcgtcagttaaagcccagtaggccgccttcgccactggtgttccttccgatatctacgcattccaccgctacaccggaaattccgcctacctctacttcactcaagttccacagttttgaacgctgatacgggttgagcccgtagattaaacgtccaacttgcagaaccgcctgcgctccctttacacccagtaattccggacaacgctcgcaccctacgttttaccgcggctgctggcacgtagttagccggtgcttcctcctcgggtaccgtcattatcgtccccgaagacagaggtttacaatacgaataccttcttccctcacgcggcgtcgctgcatcagagtttcctccattgtgcaatattcccca

SEQ ID NO: 213 - DNA, 428 bp, Unknown
  source: /note=“Description of Unknown 16S rRNA sequence”
cctgtttgctccccacgctttcgtgcctcagcgtcagctccagcccagacggccgccttcgccgctggtgttcttccatatctctatgcattttaccgctacacatggagttccgccgtcctctgctggcctccagcctgccagtttccacagcctggcaaagttgagcttcgcccttttactgcagacttaacaggccgcctgcgcaccctttacgcccaatcattccggataacgctcgccacctacgtattaccgcggctgctggcacgtagttagccgtgacttcctcgtggacttccatcaaactaaagccatttcctgctcttgtcctttttcatccacaacagggctttacactccgaaaagctccttcacccacgcggcatcgctcgttcaggcttgcgcccattgacgaagattcccta

SEQ ID NO: 214 - DNA, 428 bp, Unknown
  source: /note=“Description of Unknown 16S rRNA sequence”
cctatttgctccccacgctttcgtgcctgagcgtcagttacaggccaggcggccgccttcgccactggtgttcttccacatctctacgcattttaccgctacacgtggagttccaccgccctctcctgtcctcgagcataccagtttccaaagcctgtacaggttgagcccgtacctttcacttcagacttgatatgccgcctgcgcaccctttacgcccaatcattccggataacgctcgccacctacgtattaccgcggctgctggcacgtagttagccgtgactttctggcgaggtaccatcaaaagagaatcattccctcttctcttcctttttccctcgcaacagagctttacgatccgaagaccttcttcacccacgcggcattgctcgttcaggcttgcgcccattgacgaaaattcccta

SEQ ID NO: 215 - DNA, 423 bp, Unknown
  source: /note=“Description of Unknown 16S rRNA sequence”
cctatttgctccccacgctttcgtgcctgagcgtcagttacagactagcaagccgccttcgccaccggtgttcctccatatatctatgcattttaccgctacacatggaattccacttgccccttctgcactctagttcaccagtttctaagcctggatggggttgagccccacaatttaagcttaaacttagcaaaccgcctgcgcaccctttacgcccaataattccggataacgcttgtcaccttcgtattaccgcggctgctggcacgaagttagccgtgactttctggtaaagtaccgtcagttacatagttgatatgtaatattcttcctttacaacagagctttacaatccgaagaccttcatcactcacgcggcattgctcggtcaggctttcgcccattgccgaaaattcccta

SEQ ID NO: 216 - DNA, 428 bp, Unknown
  source: /note=“Description of Unknown 16S rRNA sequence”
cctgtttgctccccacgctttcgtgcctcagcgtcagctccagcccagacggccgccttcgccgctggtgttcttccatatctctatgcattttaccgctacacatggagttccgccgtcctctgctggcctccagcctgccagtttccacagcctggcaaagttgagcttcgcccttttactgcagacttaacaggccgcctgcgcaccctttacgcccaatcattccggataacgctcgccacctacgtattaccgcggctgctggcacgtagttagccgtgacttcctcgtggacttccatcaaaagcagaccatttcctgtctcctccctttttcatccacaacagggctttacactccgaaaagctccttcacccacgcggcatcgctcgttcaggcttgcgcccattgacgaagattcccta

SEQ ID NO: 217 - DNA, 429 bp, Unknown
  source: /note=“Description of Unknown 16S rRNA sequence”
cctatttgctccccacgctttcgtgcctcagcgtcaggtacaggccaggcggccgccttcgccgctggtgttcttccacatctctacgcattttaccgctacacatggagttccaccgccctctcctgtcctcaagccgtgcagtttccaaagcctgaaccggttgagccggtcccttttacttcagacttgcacagccgcctgcgcaccctttacgcccaatcattccggataacgcttgccacctacgtattaccgcggctgctggcacgtagttagccgtggctttctggccgggtaccatcaaacagtcagctttccactctggctatcctttgtccccggcaacagggctttacagcccgaaggccgtcttcactcacgcggcattgctcgttcagagtttcctccattgacgaaaattcccta

SEQ ID NO: 218 - DNA, 423 bp, Unknown
  source: /note=“Description of Unknown 16S rRNA sequence”
cctgttcgatacccacgctttcgtgcttcagcgtcagtagggcgccggtatgctgccttcgcaatcggagttctgcgtgatatctatgcatttcaccgctacaccacgcattccgcatacttctcgcccactcaagatctccagtttcaacggcgggatgaggttgagcctcactattttaccgctgacttaaaaatccgcctacgcaccctttaaacccaataaatccggataacgctcgcatcctccgtattaccgcggctgctggcacggagttagccgatgctttttcttcgggtaatctcgcttcaggcacacgtgccttcgttgctccccgacaaaagaggtttacaatccataggaccgtcttccctcacgcgacttggctggttcaggctctcgcccattgaccaatattcctca

SEQ ID NO: 219 - DNA, 404 bp, Unknown
  source: /note=“Description of Unknown 16S rRNA sequence”
cctgtttgctccccacgctttcgagcctcagcgtcatttacagtccagtaagccgccttcgccactggtgttcctcctaatatctacgcatttcaccgctacactaggaattccacttacctctcctgcaatctagcaatacagtttcaaaagcagtcccggagttgagccccgggctttcacttctgacttgcatcgccgcctacgctccctttacacccagtaaatccggataacgcttgccccctacgtattaccgcggctgctggcacgtagttagccggggcttcttagtcaggtaccgtcattttcttccctgctgatagaagtttacataccgaaatacttcttccttcacgcggcgtcgctgcatcagggtttcccccattgtgcaatattcccca

SEQ ID NO: 220 - DNA, 429 bp, Unknown
  source: /note=“Description of Unknown 16S rRNA sequence”
cctgtttgctacccacgctttcgcacctcagcgtcagttaccgaccagattgccgccttcgctaccggtattcctcctgatatctacgcatttcaccgctacaccaggaattctgcaatcctctccgatactctagtattatagtatcactagccttactaaagttgagctctagcctttgactaatgacttacaacaccgcctacgtgctctttacgcccagtcactccgaacaacgctcgccccctccgtattaccgcggctgctggcacggagttagccggggctgcttatacaggtaccgtcactatatatgcagttactcatatatattttcttccctgtcgaaagaactttacgacccgaaggccttcttcattcacgcggcgtcgctgcgtcactctatcgagcattgcgcaaaattcccca

SEQ ID NO: 221 - DNA, 405 bp, Unknown
  source: /note=“Description of Unknown 16S rRNA sequence”
cctgtttgctccccacgctctcgcacctgagcgtcagtatctgtccggcaggccgccttcgcctctggtattcctcctgatatctgcgcatttcaccgctacaccaggaattctgcctgcctctccagtactctagccgcatggttcgggatgcccttccagggttgggccctgatctttcacatcccgcctacacggccgcctgcgtgccctttacgcccagtcattccggacaacgctcgccccctacgtgttaccgcggctgctggcacgtagttagccggggcttgcttgttaggtaccgtcttctctcttccctaacggcagagctttacggaacgaattccttcttcactcacgcggcattgctgcgtcagagttgcctccattgcgcaatattcccca

SEQ ID NO: 222 - DNA, 412 bp, Unknown
  source: /note=“Description of Unknown 16S rRNA sequence”
cctgtttgctccccacgctttcgcacctcagcgtcagtatctgcccaggaaatcgccttcgcctctggtgttcttccagatatctacgcatttcaccgctacacctggaattccatttccctctgcaatactctagatagttagtttaaagtgcagttcccaggttgagcccggggctttcacaccttacttgactatccgcctacatgccctttacgcccagttattccgattaacgcttgcaccctccgtattaccgcggctgctggcacggagttagccggtgcttcttctgtgggtaacgtcactcttgcgatttcctccccactgaaagtgctttacaacccgaaggccttcttcacacacgcggcatggctgcatcagggttccccccattgtgcaatattcccta

SEQ ID NO: 223 - DNA, 402 bp, Unknown
  source: /note=“Description of Unknown 16S rRNA sequence”
cctgtttgctccccacgctctcgcgcctgagcgtcagtctctctccagcaggccgccttcgcctccggtattcctcctgatctctgcgtatttcaccactacaccaggaattctgcctgcctctagagtactctagctaagctggttctttctgcttccccggggtgggcccggggcttttacaaaaagcctctcttgccgcctgcgcgccctttacgcccaataattccggacaacgctcgccccctacgtattgccgcggctgctggcacgtagttagccggggctcaaaaccggctggtcttctctcctaaccggtgactgagctttacgggacgaatcccttcctcactcacgcggcatcgctgcgtcagagtttcctccattgcgcaatattcccca

SEQ ID NO: 224 - DNA, 404 bp, Unknown
  source: /note=“Description of Unknown 16S rRNA sequence”
cctgtttgctccccacgctttcgagcctcaacgtcagttaccgtccagtaagccgccttcgccactggtgttcctcctaatatctacgcatttcaccgctacactaggaattccgcttacctctccggcactcgagtatcacagtttccaatgcagtccaggggttgagcccccgcctttcacatcagacttgcaacaccgtctacgctccctttacacccagtaaatccggataacgcttgccccctacgtattaccgcggctgctggcacgtagttagccggggcttcttagtcaggtaccgtcattttcttccctgctgatagaagtttacataccgagatacttcttccttcacgcggcgtcgctgcatcagggtttcccccattgtgcaatattcccca

SEQ ID NO: 225 - DNA, 407 bp, Unknown
  source: /note=“Description of Unknown 16S rRNA sequence”
cctgtttgctccccacgctttcgcgcctcagcgtcagttaatgtccagcaggccgccttcgccactggtgttcctccgtatatctacgcatttcaccgctacacacggaattccgcctgcctctccatcactcaagaaaaacagtttcaaatgcaggccagtggttgagccactgggtttcacatctgacttgctttcccgcctacacgccctttacacccagtaaatccggataacgcttgctccctacgtattaccgcggctgctggcacgtagttagccggagcttattcattaagtaccgtcatttgtttcgtccttaataaaagaagtttacaacccgaaagccttcttccttcacgcggcgttgctgggtcaggcttgcgcccattgcccaatattcccca

SEQ ID NO: 226 - DNA, 403 bp, Unknown
  source: /note=“Description of Unknown 16S rRNA sequence”
cctgttcgctccccacgctttcgcgcctgagcgtcagtcgccagccagcaggccgccttcgcctccggtgttcctcctgatctctgcgcatttcaccgctacaccaggaattccgcctgcctctctggcactcgagccgcccggttccgccggcagccgccgggttgggcccggcggtttcaccggcggcctaaacggccgcctgcgcgccctttacgcccaatgattccggacaacgcttgccccccacgtcttaccgcagctgctggcacgtggttggctggggctcctggccgggtaccgtcattgtcttccccggcgacagagctttacggaacgagtcccttcttcgctcacgcggcattgctgcgtcagggtctcccccattgcgcaatattcccca

SEQ ID NO: 227 - DNA, 407 bp, Unknown
  source: /note=“Description of Unknown 16S rRNA sequence”
cctgtttgctccccacgctttcgcgcctcagcgtcagttgctgtccagttggccgccttcgccactggtgttcctcctaatatctacgcatttcaccgctacactaggaattccgccaacctctccagtactcaagaaccacagtttcaaacgcaggccagaggttgagcccctggttttcacgcctgacttgcagtcccgcctacacgccctttacacccagtaaatccggacaacgcttgccacctacgtattaccgcggctgctggcacgtagttagccgtggcttattcatcaggtacagtctcattcatcttccctgataaaagaagtttacaacccgaaagccttcttccttcacgcggcgttgctgggtcaggcttgcgcccattgcccaatattcccca

SEQ ID NO: 228 - DNA, 403 bp, Unknown
  source: /note=“Description of Unknown 16S rRNA sequence”
cctgtttgctacccacgctttcgtgcctcagtgtcagttgcagtccagaaagccgccttcgctactggtgttcttcccaatatctacgcatttcaccgctacactgagaattccactttcctctcctgcactcaagtcttccagtttcggcggcttaatacggttgagccgtagcctttaaccaccgacttgaaagaccacctacgcaccctttacgcccagtaaatccggataacgctagcaccctacgtattaccgcggctgctggcacgtagttagccggtgcttcctcacagggtaccgtcattatcttcccctgcaacagaactttacgatccgaagaccttcatcgttcacgcggcgttgctgcatcaggctttcgcccattgtgcaatatccccca

SEQ ID NO: 229 - DNA, 410 bp, Unknown
  source: /note=“Description of Unknown 16S rRNA sequence”
cctgtttgctcccctagctttcgcgcctcagcgtcagtgatggcccagaaggccgccttcgccaccggtgttcttcccgatatctgcgcattccaccgctacaccgggaattccgccttcccctaccacactcaagccagccggtatcgggagcgagacggggttgagccccgaaatttgactcccgacctaactggccgcctacgcgcgctttacgcccaatgaatccggataacgctcgccccctacgtattaccgcggctgctggcacgtagttagccggggcttcttctgcaggtaccgtcaccttgcggcctcgtccctgctgaaagcggtttacaacccgaaggccgtcatcccgcacgcggcgtcgctgcgtcaggctttcgcccattgcgcaagattcccca

SEQ ID NO: 230 - DNA, 423 bp, Unknown
  source: /note=“Description of Unknown 16S rRNA sequence”
cctgttcgatccccacgctttcgtgcctcagcgtcagaccggcgccggtacgctgccttcgcaatcggagttctgcgcgatatctatgcatttcaccgctacaccgcgcattccgcgtacttctcgccgtctcaagtcccccagtctaaacggctcggcggggttgagccccgcgattttaccgcttacttaagagaccgcctacgcaccctttaaacccaataaatccggataacgctcgcatcctccgtattaccgcggctgctggcacggagttagccgatgctttttctccgggtacactcggatgaggacgcgtcctcacttttgttccccggcaaaagaggtttacaacacgtagtgccgtcttccctcacgcggcttggctggttcaggatctctcccattgaccaatattcctca

SEQ ID NO: 231 - DNA, 429 bp, Unknown
  source: /note=“Description of Unknown 16S rRNA sequence”
cctgtttgctccccacgctttcgcgcctcagcgtcagttacggcccagcaagtcgccttcgccactggtgttcctcctaatatctacgcatttcaccgctacactaggaattccacttgcctcttccgcactcaagtctgccagtatcggatgcacgttaagggttgagcccttacatttcacatccgacttaacagaccgcctacgcgccctttacgcccagtgattccggacaacgcttgccccctatgttttaccgcggctgctggcacatagttagccggggcttccttcttgtctaccgtcctcagactgtcagttactacagtctgctctcttggacaaggatagagctttacaacccgaaggccttcttcactcacgcggcattgctccgtcaggctttcgcccattgcggaagattcccca

SEQ ID NO: 232 - DNA, 429 bp, Unknown
  source: /note=“Description of Unknown 16S rRNA sequence”
cctatttgctccccacgctttcgggactcagcgtctgtttcagcccagatcgtcgccttcgccactggtgttcctccatatatctacgcatttcaccgctacacatggaattccacgatcctctgctgtactctagccacacggtttctttgccttcccgatgttgagcatcgggtttttaacactagaccctcatggccgcctgctccctctttacgcccaataattccggataacgctcgccacctacgtattaccgcggctgctggcacgtagttagccgtggctttctaaccaggtaccgtcctatatgaatcatttcctattcatatctctcttccctgataacagaactttacaatccgaagaccttcttcgttcacgcggcgttgctcggtcagactttcgtccattgccgaaaattcccta

SEQ ID NO: 233 - DNA, 423 bp, Unknown
  source: /note=“Description of Unknown 16S rRNA sequence”
cctgttcgatacccacgccttcgtgcttcagcgtcagttgggcgccggtacgctgccttcgcaatcggagttctgcgtgatatctatgcatttcaccgctacaccacgcattccgcgtacttctcgcccactcaaggcacccagtttcaacggctcgacgaggttgagccccgcaattttaccgctgacttaagcgtccgcctacgcaccctttaaacccaataaatccggataacgctcgcatcctccgtattaccgcggctgctggcacggagttagccgatgctttttctccgggtactctccaaaccgcacacgtgcggcactttgctccccggcaaaagaggtttacaacccatagggccgtcatccctcacgcgacttggctggttcaggctctcgcccattgaccaatattcctca

SEQ ID NO: 234 - DNA, 404 bp, Unknown
  source: /note=“Description of Unknown 16S rRNA sequence”
cctgtttgctccccacgctttcgagcctcaacgtcagttaccgtccagtaagccgccttcgccactggtgttcttcctaatatctacgcatttcaccgctacactaggaattccgcttacctctccggcactctagctaaacagtttccaaagcagtcccggcgttgagcaccgggctttcacttcagacttgccttgccgtctacactccctttacacccagtaaatccggataacgcttgccccctacgtattaccgcggctgctggcacgtagttagccggggcttcttattcaggtaccgtcactttcttccctgctgatagagctttacataccgaaatacttcttcactcacgcggcgtcgctgcatcagggtttcccccattgtgcaatattcccca

SEQ ID NO: 235 - DNA, 406 bp, Unknown
  source: /note=“Description of Unknown 16S rRNA sequence”
cctgtttgctccccacgctttcgcgcctcaccgtcagttgtcgtccagtaatccgccttcgccactggtgttcttccttatatctacgcatttcaccgctacacaaggaattccgattacctctccgatactcaagatgcacagtttcaaatgcagtttggaggttgagcctccagatttcacatctgacttgcgcacccggctacacgccctttacacccagtaaatccggacaacgcttgccacctacgtattaccgcggctgctggcacgtagttagccgtggcttattcgtcaggtaccgtcttctactcttccctgacaaaagaagtttacaacccgaaagccttcttccttcacgcggcgttgctgggtcagacttgcgtccattgcccaatattcccca

SEQ ID NO: 236 - DNA, 429 bp, Unknown
  source: /note=“Description of Unknown 16S rRNA sequence”
cctgtttgctccccacgctttcgcgcctcagtgtcagttgcagaccaggaagccgccttcgccactggtgttcctccatatctctacgcatttcaccgctacacatggaattccacttccctcttctgcactcaagtcaaccagtttccaatgaccctccacggttaagccgtgggctttcacatcagacttaattaaccacctgcgcgctctttacgcccaataattccggataacgcttgccacctacgtattaccgcggctgctggcacgtagttagccgtggctttctcataaggtaccgtcaattgatagtcatttcctcctatcacctttcttcccttataacagaattttacaacccgaaggccttcttcattcacgcggcgttgctcggtcaggctttcgcccattgccgaagattcccta

SEQ ID NO: 237 - DNA, 404 bp, Unknown
  source: /note=“Description of Unknown 16S rRNA sequence”
cctgtttgctccccacgctttcgagcctcaacgtcagttaccgtccagtaagccgccttcgccactggtgttcctcccaatatctacgcatttcaccgctacactgggaattccgcttacctctccggcactctagatacacagtttccaaagcagtcccggggttgagccccgggctttcacttcagacttgcacatccgtctacgctccctttacacccagtaaatccggataacgcttgccccctacgtattaccgcggctgctggcacgtagttagccggggcttcttagtcaggtaccgtcattttcttccctgctgatagagctttacataccgaaatacttcttcgctcacgcggcgtcgctgcatcagggtttcccccattgtgcaatattcccca

SEQ ID NO: 238 - DNA, 400 bp, Unknown; identical to SEQ ID NO: 24
  source: /note=“Description of Unknown 16S rRNA sequence”

SEQ ID NO: 239 - DNA, 405 bp, Unknown
  source: /note=“Description of Unknown 16S rRNA sequence”
cctgttcgctccccacgctttcgcacctcagcgtcagtctccggccggcagaccgccttcgcctccggtattcctcctgatctctgcgcatttcaccgctacaccaggaattctgtctgcctctccggtactcgagttaaacggttcgcgctgaccttccggggttgagccccgggctttgacaacgcgcctgcctaaccgcctgcgtgccctttacgcccaataaatccggataacgctcgccccctacgtattaccgcagctgctggcacgtagttggctggggcttgctcgccgggtaccgtcttaactcttccccgacaacagggctttacggaacgaatcccttcttcacccacgcggcattgctgcgtcagagttgcctccattgcgcaatattcccca

SEQ ID NO: 240 - DNA, 404 bp, Unknown
  source: /note=“Description of Unknown 16S rRNA sequence”
cctgtttgctccccacgctttcgagcctcaacgtcagttaccgtccagtaagccgccttcgccactggtgttcctcctaatatctacgcatttcaccgctacactaggaattccgcttacctctccggcactcgagtatcacagtttccaatgcagtccaggggttgagcccccgcctttcacatcagacttgcaacaccgtctacgctccctttacacccagtaaatccggataacgcttgccccctacgtattaccgcggctgctggcacgtagttagccggggcttcttagtcaggtaccgtcattttcttccctgctgatagagctttacataccgaaatacttcatcgctcacgcggcgttgctgcatcaggcttgcgcccattgtgcaatattcccca

SEQ ID NO: 241 - DNA, 405 bp, Unknown
  source: /note=“Description of Unknown 16S rRNA sequence”
cctgtttgctccccacgctttcgagcctcaacgtcagttactgtccagtaagccgccttcgccactggtgttcctcctaatatctacgcatttcaccgctacactaggaattccacttacctctccagcactctagcaacacagtttccaaagcagtcccagggttgagccctgggttttcacttcagacttgcatcgccgtctacgctccctttacacccagtaaatccggataacgcttgccccctacgtattaccgcggctgctggcacgtagttagccggggcttcttagtcaggtaccgtcttgtttcttccctgctgatagagctttacataccgaaatacttcttcactcacgcggcgtcgctgcatcagggtttcccccattgtgcaatattcccca

SEQ ID NO: 242 - DNA, 428 bp, Unknown
  source: /note=“Description of Unknown 16S rRNA sequence”
cctatttgctccccacgctttcgtgcatgagcgtcagtaacagtccaggcggccgccttcgcaactggtgttcttccatatatctacgcattttaccgctacacatggagttccaccgccctctcctgtcctctagcctgccagtttccgaagcatatacaggttaagcccgtacctttcacttcagacttggcatgcagcctgcgcaccctttacgcccaatcattccggataacgcttgccacctacgtattaccgcggctgctggcacgtagttagccgtgactttctggcggggtaccatcaaaagaagaacatttcctttcctcttcctttttcccccgcaacagagctttacgatccgaagaccttcctcactcacgcggcattgctcgttcaggcttgcgcccattgacgaaaattcccta

SEQ ID NO: 243 - DNA, 423 bp, Unknown
  source: /note=“Description of Unknown 16S rRNA sequence”
cctgttcgatacccacgctttcgtgcctcagcgtccgtaggcgcccggtatgctgccttcgcaattggagttctgcgtcatatctatgcatttcaccgctacacaacgcattccgcatacctcgacgccacgctagataatcagtttcaacggctgagcggggttgagccccgcgattttaccgctgacttgactgtccgcctgcgcaccctttaaacccaataaatccggataacgctcgcatcctccgtattaccgcggctgctggcacggagttagccgatgcttattcttgcggtacactcataaggggacacgtcccctcctttgttcccgcactaaagaagtttacaacccgtagggccgtcttccttcacgcgacttggctggttcagacttccgtccattgaccaatattcctca

SEQ ID NO: 244 - DNA, 429 bp, Unknown
  source: /note=“Description of Unknown 16S rRNA sequence”
cctgtttgctccccacgctttcgtgcatgagcgtcagtgttatcccagggggctgccttcgccatcggtattcctccacatctctacgcatttcactgctacacgtggaattctacccccctctgacacactctagccgtgcagtcaccaatgcaattcccaagttaagctcggggatttcacatcggtcttgcacaaccgcctgcgcacgctttacgcccagtaattccgattaacgcttggaccctacgtattaccgcggctgctggcacgtagttagccggtccttattcttccggtaccgtcatcgaccccaggtattaaccagagccatttctttccggacaaaagtgctttacaacccgaaggccttcttcacacacgcggcattgctggatcaggctttcgcccattgtccaaaattcccca

SEQ ID NO: 245 - DNA, 404 bp, Unknown
  source: /note=“Description of Unknown 16S rRNA sequence”
cctgtttgctccccacgctctcgcgccttagcgtcagtctctctccagcaggccgccttcgcctccggtattcctcctgatctctgcgcatttcaccgctacaccaggaattctgcctgcctctcaagtactctagctggcccggttcccgtcgcctccccgggttgagcccgggaattttacaacaggcctctgccgccgcctacgcgccctttacgcccagtaattccggacaacgctcgccccctacgtattaccgcggctgctggcacgtagttagccggggctttaaaatggggtagtatctctcttctcccccacaactgagctctgcgggacgaatcccttcctcactcacgcggcatcgctgcgtcagagtttcctccattgcgcaatattcccca

SEQ ID NO: 246 - DNA, 405 bp, Unknown
  source: /note=“Description of Unknown 16S rRNA sequence”
cctgtttgctccccacgctttcgcgccttagcgtcagttgagttccagcagatcgccttcgcaatgggtattcttggtgatatctacggattttacccctacaccaccaattccatctgcctctccctcactctagactatcagtttcccaagcagtttaatggttaagccattagatttcacaagagacttgataatccgcctacgcgccctttacgcccagtgattccgagtaacgcttgcaccctccgtattaccgcggctgctggcacggagttagccggtgcttattccttaggtaccgtcagaattcttccctaagaaaaggagtttacgctccgaaaagtgtcatcctccacgcggcgttgctgcgtcagggtttcccccattgcgcaatattcccta

SEQ ID NO: 247 - DNA, 406 bp, Unknown
  source: /note=“Description of Unknown 16S rRNA sequence”
cctgttcgctccccacgctttcgtgcctcagcgtcagttacagtccagaaagccgccttcgctactggtgttcctccgaatctctacgcatttcaccgctacactcggaattccgctttcctctcctgcactctagcaactcagtttcagatgcagtctcaaggttgagccctgagttttcacatctgacttaaatcgccgcctacgcaccctttacacccagtaattccggataacgcttgccccctacgtattaccgcggctgctggcacgtagttagccggggcttcttagtcaggtaccgtcatggtttcttccctgctgatagaagtttacgatccgaagaccttcatccttcacgcggcgtcgctgcatcaggcttgcgcccattgtgcaatattcccca

SEQ ID NO: 248 - DNA, 404 bp, Unknown
  source: /note=“Description of Unknown 16S rRNA sequence”
cctgtttgctccccacgctttcgagcctcaacgtcagttacagtccagtaagccgccttcgccactggtgttcctcctaatatctacgcatttcaccgctacactaggaattccgcttacctctcctgcactctagcttaacagtttccaatgcagtcttggggttgagccccaagttttcacatcagacttgccatgccgtctacgctccctttacacccagtaaatccggataacgcttgccccctacgtattaccgcggctgctggcacgtagttagccggggcttcttagtcaggtaccgtcattttcttccctgctgatagaagtttacataccgaaatacttcttccttcacgcggcgtcgctgcatcagggtttcccccattgtgcaatattcccca

SEQ ID NO: 249 - DNA, 424 bp, Unknown
  source: /note=“Description of Unknown 16S rRNA sequence”
cctgttcgatccccacgctttcgtgcctcagcgtcagtgacgacttcgccagatgcctccgcaatcggtgttctgtgtgatatctatgcatttcaccgctacaccacacattcctccagcggcagtcgtactcgagcaaagcagtatcaacggcacggtccctgttgagcaggaagttttcaccgctgacttacctcgcagcctacgcaccctttaaacccaataaatccggataacgcttgcatcctccgtattaccgcggctgctggcacggagttagccgatgcttattcgttgcatactctcatcaaacctcacgaggtccttattgctctgcaacaaaagaagtttacaacccatagggccttaatccttcacgcggcatggctggttcagacttccgtccattgaccaatattcctca

SEQ ID NO: 250 - DNA, 407 bp, Unknown
  source: /note=“Description of Unknown 16S rRNA sequence”
cctgtttgctccccacgctttcgcgcctcagcgtcagttgtcgtccagcaagccgccttcgccactggtgttcctcctaatatctacgcatttcaccgctacactaggaattccgcttgcctctccgatactcaagggctacagtttcaaatgcagttccggggttgagccccgggatttcacatctgacttgcagctccgcctacacgccctttacacccagtaattccggacaacgcttgcaacctacgtattaccgcggctgctggcacgtagttagccgttgctttttctgtaggtaccgtcacgttcttcgtccctactaaaagaagtttacaacccgaaggccttcttccttcacgcggcgttgctgggtcagggtttcccccattgcccaatattcccca

SEQ ID NO: 251 - DNA, 403 bp, Unknown
  source: /note=“Description of Unknown 16S rRNA sequence”
cctgtttgctcccctagctttcgcgcctcagcgtcagtgacggcccagaggactgccttcgccatcggtgttcttcccgatatctgcgcattccaccgctacaccgggaattccatcctcctctaccgcactcgaggcgtgcagttcgcgacccggcttggggttgagccccaagattagaggtcgcgcatacacacccgcctacgcgctctttacgcccaatgaatccggataacgctcgctccctacgtattaccgcggctgctggcacgtagttagccggagcttcttctgcaggtactgtcgaagtcatccctgctgaaagcggtttacaacccgaaggccgtcatcccgcacgcggcgttgctgcgtcagggtttcccccattgcgcaaaattcccca

SEQ ID NO: 252 - DNA, 411 bp, Unknown
  source: /note=“Description of Unknown 16S rRNA sequence”
cctgttcgctcccctagctttcgcgcctcagcgtcagtcgtggcccagaaggccgccttcgccgccggtgttcttcccaatatctgcgcattccaccgctacactgggaattccgccttcccctaccagactcaagcctgccggtatcgggggcggaccggggttgagccccgggatttgacccccgacctaacaggccgcctacgcgcgctttacgcccaatgaatccggataacgctcgccccctacgtattaccgcggctgctggcacgtagttagccggggcttcttctgcaggtaccgtcacccagagggcctcgtccctgctgaaagcggtttacgacccgaaggccttcgtcccgcacgcggcgtcgctgcgtcaggctttcgcccattgcgcaagattcccca

SEQ ID NO: 253 - DNA, 404 bp, Unknown
  source: /note=“Description of Unknown 16S rRNA sequence”
cctgtttgctccccacgctttcgcgcatcagcgtcagtaatgttccagcaggtcgccttcgcaatgagtattcctcttgatctctacggattttacccctacaccaagaattccacctacctctcccatactctagatggtcagtttcaaatgcagttctgtagttaagctacaggatttcacatctgacttgaccacccgcctacgcgctctttacgcccagtgattccgagtaacgcttgcaccctccgtattaccgcggctgctggcacggagttagccggtgcttattcgtaagataccgtcataatcttctcttacaaaaggagtttacaatcctaaaaccttcatcctccacgcggcgttgctgcttcagggtttcccccattgagcaatattcccta

SEQ ID NO: 254 - DNA, 428 bp, Unknown
  source: /note=“Description of Unknown 16S rRNA sequence”
cctatttgctccccacgctttcgtgcctcagcgtcagtaacagtccaggcggccgccttcgcaactggtgttcttccatatatctacgcattttaccgctacacatggagttccaccgccctctcctgtcctcgagcgcgccagtttccaaagcctgcacaggttgagcctgtaccttttacttcagacttgacgcgccgcctgcgcaccctttacgcccaatcattccggataacgctcgccacctacgtattaccgcggctgctggcacgtagttagccgtgactttctggcggggtaccatcaaaagcagaccatttcctttctgcttcctttttcccccgcaacagagctttacgatccgaagaccttcctcactcacgcggcattgctcgttcaggcttgcgcccattgacgaaaattcccta

SEQ ID NO: 255 - DNA, 428 bp, Unknown
  source: /note=“Description of Unknown 16S rRNA sequence”
cctatttgctccccacgctttcgggaatgagcgtcagttacaggccagaccgtcgccttcgccactggtgttcctccatatatctacgcatttcaccgctacacatggaattccacgatcctctcctgcactctagccgcccggtttctatggcttactgaagttaagcttcagtctttcaccacagacctttgccgccgcctgctccctctttacgcccaataattccggataacgcttgccacctacgtattaccgcggctgctggcacgtagttagccgtggctttctcataaagtaccgtcactcggatatcattccctatatccgccgttcttcctttataacagaagtttacagaacgaattccttcttccttcacgcggcgttgctcggtcagggttccccccattgccgaaaattcccta

SEQ ID NO: 256 - DNA, 404 bp, Unknown
  source: /note=“Description of Unknown 16S rRNA sequence”
cctgtttgctccccacgctttcgagcctcagcgtcagttacagtccagagaatcgccttcgccactggtgttcttcctaatctctacgcatttcaccgctacactaggaattccattctcctctcctgcactctagatacccagtttggaatgcagctcccaggttaagcccaggtatttcacatcccacttaagtatccgcctacgctccctttacgcccagtaaatccggataacgcttgccacctacgtattaccgcggctgctggcacgtagttagccgtggcttcctccttaggtaccgtcattatcgtccctaaagacagagctttacaatccgaagaccttcatcactcacgcggcgttgctgcatcagggtttcccccattgtgcaatattcccca

SEQ ID NO: 257 - DNA, 429 bp, Unknown
  source: /note=“Description of Unknown 16S rRNA sequence”
cctgtttgctccccacgctttcgcgcctcagcgtcagttacggcccagcaagtcgccttcgccactggtgttcctcctaatatctacgcatttcaccgctacactaggaattccacttgcctcttccgcactcaagtctgccagtatcggatgcacgttaagggttgagcccttacatttcacatccgacttaacagaccgcctacgcgccctttacgcccagtgattccggacaacgcttgccccctatgttttaccgcggctgctggcacatagttagccggggcttccttcttgtctaccgtcctcgagtagtcagttactaccactcgctctcttggacaaggatagagctttacaacccgaaggccttcttcactcacgcggcattgctccgtcaggctttcgcccattgcggaagattcccca

SEQ ID NO: 258 - DNA, 424 bp, Unknown
  source: /note=“Description of Unknown 16S rRNA sequence”
cctgttcgatacccacgctttcgtgcctcagcgtcagttgggcgccggtatgctgccttcgcaatcggggttctgcgtgatatctatgcatttcaccgctacaccacgcattccgcatacttctcgcccactcaagtccgccagtttcaacggcaggccggcgttgagcgccgacatttgaccactgacttaacaaaccgcctacgcaccctttaaacccaataaatccggataacgctcgcatcctccgtattaccgcggctgctggcacggagttagccgatgctttttctccgggtactctccttcccagctcacgagctgggcattgctccccggcaaaagaggtttacaacccgtagggccgtcttccctcacgcgacttggctggttcaggcttccgcccattgaccaatattcctca

SEQ ID NO: 259 - DNA, 404 bp, Unknown
  source: /note=“Description of Unknown 16S rRNA sequence”
cctgtttgctccccacgctttcgagcctcagcgtcagttacaatccagagaagcgccttcgccactggtgttcttcctaatctctacgcatttcaccgctacactaggaattcccttctcctctcctgcactctagacttccagtttgaaatgcagcacccaagttaagcccgggtatttcacatctcacttaaaaatccgcctacgctccctttacgcccagtaaatccggacaacgcttgccacctacgtattaccgcggctgctggcacgtagttagccgtggcttcctcctcgggtaccgtcattatcgtccccgaagacagagctttacaacccgaaggccgtcatcactcacgcggcattgctgcatcagggtttcccccattgtgcaatattcccca

SEQ ID NO: 260 - DNA, 428 bp, Unknown
  source: /note=“Description of Unknown 16S rRNA sequence”
cctatttgctccccacgctttcgggactgagcgtcagttatgcgccagatcgtcgccttcgccactggtgttcctccatatatctacgcatttcaccgctacacatggaattccacgatcctctcacacactctagctctacggtttccatggcttaccgaagttaagcttcgatctttcaccacagacccttagtgccgcctgctccctctttacgcccaataattccggataacgctcgccacctacgtattaccgcggctgctggcacgtagttagccgtggctttcttatagagtaccgtcacttggatatcattccctatatccaccgttcttcctctatgacagaagtttacataacgaattacttcttccttcacgcggcgttgctcggtcagggtttcccccattgccgaaaattcccta

SEQ ID NO: 261 - DNA, 423 bp, Unknown
  source: /note=“Description of Unknown 16S rRNA sequence”
cctgtttgatacccgcactttcgagcctcagcgtcagtaacaccccagaagactgccttcgcaatcggagttcttcgtgatatctaagcatttcaccgctacaccacgaattccatcttccttgtgtgaactcaagacaaccagtatcaacggcggagaatggttgagccactctattttaccgctgacttaatcgtccgcctacgctccctttaaacccaataaatccggataacgctcgcatcttccgtattaccgcggctgctggcacggaattagccgatgcttattcataaggtacatacaatagagcacacgtgctcttttttattcccatataaaagaagtttacaacccatagggcagtcatccttcacgctacttggctggttcagactctcgtccattgaccaatattcctca

SEQ ID NO: 262 - DNA, 429 bp, Unknown
  source: /note=“Description of Unknown 16S rRNA sequence”
cctgtttgctccccacgctttcgtgcatgagcgtcagtcgcatcccagggggctgccttcgccattggtgttcttccaaatatctacgcatttcactgctacacttggaattccacccccctctgatgaactctagtcctgcagtcacaaatgcaattcccaggttgagcccggggatttcacatctgtcttacagaaccgcctgcgcacgctttacgcccagtaattccgattaacgcttgcaccctacgtattaccgcggctgctggcacgtagttagccggtgcttattcttcaggtaccgtcagcagatcatggtattagcacaatccttttcgtccctgacaaaagcagtttacaatccgaagaccttcatcctgcacgcggcatggctggatcagggttgcccccattgtccaaaattcccca

SEQ ID NO: 263 - DNA, 407 bp, Unknown
  source: /note=“Description of Unknown 16S rRNA sequence”
cctgtttgctccccatgctttcgcgcttcagtgtcagtattcgtccagtgagctgacttctctatcggcattcctacaaatatctacgaatttcacctctacacttgtagttccgcccacctctccgatactctagttaggcagtttccaacgcaatacggagttgagccccgcattttcacatcagacttaccaaaccacctagacgcgctttacgcccaataaatccggataacgcttgcgacatacgtattaccgcggctgctggcacgtatttagccgtcgcttcttctattggtaccgtcatttttttcttcccaattgaaagcactttacaatcctaagaccgtcatcgtgcacacagaattgctggatcagacttttggtccattgtccaatattcccca

SEQ ID NO: 264 - DNA, 404 bp, Unknown
  source: /note=“Description of Unknown 16S rRNA sequence”
cctgtttgctccccacgctttcgcacctgagcgtcagtaagtgcctggcagaccgccttcgcctccggtattcctcctgatctctgcgcatttcaccgctacaccaggaattctgtctgccccgacattactcgagttatccggtttgccatgacctgcggggttgagccccacccttgaacatcacacctggataaccgcctgcgtgccctttacgcccaataaatccggataacgctcgccccctacgtattaccgcagctgctggcacgtagttggctggggcttgctcaccgggtaccgtcaggcttcttccccggtaacagagctttacggaacgaatcccttcttcactcacgcggcattgctgcgtcagagttgcctccattgcgcaatattcccca

SEQ ID NO: 265 - DNA, 424 bp, Unknown
  source: /note=“Description of Unknown 16S rRNA sequence”
cctgtttgatccccacgctttcgtgcttcagtgtcagttatggcttagtaagctgccttcgcaatcggagttctgcgtgatatctatgcatttcaccgctacaccacgcattccgcctacctcaaacacactcaagtctcccagtttcaacggcaattttatggttaagccacaaactttcaccgctgacttaaaaaacaacctacgcaccctttaaacccaataaatccggataacgctcgcatcctccgtattaccgcggctgctggcacggagttagccgatgcttattcatagggtacatacaaaacaggacacgtcccgcactttattcccctataaaagaagtttacaaaccatagatccttcttccttcacgcgacttggctggttcaggctcgcgcccattgaccaatattcctca

SEQ ID NO: 266 - DNA, 428 bp, Unknown
  source: /note=“Description of Unknown 16S rRNA sequence”
cctatttgctccccacgctttcgtgcctcagcgtcagtaacaggccaggcggccgccttcgccactggtgttcttccatatatctacgcattttaccgctacacatggagttccaccgccctctcctgtcctccagcctgccagtttccaaagcctgtaccggttgagccggtacctttcacttcagacttaacaggccgcctacgcaccctttacgcccaatcattccggataacgctcgccacctacgtattaccgcggctgctggcacgtagttagccgtgacttcctcgaaaggtaatatcacttcaccagcatttcctctggtgttcctttttccctctcaacagaactttacgatccgaagaccttcctcgttcacgcggcattgctcgttcaggcttgcgcccattgacgaaaattcccta

SEQ ID NO: 267 - DNA, 428 bp, Unknown
  source: /note=“Description of Unknown 16S rRNA sequence”
cctatttgctccccacgctttcgtgcctcagcgtcagtcacaggccaggcggccgccttcgccactggtgttcttccatatctctgcgcattttaccgctacacatggagttccaccgccctctcctgtcctcaagtctgccagtttctgaatcatgaatgagttgagctcatccctttgccttcagacttaacagaccgcctgcgcaccctttacgcccaatcattccggataacgctcgccacctacgtattaccgcggctgctggcacgtagttagccgtggctttctggccgggtaccatccatgaaatgccatttcctgcattccctctttttccccggcaacagagctttacaatccgaagaccttcttcactcacgcggcattgctcgttcaggcttgcgcccattgacgaaaattcccta

SEQ ID NO: 268 - DNA, 406 bp, Unknown
  source: /note=“Description of Unknown 16S rRNA sequence”
cctgtttgctccccacgctttcgcgcctcagcgtcagttgtcgtccagcaggccgccttcgccactggtgttcctcctaatatctacgcatttcaccgctacactaggaattccgcctgcctctccgatactcaagaagaacagtttcaaatgcaatttatgggttaagcccatagatttcacatctgacttgctctcccgcctacacgccctttacacccagtaaatccggacaacgcttgccacctacgtattaccgcggctgctggcacgtagttagccgtggcttattcgtcaggtaccgtcttctgctcttccctgacaaaagaagtttacaacccgaaagccttcttccttcacgcggcgttgctgggtcaggcttgcgcccattgcccaatattcccca

SEQ ID NO: 269 - DNA, 406 bp, Unknown
  source: /note=“Description of Unknown 16S rRNA sequence”
cctgtttgctccccacgctttcgtgcctgagcgtcagttatggtccagaaagccgccttcgctactggtattcctttgaatctctacgcatttcaccgctacactcaaagttctactttcctctcccacactctagcctagcagtttcaaaagcagctccgaagttgagcctcggaatttcacttctgacttgcttggccgcctgcgcaccctttacacccagtaaatccggataacgcttgctccctacgtattaccgcggctgctggcacgtagttagccggagcttcttagtcaggtaccgtcattttttcttccctgctgatagaagtttacgatccgaaaaccttcttccttcacgcggcgttgctgcatcaggcttgcgcccattgtgcaatattcccca

SEQ ID NO: 270 - DNA, 403 bp, Unknown
  source: /note=“Description of Unknown 16S rRNA sequence”
cctgtttgctacccacgctttcgagcctcagcgtcagttaaagcccagtaggccgccttcgccactggtgttccttccgatatctacgcattccaccgctacaccggaaattccgcctacctctacttcactcaagccctgcagttttgaaagcgactatgggttgagcccatagattatactcccaacttgctgagccgcctacgctccctttacacccagtaattccggacaacgctcgcaccctacgttttaccgcggctgctggcacgtagttagccggtgcttcctcctcgggtaccgtcattatcgttcccgaagacagaggtttacaatacgaataccttcttccctcacgcggcgtcgctgcatcagagtttcctccattgtgcaatattcccca

SEQ ID NO: 271 - DNA, 404 bp, Unknown; identical to SEQ ID NO: 13
  source: /note=“Description of Unknown 16S rRNA sequence”

SEQ ID NO: 272 - DNA, 424 bp, Unknown
  source: /note=“Description of Unknown 16S rRNA sequence”
cctgttcgatacccacgctttcgtgcttcagcgtcagttgggcgccggtatgctgccttcgcaatcggagttctgcgtgatatctatgcatttcaccgctacaccacgcattccgcatacttctcgcccactcaagaacaccagtttcaacggcgaagcgaggttgagccccgcgatttgaccgctgacttgacattccgcctacgcaccctttaaacccaataaatccggataacgctcgcatcctccgtattaccgcggctgctggcacggagttagccgatgctttttcttcaggtactctcgtaacacctactcgtagatgttgttgctccctgacaaaagaggtttacaacccatagggcagtcttccctcacgcgacttggctggttcaggcgtaagcccattgaccaatattcctca

SEQ ID NO: 273 - DNA, 406 bp, Unknown
  source: /note=“Description of Unknown 16S rRNA sequence”
cctgtttgctccccacgctttcgcacctcagcgtcagtatgtgcccaggagaccgccttcgcctctggtgttctttcagatatctacgcattccaccgctacacctgaaattccatctccctctgcactactctagacaggcagttcagaatgaccaacccaggttgagcccgggtatttcacatcctgcttgccaatccgcctacatgccctttacgcccagttattccgattaacgcttgcaccctccgtattaccgcggctgctggcacggagttagccggtgcttcttctgtgggtaacgtcagtatttcctccccactgaaagtgctttacaacccgaaggccttcttcacacacgcggcatggctgcatcagggtttcccccattgtgcaatattcccta

SEQ ID NO: 274 - DNA, 429 bp, Unknown
  source: /note=“Description of Unknown 16S rRNA sequence”
cctgtttgctccccacgctttcgtgcatgagcgtcagtattatcccaggaggctgccttcgccatcggtgttcttccaaatatctacgcatttcactgctacacttggaattccacctccctctgacatactctagcctgacagtcacaagtgctattcccaagttgagctcggggatttcacacctgtcttatcaagccgcctgcgcacgctttacgcccagtaattccgattaacgcttgcaccctacgtattaccgcggctgctggcacgtagttagccggtgcttattctttaggtaccgtcattaggtagcattattagtactacccgtttcgttcctaacaaaagcagtttacaacccgaaggccttcttcctgcacgcggcatggctggatcagggttgcccccattgtccaaaattcccca

SEQ ID NO: 275 - DNA, 422 bp, Unknown
  source: /note=“Description of Unknown 16S rRNA sequence”
cctgttcgctccccacgctttcgctcctcagcgtcagtaacggcccagagacctgccttcgccattggtgttcttcccgatatctacacattccaccgttacaccgggaattccagtctcccctaccgcactcaagcccgcccgtacccggcgcggatccaccgttaagcgatggactttcacaccggacgcgacgaaccgcctacgagccctttacgcccaataaatccggataacgcttgcaccctacgtattaccgcggctgctggcacgtagttagccggtgcttattcgaacaatccactcaacacggccaaagaccgtgccttgcccttgaacaaaagcggtttacaacccgaaggcctccatcccgcacgcggcgtcgctgcatcaggcttgcgcccattgtgcaatattcccca

SEQ ID NO: 276 - DNA, 407 bp, Unknown
  source: /note=“Description of Unknown 16S rRNA sequence”
cctgtttgctccccacgctttcgcgcctcagcgtcagttgctgtccagcagaccgccttcgccactggtgttcctcctaatatctacgcatttcaccgctacactaggaattccgtctgcctctccagtactcaagaaccacagtttcaaatgcaggctggaggttgagcctccagttttcacatctgacttgcgatcccgcctacacgccctttacacccagtaaatccggataacgcttgccacctacgtattaccgcggctgctggcacgtagttagccgtggcttattcctcaggtaccgtcacttgcttcgtccctgagaaaagaagtttacaacccgaaagccttcttccttcacgcggcgttgctgggtcaggcttgcgcccattgcccaatattcccca

SEQ ID NO: 277 - DNA, 404 bp, Unknown
  source: /note=“Description of Unknown 16S rRNA sequence”
cctgttcgctccccgcgctttcgcacctgagcgtcagtgcccggccggcaggccgccttcgcctccggtgttcctcctgatctctgcgcatttcaccgctacaccaggagttccgcctgcctctccggcactcgagccgcgcggttcgcgccgccgttccggggttgggccccggcctttcacggcgcgccctcgcggccgcctgcgtgccctttacgcccaatgaatccggacaacgcccgccccccacgtgttaccgcagctgctggcacgtggttggctggggcttcctcgccgggtaccgtctctctcttccccggcaacagggctttacgaaccgaagtccttcatcgcccacgcggcatcgctgcgtcagagttgcctccattgcgcaatattcccca

SEQ ID NO: 278 - DNA, 403 bp, Unknown; identical to SEQ ID NO: 25
  source: /note=“Description of Unknown 16S rRNA sequence”

SEQ ID NO: 279 - DNA, 408 bp, Unknown
  source: /note=“Description of Unknown 16S rRNA sequence”
cctgtttgctccccacgctttcgagcctcagcgtcagtttcagtccagaaagccgccttcgccactggtgttcttcctaatatctacgcatttcaccgctacactaggaattccgctttcctctcctgtactctagctcgccagttccaaatgcaatcctcggggttaagccccgggctttcacatctggcttgacgtgccgcctacgctccctttacacccagtaattccggataacgcttgccccctacgtattaccgcggctgctggcacgtagttagccggggcttcttattcaggtaccgtcactttcttcgtccctgttgatagaagtttacgatccgaaaaccttcttccttcacgcggcgttgctgcatcagagtttcctccattgtgcaatattcccca

SEQ ID NO: 280 - DNA, 403 bp, Unknown
  source: /note=“Description of Unknown 16S rRNA sequence”
cctgttcgctcccccagctttcgcgcctcagcgtcagtggcggcccagcaggctgccttcgccatcggtgttcttcccgatatctgcgcattccaccgctacaccgggaattccgcctgcctctaccgcactcgagccgcccagttcggaacccggcccgaggttgagccccggggttagaggttccgcttaggcagccgcctacgcgcgctttacgcccaatgaatccggataacgctcgctccctacgtattaccgcggctgctggcacgtagttagccggagcttcttctgtgggtaccgtcatcgtcgtccccactgaaagcggtttacaacccgagggccgtcgtcccgcacgcggcgttgctgcgtcagggtttcccccattgcgcaaaattcccca

SEQ ID NO: 281 - DNA, 407 bp, Unknown
  source: /note=“Description of Unknown 16S rRNA sequence”
cctgtttgctccccacgctttcgcgcctcagcgtcagtttcagtccagaaagccgccttcgccactggtgttcctaccgatctctacgcatttcaccgctacaccggtaattccgctttcctctcctgcactccagcccaccagtatccaaagcaattcccaccttgaaagcaggactttcactccagacttagcaggccgcctacgcgccctttacgcccaatcattccggacaacgctcgccccctacgtattaccgcggctgctggcacgtagttagccggggcttcctcctatggtaccgtcacttccttcgtcccataggacagaggtttacgagccgaaacccttcttccctcacgcggcgttgctgggtcaggctttcgcccattgcccaatattcccca

SEQ ID NO: 282 - DNA, 405 bp, Unknown
  source: /note=“Description of Unknown 16S rRNA sequence”
cctgtttgctccccacgctctcgcacctgagcgtcagtatctgtccggcaggccgccttcgcctctggtattcctcctgatctctgcgcatttcaccgctacaccaggaattctgcctgcctctccagtactcgagctgcatggttcgggatgcacctccagggttgggccctggcttttcacatcccgcctacacagccgcctgcgtgccctttacgcccagtcattccggacaacgctcgccccctacgtgttaccgcggctgctggcacgtagttagccggggcttgcttgttaggtaccgtcattcttcttccctaacggcagagctttacggaacgaattccttcttcactcacgcggcattgctgcgtcagagtttcctccattgcgcaatattcccca

SEQ ID NO: 283 - DNA, 424 bp, Unknown
  source: /note=“Description of Unknown 16S rRNA sequence”
cctgttcgatacccgcaccttcgagcttcagcgtcagtcgcactcccgcaagctgccttcgcaatcggagttcttcgtgatatctaagcatttcaccgctacaccacgaattccgcctgcgttgcgtgcactcaagccccccagttcgcgctgcagtgccacggttgagccgcggcatttcacaacacgcttaaggggcggcctacgctccctttaaacccaataaatccggataacgcctggaccttccgtattaccgcggctgctggcacggaattagccggtccttattcatatggtacctgcaataagctacacgtagctcactttatccccatataaaagcagtttacaacccatagggccgtcatcctgcacgctacttggctggttcaggctctcgcccattgaccaatattcctca

SEQ ID NO: 284 - DNA, 404 bp, Unknown
  source: /note=“Description of Unknown 16S rRNA sequence”
cctgttcgctccccacgctttcgcacctgagcgtcagtatctggccggcagaccgccttcgcctccggtattcctcctgatctctgcgcatttcaccgctacaccaggaattctgtctgcctctccagcactcgagctgcccggttcacagtggctctccggggttgagccccggtctttcacactgcgcctaaacagccgcctgcgtgccctttacgcccaataaatccggataacgctcgccccccacgtattaccgcagctgctggcacgtggttggctggggcttgctcatcgggtaccgtctccctcttccccgataacagagctttacggaacgaatcccttcttcactcacgcggcattgctgcgtcagagttgcctccattgcgcaatattcccca

SEQ ID NO: 285 - DNA, 424 bp, Unknown
  source: /note=“Description of Unknown 16S rRNA sequence”
cctgtttgatccccgcactttcgtgcctcagcgtcagttgagcgccggtatgctgccttcgcaatcggggttctgcgtgatatctatgcatttcaccgctacaccacgcattccgcatacttctcgctcactcaagaccgacagtttcaacggctcggcggggttgagccccgcaattttaccgctgacttatcagtccgcctacgcaccctttaaacccaataaatccggataacgctcgcatcctccgtattaccgcggctgctggcacggagttagccgatgctttttcttcaggtactctccttctggctacgcgtagccagctttgctccctgacaaaagaggtttacgacccgtagggccttattccctcacgcgacttggctggttcagccttccggccattgaccaatattcctca

SEQ ID NO: 286 - DNA, 405 bp, Unknown
  source: /note=“Description of Unknown 16S rRNA sequence”
cctgtttgctccccatgctctcatgcctcagcgtcagttacagtccagtaagtcgccttcgccactggtgttcttcctaatctctacgcatttcaccgctacactaggaattccacttacctctcctgcactctagttacacagttttatatgccttctacaggttaagcctatatatttgacacataacttatctaaccgcctacgcatcctttacgcccagttattccgaacaacgcttgccacctacgtattaccgcggctgctggcacgtagttagccgtggcttcctcttcaggtaccgtccttcctcttccctgctaacagagctttacaatccgaaaaccttcttcactcacgcggcgttgctgcatcagggttccccccattgtgcaatatccccca

SEQ ID NO: 287 - DNA, 406 bp, Unknown
  source: /note=“Description of Unknown 16S rRNA sequence”
cctgttcgctccccgcgctctcgcgcctcggcgtcagtgtccttccggcgggccgccttcgcctctggtgttcctcctggcatctgcgcatttcaccgctacgccaggaattccgcccgcccctccggcactccagcgacccggttcggggcgctttcccggggttgggccccggattttcacaccccgcctaagccgccgcctgcgcgcgctttacgcccagtgattccggacaacgctcgccccccacgtattgccgcggctgctggcacgtggttggccggggctttcttgccgggtaccgtcatctgttcttccccggcggcggggctttacgggccgaggcccttcatcgcccacgcggcatcgctgcgtcagagttccctccattgcgcaatattcccca

SEQ ID NO: 288 - DNA, 404 bp, Unknown
  source: /note=“Description of Unknown 16S rRNA sequence”
cctgtttgctccccacgctttcgagcctcaacgtcagtaatcgtccagtaagccgccttcgccactggtgttcctcctaatatctacgcatttcaccgctacactaggaattccgcttacctctccgacactctagttgcatagtttccaatgcagtcccggggttgagccccgggttttcacatcagacttacacaaccgtctacgctccctttacacccagtaaatccggataacgcttgcaccatacgtattaccgcggctgctggcacgtatttagccggtgcttcttagtcaggtaccgtcattttcttccctgctgatagagctttacataccgaaatacttcttcgctcacgcggcgttgctgcatcagggtttcccccattgtgcaatattcccca

SEQ ID NO: 289 - DNA, 405 bp, Unknown
  source: /note=“Description of Unknown 16S rRNA sequence”
cctgtttgctccccgcgctttcgcgcctaagcgtcagtgtctgtccggcaggccgccttcgcctctggtattcctcctgatctctgcgcatttcaccgctacaccaggaattctgcctgcccctccagcacccgagccgcgcggtttggaaggcctctccggggttgggccccgggctttgacatcccacccgcgcagccgcctgcgcgccctttacgcccagtgattccggacaacgctcgcccctcacgtcttaccgcggctgctggcacgtgattggccggggcttgcttgccgggtaccgtcttctctcttccccggcggcagggctttacgggccggagcccttcttcgcccacgcggcatcgctgcgtcagagtttcctccattgcgcaatattcccca

SEQ ID NO: 290 - DNA, 404 bp, Unknown
  source: /note=“Description of Unknown 16S rRNA sequence”
cctgtttgctccccacgctctcgcgccttagcgtcagttgctgtccagcaggccgccttcgcctccggtattcctcctgatctctgcgcatttcaccgctacaccaggaattctgcctgcctctccagcactccagctgactcggttcctgtcgcctctccgggttgagcccggatattttacaacaggccttcgccgccgcctacgcgccctttacgcccagtaattccggacaacgctcgccccctacgtattaccgcggctgctggcacgtagttagccggggcttgaaaaaagggttgaatctctcttttccccttcgactgagctctgcgggacgaatcccttcctcactcacgcggcatcgctgcgtcagagttgcctccattgcgcaatattcccca

SEQ ID NO: 291 - DNA, 424 bp, Unknown
  source: /note=“Description of Unknown 16S rRNA sequence”
cctgttcgatacccacgctttcgtgcttcagcgtcagttgggcgccggtatgctgccttcgcaatcggagttctgcgtgatatctatgcatttcaccgctacaccacgcattccgcatacttctcgcccactcaagaccgtcagtttcaacggcgggcggaggttgagcccccgtattttaccgctgacttgacagtccgcctacgcaccctttaaacccaataaatccggataacgctcgcatcctccgtattaccgcggctgctggcacggagttagccgatgctttttcttcgggtactctcgactccgccacacgtggcggaatttgctccccgacaaaagaggtttacaacccgtagggccgtcatccctcacgcgacttggctggttcaggctcgcgcccattgaccaatattcctca

SEQ ID NO: 292 - DNA, 428 bp, Unknown
  source: /note=“Description of Unknown 16S rRNA sequence”
cctatttgctccccacgctttcgtgcctgagcgtcagcaacagtccaggcggtcgccttcgcaactggtgttcttccatatatctacgcattttaccgctacacatggagttccaccgccctctcctgtcctctagcgtgccagtttccaaagcctgtaccggttaagcccgtacctttcacttcagacttgacgcgcagcctgcgcaccctttacgcccaatcattccggataacgcttgccacctacgtattaccgcggctgctggcacgtagttagccgtgactttctggcggggtaccatcaaaagaaaagcatttcctctttccttcctttttcccccgcaacagagttttacgatccgaagaccttcttcgctcacgcggcattgctcgttcaggcttgcgcccattgacgaaaattcccta

SEQ ID NO: 293 - DNA, 424 bp, Unknown; identical to SEQ ID NO: 14
  source: /note=“Description of Unknown 16S rRNA sequence”